(19) United States
(12) Patent Application Publication
(10) Pub. No.: US 2022/0072024 A1
(43) Pub. Date: Mar. 10, 2022

(54) COMPOSITIONS AND METHODS FOR INHIBITING HMGB1 EXPRESSION

(71) Applicant: Dicerna Pharmaceuticals, Inc., Lexington, MA (US)

(72) Inventors: Marc ABRAMS, Lexington, MA (US); Girish CHOPDA, Lexington, MA (US); Uihye PARK, Lexington, MA (US)

(21) Appl. No.: 17/309,860

(22) PCT Filed: Dec. 20, 2019
(86) PCT No.: PCT/US2019/067883

§ 371 Date: Jun. 24, 2021

Related U.S. Application Data
(60) Provisional application No. 62/786,287, filed on Dec. 28, 2018.
(60) Provisional application No. 62/787,038, filed on Dec. 31, 2018.
(60) Provisional application No. 62/788,111, filed on Jan. 3, 2019.

(51) Int. Cl. A61K 31/713; C12N 15/113; A61K 47/54

(52) U.S. Cl. CPC A61K 31/713; C12N 15/113; A61K 47/549; C12N 2310/321; C12N 2310/11; C12N 2310/3125; C12N 2310/315; C12N 2310/14

(57) ABSTRACT

This disclosure relates to oligonucleotides, compositions and methods useful for reducing HMGB1 expression, particularly in hepatocytes. Disclosed oligonucleotides for the reduction of HMGB1 expression may be either double-stranded or single-stranded and may be modified for improved characteristics such as stronger resistance to nucleases and lower immunogenicity. Disclosed oligonucleotides for the reduction of HMGB1 expression may also be designed to include targeting ligands to target a particular cell or organ, such as the hepatocytes of the liver, and may be used to treat liver fibrosis and related conditions.

### CROSS-REFERENCE TO RELATED APPLICATIONS

This application claims the benefit under 35 U.S.C. § 119(e) of U.S. provisional application No. 62/786,287, filed Dec. 28, 2018, U.S. provisional application No. 62/787,038, filed Dec. 31, 2018, and U.S. provisional application No. 62/788,111, filed Jan. 3, 2019, the entire contents of each of which is incorporated herein by reference.

### REFERENCE TO SEQUENCE LISTING, TABLE OR COMPUTER PROGRAM

The official copy of the Sequence Listing is submitted concurrently with the specification as an ASCII formatted text file via EFS-Web, with a file name of “400930-014WO_ST25.txt,” a creation date of Dec. 12, 2019, and a size of 306 kilobytes. The Sequence Listing filed via EFS-Web is part of the specification and is hereby incorporated in its entirety by reference herein.

### FIELD OF THE INVENTION

The present application relates to oligonucleotides and uses thereof, particularly uses relating to the treatment of conditions involving fibrosis.

### BACKGROUND OF THE INVENTION

Tissue fibrosis is a condition characterized by an abnormal accumulation of extracellular matrix and inflammatory factors that result in scarring and promote chronic organ injury. In liver, fibrosis is a multi-cellular response to hepatic injury that can lead to cirrhosis and hepatocellular cancer. The response is often triggered by liver injury associated with conditions such as alcohol abuse, viral hepatitis, metabolic diseases, and liver diseases, such as a cholestatic liver disease, nonalcoholic fatty liver disease (NAFLD) and nonalcoholic steatohepatitis (NASH). Studies have implicated the high mobility group box 1 (HMGB1) protein as having a pro-fibrotic role in liver fibrosis (see, e.g., Li L-C, et al., J. Cell. Mol. Med., 2014, 18(12):2331-39). HMGB1 is a nuclear protein released from injured cells that functions as a proinflammatory mediator and has been shown to recruit hepatic stellate cells and liver endothelial cells to sites of liver injury (Seo et al., Am J Physiol Gastrointest Liver Physiol., 2013, 305:G838-G848). Hepatic stellate cells are believed to play a central role in the progression of liver fibrosis through their transformation into proliferative myofibroblastic cells that promote fibrogenic activity in the liver (see, Kao Y H, et al., Transplant Proc., 2008, 40:2704-5).

### BRIEF SUMMARY OF THE INVENTION

Aspects of the disclosure relate to compositions and methods for treating fibrosis (e.g., liver fibrosis) in a subject using oligonucleotides that selectively inhibit HMGB1 expression. In some embodiments, potent RNAi oligonucleotides have been developed for selectively inhibiting HMGB1 expression. Accordingly, in some embodiments, RNAi oligonucleotides provided herein are useful for reducing HMGB1 expression, particularly in hepatocytes, and thereby decreasing or preventing fibrosis. In some embodiments, RNAi oligonucleotides incorporating nicked tetraloop structures are conjugated with GalNAc moieties to facilitate delivery to liver hepatocytes to inhibit HMGB1 expression for the treatment of liver fibrosis (see, e.g., Examples 3 and 4, evaluating GalNAc-conjugated HMGB1 oligonucleotides in primary monkey or human hepatocytes). In some embodiments, methods are provided herein involving the use of RNAi oligonucleotides for treating subjects having or suspected of having liver conditions such as, for example, cholestatic liver disease, nonalcoholic fatty liver disease (NAFLD) and nonalcoholic steatohepatitis (NASH). In further embodiments, the disclosure is based on an identification of key targeting sequences of HMGB1 mRNA that are particularly amenable to bringing about mRNA knockdown using RNAi oligonucleotide-based approaches. Furthermore, RNAi oligonucleotides having particular modification patterns are developed herein (as outline in Table 2) and that are particularly useful for reducing HMGB1 mRNA expression level in vivo are provided herein.

Some aspects of the present disclosure provide oligonucleotides for reducing expression of HMGB1, the oligonucleotide comprising a sense strand of 15 to 50 nucleotides in length and an antisense strand of 15 to 30 nucleotides in length, wherein the sense strand forms a duplex region with the antisense strand, wherein the sense strand comprises a sequence as set forth in any one of SEQ ID NOs.: 1-13 and wherein the antisense strand comprises a complementary sequence selected from SEQ ID NOs: 14-26.

In some embodiments, the sense strand sequence comprises or consists of a sequence as set forth in any one of SEQ ID NOs: 27-39. In some embodiments, the antisense strand sequence consists of a sequence as set forth in any one of SEQ ID NOs: 14-26.

In some embodiments, the sense strand sequence comprises a sequence as set forth in SEQ ID NO: 27 and the antisense strand sequence comprises a sequence as set forth in SEQ ID NO: 14.

In some embodiments, the sense strand sequence comprises a sequence as set forth in SEQ ID NO: 28 and the antisense strand sequence comprises a sequence as set forth in SEQ ID NO: 15.

In some embodiments, the sense strand sequence comprises a sequence as set forth in SEQ ID NO: 29 and the antisense strand sequence comprises a sequence as set forth in SEQ ID NO: 16.

In some embodiments, the sense strand sequence comprises a sequence as set forth in SEQ ID NO: 30 and the antisense strand sequence comprises a sequence as set forth in SEQ ID NO: 17.

In some embodiments, the sense strand sequence comprises a sequence as set forth in SEQ ID NO: 31 and the antisense strand sequence comprises a sequence as set forth in SEQ ID NO: 18.

In some embodiments, the sense strand sequence comprises a sequence as set forth in SEQ ID NO: 32 and the antisense strand sequence comprises a sequence as set forth in SEQ ID NO: 19.

In some embodiments, the sense strand sequence comprises a sequence as set forth in SEQ ID NO: 33 and the antisense strand sequence comprises a sequence as set forth in SEQ ID NO: 20.

In some embodiments, the sense strand sequence comprises a sequence as set forth in SEQ ID NO: 34 and the antisense strand sequence comprises a sequence as set forth in SEQ ID NO: 21.

In some embodiments, the sense strand sequence comprises a sequence as set forth in SEQ ID NO: 35 and the antisense strand sequence comprises a sequence as set forth in SEQ ID NO: 22.

In some embodiments, the sense strand sequence comprises a sequence as set forth in SEQ ID NO: 36 and the antisense strand sequence comprises a sequence as set forth in SEQ ID NO: 23.

In some embodiments, the sense strand sequence comprises a sequence as set forth in SEQ ID NO: 37 and the antisense strand sequence comprises a sequence as set forth in SEQ ID NO: 24.

In some embodiments, the sense strand sequence comprises a sequence as set forth in SEQ ID NO: 38 and the antisense strand sequence comprises a sequence as set forth in SEQ ID NO: 25.

In some embodiments, the sense strand sequence comprises a sequence as set forth in SEQ ID NO: 39 and the antisense strand sequence comprises a sequence as set forth in SEQ ID NO: 26.

In some embodiments, the oligonucleotide comprises at least one modified nucleotide.

In some embodiments, all of the nucleotides of the oligonucleotide described herein are modified. In some embodiments, the modified nucleotide comprises a 2′-modification. In some embodiments, the 2′-modification is a 2′-fluoro or 2′-O-methyl.

In some embodiments, one or more of positions 1, 2, 4, 6, 7, 12, 14, 16, 18-27, and 31-36 of the sense strand, and/or one or more of positions 1, 4, 6, 8, 9, 11, 13, 15, 18, and 20-22 of the antisense strand are modified with a 2′-O-methyl. In some embodiments, all of positions 1, 2, 4, 6, 7, 12, 14, 16, 18-27, and 31-36 of the sense strand, and all of positions 1, 4, 6, 8, 9, 11, 13, 15, 18, and 20-22 of the antisense strand are modified with a 2′-O-methyl. In some embodiments, one or more of positions 3, 5, 8-11, 13, 15, and 17 of the sense strand, and/or one or more of positions 2, 3, 5, 7, 10, 12, 14, 16, 17, and 19 of the antisense strand are modified with a 2′-fluoro. In some embodiments, all of positions 3, 5, 8-11, 13, 15, and 17 of the sense strand, and all of positions 2, 3, 5, 7, 10, 12, 14, 16, 17, and 19 of the antisense strand are modified with a 2′-fluoro.

In some embodiments, one or more of positions 1-7, 12-27, and 31-36 of the sense strand, and/or one or more of positions 1, 4, 6, 8, 9, 11-13, and 15-22 of the antisense strand are modified with a 2′-O-methyl. In some embodiments, all of positions 1-7, 12-27, and 31-36 of the sense strand, and all of positions 1, 4, 6, 8, 9, 11-13, and 15-22 of the antisense strand are modified with a 2′-O-methyl. In some embodiments, one or more of positions 8-11 of the sense strand, and/or one or more of positions 2, 3, 5, 7, 10, and 14 of the antisense strand are modified with a 2′-fluoro. In some embodiments, all of positions 8-11 of the sense strand, and all of positions 2, 3, 5, 7, 10, and 14 of the antisense strand are modified with a 2′-fluoro.

In some embodiments, one or more of positions 1, 2, 4-7, 9, 11, 14-16, 18-27, and 31-36 of the sense strand, and/or one or more of positions 1, 6, 8, 9, 11, 13, 15, 18, and 20-22 of the antisense strand are modified with a 2′-O-methyl. In some embodiments, all of positions 1, 2, 4-7, 9, 11, 14-16, 18-27, and 31-36 of the sense strand, and all of positions 1, 6, 8, 9, 11, 13, 15, 18, and 20-22 of the antisense strand are modified with a 2′-O-methyl. In some embodiments, one or more of positions 3, 8, 10, 12, 13, and 17 of the sense strand, and/or one or more of positions 2-5, 7, 10, 12, 14, 16, 17, and 19 of the antisense strand are modified with a 2′-fluoro. In some embodiments, all of positions 3, 8, 10, 12, 13, and 17 of the sense strand, and all of positions 2-5, 7, 10, 12, 14, 16, 17, and 19 of the antisense strand are modified with a 2′-fluoro.

In some embodiments, the oligonucleotide comprises at least one modified internucleotide linkage. In some embodiments, the at least one modified internucleotide linkage is a phosphorothioate linkage.

In some embodiments, the oligonucleotide has a phosphorothioate linkage between one or more of: positions 1 and 2 of the sense strand, positions 1 and 2 of the antisense strand, positions 2 and 3 of the antisense strand, positions 3 and 4 of the antisense strand, positions 20 and 21 of the antisense strand, and positions 21 and 22 of the antisense strand. In some embodiments, the oligonucleotide has a phosphorothioate linkage between each of: positions 1 and 2 of the sense strand, positions 1 and 2 of the antisense strand, positions 2 and 3 of the antisense strand, positions 20 and 21 of the antisense strand, and positions 21 and 22 of the antisense strand.

In some embodiments, the uridine at the first position of the antisense strand comprises a phosphate analog. In some embodiments, the oligonucleotide comprises the following structure at position 1 of the antisense strand:

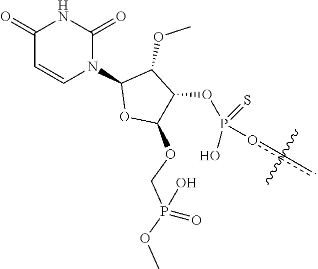

In some embodiments, one or more of the nucleotides of the -AAA- sequence at positions 28-30 on the sense strand is conjugated to a monovalent GalNAc moiety. In some embodiments, each of the nucleotides of the -AAA- sequence at positions 28-30 on the sense strand is conjugated to a monovalent GalNAc moiety. In some embodiments, the -AAA- motif at positions 28-30 on the sense strand comprises the structure:

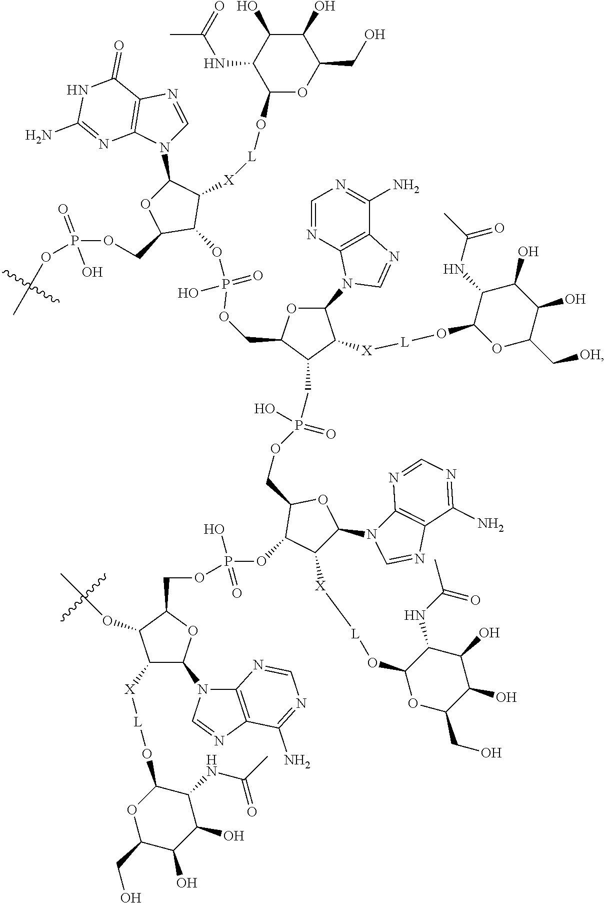

wherein:

L represents a bond, click chemistry handle, or a linker of 1 to 20, inclusive, consecutive, covalently bonded atoms in length, selected from the group consisting of substituted and unsubstituted alkylene, substituted and unsubstituted alkenylene, substituted and unsubstituted alkynylene, substituted and unsubstituted heteroalkylene, substituted and unsubstituted heteroalkenylene, substituted and unsubstituted heteroalkynylene, and combinations thereof; and X is O, S, or N.

In some embodiments, L is an acetal linker. In some embodiments, X is O.

In some embodiments, the -AAA- sequence at positions 28-30 on the sense strand comprises the structure:

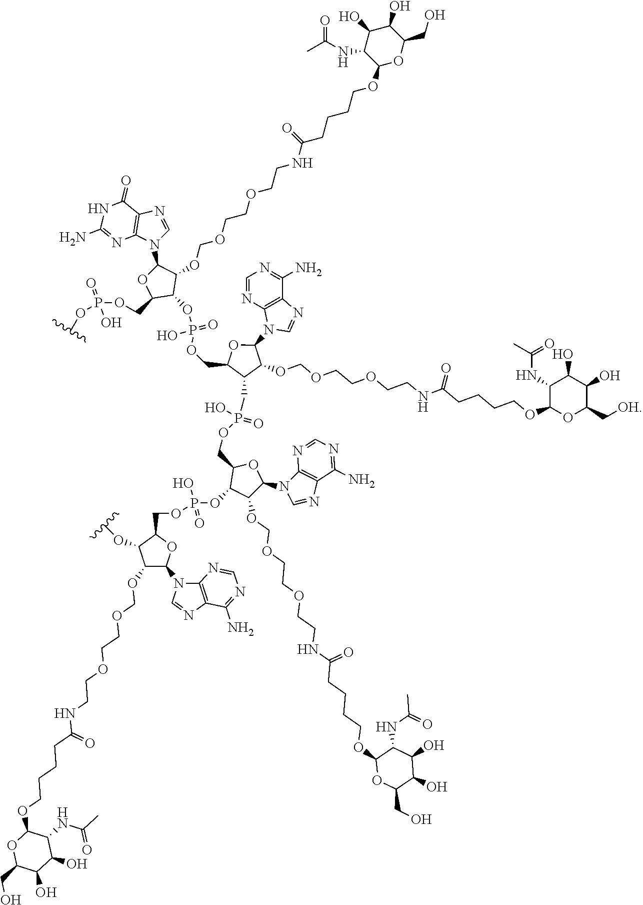 [SMILES: *O[C@H]1[C@@H](OCOCCOCCNC(=O)CCCCO[C@@H]2O[C@H](CO)[C@H](O)[C@H](O)[C@H]2NC(C)=O)[C@H](n2cnc3c(N)ncnc32)O[C@@H]1COP(=O)(O)O[C@H]1[C@@H](OCOCCOCCNC(=O)CCCCO[C@@H]2O[C@H](CO)[C@H](O)[C@H](O)[C@H]2NC(C)=O)[C@H](n2cnc3c(N)ncnc32)O[C@@H]1COP(=O)(O)C[C@H]1[C@@H](OCOCCOCCNC(=O)CCCCO[C@@H]2O[C@H](CO)[C@H](O)[C@H](O)[C@H]2NC(C)=O)[C@H](n2cnc3c(N)ncnc32)O[C@@H]1COP(=O)(O)O[C@H]1[C@@H](OCOCCOCCNC(=O)CCCCO[C@@H]2O[C@H](CO)[C@H](O)[C@H](O)[C@H]2NC(C)=O)[C@H](n2cnc3c(=O)[nH]c(N)nc32)O[C@@H]1COP(=O)(O)O*]

Other aspects of the present disclosure provide oligonucleotides for reducing expression of HMGB1, the oligonucleotide comprising an antisense strand of 15 to 30 nucleotides in length, wherein the antisense strand has a region of complementarity to HMGB1 that is complementary to at least 15 contiguous nucleotides of a sequence as set forth in any one of SEQ ID NOs: 1-13.

In some embodiments, the antisense strand is 19 to 27 nucleotides in length. In some embodiments, the antisense strand is 22 nucleotides in length. In some embodiments, the oligonucleotide further comprises a sense strand of 15 to 50 nucleotides in length, wherein the sense strand forms a duplex region with the antisense strand. In some embodiments, the sense strand is 19 to 50 nucleotides in length. In some embodiments, the duplex region is 20 nucleotides in length.

In some embodiments, the oligonucleotide for reducing expression of HMGB1 comprising a sense strand and an antisense strand, wherein:

(a) the sense strand comprises a sequence as set forth in SEQ ID NO: 788 and the antisense strand comprises a sequence as set forth in SEQ ID NO: 814;

(b) the sense strand comprises a sequence as set forth in SEQ ID NO: 789 and the antisense strand comprises a sequence as set forth in SEQ ID NO: 815;

(c) the sense strand comprises a sequence as set forth in SEQ ID NO: 790 and the antisense strand comprises a sequence as set forth in SEQ ID NO: 816;

(d) the sense strand comprises a sequence as set forth in SEQ ID NO: 791 and the antisense strand comprises a sequence as set forth in SEQ ID NO: 817;

(e) the sense strand comprises a sequence as set forth in SEQ ID NO: 792 and the antisense strand comprises a sequence as set forth in SEQ ID NO: 818;

(f) the sense strand comprises a sequence as set forth in SEQ ID NO: 793 and the antisense strand comprises a sequence as set forth in SEQ ID NO: 819;

(g) the sense strand comprises a sequence as set forth in SEQ ID NO: 794 and the antisense strand comprises a sequence as set forth in SEQ ID NO: 820;

(h) the sense strand comprises a sequence as set forth in SEQ ID NO: 795 and the antisense strand comprises a sequence as set forth in SEQ ID NO: 821;

(i) the sense strand comprises a sequence as set forth in SEQ ID NO: 796 and the antisense strand comprises a sequence as set forth in SEQ ID NO: 822;

(j) the sense strand comprises a sequence as set forth in SEQ ID NO: 797 and the antisense strand comprises a sequence as set forth in SEQ ID NO: 823;

(k) the sense strand comprises a sequence as set forth in SEQ ID NO: 798 and the antisense strand comprises a sequence as set forth in SEQ ID NO: 824;

(l) the sense strand comprises a sequence as set forth in SEQ ID NO: 799 and the antisense strand comprises a sequence as set forth in SEQ ID NO: 825;

(m) the sense strand comprises a sequence as set forth in SEQ ID NO: 800 and the antisense strand comprises a sequence as set forth in SEQ ID NO: 826;

(n) the sense strand comprises a sequence as set forth in SEQ ID NO: 801 and the antisense strand comprises a sequence as set forth in SEQ ID NO: 827;

(o) the sense strand comprises a sequence as set forth in SEQ ID NO: 802 and the antisense strand comprises a sequence as set forth in SEQ ID NO: 828;

(p) the sense strand comprises a sequence as set forth in SEQ ID NO: 803 and the antisense strand comprises a sequence as set forth in SEQ ID NO: 829;

(q) the sense strand comprises a sequence as set forth in SEQ ID NO: 804 and the antisense strand comprises a sequence as set forth in SEQ ID NO: 830;

(r) the sense strand comprises a sequence as set forth in SEQ ID NO: 805 and the antisense strand comprises a sequence as set forth in SEQ ID NO: 831;

(s) the sense strand comprises a sequence as set forth in SEQ ID NO: 806 and the antisense strand comprises a sequence as set forth in SEQ ID NO: 832;

(t) the sense strand comprises a sequence as set forth in SEQ ID NO: 807 and the antisense strand comprises a sequence as set forth in SEQ ID NO: 833;

(u) the sense strand comprises a sequence as set forth in SEQ ID NO: 808 and the antisense strand comprises a sequence as set forth in SEQ ID NO: 834;

(v) the sense strand comprises a sequence as set forth in SEQ ID NO: 809 and the antisense strand comprises a sequence as set forth in SEQ ID NO: 835;

(w) the sense strand comprises a sequence as set forth in SEQ ID NO: 810 and the antisense strand comprises a sequence as set forth in SEQ ID NO: 836;

(x) the sense strand comprises a sequence as set forth in SEQ ID NO: 811 and the antisense strand comprises a sequence as set forth in SEQ ID NO: 837;

(y) the sense strand comprises a sequence as set forth in SEQ ID NO:812 and the antisense strand comprises a sequence as set forth in SEQ ID NO: 838; or

(z) the sense strand comprises a sequence as set forth in SEQ ID NO: 813 and the antisense strand comprises a sequence as set forth in SEQ ID NO: 839.

Further provided herein are compositions comprising any of the oligonucleotides described herein and an excipient.

Further provided herein are methods of delivering an oligonucleotide to a subject, the method comprising administering the composition comprising any of the oligonucleotides described herein to the subject.

In some embodiments, the subject has or is at risk of having liver fibrosis. In some embodiments, the subject has cholestatic or autoimmune liver disease. In some embodiments, expression of HMGB1 protein is reduced by administering to the subject the oligonucleotide.

Further provided herein are methods of treating a subject having or at risk of having liver fibrosis, the method comprising administering to the subject any of the oligonucleotides described herein. In some embodiments, the subject has cholestatic or autoimmune liver disease. In some embodiments, the subject has nonalcoholic steatohepatitis (NASH). In some embodiments, the oligonucleotide is administered prior to exposure of the subject to a hepatotoxic agent. In some embodiments, the oligonucleotide is administered subsequent to exposure of the subject to a hepatotoxic agent. In some embodiments, the oligonucleotide is administered simultaneously with the subject's exposure to a hepatotoxic agent. In some embodiments, the administration results in a reduction in liver HMGB1 levels. In some embodiments, the administration results in a reduction in serum HMGB1 levels.

Further provided herein are the use of any of the oligonucleotides described herein for treating a subject having or at risk of having liver fibrosis. In some embodiments, the subject has cholestatic or autoimmune liver disease. In some embodiments, the subject has nonalcoholic steatohepatitis (NASH).

### BRIEF DESCRIPTION OF THE DRAWINGS

The accompanying drawings, which are incorporated in and constitute a part of this specification, illustrate certain embodiments, and together with the written description, serve to provide non-limiting examples of certain aspects of the compositions and methods disclosed herein.

FIG. 1A and FIG. 1B: In vivo activity evaluation of 3 GalNAc-conjugated HMGB1 oligonucleotides (FIG. 1A) with 3 different modification patterns (FIG. 1B). NM_002128.5 location numbers were used on x-axis.

FIG. 2: In vivo activity evaluation of 3 GalNAc-conjugated HMGB1 oligonucleotides at three different concentrations. NM_002128.5 location numbers were used on x-axis.

FIG. 3: In vivo activity evaluation of three GalNAc-conjugated HMGB1 oligonucleotides at 4-different time points. NM_002128.5 location numbers were used on x-axis.

FIGS. 4A-4F: Screening of 288 triple commons HMGB1 RNAi oligonucleotides in Huh-7 (Human liver) cells. The nucleotide position in NM_002128.5 that corresponds to the 3′ end of the sense strand of each siRNA is indicated on the x axis. The percent mRNA remaining is shown for each of the 5′ assay (red) and the 3′ assay (blue).

FIG. 5: In vivo activity evaluation of 22 HMGB1 RNAi oligonucleotides identified in the screen of FIGS. 4A-4F. The 22 HMGB1 oligonucleotides are GalNAc-conjugated with 2 different modification patterns (M2 and M3, see FIG. 1B for modification patterns). NM_002128.5 location numbers were used on x-axis.

FIG. 6: In vivo activity evaluation of lead GalNAc-conjugated HMGB1 oligonucleotides 21 days after administration. NM_002128.5 location numbers were used on x-axis

FIG. 7: GalNAc-conjugated HMGB1 oligonucleotides that were tested in primary monkey/human hepatocytes. Arrow indicates that the oligonucleotide is also selected to be tested in non-human primate screen.

FIGS. 8A-8D: Activity of the 6 GalNAc-conjugates HMGB1 oligonucleotides in primary monkey (cynomolgous) and human hepatocytes shown by IC50 curve. (FIG. 8A) Cyno hepatocyte #1. (FIG. 8B) Cyno hepatocyte #2. (FIG. 8C) Human hepatocyte #1. (FIG. 8D) Positive control GalNAc-conjugated LDHA oligonucleotide.

FIGS. 9A-9B: In vivo activity evaluation of GalNAc-conjugated HMGB1 oligonucleotides in non-human primates. (FIG. 9A) 4 mg/kg, one dose. (FIG. 9B) 2 mg/kg dosing, 4 repeat doses.

FIGS. 10A-10F: Screening of 6 HMGB1 RNAi oligonucleotides at 3 different concentrations (0.03 nM, 0.1 nM and 1 nM) in mouse, monkey, and human cell lines. (FIG. 10A) Mouse cell line, 5′ assay. (FIG. 10B) Mouse cell line, 3′ assay. (FIG. 10C) Monkey cell line, 5′ assay. (FIG. 10D) Monkey cell line, 3′ assay. (FIG. 10E) Human cell line, 5′ assay. (FIG. 10F) Human cell line, 3′ assay.

FIGS. 11A-11C: Activity of 4 GalNAc-conjugate HMGB1 oligonucleotides in Huh-7 cells by IC50 curve. IC50 curves of HMGB1 (FIG. 11A), HMGB2 (FIG. 11B) and HMGB3 (FIG. 11C), normalized to mock.

### DETAILED DESCRIPTION OF THE INVENTION

According to some aspects, the disclosure provides oligonucleotides targeting HMGB1 mRNA that are effective for reducing HMGB1 expression in cells, particularly liver cells (e.g., hepatocytes) for the treatment of liver fibrosis. Accordingly, in related aspects, the disclosure provides methods of treating fibrosis that involve selectively reducing HMGB1 gene expression in liver. In certain embodiments, HMGB1 targeting oligonucleotides provided herein are designed for delivery to selected cells of target tissues (e.g., liver hepatocytes) to treat fibrosis in those tissues. RNAi oligonucleotides having particular modification patterns are disclosed herein (as outlined in Table 2) that are particularly useful for knocking down HMGB1 mRNA in vivo.

Further aspects of the disclosure, including a description of defined terms, are provided below.

### I. Definitions

Approximately: As used herein, the term “approximately” or “about,” as applied to one or more values of interest, refers to a value that is similar to a stated reference value. In certain embodiments, the term “approximately” or “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

Administering: As used herein, the terms “administering” or “administration” means to provide a substance (e.g., an oligonucleotide) to a subject in a manner that is pharmacologically useful (e.g., to treat a condition in the subject).

Asialoglycoprotein receptor (ASGPR): As used herein, the term “Asialoglycoprotein receptor” or “ASGPR” refers to a bipartite C-type lectin formed by a major 48 kDa (ASGPR-1) and minor 40 kDa subunit (ASGPR-2). ASGPR is primarily expressed on the sinusoidal surface of hepatocyte cells, and has a major role in binding, internalization, and subsequent clearance of circulating glycoproteins that contain terminal galactose or N-acetylgalactosamine residues (asialoglycoproteins).

Attenuates: As used herein, the term “attenuates” means reduces or effectively halts. As a non-limiting example, one or more of the treatments provided herein may reduce or effectively halt the onset or progression of liver fibrosis or liver inflammation in a subject. This attenuation may be exemplified by, for example, a decrease in one or more aspects (e.g., symptoms, tissue characteristics, and cellular, inflammatory or immunological activity, etc.) of liver fibrosis or liver inflammation, no detectable progression (worsening) of one or more aspects of liver fibrosis or liver inflammation, or no detectable aspects of liver fibrosis or liver inflammation in a subject when they might otherwise be expected.

Complementary: As used herein, the term “complementary” refers to a structural relationship between two nucleotides (e.g., on two opposing nucleic acids or on opposing regions of a single nucleic acid strand) that permits the two nucleotides to form base pairs with one another. For example, a purine nucleotide of one nucleic acid that is complementary to a pyrimidine nucleotide of an opposing nucleic acid may base pair together by forming hydrogen bonds with one another. In some embodiments, complementary nucleotides can base pair in the Watson-Crick manner or in any other manner that allows for the formation of stable duplexes. In some embodiments, two nucleic acids may have regions of multiple nucleotides that are complementary with each other so as to form regions of complementarity, as described herein.

Deoxyribonucleotide: As used herein, the term “deoxyribonucleotide” refers to a nucleotide having a hydrogen in place of a hydroxyl at the 2′ position of its pentose sugar as compared with a ribonucleotide. A modified deoxyribonucleotide is a deoxyribonucleotide having one or more modifications or substitutions of atoms other than at the 2′ position, including modifications or substitutions in or of the sugar, phosphate group or base.

Double-stranded oligonucleotide: As used herein, the term “double-stranded oligonucleotide” refers to an oligonucleotide that is substantially in a duplex form. In some embodiments, the complementary base-pairing of duplex region(s) of a double-stranded oligonucleotide is formed between of antiparallel sequences of nucleotides of covalently separate nucleic acid strands. In some embodiments, complementary base-pairing of duplex region(s) of a double-stranded oligonucleotide is formed between antiparallel sequences of nucleotides of nucleic acid strands that are covalently linked. In some embodiments, complementary base-pairing of duplex region(s) of a double-stranded oligonucleotide is formed from single nucleic acid strand that is folded (e.g., via a hairpin) to provide complementary antiparallel sequences of nucleotides that base pair together. In some embodiments, a double-stranded oligonucleotide comprises two covalently separate nucleic acid strands that are fully duplexed with one another. However, in some embodiments, a double-stranded oligonucleotide comprises two covalently separate nucleic acid strands that are partially duplexed, e.g., having overhangs at one or both ends. In some embodiments, a double-stranded oligonucleotide comprises antiparallel sequence of nucleotides that are partially complementary, and thus, may have one or more mismatches, which may include internal mismatches or end mismatches.

Duplex: As used herein, the term “duplex,” in reference to nucleic acids (e.g., oligonucleotides), refers to a structure formed through complementary base pairing of two antiparallel sequences of nucleotides.

Excipient: As used herein, the term “excipient” refers to a non-therapeutic agent that may be included in a composition, for example, to provide or contribute to a desired consistency or stabilizing effect.

Hepatocyte: As used herein, the term “hepatocyte” or “hepatocytes” refers to cells of the parenchymal tissues of the liver. These cells make up approximately 70-85% of the liver's mass and manufacture serum albumin, fibrinogen, and the prothrombin group of clotting factors (except for Factors 3 and 4). Markers for hepatocyte lineage cells may include, but are not limited to: transthyretin (Ttr), glutamine synthetase (Glu1), hepatocyte nuclear factor 1a (Hnf1a), and hepatocyte nuclear factor 4a (Hnf4a). Markers for mature hepatocytes may include, but are not limited to: cytochrome P450 (Cyp3a11), fumarylacetoacetate hydrolase (Fah), glucose 6-phosphate (G6p), albumin (Alb), and OC2-2F8 (see, e.g., Huch et al., Nature, 2013, 494(7436):247-250, the contents of which relating to hepatocyte markers is incorporated herein by reference.

Hepatotoxic agent: As used herein, a “hepatotoxic agent” is a chemical compound, virus, or other substance that is itself toxic to the liver or can be processed to form a metabolite that is toxic to the liver. Hepatotoxic agents may include, but are not limited to, carbon tetrachloride (CC14), acetaminophen (paracetamol), vinyl chloride, arsenic, chloroform, and nonsteroidal anti-inflammatory drugs (such as aspirin and phenylbutazone).

Liver inflammation: As used herein, the term “liver inflammation” or “hepatitis” refers to a physical condition in which the liver becomes swollen, dysfunctional, and/or painful, especially as a result of injury or infection, as may be caused by exposure to a hepatotoxic agent. Symptoms may include jaundice (yellowing of the skin or eyes), fatigue, weakness, nausea, vomiting, appetite reduction, and weight loss. Liver inflammation, if left untreated, may progress to fibrosis, cirrhosis, liver failure, or liver cancer.

Liver fibrosis: As used herein, the term “liver fibrosis” or “fibrosis of the liver” refers to an excessive accumulation in the liver of extracellular matrix proteins, which could include collagens (I, III, and IV), fibronectin, undulin, elastin, laminin, hyaluronan, and proteoglycans resulting from inflammation and liver cell death. Liver fibrosis, if left untreated, may progress to cirrhosis, liver failure, or liver cancer.

Loop: As used herein the term, “loop” refers to a unpaired region of a nucleic acid (e.g., oligonucleotide) that is flanked by two antiparallel regions of the nucleic acid that are sufficiently complementary to one another, such that under appropriate hybridization conditions (e.g., in a phosphate buffer, in a cell), the two antiparallel regions, which flank the unpaired region, hybridize to form a duplex (referred to as a “stem”).

Modified Internucleotide Linkage: As used herein, the term “modified internucleotide linkage” refers to an internucleotide linkage having one or more chemical modifications compared with a reference internucleotide linkage comprising a phosphodiester bond. In some embodiments, a modified nucleotide is a non-naturally occurring linkage. Typically, a modified internucleotide linkage confers one or more desirable properties to a nucleic acid in which the modified internucleotide linkage is present. For example, a modified nucleotide may improve thermal stability, resistance to degradation, nuclease resistance, solubility, bioavailability, bioactivity, reduced immunogenicity, etc.

Modified Nucleotide: As used herein, the term “modified nucleotide” refers to a nucleotide having one or more chemical modifications compared with a corresponding reference nucleotide selected from: adenine ribonucleotide, guanine ribonucleotide, cytosine ribonucleotide, uracil ribonucleotide, adenine deoxyribonucleotide, guanine deoxyribonucleotide, cytosine deoxyribonucleotide and thymidine deoxyribonucleotide. In some embodiments, a modified nucleotide is a non-naturally occurring nucleotide. In some embodiments, a modified nucleotide has one or more chemical modification in its sugar, nucleobase, and/or phosphate group. In some embodiments, a modified nucleotide has one or more chemical moieties conjugated to a corresponding reference nucleotide. Typically, a modified nucleotide confers one or more desirable properties to a nucleic acid in which the modified nucleotide is present. For example, a modified nucleotide may improve thermal stability, resistance to degradation, nuclease resistance, solubility, bioavailability, bioactivity, reduced immunogenicity, etc.

Nicked Tetraloop Structure: A “nicked tetraloop structure” is a structure of a RNAi oligonucleotide characterized by the presence of separate sense (passenger) and antisense (guide) strands, in which the sense strand has a region of complementarity with the antisense strand, and in which at least one of the strands, generally the sense strand, has a tetraloop configured to stabilize an adjacent stem region formed within the at least one strand.

Oligonucleotide: As used herein, the term “oligonucleotide” refers to a short nucleic acid, e.g., of less than 100 nucleotides in length. An oligonucleotide may be single-stranded or double-stranded. An oligonucleotide may or may not have duplex regions. As a set of non-limiting examples, an oligonucleotide may be, but is not limited to, a small interfering RNA (siRNA), microRNA (miRNA), short hairpin RNA (shRNA), dicer substrate interfering RNA (dsiRNA), antisense oligonucleotide, short siRNA, or single-stranded siRNA. In some embodiments, a double-stranded oligonucleotide is an RNAi oligonucleotide.

Overhang: As used herein, the term “overhang” refers to terminal non-base pairing nucleotide(s) resulting from one strand or region extending beyond the terminus of a complementary strand with which the one strand or region forms a duplex. In some embodiments, an overhang comprises one or more unpaired nucleotides extending from a duplex region at the 5′ terminus or 3′ terminus of a double-stranded oligonucleotide. In certain embodiments, the overhang is a 3′ or 5′ overhang on the antisense strand or sense strand of a double-stranded oligonucleotides.

Phosphate analog: As used herein, the term “phosphate analog” refers to a chemical moiety that mimics the electrostatic and/or steric properties of a phosphate group. In some embodiments, a phosphate analog is positioned at the 5′ terminal nucleotide of an oligonucleotide in place of a 5′-phosphate, which is often susceptible to enzymatic removal. In some embodiments, a 5′ phosphate analogs contain a phosphatase-resistant linkage Examples of phosphate analogs include 5′ phosphonates, such as 5′ methylenephosphonate (5′-MP) and 5′-(E)-vinylphosphonate (5′-VP). In some embodiments, an oligonucleotide has a phosphate analog at a 4′-carbon position of the sugar (referred to as a “4′-phosphate analog”) at a 5′-terminal nucleotide. An example of a 4′-phosphate analog is oxymethylphosphonate, in which the oxygen atom of the oxymethyl group is bound to the sugar moiety (e.g., at its 4′-carbon) or analog thereof (see, e.g., International patent publication WO/2018/045317, the contents of which relating to phosphate analogs is incorporated herein by reference. Other modifications have been developed for the 5′ end of oligonucleotides (see, e.g., WO 2011/133871; U.S. Pat. No. 8,927,513; and Prakash et al., Nucleic Acids Res., 2015, 43(6):2993-3011, the contents of each of which relating to phosphate analogs are incorporated herein by reference).

Reduced expression: As used herein, the term “reduced expression” of a gene refers to a decrease in the amount of RNA transcript or protein encoded by the gene and/or a decrease in the amount of activity of the gene in a cell or subject, as compared to an appropriate reference cell or subject. For example, the act of treating a cell with a double-stranded oligonucleotide (e.g., one having an antisense strand that is complementary to HMGB1 mRNA sequence) may result in a decrease in the amount of RNA transcript, protein and/or activity (e.g., encoded by the HMGB1 gene) compared to a cell that is not treated with the double-stranded oligonucleotide. Similarly, “reducing expression” as used herein refers to an act that results in reduced expression of a gene (e.g., HMGB1).

Region of Complementarity: As used herein, the term “region of complementary” refers to a sequence of nucleotides of a nucleic acid (e.g., a double-stranded oligonucleotide) that is sufficiently complementary to an antiparallel sequence of nucleotides to permit hybridization between the two sequences of nucleotides under appropriate hybridization conditions, e.g., in a phosphate buffer, in a cell, etc.

Ribonucleotide: As used herein, the term “ribonucleotide” refers to a nucleotide having a ribose as its pentose sugar, which contains a hydroxyl group at its 2′ position. A modified ribonucleotide is a ribonucleotide having one or more modifications or substitutions of atoms other than at the 2′ position, including modifications or substitutions in or of the ribose, phosphate group or base.

RNAi Oligonucleotide: As used herein, the term “RNAi oligonucleotide” refers to either (a) a double stranded oligonucleotide having a sense strand (passenger) and antisense strand (guide), in which the antisense strand or part of the antisense strand is used by the Argonaute 2 (Ago2) endonuclease in the cleavage of a target mRNA or (b) a single stranded oligonucleotide having a single antisense strand, where that antisense strand (or part of that antisense strand) is used by the Ago2 endonuclease in the cleavage of a target mRNA.

Strand: As used herein, the term “strand” refers to a single contiguous sequence of nucleotides linked together through internucleotide linkages (e.g., phosphodiester linkages, phosphorothioate linkages). In some embodiments, a strand has two free ends, e.g., a 5′-end and a 3′-end.

Subject: As used herein, the term “subject” means any mammal, including mice, rabbits, and humans. In some embodiments, the subject is a human or non-human primate. The terms “individual” or “patient” may be used interchangeably with “subject.”

Synthetic: As used herein, the term “synthetic” refers to a nucleic acid or other molecule that is artificially synthesized (e.g., using a machine (e.g., a solid-state nucleic acid synthesizer)) or that is otherwise not derived from a natural source (e.g., a cell or organism) that normally produces the molecule.

Targeting ligand: As used herein, the term “targeting ligand” refers to a molecule (e.g., a carbohydrate, amino sugar, cholesterol, polypeptide or lipid) that selectively binds to a cognate molecule (e.g., a receptor) of a tissue or cell of interest and that is conjugatable to another substance for purposes of targeting the other substance to the tissue or cell of interest. For example, in some embodiments, a targeting ligand may be conjugated to an oligonucleotide for purposes of targeting the oligonucleotide to a specific tissue or cell of interest. In some embodiments, a targeting ligand selectively binds to a cell surface receptor. Accordingly, in some embodiments, a targeting ligand when conjugated to an oligonucleotide facilitates delivery of the oligonucleotide into a particular cell through selective binding to a receptor expressed on the surface of the cell and endosomal internalization by the cell of the complex comprising the oligonucleotide, targeting ligand and receptor. In some embodiments, a targeting ligand is conjugated to an oligonucleotide via a linker that is cleaved following or during cellular internalization such that the oligonucleotide is released from the targeting ligand in the cell.

Tetraloop: As used herein, the term “tetraloop” refers to a loop that increases stability of an adjacent duplex formed by hybridization of flanking sequences of nucleotides. The increase in stability is detectable as an increase in melting temperature (Tm) of an adjacent stem duplex that is higher than the Tm of the adjacent stem duplex expected, on average, from a set of loops of comparable length consisting of randomly selected sequences of nucleotides. For example, a tetraloop can confer a melting temperature of at least 50° C., at least 55° C., at least 56° C., at least 58° C., at least 60° C., at least 65° C. or at least 75° C. in 10 mM NaHPO₄ to a hairpin comprising a duplex of at least 2 base pairs in length. In some embodiments, a tetraloop may stabilize a base pair in an adjacent stem duplex by stacking interactions. In addition, interactions among the nucleotides in a tetraloop include but are not limited to non-Watson-Crick base pairing, stacking interactions, hydrogen bonding, and contact interactions (Cheong et al., Nature, 1990, 346(6285):680-2; Heus and Pardi, Science, 1991, 253(5016):191-4). In some embodiments, a tetraloop comprises or consists of 3 to 6 nucleotides, and is typically 4 to 5 nucleotides. In certain embodiments, a tetraloop comprises or consists of three, four, five, or six nucleotides, which may or may not be modified (e.g., which may or may not be conjugated to a targeting moiety). In one embodiment, a tetraloop consists of four nucleotides. Any nucleotide may be used in the tetraloop and standard IUPAC-IUB symbols for such nucleotides may be used, as described in Cornish-Bowden, Nucleic Acids Res., 1985, 13:3021-3030. For example, the letter “N” may be used to mean that any base may be in that position, the letter “R” may be used to show that A (adenine) or G (guanine) may be in that position, and “B” may be used to show that C (cytosine), G (guanine), or T (thymine) may be in that position. Examples of tetraloops include the UNCG family of tetraloops (e.g., UUCG), the GNRA family of tetraloops (e.g., GAAA), and the CUUG tetraloop (Woese et al., Proc Natl Acad Sci USA., 1990, 87(21):8467-71; Antao et al., Nucleic Acids Res., 1991, 19(21):5901-5). Examples of DNA tetraloops include the d(GNNA) family of tetraloops (e.g., d(GTTA), the d(GNRA)) family of tetraloops, the d(GNAB) family of tetraloops, the d(CNNG) family of tetraloops, and the d(TNCG) family of tetraloops (e.g., d(TTCG)). See, for example, Nakano et al., Biochemistry, 2002, 41(48):14281-14292; Shinji et al., Nippon Kagakkai Koen Yokoshu, 2000, 78(2):731; which are incorporated by reference herein for their relevant disclosures. In some embodiments, the tetraloop is contained within a nicked tetraloop structure.

Treat: As used herein, the term “treat” refers to the act of providing care to a subject in need thereof, e.g., through the administration a therapeutic agent (e.g., an oligonucleotide) to the subject, for purposes of improving the health and/or well-being of the subject with respect to an existing condition (e.g., a disease, disorder) or to prevent or decrease the likelihood of the occurrence of a condition. In some embodiments, treatment involves reducing the frequency or severity of at least one sign, symptom or contributing factor of a condition (e.g., disease, disorder) experienced by a subject.

### II. Oligonucleotide-Based Inhibitors of HMGB1 Expression

i. HMGB1 Target Sequences

In some embodiments, oligonucleotide-based inhibitors of HMGB1 expression are provided herein that can be used to achieve a therapeutic benefit. Through examination of the HMGB1 mRNA, including mRNAs of multiple different species (human, rhesus monkey, and mouse (see, e.g., Example 1) and in vitro and in vivo testing, it has been discovered that certain sequences of HMGB1 mRNA are useful as targeting sequences because they are more amenable than others to oligonucleotide-based inhibition. In some embodiments, a HMGB1 target sequence comprises, or consists of, a sequence as forth in any one of SEQ ID NOs: 1-13. These regions of HMGB1 mRNA may be targeted using oligonucleotides as discussed herein for purposes of inhibiting HMGB1 mRNA expression.

Accordingly, in some embodiments, oligonucleotides provided herein are designed so as to have regions of complementarity to HMGB1 mRNA (e.g., within a target sequence of HMGB1 mRNA) for purposes of targeting the mRNA in cells and inhibiting its expression. The region of complementary is generally of a suitable length and base content to enable annealing of the oligonucleotide (or a strand thereof) to HMGB1 mRNA for purposes of inhibiting its expression. In some embodiments, the region of complementarity is at least 12, at least 13, at least 14, at least 15, at least 16, at least 17, at least 18, at least 19 or at least 20 nucleotides in length. In some embodiments, an oligonucleotide provided herein has a region of complementarity to HMGB1 that is in the range of 12 to 30 (e.g., 12 to 30, 12 to 22, 15 to 25, 17 to 21, 18 to 27, 19 to 27, or 15 to 30) nucleotides in length. In some embodiments, an oligonucleotide provided herein has a region of complementarity to HMGB1 that is 12, 13, 14, 15, 16, 17, 18, 19, 20, 21, 22, 23, 24, 25, 26, 27, 28, 29 or 30 nucleotides in length.

In some embodiments, an oligonucleotide disclosed herein comprises a region of complementarity (e.g., on an antisense strand of a double-stranded oligonucleotide) that is at least partially complementary to a sequence as set forth in any one of SEQ ID NOs: 1-13. In some embodiments, an oligonucleotide disclosed herein comprises a region of complementarity (e.g., on an antisense strand of a double-stranded oligonucleotide) that is fully complementary to a sequence as set forth in any one of SEQ ID NOs: 1-13. In some embodiments, a region of complementarity of an oligonucleotide (e.g., on an antisense strand of a double-stranded oligonucleotide) is complementary to a contiguous sequence of nucleotides of a sequence as set forth in any one of SEQ ID NOs: 1-13 that is in the range of 12 to 20 nucleotides (e.g., 12 to 20, 12 to 18, 12 to 16, 12 to 14, 14 to 20, 14 to 18, 14 to 16, 16 to 20, 16 to 18, or 18 to 20) in length. In some embodiments, a region of complementarity of an oligonucleotide (e.g., on an antisense strand of a double-stranded oligonucleotide) is complementary to a contiguous sequence of nucleotides of a sequence as set forth in any one of SEQ ID NOs: 11-13 that is 10, 11, 12, 13, 14, 15, 16, 17, 18, 19, or 20 contiguous nucleotides in length.

In some embodiments, a region of complementarity of an oligonucleotide that is complementary to contiguous nucleotides of a sequence as set forth in any one of SEQ ID NOs: 1-13 spans the entire length of an antisense strand. In some embodiments, a region of complementarity of an oligonucleotide that is complementary to contiguous nucleotides of a sequence as set forth in any one of SEQ ID NOs: 1-13 spans a portion of the entire length of an antisense strand. In some embodiments, an oligonucleotide disclosed herein comprises a region of complementarity (e.g., on an antisense strand of a double-stranded oligonucleotide) that is at least partially (e.g., fully) complementary to a contiguous stretch of nucleotides spanning nucleotides 1-20 of a sequence as set forth in any one of SEQ ID NOs: 1-13.

In some embodiments, a region of complementarity to HMGB1 may have one or more mismatches compared with a corresponding sequence of HMGB1 mRNA. A region of complementarity on an oligonucleotide may have up to 1, up to 2, up to 3, up to 4, up to 5, etc. mismatches provided that it maintains the ability to form complementary base pairs with HMGB1 mRNA under appropriate hybridization conditions. Alternatively, a region of complementarity on an oligonucleotide may have no more than 1, no more than 2, no more than 3, no more than 4, or no more than 5 mismatches provided that it maintains the ability to form complementary base pairs with HMGB1 mRNA under appropriate hybridization conditions. In some embodiments, if there are more than one mismatches in a region of complementarity, they may be positioned consecutively (e.g., 2, 3, 4, or more in a row), or interspersed throughout the region of complementarity provided that the oligonucleotide maintains the ability to form complementary base pairs with HMGB1 mRNA under appropriate hybridization conditions.

ii. Types of Oligonucleotides

There are a variety of structures of oligonucleotides that are useful for targeting HMGB1 in the methods of the present disclosure, including RNAi, antisense, miRNA, etc. Any of the structures described herein or elsewhere may be used as a framework to incorporate or target a sequence described herein (e.g., a hotpot sequence of HMBG1 such as those illustrated in SEQ ID NOs: 1-13).

In some embodiments, oligonucleotides for reducing the expression of HMGB1 expression engage RNA interference (RNAi) pathways upstream or downstream of dicer involvement. For example, RNAi oligonucleotides have been developed with each strand having sizes of 19-25 nucleotides with at least one 3′ overhang of 1 to 5 nucleotides (see, e.g., U.S. Pat. No. 8,372,968). Longer oligonucleotides have also been developed that are processed by Dicer to generate active RNAi products (see, e.g., U.S. Pat. No. 8,883,996). Further work produced extended double-stranded oligonucleotides where at least one end of at least one strand is extended beyond a duplex targeting region, including structures where one of the strands includes a thermodynamically-stabilizing tetraloop structure (see, e.g., U.S. Pat. Nos. 8,513,207 and 8,927,705, as well as International patent publication WO2010033225, which are incorporated by reference herein for their disclosure of the structures and form of these oligonucleotides). Such structures may include single-stranded extensions (on one or both sides of the molecule) as well as double-stranded extensions.

In some embodiments, oligonucleotides provided herein are designed to engage in the RNA interference pathway downstream of the involvement of dicer (e.g., dicer cleavage). Such oligonucleotides may have an overhang (e.g., of 1, 2, or 3 nucleotides in length) in the 3′ end of the sense strand. Such oligonucleotides (e.g., siRNAs) may comprise a 21 nucleotide guide strand that is antisense to a target RNA and a complementary passenger strand, in which both strands anneal to form a 19-bp duplex and 2 nucleotide overhangs at either or both 3′ ends. Longer oligonucleotide designs are also available including oligonucleotides having a guide strand of 23 nucleotides and a passenger strand of 21 nucleotides, where there is a blunt end on the right side of the molecule (3′-end of passenger strand/5′-end of guide strand) and a two nucleotide 3′-guide strand overhang on the left side of the molecule (5′-end of the passenger strand/3′-end of the guide strand). In such molecules, there is a 21 base pair duplex region. See, for example, U.S. Pat. Nos. 9,012,138; 9,012,621; and 9,193,753, each of which are incorporated herein for their relevant disclosures.

In some embodiments, oligonucleotides as disclosed herein may comprise sense and antisense strands that are both in the range of 17 to 26 (e.g., 17 to 26, 20 to 25, or 21-23) nucleotides in length. In some embodiments, an oligonucleotide as disclosed herein comprises a sense and antisense strand that are both in the range of 19-22 nucleotide in length. In some embodiments, the sense and antisense strands are of equal length. In some embodiments, an oligonucleotide comprises sense and antisense strands, such that there is a 3′-overhang on either the sense strand or the antisense strand, or both the sense and antisense strand. In some embodiments, for oligonucleotides that have sense and antisense strands that are both in the range of 21-23 nucleotides in length, a 3′ overhang on the sense, antisense, or both sense and antisense strands is 1 or 2 nucleotides in length. In some embodiments, the oligonucleotide has a guide strand of 22 nucleotides and a passenger strand of 20 nucleotides, where there is a blunt end on the right side of the molecule (3′-end of passenger strand/5′-end of guide strand) and a two nucleotide 3′-guide strand overhang on the left side of the molecule (5′-end of the passenger strand/3′-end of the guide strand). In such molecules, there is a 20 base pair duplex region.

Other oligonucleotides designs for use with the compositions and methods disclosed herein include: 16-mer siRNAs (see, e.g., Nucleic Acids in Chemistry and Biology, Blackburn (ed.), Royal Society of Chemistry, 2006), shRNAs (e.g., having 19 bp or shorter stems; see, e.g., Moore et al. Methods Mol. Biol., 2010, 629:141-158), blunt siRNAs (e.g., of 19 bps in length; see, e.g., Kraynack and Baker, R N A, 2006, 12:163-176), asymmetrical siRNAs (aiRNA; see, e.g., Sun et al., Nat. Biotechnol., 2008, 26:1379-1382), asymmetric shorter-duplex siRNA (see, e.g., Chang et al., Mol Ther., 2009, 17(4):725-32), fork siRNAs (see, e.g., Hohjoh, FEBS Letters, 2004, 557(1-3):193-198), single-stranded siRNAs (Elsner, Nature Biotechnology, 2012, 30:1063), dumbbell-shaped circular siRNAs (see, e.g., Abe et al., J Am Chem Soc., 2007, 129:15108-15109), and small internally segmented interfering RNA (siRNA; see, e.g., Bramsen et al., Nucleic Acids Res., 2007, 35(17):5886-5897). Each of the foregoing references is incorporated by reference in its entirety for the related disclosures therein. Further non-limiting examples of oligonucleotide structures that may be used in some embodiments to reduce or inhibit the expression of HMGB1 are microRNA (miRNA), short hairpin RNA (shRNA), and short siRNA (see, e.g., Hamilton et al., EMBO J., 2002, 21(17):4671-4679; see also U.S. patent publication no. 20090099115).

Still, in some embodiments, an oligonucleotide for reducing HMGB1 expression as described herein is single-stranded. Such structures may include, but are not limited to single-stranded RNAi molecules. Recent efforts have demonstrated the activity of single-stranded RNAi molecules (see, e.g., Matsui et al., Molecular Therapy, 2016, 24(5):946-955). However, in some embodiments, oligonucleotides provided herein are antisense oligonucleotides (ASOs). An antisense oligonucleotide is a single-stranded oligonucleotide that has a nucleobase sequence which, when written in the 5′ to 3′ direction, comprises the reverse complement of a targeted segment of a particular nucleic acid and is suitably modified (e.g., as a gapmer) so as to induce RNaseH mediated cleavage of its target RNA in cells or (e.g., as a mixmer) so as to inhibit translation of the target mRNA in cells. Antisense oligonucleotides for use in the instant disclosure may be modified in any suitable manner known in the art including, for example, as shown in U.S. Pat. No. 9,567,587, which is incorporated by reference herein for its disclosure regarding modification of antisense oligonucleotides (including, e.g., length, sugar moieties of the nucleobase (pyrimidine, purine), and alterations of the heterocyclic portion of the nucleobase). Further, antisense molecules have been used for decades to reduce expression of specific target genes (see, e.g., Bennett et al., “Pharmacology of Antisense Drugs,” Ann Rev Pharmacol Toxicol., 2017, 57:81-105).

iii. Double-Stranded Oligonucleotides

Double-stranded oligonucleotides for targeting HMGB1 expression (e.g., via the RNAi pathway) generally have a sense strand and an antisense strand that form a duplex with one another. In some embodiments, the sense and antisense strands are not covalently linked. However, in some embodiments, the sense and antisense strands are covalently linked. In some embodiments, a duplex formed between a sense and antisense strand is at least 15 (e.g., at least 15, at least 16, at least 17, at least 18, at least 19, at least 20, or at least 21) nucleotides in length. In some embodiments, a duplex formed between a sense and antisense strand is in the range of 15-30 nucleotides in length (e.g., 15 to 30, 15 to 27, 15 to 22, 18 to 22, 18 to 25, 18 to 27, 18 to 30, or 21 to 30 nucleotides in length). In some embodiments, a duplex formed between a sense and antisense strand is 15, 16, 17, 18, 19, 20, 21, 22, 23, 24, 25, 26, 27, 28, 29, or 30 nucleotides in length. In some embodiments, the duplex region is 20 nucleotides in length. In some embodiments a duplex formed between a sense and antisense strand does not span the entire length of the sense strand and/or antisense strand. In some embodiments, a duplex between a sense and antisense strand spans the entire length of either the sense or antisense strands. In certain embodiments, a duplex between a sense and antisense strand spans the entire length of both the sense strand and the antisense strand.

In some embodiments, an oligonucleotide provided herein comprises a sense strand having a sequence as set forth in any one of SEQ ID NOs: 1-13 and an antisense strand comprising a complementary sequence selected from SEQ ID NOs: 14-26, as is arranged Table 1.

In some embodiments, the sense strand comprising a sequence as set forth in SEQ ID NO: 1 and the antisense strand comprising a sequence as set forth in SEQ ID NO: 14. In some embodiments, the sense strand comprises a sequence as set forth in SEQ ID NO: 2 and the antisense strand comprises a sequence as set forth in SEQ ID NO: 15. In some embodiments, the sense strand comprises a sequence as set forth in SEQ ID NO: 3 and the antisense strand comprises a sequence as set forth in SEQ ID NO: 16. In some embodiments, the sense strand comprises a sequence as set forth in SEQ ID NO: 4 and the antisense strand comprises a sequence as set forth in SEQ ID NO: 17. In some embodiments, the sense strand comprises a sequence as set forth in SEQ ID NO: 5 and the antisense strand comprises a sequence as set forth in SEQ ID NO: 18. In some embodiments, the sense strand comprises a sequence as set forth in SEQ ID NO: 6 and the antisense strand comprises a sequence as set forth in SEQ ID NO: 19. In some embodiments, the sense strand comprises a sequence as set forth in SEQ ID NO: 7 and the antisense strand comprises a sequence as set forth in SEQ ID NO: 20. In some embodiments, the sense strand comprises a sequence as set forth in SEQ ID NO: 8 and the antisense strand comprises a sequence as set forth in SEQ ID NO: 21. In some embodiments, the sense strand comprises a sequence as set forth in SEQ ID NO: 9 and the antisense strand comprises a sequence as set forth in SEQ ID NO: 22. In some embodiments, the sense strand comprises a sequence as set forth in SEQ ID NO: 10 and the antisense strand comprises a sequence as set forth in SEQ ID NO: 23. In some embodiments, the sense strand comprises a sequence as set forth in SEQ ID NO: 11 and the antisense strand comprises a sequence as set forth in SEQ ID NO: 24. In some embodiments, the sense strand comprises a sequence as set forth in SEQ ID NO: 12 and the antisense strand comprises a sequence as set forth in SEQ ID NO: 25. In some embodiments, the sense strand comprises a sequence as set forth in SEQ ID NO: 13 and the antisense strand comprises a sequence as set forth in SEQ ID NO: 26.

In some embodiments, an oligonucleotide provided herein comprises a sense strand comprising a sequence as set forth in any one of SEQ ID NOs: 27-39 and an antisense strand comprising a complementary sequence selected from SEQ ID NOs: 14-26, as is also arranged in Table 2, including modifications to the sense sequence and antisense sequences. In some embodiments, the sense strand comprises a sequence as set forth in SEQ ID NO: 27 and the antisense strand comprises a sequence as set forth in SEQ ID NO: 14. In some embodiments, the sense strand comprises a sequence as set forth in SEQ ID NO: 28 and the antisense strand comprises a sequence as set forth in SEQ ID NO: 15. In some embodiments, the sense strand comprises a sequence as set forth in SEQ ID NO: 29 and the antisense strand comprises a sequence as set forth in SEQ ID NO: 16. In some embodiments, the sense strand comprises a sequence as set forth in SEQ ID NO: 30 and the antisense strand comprises a sequence as set forth in SEQ ID NO: 17. In some embodiments, the sense strand comprises a sequence as set forth in SEQ ID NO: 31 and the antisense strand comprises a sequence as set forth in SEQ ID NO: 18. In some embodiments, the sense strand comprises a sequence as set forth in SEQ ID NO: 32 and the antisense strand comprises a sequence as set forth in SEQ ID NO: 19. In some embodiments, the sense strand comprises a sequence as set forth in SEQ ID NO: 33 and the antisense strand comprises a sequence as set forth in SEQ ID NO: 20. In some embodiments, the sense strand comprises a sequence as set forth in SEQ ID NO: 34 and the antisense strand comprises a sequence as set forth in SEQ ID NO: 21. In some embodiments, the sense strand comprises a sequence as set forth in SEQ ID NO: 35 and the antisense strand comprises a sequence as set forth in SEQ ID NO: 22. In some embodiments, the sense strand comprises a sequence as set forth in SEQ ID NO: 36 and the antisense strand comprises a sequence as set forth in SEQ ID NO: 23. In some embodiments, the sense strand comprises a sequence as set forth in SEQ ID NO: 37 and the antisense strand comprises a sequence as set forth in SEQ ID NO: 24. In some embodiments, the sense strand comprises a sequence as set forth in SEQ ID NO: 38 and the antisense strand comprises a sequence as set forth in SEQ ID NO: 25. In some embodiments, the sense strand comprises a sequence as set forth in SEQ ID NO: 39 and the antisense strand comprises a sequence as set forth in SEQ ID NO: 26.

It should be appreciated that, in some embodiments, sequences presented in the sequence listing may be referred to in describing the structure of an oligonucleotide or other nucleic acid. In such embodiments, the actual oligonucleotide or other nucleic acid may have one or more alternative nucleotides (e.g., an RNA counterpart of a DNA nucleotide or a DNA counterpart of an RNA nucleotide) and/or one or more modified nucleotides and/or one or more modified internucleotide linkages and/or one or more other modification compared with the specified sequence while retaining essentially same or similar complementary properties as the specified sequence.

In some embodiments, a double-stranded oligonucleotide comprises a 25-nucleotide sense strand and a 27-nucleotide antisense strand that when acted upon by a dicer enzyme results in an antisense strand that is incorporated into the mature RISC. In some embodiments, a sense strand of an oligonucleotide is longer than 27 nucleotides (e.g., 28, 29, 30, 31, 32, 33, 34, 35, 36, 37, 38, 39 or 40 nucleotides). In some embodiments, a sense strand of an oligonucleotide is longer than 25 nucleotides (e.g., 26, 27, 28, 29 or 30 nucleotides).

In some embodiments, the length of a duplex formed between a sense and antisense strand of an oligonucleotide may be 12 to 30 nucleotides (e.g., 12 to 30, 12 to 27, 15 to 25, 18 to 30 or 19 to 30 nucleotides) in length. In some embodiments, the length of a duplex formed between a sense and antisense strand of an oligonucleotide is at least 12 nucleotides long (e.g., at least 12, at least 15, at least 20, or at least 25 nucleotides long). In some embodiments, the length of a duplex formed between a sense and antisense strand of an oligonucleotide is 12, 13, 14, 15, 16, 17, 18, 19, 20, 21, 22, 23, 24, 25, 26, 27, 28, 29, or 30 nucleotides in length.

In some embodiments, oligonucleotides provided herein have one 5′end that is thermodynamically less stable compared to the other 5′ end. In some embodiments, an asymmetry oligonucleotide is provided that includes a blunt end at the 3′ end of a sense strand and an overhang at the 3′ end of an antisense strand. In some embodiments, a 3′ overhang on an antisense strand is 1-8 nucleotides in length (e.g., 1, 2, 3, 4, 5, 6, 7 or 8 nucleotides in length). Typically, an oligonucleotide for RNAi has a two-nucleotide overhang on the 3′ end of the antisense (guide) strand. However, other overhangs are possible. In some embodiments, an overhang is a 3′ overhang comprising a length of between one and six nucleotides, optionally one to five, one to four, one to three, one to two, two to six, two to five, two to four, two to three, three to six, three to five, three to four, four to six, four to five, five to six nucleotides, or one, two, three, four, five or six nucleotides. However, in some embodiments, the overhang is a 5′ overhang comprising a length of between one and six nucleotides, optionally one to five, one to four, one to three, one to two, two to six, two to five, two to four, two to three, three to six, three to five, three to four, four to six, four to five, five to six nucleotides, or one, two, three, four, five or six nucleotides.

In some embodiments, two terminal nucleotides on the 3′ end of an antisense strand are modified. In some embodiments, the two terminal nucleotides on the 3′ end of the antisense strand are complementary with the target. In some embodiments, the two terminal nucleotides on the 3′ end of the antisense strand are not complementary with the target. In some embodiments, two terminal nucleotides on each 3′ end of an oligonucleotide in the nicked tetraloop structure are GG. Typically, one or both of the two terminal GG nucleotides on each 3′ end of an oligonucleotide is not complementary with the target.

In some embodiments, there is one or more (e.g., 1, 2, 3, 4, 5) mismatches between a sense and antisense strand. If there is more than one mismatch between a sense and antisense strand, they may be positioned consecutively (e.g., 2, 3 or more in a row), or interspersed throughout the region of complementarity. In some embodiments, the 3′-terminus of the sense strand contains one or more mismatches. In one embodiment, two mismatches are incorporated at the 3′ terminus of the sense strand. In some embodiments, base mismatches or destabilization of segments at the 3′-end of the sense strand of the oligonucleotide improved the potency of synthetic duplexes in RNAi, possibly through facilitating processing by Dicer.

a. Antisense Strands

In some embodiments, an antisense strand of an oligonucleotide may be referred to as a “guide strand.” For example, if an antisense strand can engage with RNA-induced silencing complex (RISC) and bind to an Argonaut protein, or engage with or bind to one or more similar factors, and direct silencing of a target gene, it may be referred to as a guide strand. In some embodiments a sense strand complementary with a guide strand may be referred to as a “passenger strand.”

In some embodiments, an oligonucleotide provided herein comprises an antisense strand that is up to 50 nucleotides in length (e.g., up to 30, up to 27, up to 25, up to 21, or up to 19 nucleotides in length). In some embodiments, an oligonucleotide provided herein comprises an antisense strand is at least 12 nucleotides in length (e.g., at least 12, at least 15, at least 19, at least 21, at least 25, or at least 27 nucleotides in length). In some embodiments, an antisense strand of an oligonucleotide disclosed herein is in the range of 12 to 50 or 12 to 30 (e.g., 12 to 30, 11 to 27, 11 to 25, 15 to 21, 15 to 27, 17 to 21, 17 to 25, 19 to 27, or 19 to 30) nucleotides in length. In some embodiments, an antisense strand of any one of the oligonucleotides disclosed herein is 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 nucleotides in length.

In some embodiments an oligonucleotide disclosed herein comprises an antisense strand comprising a sequence as set forth in any one of SEQ ID NOs: 14-26. In some embodiments, an oligonucleotide comprises an antisense strand comprising a contiguous sequence of nucleotides that is in the range of 12 to 20 nucleotides (e.g., 12 to 20, 12 to 18, 12 to 16, 12 to 14, 14 to 20, 14 to 18, 14 to 16, 16 to 20, 16 to 18, or 18 to 20 nucleotides) in length of any of the sequences as set forth in any one of SEQ ID NOs: 14-26. In some embodiments, an oligonucleotide comprises an antisense strand comprising a contiguous sequence of nucleotides of a sequence as set forth in any one of SEQ ID NOs: 14-26 that is 12, 13, 14, 15, 16, 17, 18, 19, or 20 contiguous nucleotides in length. In some embodiments, an oligonucleotide comprises an antisense strand that consists of a sequence as set forth in any one of SEQ ID NOs: 14-26.

b. Sense Strands

In some embodiments, a double-stranded oligonucleotide may have a sense strand of up to 40 nucleotides in length (e.g., up to 40, up to 35, up to 30, up to 27, up to 25, up to 21, up to 19 up to 17, or up to 12 nucleotides in length). In some embodiments, an oligonucleotide may have a sense strand of at least 12 nucleotides in length (e.g., at least 12, at least 15, at least 19, at least 21, at least 25, at least 27, at least 30, at least 35, or at least 38 nucleotides in length). In some embodiments, an oligonucleotide may have a sense strand in a range of 12 to 50 (e.g., 12 to 40, 12 to 36, 12 to 32, 12 to 28, 15 to 40, 15 to 36, 15 to 32, 15 to 28, 17 to 21, 17 to 25, 19 to 27, 19 to 30, 20 to 40, 22 to 40, 25 to 40, or 32 to 40) nucleotides in length. In some embodiments, an oligonucleotide may have a sense strand of 12, 13, 14, 15, 16, 17, 18, 19, 20, 21, 22, 23, 24, 25, 26, 27, 28, 29, 30, 31, 32, 33, 34, 35, 36, 37, 38, 39, or 40 nucleotides in length. In some embodiments, a sense strand of an oligonucleotide is longer than 27 nucleotides (e.g., 28, 29, 30, 31, 32, 33, 34, 35, 36, 37, 38, 39 or 40 nucleotides). In some embodiments, a sense strand of an oligonucleotide is longer than 25 nucleotides (e.g., 26, 27, 28, 29, 30, 31, 32, 33, 34, 35, or 36 nucleotides). In some embodiments, the sense strand is 20 nucleotides in length. In some embodiments, the sense strand is 36 nucleotides in length.

In some embodiments, an oligonucleotide disclosed herein comprises a sense strand sequence as set forth in in any one of SEQ ID NOs: 1-13 and 27-39. In some embodiments, an oligonucleotide has a sense strand that comprises at least 12 (e.g., at least 13, at least 14, at least 15, at least 16, at least 17, at least 18, at least 19, at least 20, at least 21, at least 22, or at least 23) contiguous nucleotides of a sequence as set forth in in any one of SEQ ID NOs: 1-13 and 27-39. In some embodiments, an oligonucleotide has a sense strand that comprises a contiguous sequence of nucleotides that is in the range of 7 to 36 nucleotides (e.g., 12 to 30, 12 to 27, 12 to 22, 15 to 25, 17 to 21, 18 to 27, 19-27, 20-36, or 15 to 36 nucleotides) in length of any of the sequences as set forth in any one of SEQ ID NOs: 1-13 and 27-39. In some embodiments, an oligonucleotide has a sense strand that comprises a contiguous sequence of nucleotides of a sequence as set forth in any one of SEQ ID NOs: 1-13 and 27-39 that is 12, 13, 14, 15, 16, 17, 18, 19, 20, 21, 22, 23, 24, 25, 26, 27, 28, 29, 30, 31, 32, 33, 34, 35, or 36 nucleotides in length. In some embodiments, an oligonucleotide has a sense strand that consists of a sequence as set forth in any one of SEQ ID NOs: 1-13 and 27-39.

In some embodiments, a sense strand comprises a stem-loop at its 3′-end. In some embodiments, a sense strand comprises a stem-loop at its 5′-end. In some embodiments, a strand comprising a stem loop is in the range of 2 to 66 nucleotides long (e.g., 2 to 66, 10 to 52, 14 to 40, 2 to 30, 4 to 26, 8 to 22, 12 to 18, 10 to 22, 14 to 26, or 14 to 30 nucleotides long). In some embodiments, a strand comprising a stem loop is 8, 9, 10, 11, 12, 13, 14, 15, 16, 17, 18, 19, 20, 21, 22, 23, 24, 25, 26, 27, 28, 29, or 30 nucleotides in length. In some embodiments, a stem comprises a duplex of 1, 2, 3, 4, 5, 6, 7, 8, 9, 10, 11, 12, 13, or 14 nucleotides in length. In some embodiments, a stem-loop provides the molecule better protection against degradation (e.g., enzymatic degradation) and facilitates targeting characteristics for delivery to a target cell. For example, in some embodiments, a loop provides added nucleotides on which modification can be made without substantially affecting the gene expression inhibition activity of an oligonucleotide. In certain embodiments, an oligonucleotide is provided herein in which the sense strand comprises (e.g., at its 3′-end) a stem-loop set forth as: S₁-L-S₂, in which S₁ is complementary to S₂, and in which L forms a loop between S₁ and S₂ of up to 10 nucleotides in length (e.g., 1, 2, 3, 4, 5, 6, 7, 8, 9, or 10 nucleotides in length).

In some embodiments, a loop (L) of a stem-loop is a tetraloop (e.g., within a nicked tetraloop structure). A tetraloop may contain ribonucleotides, deoxyribonucleotides, modified nucleotides, and combinations thereof. Typically, a tetraloop has 4 to 5 nucleotides. However, in some embodiments, a tetraloop comprises or consists of 3 to 6 nucleotides, and typically consists of 4 to 5 nucleotides. In certain embodiments, a tetraloop comprises or consists of three, four, five, or six nucleotides.

iv. Oligonucleotide Modifications

Oligonucleotides may be modified in various ways to improve or control specificity, stability, delivery, bioavailability, resistance from nuclease degradation, immunogenicity, base-paring properties, RNA distribution and cellular uptake and other features relevant to therapeutic or research use (see, e.g., Bramsen et al., Nucleic Acids Res., 2009, 37:2867-2881; Bramsen and Kjems, Frontiers in Genetics, 2012, 3:1-22). Accordingly, in some embodiments, oligonucleotides of the present disclosure may include one or more suitable modifications. In some embodiments, a modified nucleotide has a modification in its base (or nucleobase), the sugar (e.g., ribose, deoxyribose), or the phosphate group.

The number of modifications on an oligonucleotide and the positions of those nucleotide modifications may influence the properties of an oligonucleotide. For example, oligonucleotides maybe be delivered in vivo by conjugating them to or encompassing them in a lipid nanoparticle (LNP) or similar carrier. However, when an oligonucleotide is not protected by an LNP or similar carrier, it may be advantageous for at least some of the nucleotides to be modified. Accordingly, in certain embodiments of any of the oligonucleotides provided herein, all or substantially all the nucleotides of an oligonucleotide are modified. In certain embodiments, more than half of the nucleotides are modified. In certain embodiments, less than half of the nucleotides are modified. Typically, with naked delivery, every sugar is modified at the 2′-position. These modifications may be reversible or irreversible. In some embodiments, an oligonucleotide as disclosed herein has a number and type of modified nucleotides sufficient to cause the desired characteristic (e.g., protection from enzymatic degradation, capacity to target a desired cell after in vivo administration, and/or thermodynamic stability).

a. Sugar Modifications

In some embodiments, a modified sugar (also referred herein to a sugar analog) includes a modified deoxyribose or ribose moiety, e.g., in which one or more modifications occur at the 2′, 3′, 4′, and/or 5′ carbon position of the sugar. In some embodiments, a modified sugar may also include non-natural alternative carbon structures such as those present in locked nucleic acids (“LNA”) (see, e.g., Koshkin et al., Tetrahedron, 1998, 54:3607-3630), unlocked nucleic acids (“UNA”) (see, e.g., Snead et al., Molecular Therapy—Nucleic Acids, 2013, 2, e103), and bridged nucleic acids (“BNA”) (see, e.g., Imanishi and Obika, The Royal Society of Chemistry, Chem. Commun., 2002, 16:1653-1659). Koshkin et al., Snead et al., and Imanishi and Obika are incorporated by reference herein for their disclosures relating to sugar modifications.

In some embodiments, a nucleotide modification in a sugar comprises a 2′-modification. A 2′-modification may be 2′-aminoethyl, 2′-fluoro, 2′-O-methyl, 2′-O-methoxyethyl, and 2′-deoxy-2′-fluoro-β-d-arabinonucleic acid. Typically, the modification is 2′-fluoro, 2′-O-methyl, or 2′-O-methoxyethyl. In some embodiments a modification in a sugar comprises a modification of the sugar ring, which may comprise modification of one or more carbons of the sugar ring. For example, a modification of a sugar of a nucleotide may comprise a 2′-oxygen of a sugar is linked to a 1′-carbon or 4′-carbon of the sugar, or a 2′-oxygen is linked to the 1′-carbon or 4′-carbon via an ethylene or methylene bridge. In some embodiments, a modified nucleotide has an acyclic sugar that lacks a 2′-carbon to 3′-carbon bond. In some embodiments, a modified nucleotide has a thiol group, e.g., in the 4′ position of the sugar.

In some embodiments, the oligonucleotide described herein comprises at least one modified nucleotide (e.g., at least 1, at least 5, at least 10, at least 15, at least 20, at least 25, at least 30, at least 35, at least 40, at least 45, at least 50, at least 55, at least 60, or more). In some embodiments, the sense strand of the oligonucleotide comprises at least one modified nucleotide (e.g., at least 1, at least 5, at least 10, at least 15, at least 20, at least 25, at least 30, at least 35, or more). In some embodiments, the antisense strand of the oligonucleotide comprises at least one modified nucleotide (e.g., at least 1, at least 5, at least 10, at least 15, at least 20, or more).

In some embodiments, all the nucleotides of the sense strand of the oligonucleotide are modified. In some embodiments, all the nucleotides of the antisense strand of the oligonucleotide are modified. In some embodiments, all the nucleotides of the oligonucleotide (i.e., both the sense strand and the antisense strand) are modified. In some embodiments, the modified nucleotide comprises a 2′-modification (e.g., a 2′-fluoro or 2′-O-methyl).

The present disclosure provides oligonucleotides having different modification patterns. In some embodiments, the modified oligonucleotides comprise a sense strand sequence having a sequence as set forth in any one of SEQ ID NOs: 27-39, and an antisense strand sequence having a sequence as set forth in any one of SEQ ID NOs: 14-26. In some embodiments, for these oligonucleotides, one or more of positions 1, 2, 4, 6, 7, 12, 14, 16, 18-27, and 31-36 of the sense strand, and/or one or more of positions 1, 4, 6, 8, 9, 11, 13, 15, 18, and 20-22 of the antisense strand are modified with a 2′-O-methyl. In some embodiments, all of positions 1, 2, 4, 6, 7, 12, 14, 16, 18-27, and 31-36 of the sense strand, and all of positions 1, 4, 6, 8, 9, 11, 13, 15, 18, and 20-22 of the antisense strand are modified with a 2′-O-methyl. In some embodiments, one or more of positions 3, 5, 8-11, 13, 15, and 17 of the sense strand, and/or one or more of positions 2, 3, 5, 7, 10, 12, 14, 16, 17, and 19 of the antisense strand are modified with a 2′-fluoro. In some embodiments, all of positions 3, 5, 8-11, 13, 15, and 17 of the sense strand, and all of positions 2, 3, 5, 7, 10, 12, 14, 16, 17, and 19 of the antisense strand are modified with a 2′-fluoro. In some embodiments, all of positions 1, 2, 4, 6, 7, 12, 14, 16, 18-27, and 31-36 of the sense strand, all of positions 1, 4, 6, 8, 9, 11, 13, 15, 18, and 20-22 of the antisense strand are modified with a 2′-O-methyl, all of positions 3, 5, 8-11, 13, 15, and 17 of the sense strand, and all of positions 2, 3, 5, 7, 10, 12, 14, 16, 17, and 19 of the antisense strand are modified with a 2′-fluoro.

In some embodiments, for oligonucleotides comprising a sense strand having a sequence as set forth in any one of SEQ ID NOs: 27-39, and an antisense strand having a sequence as set forth in any one of SEQ ID NOs: 14-26, one or more of positions 1-7, 12-27, and 31-36 of the sense strand, and/or one or more of positions 1, 4, 6, 8, 9, 11-13, and 15-22 of the antisense strand are modified with a 2′-O-methyl. In some embodiments, all of positions 1-7, 12-27, and 31-36 of the sense strand, and all of positions 1, 4, 6, 8, 9, 11-13, and 15-22 of the antisense strand are modified with a 2′-O-methyl. In some embodiments, one or more of positions 8-11 of the sense strand, and/or one or more of positions 2, 3, 5, 7, 10, and 14 of the antisense strand are modified with a 2′-fluoro. In some embodiments, all of positions 8-11 of the sense strand, and all of positions 2, 3, 5, 7, 10, and 14 of the antisense strand are modified with a 2′-fluoro. In some embodiments, all of positions 1-7, 12-27, and 31-36 of the sense strand, all of positions 1, 4, 6, 8, 9, 11-13, and 15-22 of the antisense strand are modified with a 2′-O-methyl, all of positions 8-11 of the sense strand, and all of positions 2, 3, 5, 7, 10, and 14 of the antisense strand are modified with a 2′-fluoro.

In some embodiments, for oligonucleotides comprising a sense strand having a sequence as set forth in any one of SEQ ID NOs: 27-39, and an antisense strand having a sequence as set forth in any one of SEQ ID NOs: 14-26, one or more of positions 1, 2, 4-7, 9, 11, 14-16, 18-27, and 31-36 of the sense strand, and/or one or more of positions 1, 6, 8, 9, 11, 13, 15, 18, and 20-22 of the antisense strand are modified with a 2′-O-methyl. In some embodiments, all of positions 1, 2, 4-7, 9, 11, 14-16, 18-27, and 31-36 of the sense strand, and all of positions 1, 6, 8, 9, 11, 13, 15, 18, and 20-22 of the antisense strand are modified with a 2′-O-methyl. In some embodiments, one or more of positions 3, 8, 10, 12, 13, and 17 of the sense strand, and/or one or more of positions 2-5, 7, 10, 12, 14, 16, 17, and 19 of the antisense strand are modified with a 2′-fluoro. In some embodiments, all of positions 3, 8, 10, 12, 13, and 17 of the sense strand, and all of positions 2-5, 7, 10, 12, 14, 16, 17, and 19 of the antisense strand are modified with a 2′-fluoro.

In some embodiments, the terminal 3′-end group (e.g., a 3′-hydroxyl) with a phosphate group or other group, which can be used, for example, to attach linkers, adapters or labels or for the direct ligation of an oligonucleotide to another nucleic acid.

b. 5′ Terminal Phosphates

In some embodiments, 5′-terminal phosphate groups of oligonucleotides enhance the interaction with Argonaute 2. However, oligonucleotides comprising a 5′-phosphate group may be susceptible to degradation via phosphatases or other enzymes, which can limit their bioavailability in vivo. In some embodiments, oligonucleotides include analogs of 5′ phosphates that are resistant to such degradation. In some embodiments, a phosphate analog may be oxymethylphosphonate, vinylphosphonate, or malonylphosphonate. In certain embodiments, the 5′ end of an oligonucleotide strand is attached to chemical moiety that mimics the electrostatic and steric properties of a natural 5′-phosphate group (“phosphate mimic”) (see, e.g., Prakash et al., Nucleic Acids Res., 2015, 43(6):2993-3011, the contents of which relating to phosphate analogs are incorporated herein by reference). Many phosphate mimics have been developed that can be attached to the 5′ end (see, e.g., U.S. Pat. No. 8,927,513, the contents of which relating to phosphate analogs are incorporated herein by reference). Other modifications have been developed for the 5′ end of oligonucleotides (see, e.g., International patent publication WO2011133871, the contents of which relating to phosphate analogs are incorporated herein by reference). In certain embodiments, a hydroxyl group is attached to the 5′ end of the oligonucleotide.

In some embodiments, an oligonucleotide has a phosphate analog at a 4′-carbon position of the sugar (referred to as a “4′-phosphate analog”) (see, e.g., International patent publication WO2018045317, entitled 4′-Phosphate Analogs and Oligonucleotides Comprising the Same, which content relating to phosphate analogs is incorporated herein by reference). In some embodiments, an oligonucleotide provided herein comprise a 4′-phosphate analog at a 5′-terminal nucleotide. In some embodiments, a phosphate analog is an oxymethylphosphonate in which the oxygen atom of the oxymethyl group is bound to the sugar moiety (e.g., at its 4′-carbon) or analog thereof. In other embodiments, a 4′-phosphate analog is a thiomethylphosphonate or an aminomethylphosphonate in which the sulfur atom of the thiomethyl group or the nitrogen atom of the aminomethyl group is bound to the 4′-carbon of the sugar moiety or analog thereof. In certain embodiments, a 4′-phosphate analog is an oxymethylphosphonate. In some embodiments, an oxymethylphosphonate is represented by the formula —O—CH₂—PO(OH)₂ or —O—CH₂—PO(OR)₂, in which R is independently selected from H, CH₃, an alkyl group, CH₂CH₂CN, CH₂OCOC(CH₃)₃, CH₂OCH₂CH₂Si(CH₃)₃, or a protecting group. In certain embodiments, the alkyl group is CH₂CH₃. More typically, R is independently selected from H, CH₃, or CH₂CH₃.

In certain embodiments, a phosphate analog attached to the oligonucleotide is a methoxy phosphonate (MOP). In certain embodiments, a phosphate analog attached to the oligonucleotide is a 5′ mono-methyl protected MOP. In some embodiments, the following uridine nucleotide comprising a phosphate analog may be used, e.g., at the first position of a guide (antisense) strand:

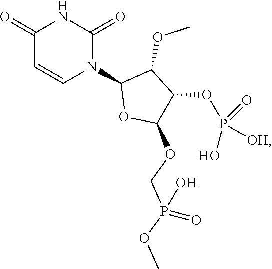 [SMILES: CO[C@@H]1[C@H](OP(=O)(O)O)[C@@H](OCP(=O)(O)OC)O[C@H]1n1ccc(=O)[nH]c1=O]

which modified nucleotide is referred to as [MePhosphonate-4O-mU] or 5′-Methoxy, Phosphonate-4′oxy-2′-O-methyluridine.

c. Modified Intranucleotide Linkages

In some embodiments, phosphate modifications or substitutions may result in an oligonucleotide comprises at least one (e.g., at least 1, at least 2, at least 3 or at least 5) comprising a modified internucleotide linkage. In some embodiments, any one of the oligonucleotides disclosed herein comprises 1 to 10 (e.g., 1 to 10, 2 to 8, 4 to 6, 3 to 10, 5 to 10, 1 to 5, 1 to 3 or 1 to 2) modified internucleotide linkages. In some embodiments, any one of the oligonucleotides disclosed herein comprises 1, 2, 3, 4, 5, 6, 7, 8, 9, or 10 modified internucleotide linkages.

A modified internucleotide linkage may be a phosphorodithioate linkage, a phosphorothioate linkage, a phosphotriester linkage, a thionoalkylphosphonate linkage, a thionalkylphosphotriester linkage, a phosphoramidite linkage, a phosphonate linkage or a boranophosphate linkage. In some embodiments, at least one modified internucleotide linkage of any one of the oligonucleotides as disclosed herein is a phosphorothioate linkage.

In some embodiments, the oligonucleotide described herein has a phosphorothioate linkage between one or more of: positions 1 and 2 of the sense strand, positions 1 and 2 of the antisense strand, positions 2 and 3 of the antisense strand, positions 3 and 4 of the antisense strand, positions 20 and 21 of the antisense strand, and positions 21 and 22 of the antisense strand. In some embodiments, the oligonucleotide described herein has a phosphorothioate linkage between each of: positions 1 and 2 of the sense strand, positions 1 and 2 of the antisense strand, positions 2 and 3 of the antisense strand, positions 20 and 21 of the antisense strand, and positions 21 and 22 of the antisense strand.

d. Base Modifications

In some embodiments, oligonucleotides provided herein have one or more modified nucleobases. In some embodiments, modified nucleobases (also referred to herein as base analogs) are linked at the 1′ position of a nucleotide sugar moiety. In certain embodiments, a modified nucleobase is a nitrogenous base. In certain embodiments, a modified nucleobase does not contain nitrogen atom (see, e.g., U.S. patent publication 20080274462). In some embodiments, a modified nucleotide comprises a universal base. However, in certain embodiments, a modified nucleotide does not contain a nucleobase (abasic).

In some embodiments a universal base is a heterocyclic moiety located at the 1′ position of a nucleotide sugar moiety in a modified nucleotide, or the equivalent position in a nucleotide sugar moiety substitution, that, when present in a duplex, can be positioned opposite more than one type of base without substantially altering structure of the duplex. In some embodiments, compared to a reference single-stranded nucleic acid (e.g., oligonucleotide) that is fully complementary to a target nucleic acid, a single-stranded nucleic acid containing a universal base forms a duplex with the target nucleic acid that has a lower Tₘ than a duplex formed with the complementary nucleic acid. However, in some embodiments, compared to a reference single-stranded nucleic acid in which the universal base has been replaced with a base to generate a single mismatch, the single-stranded nucleic acid containing the universal base forms a duplex with the target nucleic acid that has a higher Tₘ than a duplex formed with the nucleic acid comprising the mismatched base.

Non-limiting examples of universal-binding nucleotides include inosine, 1-β-D-ribofuranosyl-5-nitroindole, and/or 1-β-D-ribofuranosyl-3-nitropyrrole (U.S. patent publication no. 20070254362; Van Aerschot et al., Nucleic Acids Res. 1995, 23(21):4363-70; Loakes et al., Nucleic Acids Res. 1995, 23(13):2361-6; Loakes and Brown, Nucleic Acids Res., 1994, 22(20):4039-43. Each of the foregoing is incorporated by reference herein for their disclosures relating to base modifications).

e. Reversible Modifications

While certain modifications to protect an oligonucleotide from the in vivo environment before reaching target cells can be made, they can reduce the potency or activity of the oligonucleotide once it reaches the cytosol of the target cell. Reversible modifications can be made such that the molecule retains desirable properties outside of the cell, which are then removed upon entering the cytosolic environment of the cell. Reversible modification can be removed, for example, by the action of an intracellular enzyme or by the chemical conditions inside of a cell (e.g., through reduction by intracellular glutathione).

In some embodiments, a reversibly modified nucleotide comprises a glutathione-sensitive moiety. Typically, nucleic acid molecules have been chemically modified with cyclic disulfide moieties to mask the negative charge created by the internucleotide diphosphate linkages and improve cellular uptake and nuclease resistance (see, e.g., U.S. patent publication 20110294869, International patent publication WO2015188197; Meade et al., Nature Biotechnology, 2014, 32:1256-1263; International patent publication WO2014088920; each of which are incorporated by reference for their disclosures of such modifications). This reversible modification of the internucleotide diphosphate linkages is designed to be cleaved intracellularly by the reducing environment of the cytosol (e.g., glutathione). Earlier examples include neutralizing phosphotriester modifications that were reported to be cleavable inside cells (Dellinger et al., J. Am. Chem. Soc., 2003, 125:940-950).

In some embodiments, such a reversible modification allows protection during in vivo administration (e.g., transit through the blood and/or lysosomal/endosomal compartments of a cell) where the oligonucleotide will be exposed to nucleases and other harsh environmental conditions (e.g., pH). When released into the cytosol of a cell where the levels of glutathione are higher compared to extracellular space, the modification is reversed and the result is a cleaved oligonucleotide. Using reversible, glutathione sensitive moieties, it is possible to introduce sterically larger chemical groups into the oligonucleotide of interest as compared to the options available using irreversible chemical modifications. This is because these larger chemical groups will be removed in the cytosol and, therefore, should not interfere with the biological activity of the oligonucleotides inside the cytosol of a cell. As a result, these larger chemical groups can be engineered to confer various advantages to the nucleotide or oligonucleotide, such as nuclease resistance, lipophilicity, charge, thermal stability, specificity, and reduced immunogenicity. In some embodiments, the structure of the glutathione-sensitive moiety can be engineered to modify the kinetics of its release.

In some embodiments, a glutathione-sensitive moiety is attached to the sugar of the nucleotide. In some embodiments, a glutathione-sensitive moiety is attached to the 2′carbon of the sugar of a modified nucleotide. In some embodiments, the glutathione-sensitive moiety is located at the 5′-carbon of a sugar, particularly when the modified nucleotide is the 5′-terminal nucleotide of the oligonucleotide. In some embodiments, the glutathione-sensitive moiety is located at the 3′-carbon of sugar, particularly when the modified nucleotide is the 3′-terminal nucleotide of the oligonucleotide. In some embodiments, the glutathione-sensitive moiety comprises a sulfonyl group (see, e.g., International patent publication WO2018039364, the contents of which are incorporated by reference herein for its relevant disclosures).

v. Targeting Ligands

In some embodiments, it may be desirable to target the oligonucleotides of the disclosure to one or more cells or one or more organs. Such a strategy may help to avoid undesirable effects in other organs or may avoid undue loss of the oligonucleotide to cells, tissue or organs that would not benefit for the oligonucleotide. Accordingly, in some embodiments, oligonucleotides disclosed herein may be modified to facilitate targeting of a particular tissue, cell or organ, e.g., to facilitate delivery of the oligonucleotide to the liver. In certain embodiments, oligonucleotides disclosed herein may be modified to facilitate delivery of the oligonucleotide to the hepatocytes of the liver. In some embodiments, an oligonucleotide comprises a nucleotide that is conjugated to one or more targeting ligand.

A targeting ligand may comprise a carbohydrate, amino sugar, cholesterol, peptide, polypeptide, protein or part of a protein (e.g., an antibody or antibody fragment) or lipid. In some embodiments, a targeting ligand is an aptamer. For example, a targeting ligand may be an RGD peptide that is used to target tumor vasculature or glioma cells, CREKA peptide to target tumor vasculature or stoma, transferring, lactoferrin, or an aptamer to target transferrin receptors expressed on CNS vasculature, or an anti-EGFR antibody to target EGFR on glioma cells. In certain embodiments, the targeting ligand is one or more GalNAc moieties.

In some embodiments, 1 or more (e.g., 1, 2, 3, 4, 5 or 6) nucleotides of an oligonucleotide are each conjugated to a separate targeting ligand. In some embodiments, 2 to 4 nucleotides of an oligonucleotide are each conjugated to a separate targeting ligand. In some embodiments, targeting ligands are conjugated to 2 to 4 nucleotides at either ends of the sense or antisense strand (e.g., ligands are conjugated to a 2 to 4 nucleotide overhang or extension on the 5′ or 3′ end of the sense or antisense strand) such that the targeting ligands resemble bristles of a toothbrush and the oligonucleotide resembles a toothbrush. For example, an oligonucleotide may comprise a stem-loop at either the 5′ or 3′ end of the sense strand and 1, 2, 3 or 4 nucleotides of the loop of the stem may be individually conjugated to a targeting ligand.

In some embodiments, it is desirable to target an oligonucleotide that reduces the expression of HMGB1 to the hepatocytes of the liver of the subject. Any suitable hepatocyte targeting moiety may be used for this purpose.

GalNAc is a high affinity ligand for asialoglycoprotein receptor (ASGPR), which is primarily expressed on the sinusoidal surface of hepatocyte cells and has a major role in binding, internalization, and subsequent clearance of circulating glycoproteins that contain terminal galactose or N-acetylgalactosamine residues (asialoglycoproteins). Conjugation (either indirect or direct) of GalNAc moieties to oligonucleotides of the instant disclosure may be used to target these oligonucleotides to the ASGPR expressed on these hepatocyte cells.

In some embodiments, an oligonucleotide of the instant disclosure is conjugated directly or indirectly to a monovalent GalNAc. In some embodiments, the oligonucleotide is conjugated directly or indirectly to more than one monovalent GalNAc (e.g., is conjugated to 2, 3, or 4 monovalent GalNAc moieties, and is typically conjugated to 3 or 4 monovalent GalNAc moieties). In some embodiments, an oligonucleotide of the instant disclosure is conjugated to a one or more bivalent GalNAc, trivalent GalNAc, or tetravalent GalNAc moieties.

In some embodiments, 1 or more (e.g., 1, 2, 3, 4, 5 or 6) nucleotides of an oligonucleotide are each conjugated to a GalNAc moiety. In some embodiments, 2 to 4 nucleotides of the loop (L) of the stem-loop are each conjugated to a separate GalNAc. In some embodiments, targeting ligands are conjugated to 2 to 4 nucleotides at either ends of the sense or antisense strand (e.g., ligands are conjugated to a 2 to 4 nucleotide overhang or extension on the 5′ or 3′ end of the sense or antisense strand) such that the GalNAc moieties resemble bristles of a toothbrush and the oligonucleotide resembles a toothbrush. For example, an oligonucleotide may comprise a stem-loop at either the 5′ or 3′ end of the sense strand and 1, 2, 3 or 4 nucleotides of the loop of the stem may be individually conjugated to a GalNAc moiety. In some embodiments, GalNAc moieties are conjugated to a nucleotide of the sense strand. For example, four GalNAc moieties can be conjugated to nucleotides in the tetraloop of the sense strand where each GalNAc moiety is conjugated to one nucleotide.

In some embodiments, an oligonucleotide herein comprises a monovalent GalNAc attached to a Guanidine nucleotide, referred to as [ademG-GalNAc] or 2′-aminodiethoxymethanol-Guanidine-GalNAc, as depicted below:

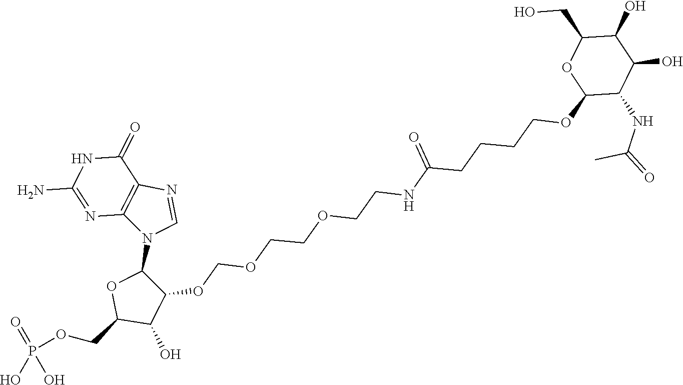 [SMILES: CC(=O)N[C@@H]1[C@@H](OCCCCC(=O)NCCOCCOCO[C@@H]2[C@H](O)[C@@H](COP(=O)(O)O)O[C@H]2n2cnc3c(=O)[nH]c(N)nc32)O[C@@H](CO)[C@@H](O)[C@H]1O]

In some embodiments, an oligonucleotide herein comprises a monovalent GalNAc attached to an adenine nucleotide, referred to as [ademA-GalNAc] or 2′-aminodiethoxymethanol-Adenine-GalNAc, as depicted below.

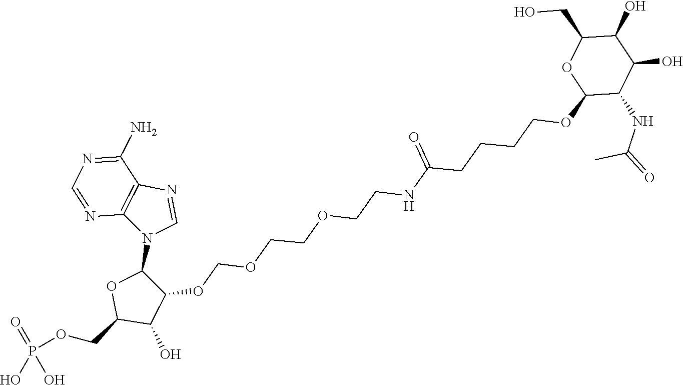

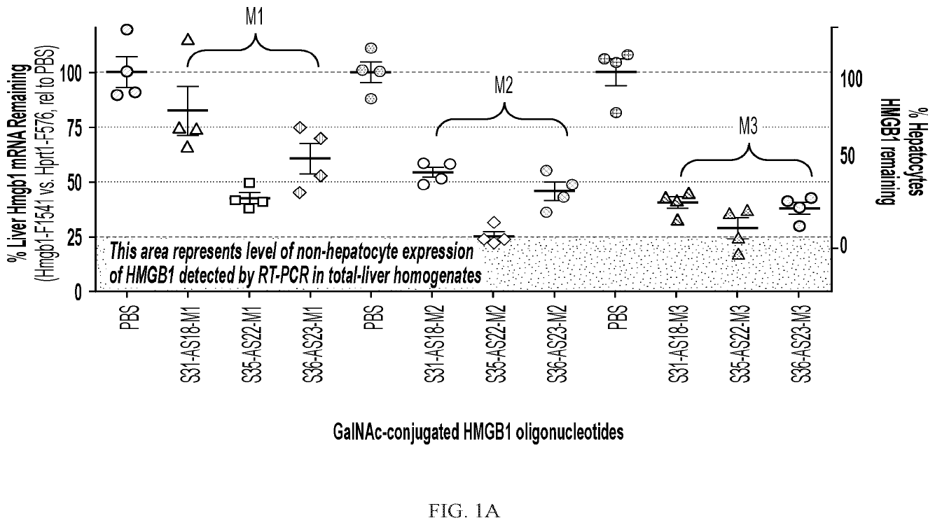

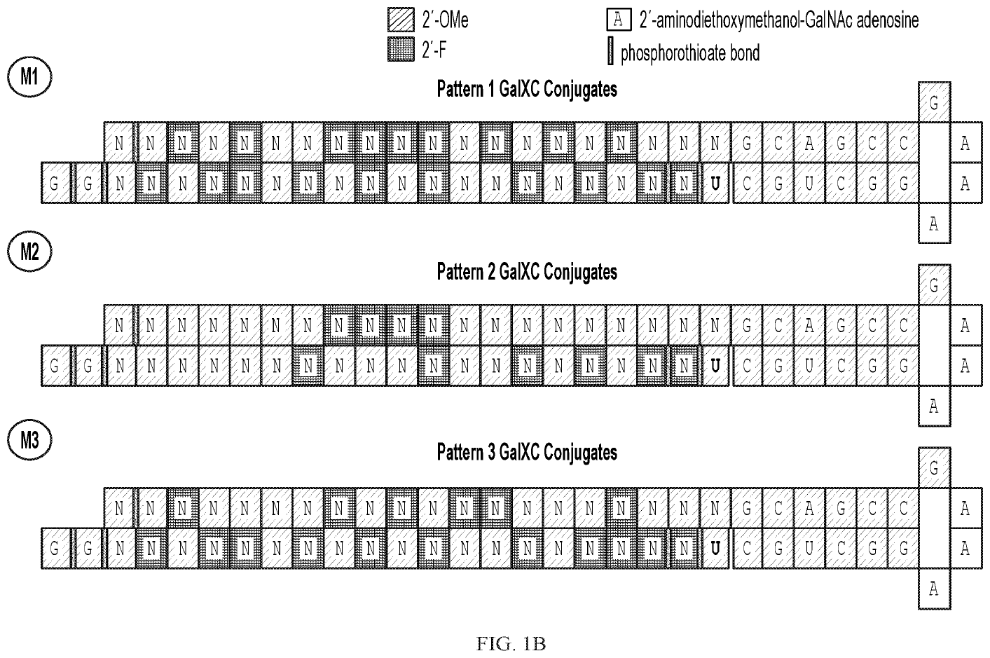

An example of such conjugation is shown below for a loop comprising from 5′ to 3′ the nucleotide sequence GAAA (L=linker, X=heteroatom) stem attachment points are shown. Such a loop may be present, for example, at positions 27-30 of the molecules shown in FIG. 1B. In the chemical formula,

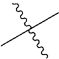

is used to describe an attachment point to the oligonucleotide strand.

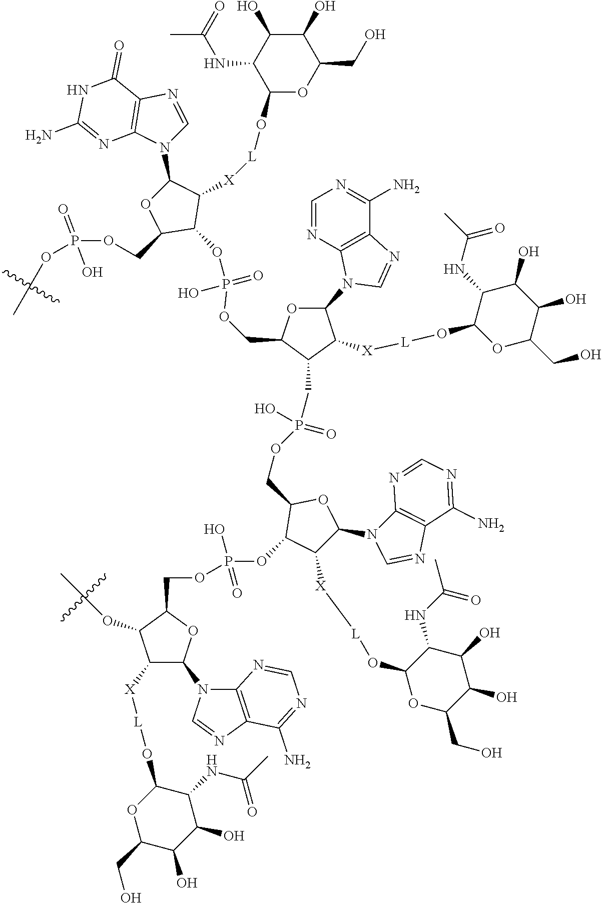

Appropriate methods or chemistry (e.g., click chemistry) can be used to link a targeting ligand to a nucleotide. In some embodiments, a targeting ligand is conjugated to a nucleotide using a click linker. In some embodiments, an acetal-based linker is used to conjugate a targeting ligand to a nucleotide of any one of the oligonucleotides described herein. Acetal-based linkers are disclosed, for example, in International patent publication WO2016100401, the contents of which relating to such linkers are incorporated herein by reference. In some embodiments, the linker is a labile linker. However, in other embodiments, the linker is stable. A “labile linker” refers to a linker that can be cleaved, e.g., by acidic pH. A “stable linker” refers to a linker that cannot be cleaved.

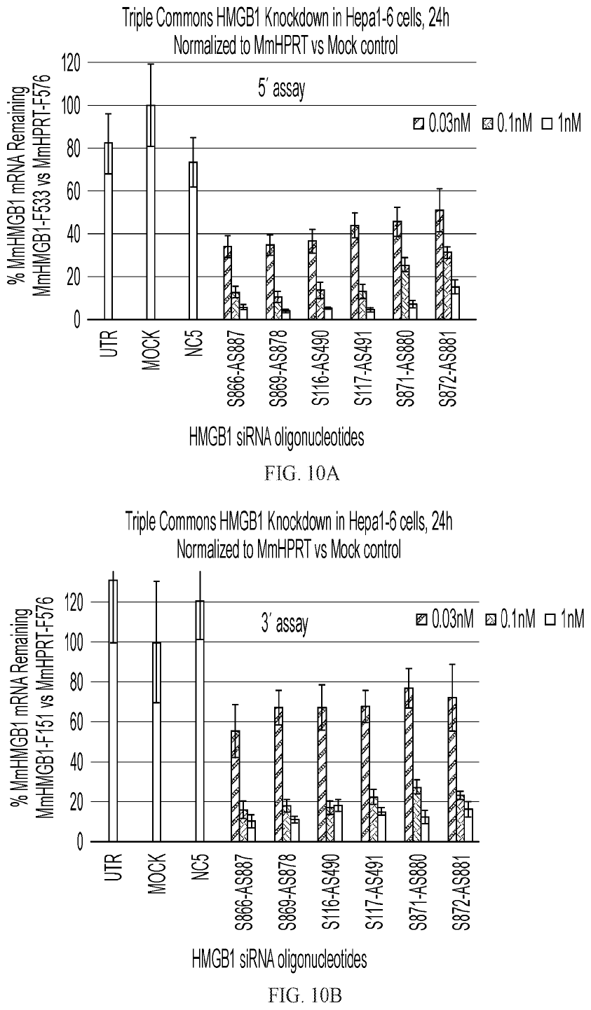

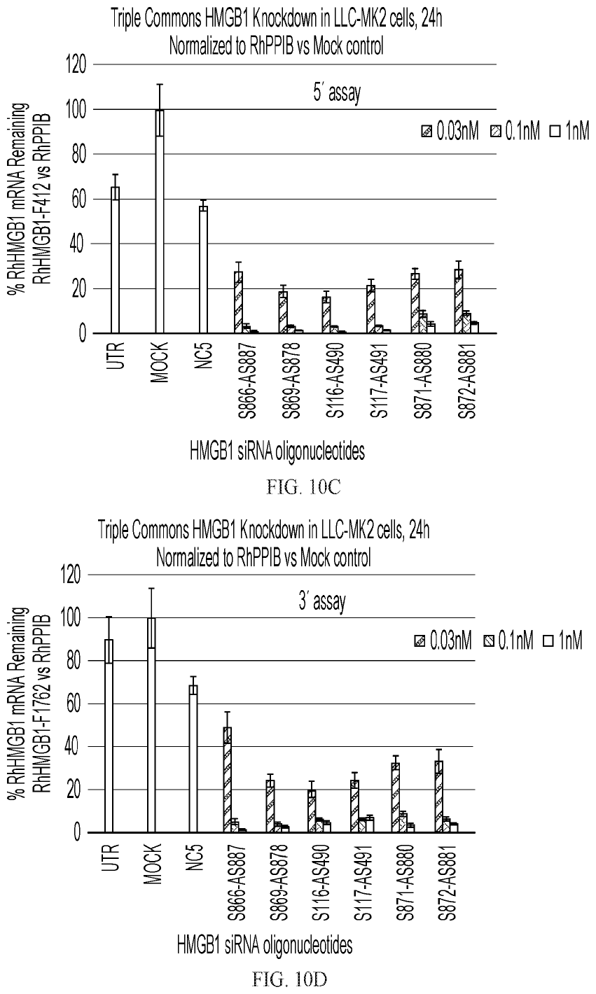

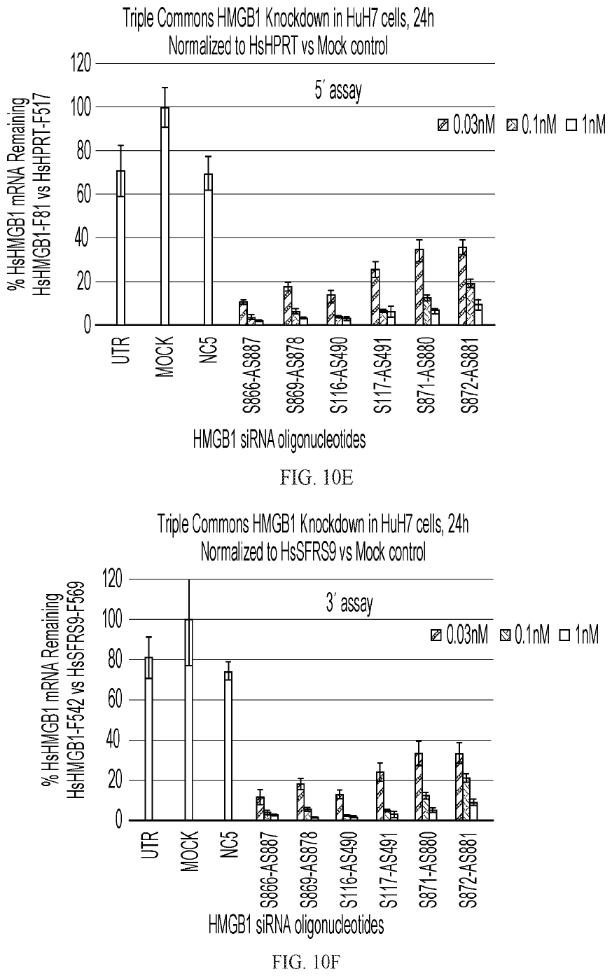

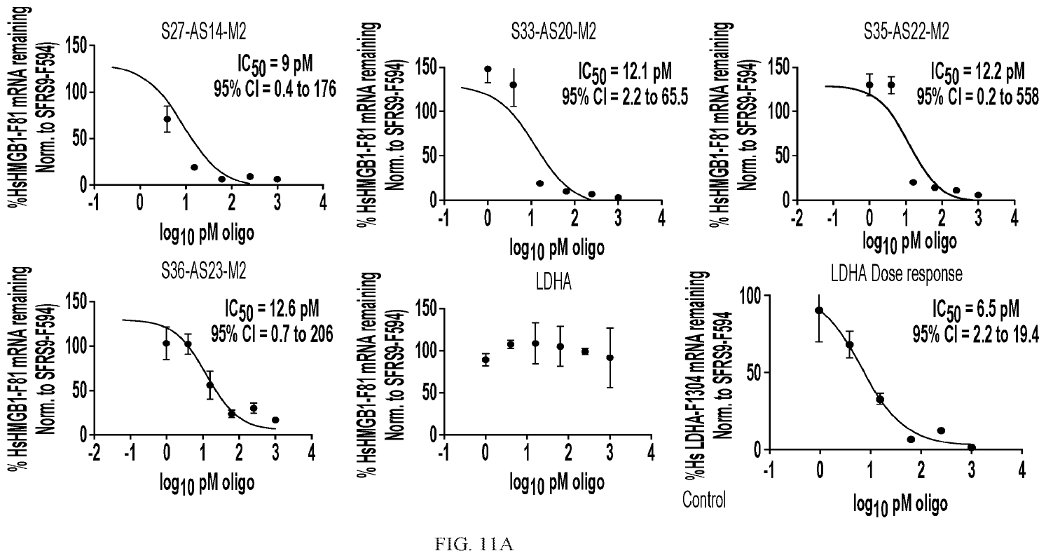

An example is shown below for a loop comprising from 5′ to 3′ the nucleotides GAAA, in which GalNAc moieties are attached to nucleotides of the loop using an acetal linker. Such a loop may be present, for example, at positions 27-30 of the molecules described in FIG. 10. In the chemical formula,

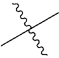

is an attachment point to the oligonucleotide strand.

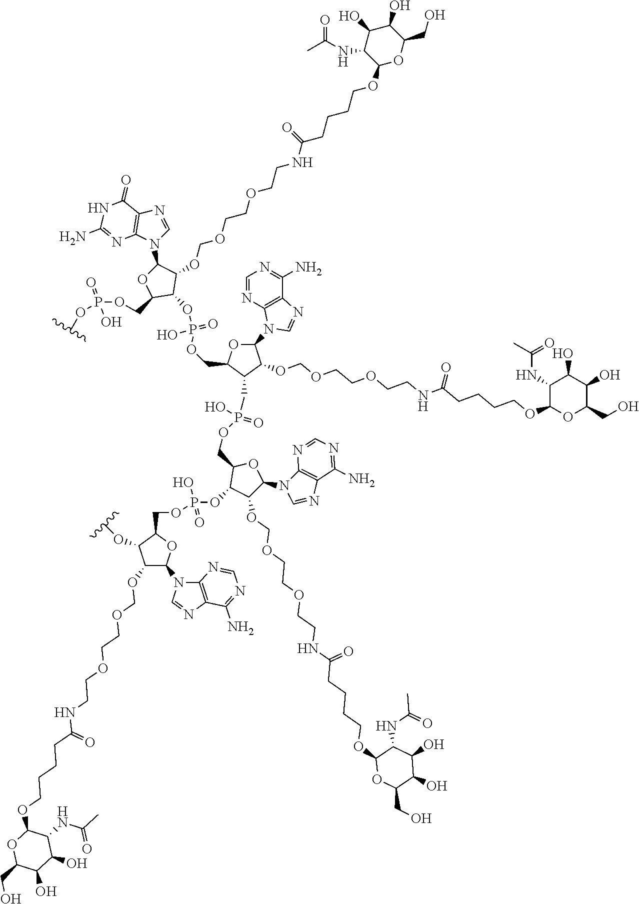 [SMILES: *O[C@H]1[C@@H](OCOCCOCCNC(=O)CCCCO[C@@H]2O[C@H](CO)[C@H](O)[C@H](O)[C@H]2NC(C)=O)[C@H](n2cnc3c(N)ncnc32)O[C@@H]1COP(=O)(O)O[C@H]1[C@@H](OCOCCOCCNC(=O)CCCCO[C@@H]2O[C@H](CO)[C@H](O)[C@H](O)[C@H]2NC(C)=O)[C@H](n2cnc3c(N)ncnc32)O[C@@H]1COP(=O)(O)C[C@H]1[C@@H](OCOCCOCCNC(=O)CCCCO[C@@H]2O[C@H](CO)[C@H](O)[C@H](O)[C@H]2NC(C)=O)[C@H](n2cnc3c(N)ncnc32)O[C@@H]1COP(=O)(O)O[C@H]1[C@@H](OCOCCOCCNC(=O)CCCCO[C@@H]2O[C@H](CO)[C@H](O)[C@H](O)[C@H]2NC(C)=O)[C@H](n2cnc3c(=O)[nH]c(N)nc32)O[C@@H]1COP(=O)(O)O*]

Any appropriate method or chemistry (e.g., click chemistry) can be used to link a targeting ligand to a nucleotide. In some embodiments, a targeting ligand is conjugated to a nucleotide using a click linker. In some embodiments, an acetal-based linker is used to conjugate a targeting ligand to a nucleotide of any one of the oligonucleotides described herein. Acetal-based linkers are disclosed, for example, in International patent publication WO2016100401, the contents of which relating to such linkers are incorporated herein by reference. In some embodiments, the linker is a labile linker. In other embodiments, the linker is stable.

In some embodiments, a duplex extension (e.g., of up to 3, 4, 5, or 6 base pairs in length) is provided between a targeting ligand (e.g., a GalNAc moiety) and a double-stranded oligonucleotide.

### III. Formulations

Various formulations have been developed to facilitate oligonucleotide use. For example, oligonucleotides can be delivered to a subject or a cellular environment using a formulation that minimizes degradation, facilitates delivery and/or uptake, or provides another beneficial property to the oligonucleotides in the formulation. In some embodiments, provided herein are compositions comprising oligonucleotides (e.g., single-stranded or double-stranded oligonucleotides) to reduce the expression of HMGB1. Such compositions can be suitably formulated such that when administered to a subject, either into the immediate environment of a target cell or systemically, a sufficient portion of the oligonucleotides enter the cell to reduce HMGB1 expression. Any of a variety of suitable oligonucleotide formulations can be used to deliver oligonucleotides for the reduction of HMGB1 as disclosed herein. In some embodiments, an oligonucleotide is formulated in buffer solutions such as phosphate buffered saline solutions, liposomes, micellar structures, and capsids.

Formulations of oligonucleotides with cationic lipids can be used to facilitate transfection of the oligonucleotides into cells. For example, cationic lipids, such as lipofectin, cationic glycerol derivatives, and polycationic molecules (e.g., polylysine, can be used. Suitable lipids include Oligofectamine, Lipofectamine (Life Technologies), NC388 (Ribozyme Pharmaceuticals, Inc., Boulder, Colo.), or FuGene 6 (Roche) all of which can be used according to the manufacturer's instructions.

Accordingly, in some embodiments, a formulation comprises a lipid nanoparticle. In some embodiments, an excipient comprises a liposome, a lipid, a lipid complex, a microsphere, a microparticle, a nanosphere, or a nanoparticle, or may be otherwise formulated for administration to the cells, tissues, organs, or body of a subject in need thereof (see, e.g., Remington: The Science and Practice of Pharmacy, 22nd Ed., Pharmaceutical Press, 2013).

In some embodiments, formulations as disclosed herein comprise an excipient. In some embodiments, an excipient confers to a composition improved stability, improved absorption, improved solubility and/or therapeutic enhancement of the active ingredient. In some embodiments, an excipient is a buffering agent (e.g., sodium citrate, sodium phosphate, a tris base, or sodium hydroxide) or a vehicle (e.g., a buffered solution, petrolatum, dimethyl sulfoxide, or mineral oil). In some embodiments, an oligonucleotide is lyophilized for extending its shelf-life and then made into a solution before use (e.g., administration to a subject). Accordingly, an excipient in a composition comprising any one of the oligonucleotides described herein may be a lyoprotectant (e.g., mannitol, lactose, polyethylene glycol, or polyvinyl pyrolidone), or a collapse temperature modifier (e.g., dextran, ficoll, or gelatin).

In some embodiments, a pharmaceutical composition is formulated to be compatible with its intended route of administration. Examples of routes of administration include parenteral, e.g., intravenous, intradermal, subcutaneous, oral (e.g., inhalation), transdermal (topical), transmucosal, and rectal administration.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J., USA) or phosphate buffered saline (PBS). The carrier can be a solvent or dispersion medium containing, for example, water, ethanol, polyol (for example, glycerol, propylene glycol, and liquid polyetheylene glycol, and the like), and suitable mixtures thereof. In many cases, it will be preferable to include isotonic agents, for example, sugars, polyalcohols such as mannitol, sorbitol, sodium chloride in the composition. Sterile injectable solutions can be prepared by incorporating the oligonucleotides in a required amount in a selected solvent with one or a combination of ingredients enumerated above, as required, followed by filtered sterilization.

In some embodiments, a composition may contain at least about 0.1% of the therapeutic agent (e.g., an oligonucleotide for reducing HMGB1 expression) or more, although the percentage of the active ingredient(s) may be about 1% to about 80% or more of the weight or volume of the total composition. Factors such as solubility, bioavailability, biological half-life, route of administration, product shelf life, as well as other pharmacological considerations will be contemplated by one skilled in the art of preparing such pharmaceutical formulations, and as such, a variety of dosages and treatment regimens may be desirable.

Even though a number of embodiments are directed to liver-targeted delivery of any of the oligonucleotides disclosed herein, targeting of other tissues is also contemplated.

### IV. Methods of Use

i. Reducing HMGB1 Expression in Cells

In some embodiments, methods are provided for delivering to a cell an effective amount any one of oligonucleotides disclosed herein for purposes of reducing expression of HMGB1 in the cell. Methods provided herein are useful in any appropriate cell type. In some embodiments, a cell is any cell that expresses HMGB1 (e.g., hepatocytes, macrophages, monocyte-derived cells, prostate cancer cells, cells of the brain, endocrine tissue, bone marrow, lymph nodes, lung, gall bladder, liver, duodenum, small intestine, pancreas, kidney, gastrointestinal tract, bladder, adipose and soft tissue and skin). In some embodiments, the cell is a primary cell that has been obtained from a subject and that may have undergone a limited number of a passages, such that the cell substantially maintains is natural phenotypic properties. In some embodiments, a cell to which the oligonucleotide is delivered is ex vivo or in vitro (e.g., can be delivered to a cell in culture or to an organism in which the cell resides). In specific embodiments, methods are provided for delivering to a cell an effective amount any one of oligonucleotides disclosed herein for purposes of reducing expression of HMGB1 solely in hepatocytes.

In some embodiments, oligonucleotides disclosed herein can be introduced using appropriate nucleic acid delivery methods including injection of a solution containing the oligonucleotides, bombardment by particles covered by the oligonucleotides, exposing the cell or organism to a solution containing the oligonucleotides, or electroporation of cell membranes in the presence of the oligonucleotides. Other appropriate methods for delivering oligonucleotides to cells may be used, such as lipid-mediated carrier transport, chemical-mediated transport, and cationic liposome transfection such as calcium phosphate, and others.

The consequences of inhibition can be confirmed by an appropriate assay to evaluate one or more properties of a cell or subject, or by biochemical techniques that evaluate molecules indicative of HMGB1 expression (e.g., RNA, protein). In some embodiments, the extent to which an oligonucleotide provided herein reduces levels of expression of HMGB1 is evaluated by comparing expression levels (e.g., mRNA or protein levels of HMGB1 to an appropriate control (e.g., a level of HMGB1 expression in a cell or population of cells to which an oligonucleotide has not been delivered or to which a negative control has been delivered). In some embodiments, an appropriate control level of HMGB1 expression may be a predetermined level or value, such that a control level need not be measured every time. The predetermined level or value can take a variety of forms. In some embodiments, a predetermined level or value can be single cut-off value, such as a median or mean.

In some embodiments, administration of an oligonucleotide as described herein results in a reduction in the level of HMGB1 expression in a cell. In some embodiments, the reduction in levels of HMGB1 expression may be a reduction to 1% or lower, 5% or lower, 10% or lower, 15% or lower, 20% or lower, 25% or lower, 30% or lower, 35% or lower, 40% or lower, 45% or lower, 50% or lower, 55% or lower, 60% or lower, 70% or lower, 80% or lower, or 90% or lower compared with an appropriate control level of HMGB1. The appropriate control level may be a level of HMGB1 expression in a cell or population of cells that has not been contacted with an oligonucleotide as described herein. In some embodiments, the effect of delivery of an oligonucleotide to a cell according to a method disclosed herein is assessed after a finite period of time. For example, levels of HMGB1 may be analyzed in a cell at least 8 hours, 12 hours, 18 hours, 24 hours; or at least one, two, three, four, five, six, seven, or fourteen days after introduction of the oligonucleotide into the cell.

In some embodiments, an oligonucleotide is delivered in the form of a transgene that is engineered to express in a cell the oligonucleotides (e.g., its sense and antisense strands). In some embodiments, an oligonucleotide is delivered using a transgene that is engineered to express any oligonucleotide disclosed herein. Transgenes may be delivered using viral vectors (e.g., adenovirus, retrovirus, vaccinia virus, poxvirus, adeno-associated virus or herpes simplex virus) or non-viral vectors (e.g., plasmids or synthetic mRNAs). In some embodiments, transgenes can be injected directly to a subject.

ii. Treatment Methods

Aspects of the disclosure relate to methods for reducing HMGB1 expression in for attenuating the onset or progression of liver fibrosis in a subject. In some embodiments, the methods may comprise administering to a subject in need thereof an effective amount of any one of the oligonucleotides disclosed herein. Such treatments could be used, for example, to slow or halt any type of liver fibrosis. The present disclosure provides for both prophylactic and therapeutic methods of treating a subject at risk of (or susceptible to) a disease or disorder associated with liver fibrosis and/or liver inflammation.

The compounds of the invention selectively target HMGB1 mRNA exclusively in the liver. Compared to other NASH therapies, this selectivity is expected to increase the potency of the HMGB1 inhibitors of the inventions while decreasing off-target interactions and potential side effects.

In certain aspects, the disclosure provides a method for preventing in a subject, a disease or disorder as described herein by administering to the subject a therapeutic agent (e.g., an oligonucleotide or vector or transgene encoding same). In some embodiments, the subject to be treated is a subject who will benefit therapeutically from a reduction in the amount of HMGB1 protein, e.g., in the liver. Subjects at risk for the disease or disorder can be identified by, for example, one or a combination of diagnostic or prognostic assays known in the art (e.g., identification of liver fibrosis and/or liver inflammation). Administration of a prophylactic agent can occur prior to the detection of or the manifestation of symptoms characteristic of the disease or disorder, such that the disease or disorder is prevented or, alternatively, delayed in its progression.

In some embodiments, the disclosure provides methods for using RNAi oligonucleotides of the invention for treating subjects having or suspected of having liver conditions such as, for example, cholestatic liver disease, nonalcoholic fatty liver disease (NAFLD) and nonalcoholic steatohepatitis (NASH).

In some embodiments, the disclosure provides RNAi oligonucleotides described herein for use in treating subjects having or suspected of having liver conditions such as, for example, cholestatic liver disease, NAFLD and NASH.

In some embodiments, the disclosure provides RNAi for the preparation of a medicament for treatment of subjects having or suspected of having liver conditions such as, for example, cholestatic liver disease, NAFLD and nonalcoholic steatohepatitis NASH.

Methods described herein are typically involve administering to a subject in an effective amount of an oligonucleotide, that is, an amount capable of producing a desirable therapeutic result. A therapeutically acceptable amount may be an amount that is capable of treating a disease or disorder. The appropriate dosage for any one subject will depend on certain factors, including the subject's size, body surface area, age, the particular composition to be administered, the active ingredient(s) in the composition, time and route of administration, general health, and other drugs being administered concurrently.

In some embodiments, a subject is administered any one of the compositions disclosed herein either enterally (e.g., orally, by gastric feeding tube, by duodenal feeding tube, via gastrostomy or rectally), parenterally (e.g., subcutaneous injection, intravenous injection or infusion, intra-arterial injection or infusion, intraosseous infusion, intramuscular injection, intracerebral injection, intracerebroventricular injection, intrathecal), topically (e.g., epicutaneous, inhalational, via eye drops, or through a mucous membrane), or by direct injection into a target organ (e.g., the liver of a subject). Typically, oligonucleotides disclosed herein are administered intravenously or subcutaneously.

As a non-limiting set of examples, the oligonucleotides of the instant disclosure would typically be administered quarterly (once every three months), bi-monthly (once every two months), monthly, or weekly. For example, the oligonucleotides may be administered every week or at intervals of two, or three weeks. In some embodiments, the oligonucleotides may be administered daily.

In some embodiments, the subject to be treated is a human or non-human primate or other mammalian subject. Other exemplary subjects include domesticated animals such as dogs and cats; livestock such as horses, cattle, pigs, sheep, goats, and chickens; and animals such as mice, rats, guinea pigs, and hamsters.

### EXAMPLES

### Example 1: In Vivo Activity of GalNAc-Conjugated HMGB1 Oligonucleotides

HMGB1 oligonucleotides used in this example were designed to bind to conserved sequences identified by the algorithm in the human, monkey (both rhesus), and mouse sequences (“triple common” sequences). In this study, three triple-common oligonucleotide sequences (S31-AS18, S35-AS22, and S36-AS23) were tested in 3 different modification patterns (M1, M2 and M3, see FIGS. 1A and 1B). Oligonucleotides were subcutaneously administered to CD-1 mice at 1 mg/kg and mice were euthanized on day 5 following administration. Liver samples were obtained and RNA was extracted to evaluate HMGB1 mRNA levels by qPCR (normalized to HPRT1-F576 (Housekeeping gene). The levels of remaining HMGB1 mRNA were interrogated using TAQMAN®-based qPCR assays.

The qPCR was performed using two different primers specific to different regions in the HMGB1 mRNA. The qPCR performed using the primer at the 5′ end relative to the other primer was designated “5′ qPCR.” Similarly, the qPCR performed using the primer at the 3′ end relative to the other primer was designated “3′ qPCR.” In this experiment, a 3′ qPCR assay (using primer MmHMGB1-F1541) was used. The data showed that all tested HMGB1 oligonucleotides were potent in knockdown HMGB1 5 days after administration, as indicated by the reduced amount of HMGB1 mRNA remaining in mice liver (normalized to a PBS control treatment) (FIG. 1A).

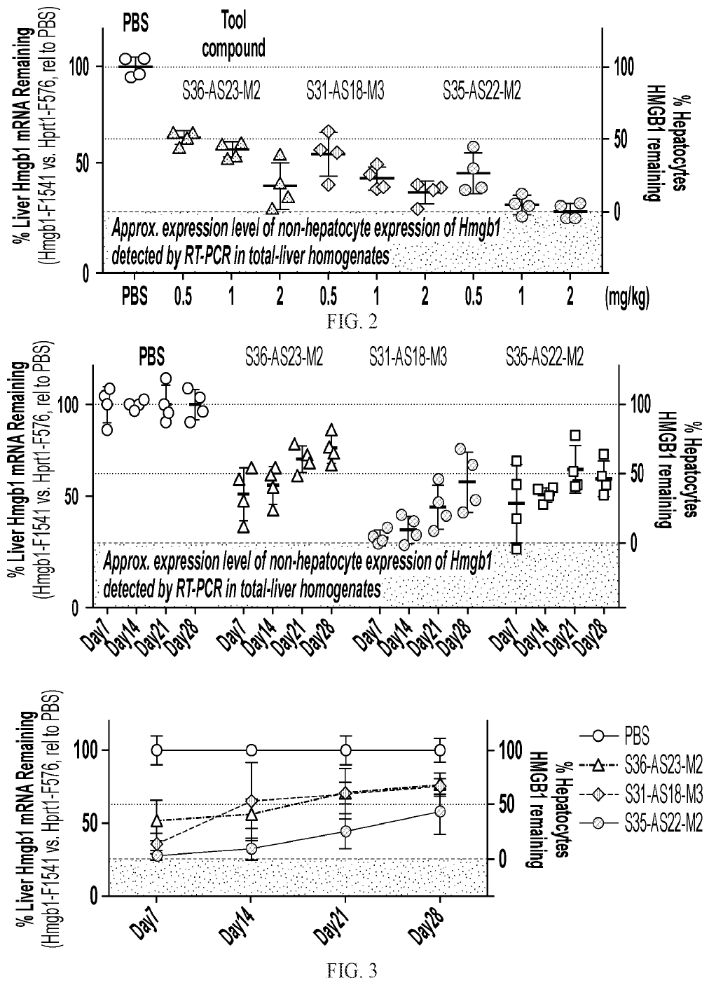

Two GalNAc-conjugated HMGB1 oligonucleotides with different modification patterns (S31-AS18-M3 and S35-AS22-M2) were further tested in a dose response analysis, using the tool compound S36-AS23-M2 as control. The three oligonucleotides were subcutaneously administered to CD-1 mice at three different dosages (0.5 mg/kg, 1 mg/kg, and 2 mg/kg). Mice were euthanized on day 5 following administration. Liver samples were obtained, and RNA was extracted to evaluate HMGB1 mRNA levels by qPCR (normalized to HPRT1-F576 (Housekeeping gene). The levels of remaining HMGB1 mRNA were interrogated using TAQMAN®-based qPCR assays (a 3′assay as described above was used). The results show that the two tested GalNAc-conjugated HMGB1 oligonucleotides, in both modification patterns, reduced liver HMGB1 mRNA level in mice liver (FIG. 2). All oligonucleotides showed ED₅₀s of ˜0.5-1.0 mg/kg, especially if non-hepatocyte ‘floor’ is considered.

The two GalNAc-conjugated HMGB1 oligonucleotides with different modification patterns (S31-AS18-M3 and S35-AS22-M2) were then tested in vivo in a duration study to evaluate their activity in inhibiting HMGB1 expression in mice. PBS was used as negative control, and the tool compound S36-AS23-M2 was used as positive control in this experiment. Oligonucleotides were subcutaneously administered to CD-1 mice at 1 mg/kg and mice were euthanized on days 7, 14, 21, and 28 following administration. Liver samples were obtained and RNA was extracted to evaluate HMGB1 mRNA levels by qPCR (normalized to HPRT1-F576 housekeeping gene). A 3′assay as described above was used. The data showed that all tested HMGB1 oligonucleotides were potent in knockdown HMGB1 3 weeks after injection, even with 1 mg/kg single dose, as indicated by the reduced amount of HMGB1 mRNA remaining in mice liver at days 7, 14, 21, and 28 (normalized to a PBS control treatment) (FIG. 3).

### Example 2: Additional In Vitro Screening of New HMGB1 RNAi Oligonucleotides Sequences to Identify Additional RNAi Oligonucleotides that Inhibit HMGB1 Expression

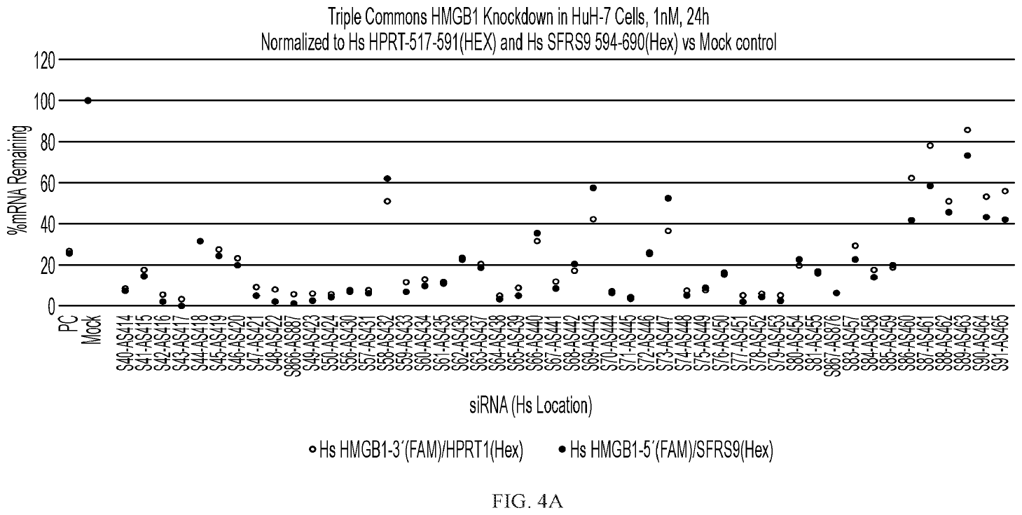

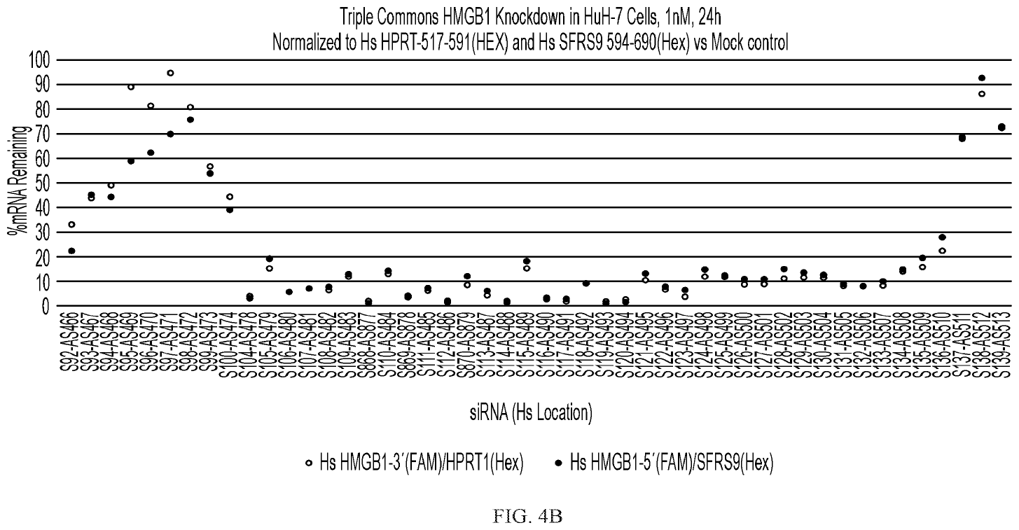

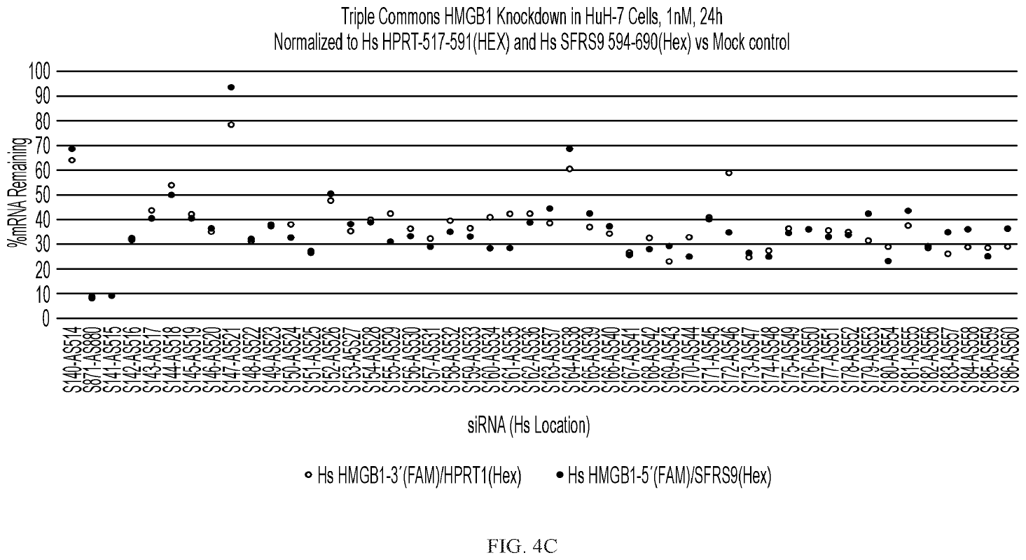

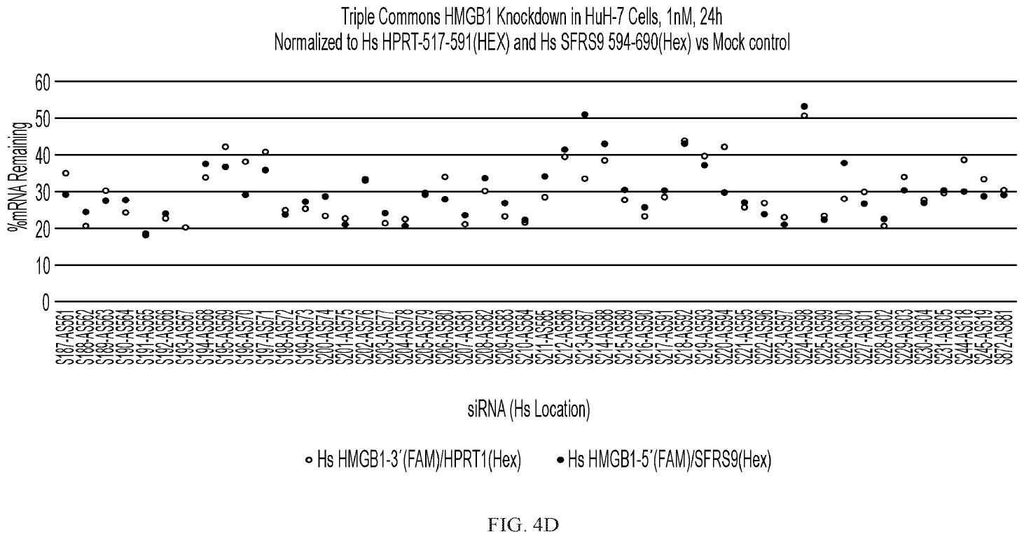

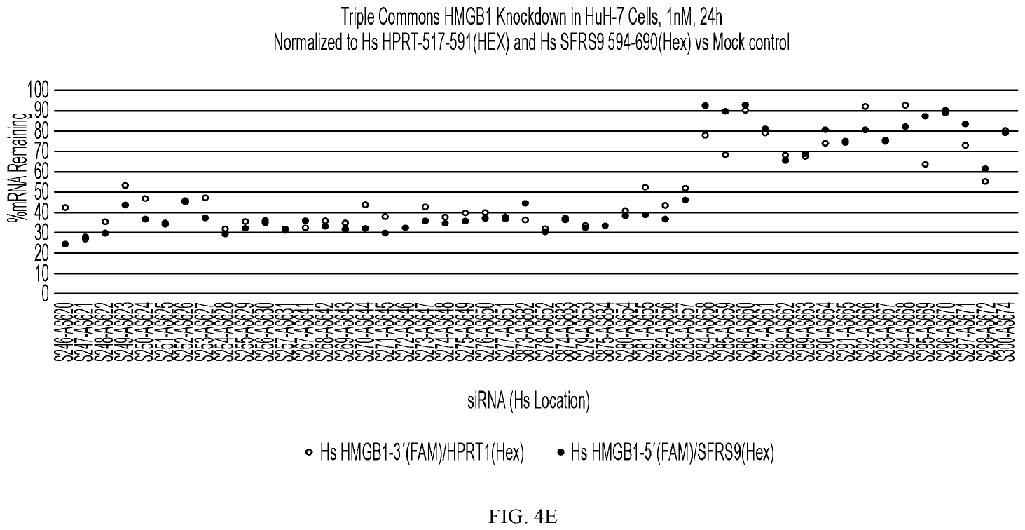

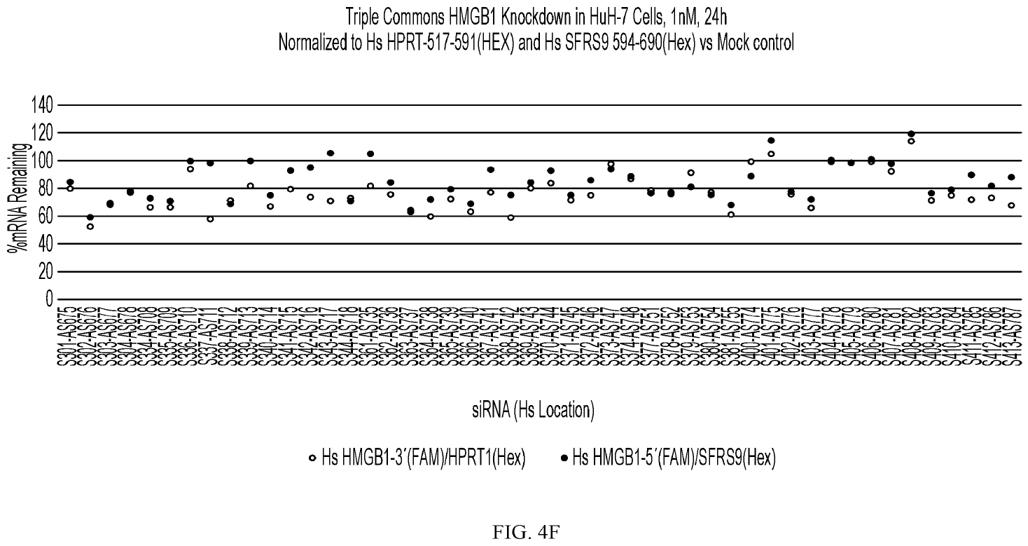

Additional 288 triple commons HMGB1 RNAi oligonucleotides (see Table 4 for sequences) were screened in an in vitro activity assay to identify additional RNAi oligonucleotides sequences that are effective at inhibiting HMGB1 expression (FIGS. 4A-4F). In this assay, Huh-7 cells were transfected with the indicated oligonucleotides. Cells were maintained for 24 h following transfection, RNAs were isolated using the iScript R2′-qPCR sample preparation buffer. HMGB1 mRNA were interrogated using TAQMAN®-based qPCR assays. Two qPCR assays, a 5′ assay and a 3′ assay, were used to determine mRNA levels as measured by HEX (housekeeping gene—HPR2′-F576/SFRS9-F594) and FAM probes, respectively.

The percent mRNA remaining is shown for each of the 5′ assay (red) and the 3′ assay (blue). Oligonucleotides with the lowest percentage of mRNA remaining compared to mock transfection controls were considered hits. Oligonucleotides with low complementarity to the human genome were used as negative controls.

Twenty-two (22) new sequences identified in the screen were made into GalNAc-conjugated tetraloop oligonucleotides (see Table 2 and Table 5 for sequences) with two different modification patterns (M2 and M3, respectively) and their in vivo activities in reduce liver HMGB1 mRNA level were tested. PBS was used as negative control, and the tool compound in two different modification patterns (S36-AS23-M2 and S36-AS23-M3) were used as positive control in this experiment. Oligonucleotides were subcutaneously administered to CD-1 mice at 1 mg/kg, and mice were euthanized on day 5 following administration. Liver samples were obtained and RNA was extracted to evaluate HMGB1 mRNA levels by qPCR (normalized to HPRT1-F576 (Housekeeping gene). The levels of remaining HMGB1 mRNA were interrogated using TAQMAN®-based qPCR assays (3′assay was used). The results show that the new GalNAc-conjugated oligonucleotides had different levels of activity in inhibiting liver HMGB1 and 7 new GalNAc-conjugated HMGB1 oligonucleotides (as indicated by the arrow, S27-AS14, S28-AS15, S29-AS16, S30-AS17, S32-AS19, S33-AS20, and S34-AS21) were selected to be included in knockdown duration study, if at least 50% suppression of HMGB1 mRNA is achieved 3 weeks after a single dose of 3 to 5 mg/kg in mice (FIG. 5).

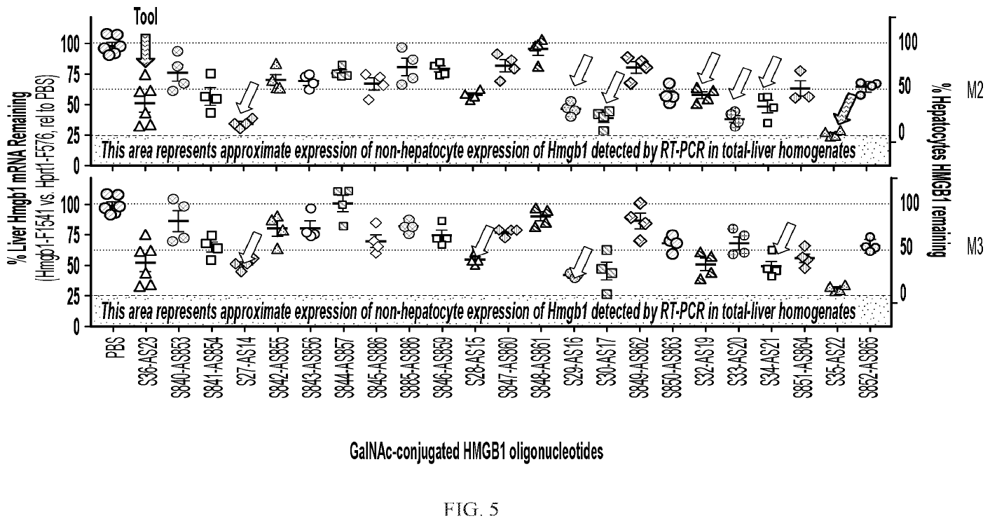

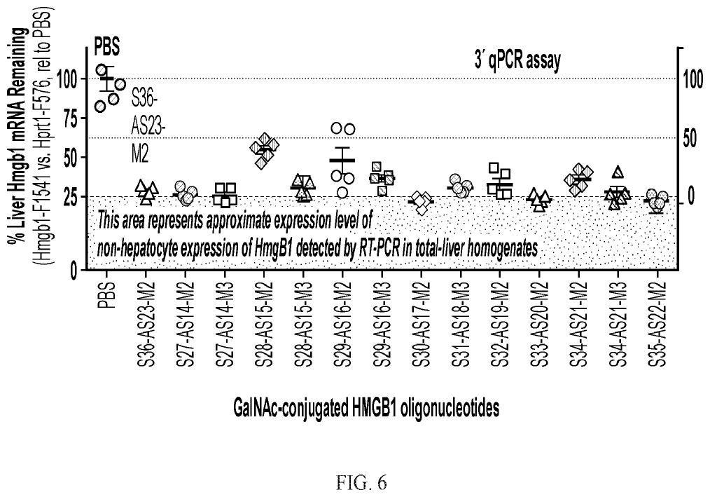

For the knockdown duration assay, the 7 new GalNAc-conjugated HMGB1 oligonucleotides (S27-AS14, S28-AS15, S29-AS16, S30-AS17, S32-AS19, S33-AS20, and S34-AS21) with different modification patterns (M2 or M3) were tested and compared with two previously identified GalNAc-conjugated HMGB1 oligonucleotides (S31-AS18-M3 and S35-AS22-M2). PBS was used as negative control, and the tool compound S36-AS23-M2 was used as positive control. Oligonucleotides were subcutaneously administered to CD-1 mice at 4 mg/kg and mice were euthanized on day 21 following administration. Liver samples were obtained and RNA was extracted to evaluate HMGB1 mRNA levels by qPCR (normalized to HPRT1-F576 (Housekeeping gene). The levels of remaining HMGB1 mRNA were interrogated using TAQMAN®-based qPCR assays (3′ assay was used). The percent remaining HMGB1 mRNA in the liver 21 days after the administration of the oligonucleotides, normalized to PBS control treatment, is shown in FIG. 6. All tested HMGB1 oligonucleotides were potent in knockdown HMGB1 3 weeks after injection.

### Example 3: Testing GalNAc-Conjugated HMGB1 Oligonucleotides in Primary Monkey or Human Hepatocytes

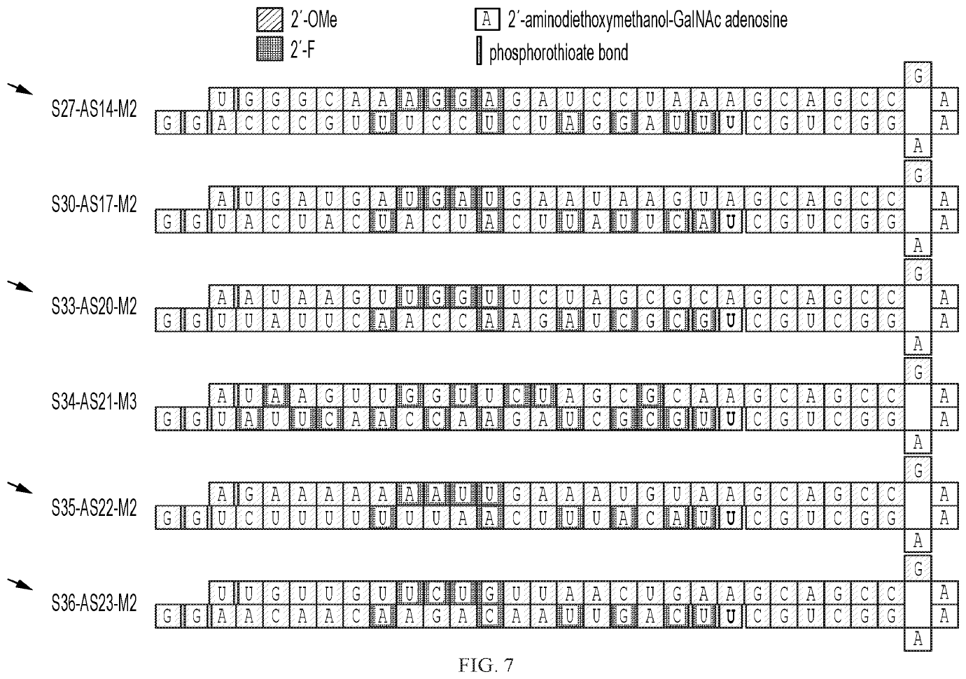

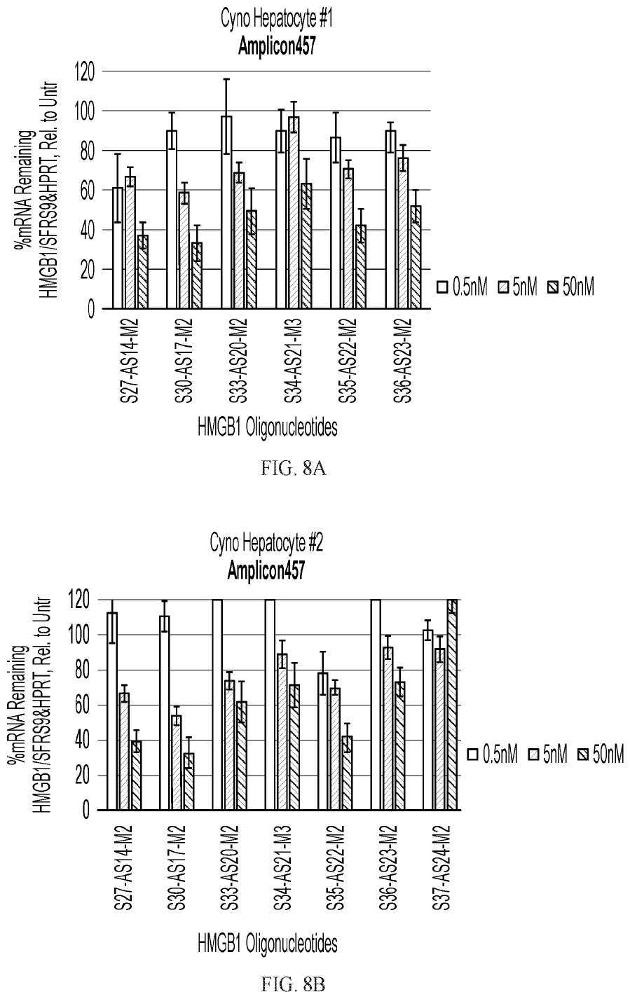

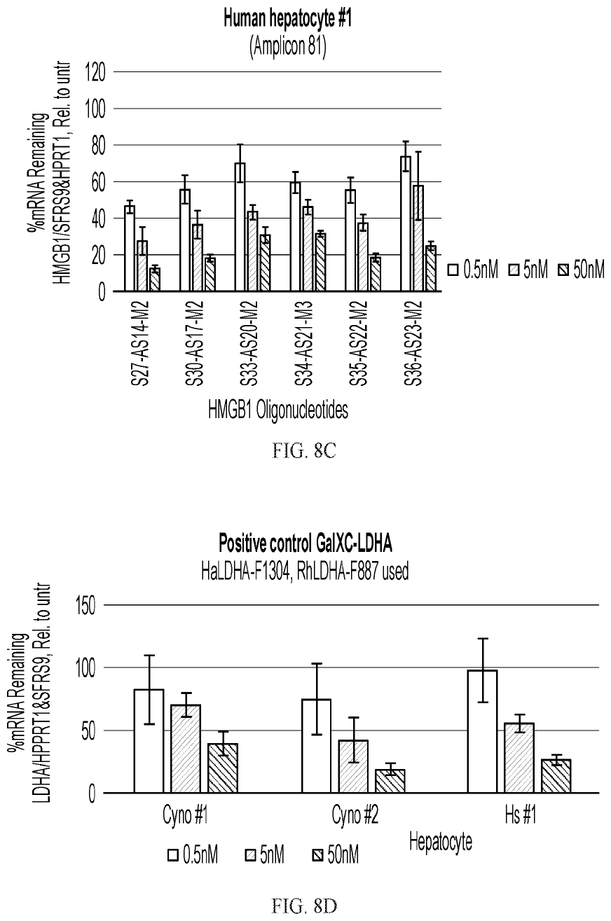

The GalNAc-conjugated HMGB1 oligonucleotides tested in this experiment were shown in FIG. 7. Also shown in FIG. 7 is a GalNAc-conjugated LDHA oligonucleotide for using as positive control. In this assay, GalNAc-conjugates HMGB1 oligonucleotides were delivered by ASGPR receptor-mediated uptake into the monkey primary hepatocyte (FIGS. 8A and 8B) or the human hepatocyte (FIG. 8C) cells.

Cells were maintained for 24 h following oligonucleotides delivery. RNA was extracted to evaluate HMGB1 mRNA levels by qPCR (normalized to RhPPIB (Housekeeping gene) for monkey hepatocytes and HPRT1-F576 (Housekeeping gene) for human hepatocytes). The levels of remaining HMGB1 mRNA were interrogated using TAQMAN®-based qPCR assays. RhMHGB1-F457 qPCR assay (FIGS. 8A and 8B) and HsHMGB1-F81 assay (FIG. 8C) were used. GalNAc-conjugate LDHA-1360 oligonucleotide was used as assay control (FIG. 8D) and the level of remaining LDHA mRNA was measured by RhLDHA-F887 qPCR assay. IC50 curves of RhHMGB1, normalized to Mock treatment, are shown.

Four exemplary GalNAc-conjugated HMGB1 oligonucleotides (S27-AS14-M2, S33-AS20-M2, S35-AS22-M2, and S37-AS24-M2) were further tested in vivo in non-human primates (monkeys) for their activity in HMGB1 knockdown. PBS was used as negative control, and the tool compound S36-AS23-M2 was used as positive control in this experiment. The structures of S27-AS14-M2, S33-AS20-M2, S35-AS22-M2, and S36-AS23-M2 are depicted in FIG. 7.

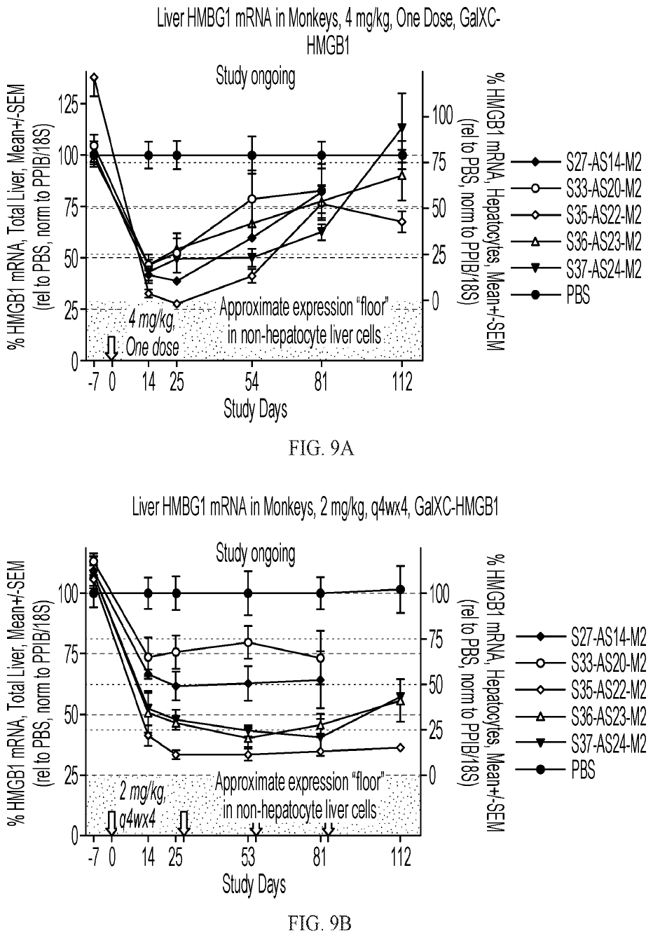

Oligonucleotides were subcutaneously administered to non-human primate at 4 mg/kg, one single dose, or 2 mg/kg, 4 repeat doses. Monkey liver biopsies were taken each time point following administration. Liver samples were obtained and RNA was extracted to evaluate HMGB1 mRNA levels by qPCR. The percentages of remaining HMGB1 mRNA in monkey liver 7, 14, 25, 54, 81 and 112 days after the administration, normalized to PBS control treatment, are shown (FIGS. 9A-9B). The results show that all oligonucleotides tested significantly reduced liver HMGB1 mRNA level at 25 days post administration. With 4 repeated 2 mg/kg doses, the HMGB1 knockdown effect lasted through 112 days (FIG. 9B).

### Example 4: Comparing HMGB1 RNAi Oligonucleotides in Primary Monkey/Human Hepatocytes

The activity of five HMGB1 double strand RNAi oligonucleotides (5866-AS887, S869-AS878, 5116-AS490, 5117-AS491, 5871-AS880, and 5872-AS881 in Table 4) were compared in mouse, monkey, and human cell lines. PBS was used as negative control, and the tool compound S36-AS23-M2 was used as positive control in this experiment. In this assay, mouse cells (Hepa1-6 cells, FIGS. 10A and 10B), monkey cells (LLC-MK2 cells, FIGS. 10C and 10D), and human cells (Huh-7, FIGS. 10E and 10F) were transfected with the indicated oligonucleotides, respectively. Cells were maintained for 24 h following transfection, RNAs were isolated using the iScript R2′-qPCR sample preparation buffer. HMGB1 mRNA were interrogated using TAQMAN®-based qPCR assays. Two qPCR assays, a 5′ assay (FIGS. 10A, 10C, and 10E) and a 3′ assay (FIGS. 10B, 10D, and 10F), were used to determine mRNA levels as measured by HEX (housekeeping gene—HPR2′-F576) and FAM probes, respectively. Oligonucleotides 210, 840, 852, 853 show more than 80% knockdown at 0.1 nM and 1 nM concentrations in both assays in mouse cell line (FIGS. 10A and 10B). All RNAi oligonucleotides display more than 80% KD at 0.1 nM and 1 nM concentrations (FIGS. 10C and 10D). Oligonucleotides 210, 840, 852, 853, 932 show better potency with more than −90% KD at 0.1 nM and 1 nM concentrations in human cell line (FIGS. 10E and 10F).

### Example 5: Testing GalXC-HMGB1 for HMGB1 Selectivity

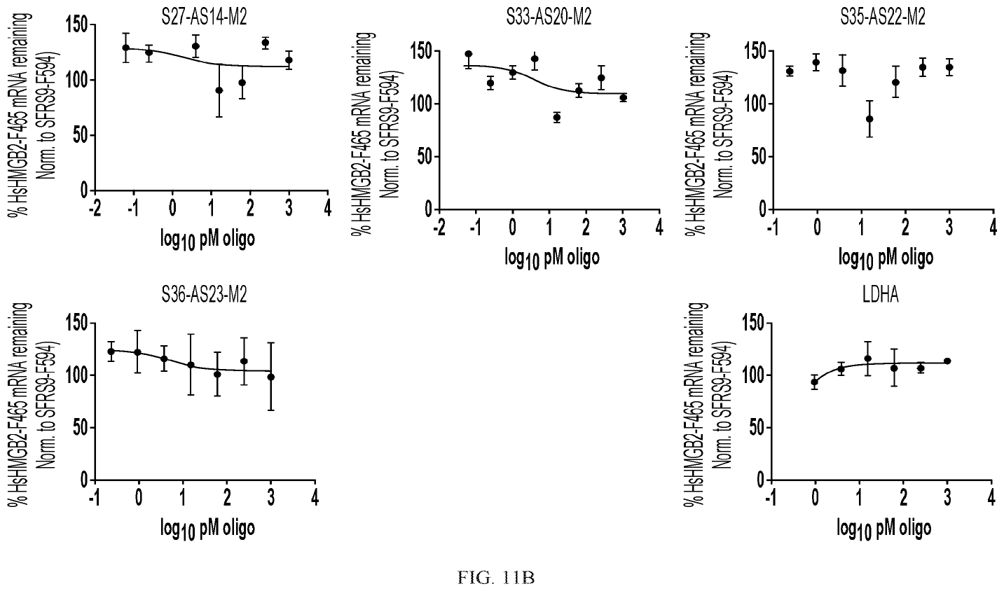

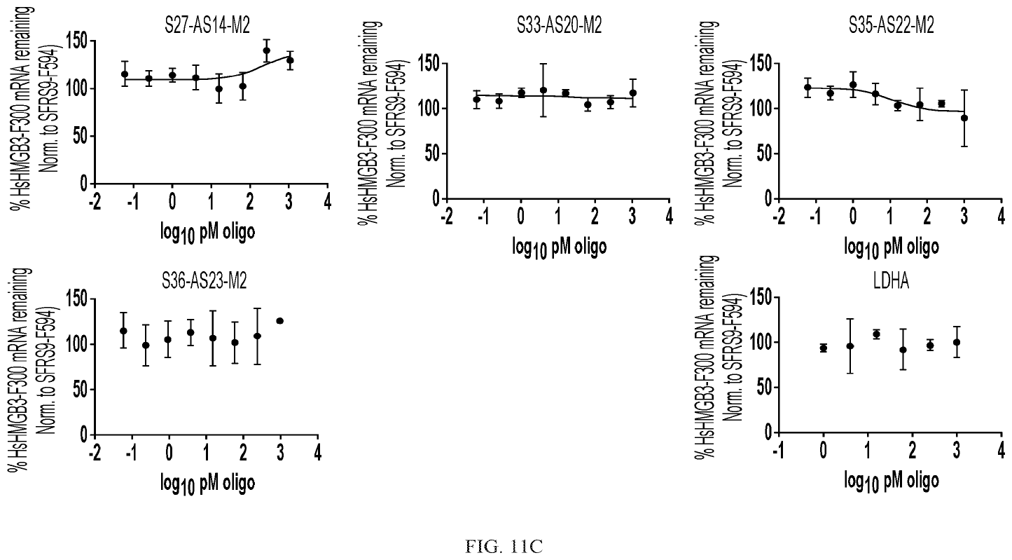

The selectivity of GalNAc-conjugated HMGB1 oligonucleotides S27-AS14-M2, S33-AS20-M2, S35-AS22-M2, and S36-AS23-M2 against HMGB1, HMGB2, and HMGB3 were tested in human hepatocytes. The structures of S27-AS14-M2, S33-AS20-M2, S35-AS22-M2, and S36-AS23-M2 are depicted in FIG. 7. A GalNAc-conjugated LDHA oligonucleotide for using as positive control. Huh-7 cells were transfected with the indicated oligonucleotides with 8 different concentration (4-fold dilution with 8-points, highest concentration is 1 nM). Cells were maintained for 24 h following transfection, RNAs were isolated using the iScript R2′-qPCR sample preparation buffer. HMGB1 mRNA were interrogated using TAQMAN®-based qPCR assays. 5′ qPCR assay used to determine mRNA levels as measured by HEX (housekeeping gene—SFRS9) and FAM probes, respectively. IC50 curves of HMGB1 (FIG. 11A), HMGB2 (FIG. 11B) and HMGB3 (FIG. 11C), normalized to mock treatment, are shown. All four tested conjugates had similar IC50 value for HMGB1 (FIG. 11A). None of the conjugates (including the LDHA control oligonucleotide) had any effect of HMGB2 mRNA level (FIG. 11B) or HMGB3 mRNA level (FIG. 11C).

### Example 6: Materials and Methods

### Transfection

For the first screen, Lipofectamine RNAiMAX™ was used to complex the oligonucleotides for efficient transfection. Oligonucleotides, RNAiMAX and Opti-MEM were added to a plate and incubated at room temperature for 20 minutes prior to transfection. Media was aspirated from a flask of actively passaging cells and the cells are incubated at 37° C. in the presence of trypsin for 3-5 minutes. After cells no longer adhered to the flask, cell growth media (lacking penicillin and streptomycin) was added to neutralize the trypsin and to suspend the cells. A 10 μL aliquot was removed and counted with a hemocytometer to quantify the cells on a per millimeter basis. For HeLa cells, 20,000 cells were seeded per well in 100 μL of media. The suspension was diluted with the known cell concentration to obtain the total volume required for the number of cells to be transfected. The diluted cell suspension was added to the 96 well transfection plates, which already contained the oligonucleotides in Opti-MEM. The transfection plates were then incubated for 24 hours at 37° C. After 24 hours of incubation, media was aspirated from each well. Cells were lysed using the lysis buffer from the Promega RNA Isolation kit. The lysis buffer was added to each well. The lysed cells were then transferred to the Corbett XtractorGENE (QIAxtractor) for RNA isolation or stored at -80° C.

For subsequent screens and experiments, e.g., the secondary screen, Lipofectamine RNAiMAx was used to complex the oligonucleotides for reverse transfection. The complexes were made by mixing RNAiMAX and siRNAs in OptiMEM medium for 15 minutes. The transfection mixture was transferred to multi-well plates and cell suspension was added to the wells. After 24 hours incubation the cells were washed once with PBS and then lysed using lysis buffer from the Promega SV96 kit. The RNA was purified using the SV96 plates in a vacuum manifold. Four microliters of the purified RNA was then heated at 65° C. for 5 minutes and cooled to 4° C. The RNA was then used for reverse transcription using the High Capacity Reverse Transcription kit (Life Technologies) in a 10-microliter reaction. The cDNA was then diluted to 50 μL with nuclease free water and used for quantitative PCR with multiplexed 5′-endonuclease assays and SSoFast qPCR mastermix (Bio-Rad laboratories).

cDNA Synthesis

RNA was isolated from mammalian cells in tissue culture using the Corbett X-tractor Gene™ (QIAxtractor). A modified SuperScript II protocol was used to synthesize cDNA from the isolated RNA. Isolated RNA (approximately 5 ng/μL) was heated to 65° C. for five minutes and incubated with dNPs, random hexamers, oligo dTs and water. The mixture was cooled for 15 seconds. An “enzyme mix,” consisting of water, 5× first strand buffer, DTT, SUPERase⋅In™ (an RNA inhibitor), and SuperScript II RTase was added to the mixture. The contents were heated to 42° C. for one hour, then to 70° C. for 15 minutes, and then cooled to 4° C. using a thermocycler. The resulting cDNA was then subjected to SYBR®-based qPCR. The qPCR reactions were multiplexed, containing two 5′ endonuclease assays per reaction.

qPCR Assays

Primer sets were initially screened using SYBR®-based qPCR. Assay specificity was verified by assessing melt curves as well as “minus RT” controls. Dilutions of cDNA template (10-fold serial dilutions from 20 ng and to 0.02 ng per reaction) from HeLa and Hepa1-6 cells are used to test human (Hs) and mouse (Mm) assays, respectively. qPCR assays were set up in 384-well plates, covered with MicroAmp film, and run on the 7900HT from Applied Biosystems. Reagent concentrations and cycling conditions included the following: 2×SYBR mix, 10 μM forward primer, 10 μM reverse primer, DD H₂O, and cDNA template up to a total volume of 10 μL.

In some cases, as noted, qPCR was performed using TAQMAN®-based qPCR assays. TAQMAN® probes target two different positions (5′ and 3′ to one another) within the coding region of the target mRNA (e.g., HMGB1) were generally used to provide additional confirmation of mRNA levels in the analysis.

### Cloning

PCR amplicons that displayed a single melt-curve were ligated into the pGEM®-T Easy vector kit from Promega according to the manufacturer's instructions. Following the manufacturer's protocol, JM109 High Efficiency cells were transformed with the newly ligated vectors. The cells were then plated on LB plates containing ampicillin and incubated at 37° C. overnight for colony growth.

### PCR Screening and Plasmid Mini-Prep

PCR was used to identify colonies of E. coli that had been transformed with a vector containing the ligated amplicon of interest. Vector-specific primers that flank the insert were used in the PCR reaction. All PCR products were then run on a 1% agarose gel and imaged by a transilluminator following staining Gels were assessed qualitatively to determine which plasmids appeared to contain a ligated amplicon of the expected size (approximately 300 bp, including the amplicon and the flanking vector sequences specific to the primers used).

The colonies that were confirmed transformants by PCR screening were then incubated overnight in cultures consisting of 2 mL LB broth with ampicillin at 37° C. with shaking. E. coli cells were then lysed, and the plasmids of interest were isolated using Promega's Mini-Prep kit. Plasmid concentration was determined by UV absorbance at 260 nm.

### Plasmid Sequencing and Quantification

Purified plasmids were sequenced using the BigDye® Terminator sequencing kit. The vector-specific primer, T7, was used to give read lengths that span the insert. The following reagents were used in the sequencing reactions: water, 5× sequencing buffer, BigDye terminator mix, T7 primer, and plasmid (100 ng/μL) to a volume of 10 μL. The mixture was held at 96° C. for one minute, then subjected to 15 cycles of 96° C. for 10 seconds, 50° C. for 5 seconds, 60° C. for 1 minute, 15 seconds; 5 cycles of 96° C. for 10 seconds, 50° C. for 5 seconds, 60° C. for 1 minute, 30 seconds; and 5 cycles of 96° C. for 10 seconds, 50° C. for 5 seconds, and 60° C. for 2 minutes. Dye termination reactions were then sequenced using Applied Biosystems' capillary electrophoresis sequencers.

Sequence-verified plasmids were then quantified. They were linearized using a single cutting restriction endonuclease. Linearity was confirmed using agarose gel electrophoresis. All plasmid dilutions were made in TE buffer (pH 7.5) with 100 μg of tRNA per mL buffer to reduce non-specific binding of plasmid to the polypropylene vials.

The linearized plasmids were then serially diluted from 1,000,000 to 01 copies per μL and subjected to qPCR. Assay efficiency was calculated, and the assays were deemed acceptable if the efficiency was in the range of 90-110%.

### Multi-Plexing Assays

For each target, mRNA levels were quantified by two 5′ nuclease assays. In general, several assays are screened for each target. The two assays selected displayed a combination of good efficiency, low limit of detection, and broad 5′43′ coverage of the gene of interest (GOI). Both assays against one GOI could be combined in one reaction when different fluorophores were used on the respective probes. Thus, the final step in assay validation was to determine the efficiency of the selected assays when they were combined in the same qPCR or “multi-plexed”.

Linearized plasmids for both assays in 10-fold dilutions were combined and qPCR was performed. The efficiency of each assay was determined as described above. The accepted efficiency rate was 90-110%.

While validating multi-plexed reactions using linearized plasmid standards, C_{q} values for the target of interest were also assessed using cDNA as the template. For human or mouse targets, HeLa and Hepa1-6 cDNA were used, respectively. The cDNA, in this case, was derived from RNA isolated on the Corbett (˜5 ng/μL in water) from untransfected cells. In this way, the observed C_{q} values from this sample cDNA were representative of the expected C_{q} values from a 96-well plate transfection. In cases where C_{q} values were greater than 30, other cell lines were sought that exhibit higher expression levels of the gene of interest. A library of total RNA isolated from via high-throughput methods on the Corbett from each human and mouse line was generated and used to screen for acceptable levels of target expression.

### Description of Oligonucleotide Nomenclature

All oligonucleotides described herein are designated either SN₁-ASN₂-MN₃. The following designations apply:
N₁: sequence identifier number of the sense strand sequence
N₂: sequence identifier number of the antisense strand sequence
N₃: reference number of modification pattern, in which each number represents a pattern of modified nucleotides in the oligonucleotide.

For example, S1-AS14-M1 represents an oligonucleotide with a sense sequence that is set forth by SEQ ID NO: 1, an antisense sequence that is set forth by SEQ ID NO: 14, and which is adapted to modification pattern number 1.

TABLE 1
Lead HMGB1 RNAi Oligonucleotide Sequences

| Sense (S)- | | S | | AS |
|---|---|---|---|---|
| Antisense (AS) | | SEQ | | SEQ |
| Designation | Sense Sequence/mRNA Sequence | ID NO: | Antisense Sequence | ID NO: |
| S1-AS14 | UGGGCAAAGGAGAUCCUAAA | 1 | UUUAGGAUCUCCUUUGCCCAGG | 14 |
| S2-AS15 | AAAGAGAAAUGAAAACCUAA | 2 | UUAGGUUUUCAUUUCUCUUUGG | 15 |
| S3-AS16 | AAGAAGAUGAUGAUGAUGAA | 3 | UUCAUCAUCAUCAUCUUCUUGG | 16 |
| S4-AS17 | AUGAUGAUGAUGAAUAAGUA | 4 | UACUUAUUCAUCAUCAUCAUGG | 17 |
| S5-AS18 | GAUGAUGAAUAAGUUGGUUC | 5 | GAACCAACUUAUUCAUCAUCGG | 18 |
| S6-AS19 | UGAAUAAGUUGGUUCUAGCA | 6 | UGCUAGAACCAACUUAUUCAGG | 19 |
| S7-AS20 | AAUAAGUUGGUUCUAGCGCA | 7 | UGCGCUAGAACCAACUUAUUGG | 20 |
| S8-AS21 | AUAAGUUGGUUCUAGCGCAA | 8 | UUGCGCUAGAACCAACUUAUGG | 21 |
| S9-AS22 | AGAAAAAAAUUGAAAUGUAA | 9 | UUACAUUUCAAUUUUUUUCUGG | 22 |
| S10-AS23 | UUGUUGUUCUGUUAACUGAA | 10 | UUCAGUUAACAGAACAACAAGG | 23 |
| S11-AS24 | UUCUGAAUGCUUCUAAGUAA | 11 | UUACUUAGAAGCAUUCAGAAGG | 24 |
| S12-AS25 | CUGAAUGCUUCUAAGUAAAA | 12 | UUUUACUUAGAAGCAUUCAGGG | 25 |
| S13-AS26 | GAAUGCUUCUAAGUAAAUAA | 13 | UUAUUUACUUAGAAGCAUUCGG | 26 |

TABLE 2
Lead GalNAc-conjugated HMGB1 oligonucleotide sequences with modifications

| GalNA | Sense | | Antisense | |
|---|---|---|---|---|
| conjugated | sequence | | sequence | Antisense sequence |
| oligonucleotides | (unmodified) | Sense Sequence (modified) | (unmodified) | (modified) |
| S27-AS14-M2 | UGGGCAAAGGAGA | [mUs][mG][mG][mG][mC][mA] | UUUAGGAUCUCC | [MePhosphonate-4O- |
| | UCCUAAAGCAGCC | [mA][fA][fG][fG][fA][mG] | UUUGCCCAGG | [mUs][fUs][fU][mA] |
| | GAAAGGCUGC | [mA][mU][mC][mC][mU][mA] | (SEQ ID | [fG][mG][fA][mU][mC] |
| | (SEQ ID | [mA][mA][mG][mC][mA][mG] | NO: 14) | [FR][mC][mC][mU][fU] |
| | NO: 27) | [mC][mC][mG][ademA- | | [mU][mG][mC][mC][mC] |
| | | GalNAc][ademA-GalNAc] | | [mAs][mGs][mG] |
| | | [ademA-GalNAc][mG][mG][mC] | | (SEQ ID NO: 814) |
| | | [mU][mG][mC] | | |
| | | (SEQ ID NO: 788) | | |
| S27-AS14-M3 | UGGGCAAAGGAGA | [mUs][mG][fG][mG][mC][mA] | UUUAGGAUCUCC | [MePhosphonate-4O- |
| | UCCUAAAGCAGCC | [mA][fA][mG][fG][mA][fG] | UUUGCCCAGG | [mUs][fUs][fU][fA] |
| | GAAAGGCUGC | [fA][mU][mC][mC][fU][mA] | (SEQ ID | [fG][mG][fA][mU][mC] |
| | (SEQ ID | [mA][mA][mG][mC][mA][mG] | NO: 14) | [fU][mC][fC][mU][fU] |
| | NO: 27) | [mC][mC][mG][ademA- | | [mU][fG][fC][mC][fC] |
| | | GalNAc][ademA-GalNAc] | | [mAs][mGs][mG] |
| | | [ademA-GalNAc][mG][mG][mC] | | (SEQ ID NO: 815) |
| | | [mU][mG][mC] | | |
| | | (SEQ ID NO: 789) | | |
| S28-AS15-M2 | AAAGAGAAAUGAA | [mAs][mA][mA][mG][mA][mG] | UUAGGUUUUCAU | [MePhosphonate-4O- |
| | AACCUAAGCAGCC | [mA][fA][fA][fU][fG][mA] | UUCUCUUUGG | [mUs][fUs][fA][mG] |
| | GAAAGGCUGC | [mA][mA][mA][mC][mC][mU] | (SEQ ID | [fG][mU][fU][mU][mU] |
| | (SEQ ID | [mA][mA][mG][mC][mA][mG] | NO: 15) | [fC][mA][mU][mU][fU] |
| | NO: 28) | [mC][mC][mG][ademA- | | [mC][mU][mC][mU][mU] |
| | | GalNAc][ademA-GalNAc] | | [mUs][mGs][mG] |
| | | [ademA-GalNAc][mG][mG][mC] | | (SEQ ID NO: 816) |
| | | [mU][mG][mC] | | |
| | | (SEQ ID NO: 790) | | |
| S28-AS15-M3 | AAAGAGAAAUGAA | [mAs][mA][fA][mG][mA][mG] | UUAGGUUUUCAU | [MePhosphonate-4O- |
| | AACCUAAGCAGCC | [mA][fA][mA][fU][mG][fA] | UUCUCUUUGG | [mUs][fUs][fA][fG] |
| | GAAAGGCUGC | [fA][mA][mA][mC][fC][mU] | (SEQ ID | [fG][mU][fU][mU][mU] |
| | (SEQ ID | [mA][mA][mG][mC][mA][mG] | NO: 15) | [fC][mA][fU][mU][fU] |
| | NO: 28) | [mC][mC][mG][ademA- | | [mC][fU][fC][mU][fU] |
| | | GalNAc][ademA-GalNAc] | | [mUs][mGs][mG] |
| | | [ademA-GalNAc][mG][mG][mC] | | (SEQID NO: 817) |
| | | [mU][mG][mC] | | |
| | | (SEQ ID NO: 791) | | |
| S29-AS16-M2 | AAGAAGAUGAUGA | [mAs][mA][mG][mA][mA][mG] | UUCAUCAUCAUC | [MePhosphonate-4O- |
| | UGAUGAAGCAGCC | [mA][fU][fG][fA][fU][mG] | AUCUUCUUGG | [mUs][fUs][fC][mA] |
| | GAAAGGCUGC | [mA][mU][mG][mA][mU][mG] | (SEQ ID | [fU][mC][fA][mU][mC] |
| | (SEQ ID | [mA][mA][mG][mC][mA][mG] | NO: 16) | [fA][mU][mC][mA][fU] |
| | NO: 29) | [mC][mC][mG][ademA- | | [mC][mU][mU][mC][mU] |
| | | GalNAc][ademA-GalNAc] | | [mUs][mGs][mG] |
| | | [ademA-GalNAc][mG][mG][mC] | | (SEQ ID NO: 818) |
| | | [mU][mG][mC] | | |
| | | (SEQ ID NO: 792) | | |
| S29-AS16-M3 | AAGAAGAUGAUGA | [mAs][mA][fG][mA][mA][mG] | UUCAUCAUCAUC | [MePhosphonate-4O- |
| | UGAUGAAGCAGCC | [mA][fU][mG][fA][mU][fG] | AUCUUCUUGG | [mUs][fUs][fC][fA] |
| | GAAAGGCUGC | [fA][mU][mG][mA][fU][mG] | (SEQ ID | [fU][mC][fA][mU][mC] |
| | (SEQ ID | [mA][mA][mG][mC][mA][mG] | NO: 16) | [fA][mU][fC][mA][fU] |
| | NO: 29) | [mC][mC][mG][ademA- | | [mC][fU][fU][mC][fU] |
| | | GalNAc][ademA-GalNAc] | | [mUs][mGs][mG] |
| | | [ademA-GalNAc][mG][mG][mC] | | (SEQ ID NO: 819) |
| | | [mU][mG][mC] | | |
| | | (SEQ ID NO: 793) | | |
| S30-AS17-M2 | AUGAUGAUGAUGA | [mAs][mU][mG][mA][mU][mG] | UACUUAUUCAUC | [MePhosphonate-4O- |
| | AUAAGUAGCAGCC | [mA][fU][fG][fA][fU][mG] | AUCAUCAUGG | [mUs][FAS][fC][mU] |
| | GAAAGGCUGC | [mA][mA][mU][mA][mA][mG] | (SEQ ID | [fU][mA][fU][mU][mC] |
| | (SEQ ID | [mU][mA][mG][mC][mA][mG] | NO: 17) | [fA][mU][mC][mA][fU] |
| | NO: 30) | [mC][mC][mG][ademA- | | [mC][mA][mU][mC][mA] |
| | | GalNAc][ademA-GalNAc] | | [mUs][mGs][mG] |
| | | [ademA-GalNAc][mG][mG][mC] | | (SEQ ID NO: 820) |
| | | [mU][mG][mC] | | |
| | | (SEQ ID NO: 794) | | |
| S30-AS17-M3 | AUGAUGAUGAUGA | [mAs][mU][fG][mA][mU][mG] | UACUUAUUCAUC | [MePhosphonate-4O- |
| | AUAAGUAGCAGCC | [mA][fU][mG][fA][mU][fG] | AUCAUCAUGG | [mUs][FAS][fC][fU] |
| | GAAAGGCUGC | [fA][mA][mU][mA][fA][mG] | (SEQ ID | [fU][mA][fU][mU][mC] |
| | (SEQ ID | [mU][mA][mG][mC][mA][mG] | NO: 17) | [fA][mU][fC][mA][fU] |
| | NO: 30) | [mC][mC][mG][ademA- | | [mC][fA][fU][mC][fA] |
| | | GalNAc][ademA-GalNAc] | | [mUs][FGS][mG] |
| | | [ademA-GalNAc][mG][mG][mC] | | (SEQ ID NO: 821) |
| | | [mU][mG][mC] | | |
| | | (SEQ ID NO: 795) | | |
| S31-AS18-M1 | GAUGAUGAAUAAG | [mGs][mA][fU][mG][fA][mU] | GAACCAACUUAU | [MePhosphonate-4O- |
| | UUGGUUAGCAGCC | [mG][fA][fA][fU][fA][mA] | UCAUCAUCGG | [mUs][FAS][fA][mC] |
| | GAAAGGCUGC | [fG][mU][fU][mG][fG][mU] | (SEQ ID | [fC][mA][fA][mC][mU] |
| | (SEQ ID | [mU][mA][mG][mC][mA][mG] | NO: 18) | [fU][mA][fU][mU][fC] |
| | NO: 31) | [mC][mC][mG][ademA- | | [mA][fU][fC][mA][fU] |
| | | GalNAc][ademA-GalNAc] | | [mCs][mGs][mG] |
| | | [ademA-GalNAc][mG][mG][mC] | | (SEQ ID NO: 822) |
| | | [mU][mG][mC] | | |
| | | (SEQ ID NO: 796) | | |
| S31-AS18-M2 | GAUGAUGAAUAAG | [mGs][mA][mU][mG][mA][mU] | GAACCAACUUAU | [MePhosphonate-4O- |
| | UUGGUUAGCAGCC | [mG][fA][fA][fU][fA][mA] | UCAUCAUCGG | [mUs][FAS][fA][mC] |
| | GAAAGGCUGC | [mG][mU][mU][mG][mG][mU] | (SEQ ID | [fC][mA][fA][mC][mU] |
| | (SEQ ID | [mU][mA][mG][mC][mA][mG] | NO: 18) | [fU][mA][mU][mU][fC] |
| | NO: 31) | [mC][mC][mG][ademA- | | [mA][mU][mC][mA][mU] |
| | | GalNAc][ademA-GalNAc] | | [mCs][mGs][mG] |
| | | [ademA-GalNAc][mG][mG][mC] | | (SEQ ID NO: 823) |
| | | [mU][mG][mC] | | |
| | | (SEQ ID NO: 797) | | |
| S31-AS18-M3 | GAUGAUGAAUAAG | [mGs][mA][fU][mG][mA][mU] | GAACCAACUUAU | [MePhosphonate-4O- |
| | UUGGUUAGCAGCC | [mG][fA][mA][fU][mA][fA] | UCAUCAUCGG | [mUs][FAS][fA][fC] |
| | GAAAGGCUGC | [fG][mU][mU][mG][fG][mU] | (SEQ ID | [fC][mA][fA][mC][mU] |
| | (SEQ ID | [mU][mA][mG][mC][mA][mG] | NO: 18) | [fU][mA][fU][mU][fC] |
| | NO: 31) | [mC][mC][mG][ademA- | | [mA][fU][fC][mA][fU] |
| | | GalNAc][ademA-GalNAc] | | [mCs][mGs][mG] |
| | | [ademA-GalNAc][mG][mG][mC] | | (SEQ ID NO: 824) |
| | | [mU][mG][mC] | | |
| | | (SEQ ID NO: 798) | | |
| S32-AS19-M2 | UGAAUAAGUUGGU | [mUs][mG][mA][mA][mU][mA] | UGCUAGAACCAA | [MePhosphonate-4O- |
| | UCUAGCAGCAGCC | [mA][fG][fU][fU][fG][mG] | CUUAUUCAGG | [mUs][FGS][fC][mU] |
| | GAAAGGCUGC | [mU][mU][mC][mU][mA][mG] | (SEQ ID | [fA][mG][fA][mA][mC] |
| | (SEQ ID | [mC][mA][mG][mC][mA][mG] | NO: 19) | [fC][mA][mA][mC][fU] |
| | NO: 32) | [mC][mC][mG][ademA- | | [mU][mA][mU][mU][mC] |
| | | GalNAc][ademA-GalNAc] | | [mAs][mGs][mG] |
| | | [ademA-GalNAc][mG][mG][mC] | | (SEQ ID NO: 825) |
| | | [mU][mG][mC] | | |
| | | (SEQ ID NO: 799) | | |
| S32-AS19-M3 | UGAAUAAGUUGGU | [mUs][mG][fA][mA][mU][mA] | UGCUAGAACCAA | [MePhosphonate-4O- |
| | UCUAGCAGCAGCC | [mA][fG][mU][fU][mG][fG] | CUUAUUCAGG | [mUs][FGS][fC][fU] |
| | GAAAGGCUGC | [fU][mU][mC][mU][fA][mG] | (SEQ ID | [fA][mG][fA][mA][mC] |
| | (SEQ ID | [mC][mA][mG][mC][mA][mG] | NO: 19) | [fC][mA][fA][mC][fU] |
| | NO: 32) | [mC][mC][mG][ademA- | | [mU][fA][fU][mU][fC] |
| | | GalNAc][ademA-GalNAc] | | [mAs][mGs][mG] |
| | | [ademA-GalNAc][mG][mG][mC] | | (SEQ ID NO: 826) |
| | | [mU][mG][mC] | | |
| | | (SEQ ID NO: 800) | | |
| S33-AS20-M2 | AAUAAGUUGGUUC | [mUs][mG][fA][mA][mU][mA] | UGCGCUAGAACC | [MePhosphonate-4O- |
| | UAGCGCAGCAGCC | [mA][fG][mU][fU][mG][fG] | AACUUAUUGG | [mUs][FGS][fC][mG] |
| | GAAAGGCUGC | [fU][mU][mC][mU][fA][mG] | (SEQ ID | [fC][mU][fA][mG][mA] |
| | (SEQ ID | [mC][mA][mG][mC][mA][mG] | NO: 20) | [fA][mC][mC][mA][fA] |
| | NO: 33) | [mC][mC][mG][ademA- | | [mC][mU][mU][mA][mU] |
| | | GalNAc][ademA-GalNAc] | | [mUs][mGs][mG] |
| | | [ademA-GalNAc][mG][mG][mC] | | (SEQ ID NO: 827) |
| | | [mU][mG][mC] | | |
| | | (SEQ ID NO: 801) | | |
| S33-AS20-M3 | AAUAAGUUGGUUC | [mAs][mA][fU][mA][mA][mG] | UGCGCUAGAACC | [MePhosphonate-4O- |
| | UAGCGCAGCAGCC | [mU][fU][mG][fG][mU][fU] | AACUUAUUGG | [mUs][FGS][fC][fG] |
| | GAAAGGCUGC | [fC][mU][mA][mG][fC][mG] | (SEQ ID | [fC][mU][fA][mG][mA] |
| | (SEQ ID | [mC][mA][mG][mC][mA][mG] | NO: 20) | [fA][mC][fC][mA][fA] |
| | NO: 33) | [mC][mC][mG][ademA- | | [mC][fU][fU][mA][fU] |
| | | GalNAc][ademA-GalNAc] | | [mUs][mGs][mG] |
| | | [ademA-GalNAc][mG][mG][mC] | | (SEQ ID NO: 828) |
| | | [mU][mG][mC] | | |
| | | (SEQ ID NO: 802) | | |
| S34-AS21-M2 | AUAAGUUGGUUCU | [mAs][mU][mA][mA][mG][mU] | UUGCGCUAGAAC | [MePhosphonate-4O- |
| | AGCGCAAGCAGCC | [mU][fG][fG][fU][fU][mC] | CAACUUAUGG | [mUs][fUs][fG][mC] |
| | GAAAGGCUGC | [mU][mA][mG][mC][mG][mC] | (SEQ ID | [fG][mC][fU][mA][mG] |
| | (SEQ ID | [mA][mA][mG][mC][mA][mG] | NO: 21) | [fA][mA][mC][mC][fA] |
| | NO: 34) | [mC][mC][mG][ademA- | | [mA][mC][mU][mU][mA] |
| | | GalNAc][ademA-GalNAc] | | [mUs][mGs][mG] |
| | | [ademA-GalNAc][mG][mG][mC] | | (SEQ ID NO: 829) |
| | | [mU][mG][mC] | | |
| | | (SEQ ID NO: 803) | | |
| S34-AS21-M3 | AUAAGUUGGUUCU | [mAs][mU][fA][mA][mG][mU] | UUGCGCUAGAAC | [MePhosphonate-4O- |
| | AGCGCAAGCAGCC | [mU][fG][mG][fU][mU][fC] | CAACUUAUGG | [mUs][fUs][fG][fC] |
| | GAAAGGCUGC | [fU][mA][mG][mC][fG][mC] | (SEQ ID | [fG][mC][fU][mA][mG] |
| | (SEQ ID | [mA][mA][mG][mC][mA][mG] | NO: 21) | [fA][mA][fC][mC][fA] |
| | NO: 34) | [mC][mC][mG][ademA- | | [mA][fC][fU][mU][fA] |
| | | GalNAc][ademA-GalNAc] | | [mUs][mGs][mG] |
| | | [ademA-GalNAc][mG][mG][mC] | | (SEQ ID NO: 830) |
| | | [mU][mG][mC] | | |
| | | (SEQ ID NO: 804) | | |
| S35-AS22-M1 | AGAAAAAAAUUGA | [mAs][mG][fA][mA][fA][mA] | UUACAUUUCAAU | [MePhosphonate-4O- |
| | AAUGUAAGCAGCC | [mA][fA][fA][fU][fU][mG] | UUUUUUCUGG | [mUs][fUs][fA][mC] |
| | GAAAGGCUGC | [fA][mA][fA][mU][fG][mU] | (SEQ ID | [fA][mU][fU][mU][mC] |
| | (SEQ ID | [mA][mA][mG][mC][mA][mG] | NO: 22) | [fA][mA][fU][mU][fU] |
| | NO: 35) | [mC][mC][mG][ademA- | | [mU][fU][fU][mU][fC] |
| | | GalNAc][ademA-GalNAc] | | [mUs][mGs][mG] |
| | | [ademA-GalNAc][mG][mG][mC] | | (SEQ ID NO: 831) |
| | | [mU][mG][mC] | | |
| | | (SEQ ID NO: 805) | | |
| S35-AS22-M2 | AGAAAAAAAUUGA | [mAs][mG][mA][mA][mA][mA] | UUACAUUUCAAU | [MePhosphonate-4O- |
| | AAUGUAAGCAGCC | [mA][fA][fA][fU][fU][mG] | UUUUUUCUGG | [mUs][fUs][fA][mC] |
| | GAAAGGCUGC | [mA][mA][mA][mU][mG][mU] | (SEQ ID | [fA][mU][fU][mU][mC] |
| | (SEQ ID | [mA][mA][mG][mC][mA][mG] | NO: 22) | [fA][mA][mU][mU][fU] |
| | NO: 35) | [mC][mC][mG][ademA- | | [mU][mU][mU][mU][mC] |
| | | GalNAc][ademA-GalNAc] | | [mUs][mGs][mG] |
| | | [ademA-GalNAc][mG][mG][mC] | | (SEQ ID NO: 832) |
| | | [mU][mG][mC] | | |
| | | (SEQ ID NO: 806) | | |
| S35-AS22-M3 | AGAAAAAAAUUGA | [mAs][mG][fA][mA][mA][mA] | UUACAUUUCAAU | [MePhosphonate-4O- |
| | AAUGUAAGCAGCC | [mA][fA][mA][fU][mU][fG] | UUUUUUCUGG | [mUs][fUs][fA][fC] |
| | GAAAGGCUGC | [fA][mA][mA][mU][fG][mU] | (SEQ ID | [fA][mU][fU][mU][mC] |
| | (SEQ ID | [mA][mA][mG][mC][mA][mG] | NO: 22) | [fA][mA][fU][mU][fU] |
| | NO: 35) | [mC][mC][mG][ademA- | | [mU][fU][fU][mU][fC] |
| | | GalNAc][ademA-GalNAc] | | [mUs][mGs][mG] |
| | | [ademA-GalNAc][mG][mG][mC] | | (SEQ ID NO: 833) |
| | | [mU][mG][mC] | | |
| | | (SEQ ID NO: 807) | | |
| S36-AS23-M1 | UUGUUGUUCUGUU | [mUs][mU][fG][mU][fU][mG] | UUCAGUUAACAG | [MePhosphonate-4O- |
| | AACUGAAGCAGCC | [mU][fU][fC][fU][fG][mU] | AACAACAAGG | [mUs][fUs][fC][mA] |
| | GAAAGGCUGC | [fU][mA][fA][mC][fU][mG] | (SEQ ID | [fG][mU][fU][mA][mA] |
| | (SEQ ID | [mA][mA][mG][mC][mA][mG] | NO: 23) | [fC][mA][fG][mA][fA] |
| | NO: 36) | [mC][mC][mG][ademA- | | [mC][fA][fA][mC][fA] |
| | | GalNAc][ademA-GalNAc] | | [mAs][mGs][mG] |
| | | [ademA-GalNAc][mG][mG][mC] | | (SEQ ID NO: 834) |
| | | [mU][mG][mC] | | |
| | | (SEQ ID NO: 808) | | |
| S36-AS23-M2 | UUGUUGUUCUGUU | [mUs][mU][mG][mU][mU][mG] | UUCAGUUAACAG | [MePhosphonate-4O- |
| | AACUGAAGCAGCC | [mU][fU][fC][fU][fG][mU] | AACAACAAGG | [mUs][fUs][fC][mA] |
| | GAAAGGCUGC | [mU][mA][mA][mC][mU][mG] | (SEQ ID | [fG][mU][fU][mA][mA] |
| | (SEQ ID | [mA][mA][mG][mC][mA][mG] | NO: 23) | [fC][mA][mG][mA][fA] |
| | NO: 36) | [mC][mC][mG][ademA- | | [mC][mA][mA][mC][mA] |
| | | GalNAc][ademA-GalNAc] | | [mAs][mGs][mG] |
| | | [ademA-GalNAc][mG][mG][mC] | | (SEQ ID NO: 835) |
| | | [mU][mG][mC] | | |
| | | (SEQ ID NO: 809) | | |
| S36-AS23-M3 | UUGUUGUUCUGUU | [mUs][mU][fG][mU][mU][mG] | UUCAGUUAACAG | [MePhosphonate-4O- |
| | AACUGAAGCAGCC | [mU][fU][mC][fU][mG][fU] | AACAACAAGG | [mUs][fUs][fC][fA] |
| | GAAAGGCUGC | [fU][mA][mA][mC][fU][mG] | (SEQ ID | [fG][mU][fU][mA][mA] |
| | (SEQ ID | [mA][mA][mG][mC][mA][mG] | NO: 23) | [fC][mA][fG][mA][fA] |
| | NO: 36) | [mC][mC][mG][ademA- | | [mC][fA][fA][mC][fA] |
| | | GalNAc][ademA-GalNAc] | | [mAs][mGs][mG] |
| | | [ademA-GalNAc][mG][mG][mC] | | (SEQ ID NO: 836) |
| | | [mU][mG][mC] | | |
| | | (SEQ ID NO: 810) | | |
| S37-AS24-M2 | UUCUGAAUGCUUC | [mUs][mU][mC][mU][mG][mA] | UUACUUAGAAGC | [MePhosphonate-4O- |
| | UAAGUAAGCAGCC | [mA][fU][fG][fC][fU][mU] | AUUCAGAAGG | [mUs][fUs][fA][mC] |
| | GAAAGGCUGC | [mC][mU][mA][mA][mG][mU] | (SEQ ID | [fU][mU][fA][mG][mA] |
| | (SEQ ID | [mA][mA][mG][mC][mA][mG] | NO: 24) | [fA][mG][mC][mA][fU] |
| | NO: 37) | [mC][mC][mG][ademA- | | [mU][mC][mA][mG][mA] |
| | | GalNAc][ademA-GalNAc] | | [mAs][mGs][mG] |
| | | [ademA-GalNAc][mG][mG][mC] | | (SEQ ID NO: 837) |
| | | [mU][mG][mC] | | |
| | | (SEQ ID NO: 811) | | |
| S38-AS25-M2 | CUGAAUGCUUCUA | [mCs][mU][mG][mA][mA][mU] | UUUUACUUAGAA | [MePhosphonate-4O- |
| | AGUAAAAGCAGCC | [mG][fC][fU][fU][fC][mU] | GCAUUCAGGG | [mUs][fUs][fU][mU] |
| | GAAAGGCUGC | [mA][mA][mG][mU][mA][mA] | (SEQ ID | [fA][mC][fU][mU][mA] |
| | (SEQ ID | [mA][mA][mG][mC][mA][mG] | NO: 25) | [fG][mA][mA][mG][fC] |
| | NO: 38) | [mC][mC][mG][ademA- | | [mA][mU][mU][mC][mA] |
| | | GalNAc][ademA-GalNAc] | | [mGs][mGs][mG] |
| | | [ademA-GalNAc][mG][mG][mC] | | (SEQ ID NO: 838) |
| | | [mU][mG][mC] | | |
| | | (SEQ ID NO: 812) | | |
| S39-AS26-M2 | GAAUGCUUCUAAG | [mGs][mA][mA][mU][mG][mC] | UUAUUUACUUAG | [MePhosphonate-4O- |
| | UAAAUAAGCAGCC | [mU][fU][fC][fU][fA][mA] | AAGCAUUCGG | [mUs][fUs][fA][mU] |
| | GAAAGGCUGC | [mG][mU][mA][mA][mA][mU] | (SEQ ID | [fU][mU][fA][mC][mU] |
| | (SEQ ID | [mA][mA][mG][mC][mA][mG] | NO: 26) | [fU][mA][mG][mA][fA] |
| | NO: 39) | [mC][mC][mG][ademA- | | [mG][mC][mA][mU][mU] |
| | | GalNAc][ademA-GalNAc] | | [mCs][mGs][mG] |
| | | [ademA-GalNAc][mG][mG][mC] | | (SEQ ID NO: 839) |
| | | [mU][mG][mC] | | |
| | | (SEQ ID NO: 813) | | |

TABLE 3
Modification Key for RNAi Oligonucleotides

| Symbol | Modification Description |
|---|---|
| ademA-GalNAc | 2′-aminodiethoxymethanol-GalNAc adenosine |
| fA | 2′-fluoro-deoxyadenosine |
| fAs | 2′-fluoro-deoxyadenosine followed by a |
| | phosphorothioate linkage |
| fC | 2′-fluoro-deoxycytosine |
| fG | 2′-fluoro-deoxyguanosine |
| fGs | 2′-fluoro-deoxyguanosine followed by a |
| | phosphorothioate linkage |
| fU | 2′-fluoro-uridine |
| fUs | 2′-fluoro-uridine followed by a |
| | phosphorothioate linkage |
| mA | 2′-O-methyl adenosine |
| mAs | 2′-O-methyl adenosine followed by a |
| | phosphorothioate linkage |
| mC | 2′-O-methyl cytosine |
| mCs | 2′-O-methyl cytosine followed by a |
| | phosphorothioate linkage |
| MePhosphonate-4O-mU | Methyl-4′-O-methylphosphonate-2′-O- |
| | methyl uridine |
| MePhosphonate-4O-mUs | Methyl-4′-O-methylphosphonate-2′-O- |
| | methyl uridine followed by |
| | a phosphorothioate linkage |
| mG | 2′-O-methyl guanosine |
| mGs | 2′-O-methyl guanosine followed by a |
| | phosphorothioate linkage |
| mU | 2′-O-methyl uridine |
| mUs | 2′-O-methyl uridine followed by a |
| | phosphorothioate linkage |
| s | Phosphorothioate linkage |

TABLE 4
HMGB1 RNAi Oligonucleotide Sequences Included in Screen

| | | S | | AS |
|---|---|---|---|---|
| RNAi | Sense Sequence/ | SEQ | | SEQ |
| oligonucleotides | mRNA seq | ID NO | Antisense | ID NO |
| S40-AS414 | AACUAAACAUGGGCAAA | 40 | GGAUCUCCUUUGCCCAUGU | 414 |
| | GGAGAUCC | | UUAGUUAU | |
| S41-AS415 | ACUAAACAUGGGCAAAG | 41 | AGGAUUUCCUUUGCCCAUG | 415 |
| | GAAAUCCT | | UUUAGUUA | |
| S42-AS416 | CUAAACAUGGGCAAAGG | 42 | UAGGAUCUCCUUUGCCCAU | 416 |
| | AGAUCCTA | | GUUUAGUU | |
| S43-AS417 | UAAACAUGGGCAAAGGA | 43 | UUAGGUUCUCCUUUGCCCA | 417 |
| | GAACCUAA | | UGUUUAGU | |
| S44-AS418 | AAACAUGGGCAAAGGAG | 44 | CUUAGUAUCUCCUUUGCCC | 418 |
| | AUACUAAG | | AUGUUUAG | |
| S45-AS419 | AACAUGGGCAAAGGAGA | 45 | UCUUAUGAUCUCCUUUGCC | 419 |
| | UCAUAAGA | | CAUGUUUA | |
| S46-AS420 | ACAUGGGCAAAGGAGAU | 46 | UUCUUUGGAUCUCCUUUGC | 420 |
| | CCAAAGAA | | CCAUGUUU | |
| S47-AS421 | CAUGGGCAAAGGAGAUC | 47 | CUUCUUAGGAUCUCCUUUG | 421 |
| | CUAAGAAG | | CCCAUGUU | |
| S48-AS422 | AUGGGCAAAGGAGAUCC | 48 | GCUUCUUAGGAUCUCCUUU | 422 |
| | UAAGAAGC | | GCCCAUGU | |
| S49-AS423 | AAGCCGAGAGGCAAAAU | 49 | AUGAUUACAUUUUGCCUCU | 423 |
| | GUAAUCAT | | CGGCUUCU | |
| S50-AS424 | AGCCGAGAGGCAAAAUG | 50 | UAUGAUGACAUUUUGCCUC | 424 |
| | UCAUCATA | | UCGGCUUC | |
| S51-AS425 | AAAUGUCAUCAUAUGCA | 51 | ACAAAUAAUGCAUAUGAUG | 425 |
| | UUAUUUGT | | ACAUUUUG | |
| S52-AS426 | CAUCAUAUGCAUUUUUU | 52 | GUUUGUACAAAAAAUGCAU | 426 |
| | GUACAAAC | | AUGAUGAC | |
| S53-AS427 | AUCAUAUGCAUUUUUUG | 53 | AGUUUUCACAAAAAAUGCA | 427 |
| | UGAAAACT | | UAUGAUGA | |
| S54-AS428 | AUAUGCAUUUUUUGUGC | 54 | ACAAGUUUGCACAAAAAAU | 428 |
| | AAACUUGT | | GCAUAUGA | |
| S55-AS429 | GUCAACUUCUCAGAGUU | 55 | UCUUAUAAAACUCUGAGAA | 429 |
| | UUAUAAGA | | GUUGACUG | |
| S56-AS430 | AAGAAGUGCUCAGAGAG | 56 | UCUUCUACCUCUCUGAGCA | 430 |
| | GUAGAAGA | | CUUCUUAG | |
| S57-AS431 | AGAAGUGCUCAGAGAGG | 57 | GUCUUUCACCUCUCUGAGC | 431 |
| | UGAAAGAC | | ACUUCUUA | |
| S58-AS432 | GAAGUGCUCAGAGAGGU | 58 | GGUCUUCCACCUCUCUGAG | 432 |
| | GGAAGACC | | CACUUCUU | |
| S59-AS433 | AAGUGCUCAGAGAGGUG | 59 | UGGUCUUCCACCUCUCUGA | 433 |
| | GAAGACCA | | GCACUUCU | |
| S60-AS434 | AGUGCUCAGAGAGGUGG | 60 | AUGGUUUUCCACCUCUCUG | 434 |
| | AAAACCAT | | AGCACUUC | |
| S61-AS435 | GUGCUCAGAGAGGUGGA | 61 | CAUGGUCUUCCACCUCUCU | 435 |
| | AGACCATG | | GAGCACUU | |
| S62-AS436 | UGCUCAGAGAGGUGGAA | 62 | ACAUGUUCUUCCACCUCUC | 436 |
| | GAACAUGT | | UGAGCACU | |
| S63-AS437 | GCUCAGAGAGGUGGAAG | 63 | GACAUUGUCUUCCACCUCU | 437 |
| | ACAAUGTC | | CUGAGCAC | |
| S64-AS438 | CUCAGAGAGGUGGAAGA | 64 | AGACAUGGUCUUCCACCUC | 438 |
| | CCAUGUCT | | UCUGAGCA | |
| S65-AS439 | UCAGAGAGGUGGAAGAC | 65 | CAGACUUGGUCUUCCACCU | 439 |
| | CAAGUCTG | | CUCUGAGC | |
| S66-AS440 | CAGAGAGGUGGAAGACC | 66 | GCAGAUAUGGUCUUCCACC | 440 |
| | AUAUCUGC | | UCUCUGAG | |
| S67-AS441 | AGAGAGGUGGAAGACCA | 67 | AGCAGUCAUGGUCUUCCAC | 441 |
| | UGACUGCT | | CUCUCUGA | |
| S68-AS442 | GAGAGGUGGAAGACCAU | 68 | UAGCAUACAUGGUCUUCCA | 442 |
| | GUAUGCTA | | CCUCUCUG | |
| S69-AS443 | AGAGGUGGAAGACCAUG | 69 | UUAGCUGACAUGGUCUUCC | 443 |
| | UCAGCUAA | | ACCUCUCU | |
| S70-AS444 | GAGGUGGAAGACCAUGU | 70 | UUUAGUAGACAUGGUCUUC | 444 |
| | CUACUAAA | | CACCUCUC | |
| S71-AS445 | AGGUGGAAGACCAUGUC | 71 | CUUUAUCAGACAUGGUCUU | 445 |
| | UGAUAAAG | | CCACCUCU | |
| S72-AS446 | GGUGGAAGACCAUGUCU | 72 | UCUUUUGCAGACAUGGUCU | 446 |
| | GCAAAAGA | | UCCACCUC | |
| S73-AS447 | GUGGAAGACCAUGUCUG | 73 | CUCUUUAGCAGACAUGGUC | 447 |
| | CUAAAGAG | | UUCCACCU | |
| S74-AS448 | UGGAAGACCAUGUCUGC | 74 | UCUCUUUAGCAGACAUGGU | 448 |
| | UAAAGAGA | | CUUCCACC | |
| S75-AS449 | GGAAGACCAUGUCUGCU | 75 | UUCUCUUUAGCAGACAUGG | 449 |
| | AAAGAGAA | | UCUUCCAC | |
| S76-AS450 | GAAGACCAUGUCUGCUA | 76 | UUUCUUUUUAGCAGACAUG | 450 |
| | AAAAGAAA | | GUCUUCCA | |
| S77-AS451 | AAGACCAUGUCUGCUAA | 77 | CUUUCUCUUUAGCAGACAU | 451 |
| | AGAGAAAG | | GGUCUUCC | |
| S78-AS452 | AGACCAUGUCUGCUAAA | 78 | CCUUUUUCUUUAGCAGACA | 452 |
| | GAAAAAGG | | UGGUCUUC | |
| S79-AS453 | AAAUUUGAAGAUAUGGC | 79 | CCGCUUUUGCCAUAUCUUC | 453 |
| | AAAAGCGG | | AAAUUUUC | |
| S80-AS454 | AAUUUGAAGAUAUGGCA | 80 | UCCGCUUUUGCCAUAUCUU | 454 |
| | AAAGCGGA | | CAAAUUUU | |
| S81-AS455 | GAAAGAGAAAUGAAAAC | 81 | GGAUAUAGGUUUUCAUUUC | 455 |
| | CUAUAUCC | | UCUUUCAU | |
| S82-AS456 | AAAAAAGAAGUUCAAGG | 82 | AUUGGUAUCCUUGAACUUC | 456 |
| | AUACCAAT | | UUUUUUGU | |
| S83-AS457 | CCCAAUGCACCCAAGAG | 83 | AAGGAUGCCUCUUGGGUGC | 457 |
| | GCAUCCTT | | AUUGGGAU | |
| S84-AS458 | CCAAUGCACCCAAGAGG | 84 | GAAGGUGGCCUCUUGGGUG | 458 |
| | CCACCUTC | | CAUUGGGA | |
| S85-AS459 | CAAUGCACCCAAGAGGC | 85 | CGAAGUAGGCCUCUUGGGU | 459 |
| | CUACUUCG | | GCAUUGGG | |
| S86-AS460 | AAUGCACCCAAGAGGCC | 86 | CCGAAUGAGGCCUCUUGGG | 460 |
| | UCAUUCGG | | UGCAUUGG | |
| S87-AS461 | AUGCACCCAAGAGGCCU | 87 | GCCGAUGGAGGCCUCUUGG | 461 |
| | CCAUCGGC | | GUGCAUUG | |
| S88-AS462 | UGCACCCAAGAGGCCUC | 88 | GGCCGUAGGAGGCCUCUUG | 462 |
| | CUACGGCC | | GGUGCAUU | |
| S89-AS463 | GCACCCAAGAGGCCUCC | 89 | AGGCCUAAGGAGGCCUCUU | 463 |
| | UUAGGCCT | | GGGUGCAU | |
| S90-AS464 | CACCCAAGAGGCCUCCU | 90 | AAGGCUGAAGGAGGCCUCU | 464 |
| | UCAGCCTT | | UGGGUGCA | |
| S91-AS465 | ACCCAAGAGGCCUCCUU | 91 | GAAGGUCGAAGGAGGCCUC | 465 |
| | CGACCUTC | | UUGGGUGC | |
| S92-AS466 | CCCAAGAGGCCUCCUUC | 92 | AGAAGUCCGAAGGAGGCCU | 466 |
| | GGACUUCT | | CUUGGGUG | |
| S93-AS467 | CCAAGAGGCCUCCUUCG | 93 | AAGAAUGCCGAAGGAGGCC | 467 |
| | GCAUUCTT | | UCUUGGGU | |
| S94-AS468 | CAAGAGGCCUCCUUCGG | 94 | GAAGAUGGCCGAAGGAGGC | 468 |
| | CCAUCUTC | | CUCUUGGG | |
| S95-AS469 | AAGAGGCCUCCUUCGGC | 95 | GGAAGUAGGCCGAAGGAGG | 469 |
| | CUACUUCC | | CCUCUUGG | |
| S96-AS470 | AGAGGCCUCCUUCGGCC | 96 | AGGAAUAAGGCCGAAGGAG | 470 |
| | UUAUUCCT | | GCCUCUUG | |
| S97-AS471 | GAGGCCUCCUUCGGCCU | 97 | GAGGAUGAAGGCCGAAGGA | 471 |
| | UCAUCCTC | | GGCCUCUU | |
| S98-AS472 | AGGCCUCCUUCGGCCUU | 98 | AGAGGUAGAAGGCCGAAGG | 472 |
| | CUACCUCT | | AGGCCUCU | |
| S99-AS473 | GGCCUCCUUCGGCCUUC | 99 | AAGAGUAAGAAGGCCGAAG | 473 |
| | UUACUCTT | | GAGGCCUC | |
| S100-AS474 | GCCUCCUUCGGCCUUCU | 100 | GAAGAUGAAGAAGGCCGAA | 474 |
| | UCAUCUTC | | GGAGGCCU | |
| S101-AS475 | GGAAUAACACUGCUGCA | 101 | UUGUCUUCUGCAGCAGUGU | 475 |
| | GAAGACAA | | UAUUCCAC | |
| S102-AS476 | GAUGACAAGCAGCCUUA | 102 | UCUUUUCAUAAGGCUGCUU | 476 |
| | UGAAAAGA | | GUCAUCUG | |
| S103-AS477 | GGAUAUUGCUGCAUAUC | 103 | UUUAGUUCGAUAUGCAGCA | 477 |
| | GAACUAAA | | AUAUCCUU | |
| S104-AS478 | AGCAAGAAAAAGAAGGA | 104 | CCUCCUCUUCCUUCUUUUU | 478 |
| | AGAGGAGG | | CUUGCUUU | |
| S105-AS479 | GCAAGAAAAAGAAGGAA | 105 | UCCUCUUCUUCCUUCUUUU | 479 |
| | GAAGAGGA | | UCUUGCUU | |
| S106-AS480 | CAAGAAAAAGAAGGAAG | 106 | UUCCUUCUCUUCCUUCUUU | 480 |
| | AGAAGGAA | | UUCUUGCU | |
| S107-AS481 | AAGAAAAAGAAGGAAGA | 107 | CUUCCUCCUCUUCCUUCUU | 481 |
| | GGAGGAAG | | UUUCUUGC | |
| S108-AS482 | AGAAAAAGAAGGAAGAG | 108 | UCUUCUUCCUCUUCCUUCU | 482 |
| | GAAGAAGA | | UUUUCUUG | |
| S109-AS483 | GAAGAAGAUGAUGAUGA | 109 | CUUAUUCAUCAUCAUCAUC | 483 |
| | UGAAUAAG | | UUCUUCUU | |
| S110-AS484 | GAUGAUGAUGAUGAAUA | 110 | AACCAUCUUAUUCAUCAUC | 484 |
| | AGAUGGTT | | AUCAUCUU | |
| S111-AS485 | GAUGAUGAUGAAUAAGU | 111 | UAGAAUCAACUUAUUCAUC | 485 |
| | UGAUUCTA | | AUCAUCAU | |
| S112-AS486 | AUGAUGAUGAAUAAGUU | 112 | CUAGAUCCAACUUAUUCAU | 486 |
| | GGAUCUAG | | CAUCAUCA | |
| S113-AS487 | GAUGAAUAAGUUGGUUC | 113 | CUGCGUUAGAACCAACUUA | 487 |
| | UAACGCAG | | UUCAUCAU | |
| S114-AS488 | UGAAUAAGUUGGUUCUA | 114 | AACUGUGCUAGAACCAACU | 488 |
| | GCACAGTT | | UAUUCAUC | |
| S115-AS489 | GAAUAAGUUGGUUCUAG | 115 | AAACUUCGCUAGAACCAAC | 489 |
| | CGAAGUTT | | UUAUUCAU | |
| S116-AS490 | AAUAAGUUGGUUCUAGC | 116 | AAAACUGCGCUAGAACCAA | 490 |
| | GCAGUUTT | | CUUAUUCA | |
| S117-AS491 | AUAAGUUGGUUCUAGCG | 117 | AAAAAUUGCGCUAGAACCA | 491 |
| | CAAUUUTT | | ACUUAUUC | |
| S118-AS492 | UAAGUUGGUUCUAGCGC | 118 | AAAAAUCUGCGCUAGAACC | 492 |
| | AGAUUUTT | | AACUUAUU | |
| S119-AS493 | AAGUUGGUUCUAGCGCA | 119 | AAAAAUACUGCGCUAGAAC | 493 |
| | GUAUUUTT | | CAACUUAU | |
| S120-AS494 | UUUUUCUUGUCUAUAAA | 120 | UUAAAUGCUUUAUAGACAA | 494 |
| | GCAUUUAA | | GAAAAAAA | |
| S121-AS495 | UUUUCUUGUCUAUAAAG | 121 | GUUAAUUGCUUUAUAGACA | 495 |
| | CAAUUAAC | | AGAAAAAA | |
| S122-AS496 | UUUCUUGUCUAUAAAGC | 122 | GGUUAUAUGCUUUAUAGAC | 496 |
| | AUAUAACC | | AAGAAAAA | |
| S123-AS497 | UUCUUGUCUAUAAAGCA | 123 | GGGUUUAAUGCUUUAUAGA | 497 |
| | UUAAACCC | | CAAGAAAA | |
| S124-AS498 | UCUUGUCUAUAAAGCAU | 124 | GGGGUUAAAUGCUUUAUAG | 498 |
| | UUAACCCC | | ACAAGAAA | |
| S125-AS499 | CAACUCACUCCUUUUAA | 125 | UUUUUUCUUUAAAAGGAGU | 499 |
| | AGAAAAAA | | GAGUUGUG | |
| S126-AS500 | AACUCACUCCUUUUAAA | 126 | UUUUUUUCUUUAAAAGGAG | 500 |
| | GAAAAAAA | | UGAGUUGU | |
| S127-AS501 | ACUCACUCCUUUUAAAG | 127 | AUUUUUUUCUUUAAAAGGA | 501 |
| | AAAAAAAT | | GUGAGUUG | |
| S128-AS502 | CUCACUCCUUUUAAAGA | 128 | AAUUUUUUUCUUUAAAAGG | 502 |
| | AAAAAATT | | AGUGAGUU | |
| S129-AS503 | UCACUCCUUUUAAAGAA | 129 | CAAUUUUUUUCUUUAAAAG | 503 |
| | AAAAAUTG | | GAGUGAGU | |
| S130-AS504 | CACUCCUUUUAAAGAAA | 130 | UCAAUUUUUUUCUUUAAAA | 504 |
| | AAAAUUGA | | GGAGUGAG | |
| S131-AS505 | ACUCCUUUUAAAGAAAA | 131 | UUCAAUUUUUUUCUUUAAA | 505 |
| | AAAUUGAA | | AGGAGUGA | |
| S132-AS506 | CUCCUUUUAAAGAAAAA | 132 | UUUCAUUUUUUUUCUUUAA | 506 |
| | AAAUGAAA | | AAGGAGUG | |
| S133-AS507 | UCCUUUUAAAGAAAAAA | 133 | AUUUCUAUUUUUUUCUUUA | 507 |
| | AUAGAAAT | | AAAGGAGU | |
| S134-AS508 | CCUUUUAAAGAAAAAAA | 134 | CAUUUUAAUUUUUUUCUUU | 508 |
| | UUAAAATG | | AAAAGGAG | |
| S135-AS509 | CUUUUAAAGAAAAAAAU | 135 | ACAUUUCAAUUUUUUUCUU | 509 |
| | UGAAAUGT | | UAAAAGGA | |
| S136-AS510 | UUUUAAAGAAAAAAAUU | 136 | UACAUUUCAAUUUUUUUCU | 510 |
| | GAAAUGTA | | UUAAAAGG | |
| S137-AS511 | UUUAAAGAAAAAAAUUG | 137 | UUACAUUUCAAUUUUUUUC | 511 |
| | AAAUGUAA | | UUUAAAAG | |
| S138-AS512 | UUAAAGAAAAAAAUUGA | 138 | CUUACUUUUCAAUUUUUUU | 512 |
| | AAAGUAAG | | CUUUAAAA | |
| S139-AS513 | UAAAGAAAAAAAUUGAA | 139 | CCUUAUAUUUCAAUUUUUU | 513 |
| | AUAUAAGG | | UCUUUAAA | |
| S140-AS514 | AAAGAAAAAAAUUGAAA | 140 | GCCUUUCAUUUCAAUUUUU | 514 |
| | UGAAAGGC | | UUCUUUAA | |
| S141-AS515 | GAAAAAAAUUGAAAUGU | 141 | ACAGCUUUACAUUUCAAUU | 515 |
| | AAAGCUGT | | UUUUUCUU | |
| S142-AS516 | AAAAAAAUUGAAAUGUA | 142 | CACAGUCUUACAUUUCAAU | 516 |
| | AGACUGTG | | UUUUUUCU | |
| S143-AS517 | GUAAGAUUUGUUUUUAA | 143 | UGUACUGUUUAAAAACAAA | 517 |
| | ACAGUACA | | UCUUACAC | |
| S144-AS518 | UAAGAUUUGUUUUUAAA | 144 | CUGUAUAGUUUAAAAACAA | 518 |
| | CUAUACAG | | AUCUUACA | |
| S145-AS519 | AAGAUUUGUUUUUAAAC | 145 | ACUGUUCAGUUUAAAAACA | 519 |
| | UGAACAGT | | AAUCUUAC | |
| S146-AS520 | AUUUGUUUUUAAACUGU | 146 | GACACUGUACAGUUUAAAA | 520 |
| | ACAGUGTC | | ACAAAUCU | |
| S147-AS521 | UUGUUUUUAAACUGUAC | 147 | AAGACUCUGUACAGUUUAA | 521 |
| | AGAGUCTT | | AAACAAAU | |
| S148-AS522 | UGUUUUUAAACUGUACA | 148 | AAAGAUACUGUACAGUUUA | 522 |
| | GUAUCUTT | | AAAACAAA | |
| S149-AS523 | GUUUUUAAACUGUACAG | 149 | AAAAGUCACUGUACAGUUU | 523 |
| | UGACUUTT | | AAAAACAA | |
| S150-AS524 | UUUUUAAACUGUACAGU | 150 | AAAAAUACACUGUACAGUU | 524 |
| | GUAUUUTT | | UAAAAACA | |
| S151-AS525 | UUUUAAACUGUACAGUG | 151 | AAAAAUGACACUGUACAGU | 525 |
| | UCAUUUTT | | UUAAAAAC | |
| S152-AS526 | UUUAAACUGUACAGUGU | 152 | AAAAAUAGACACUGUACAG | 526 |
| | CUAUUUTT | | UUUAAAAA | |
| S153-AS527 | UUAAACUGUACAGUGUC | 153 | CAAAAUAAGACACUGUACA | 527 |
| | UUAUUUTG | | GUUUAAAA | |
| S154-AS528 | UAAACUGUACAGUGUCU | 154 | ACAAAUAAAGACACUGUAC | 528 |
| | UUAUUUGT | | AGUUUAAA | |
| S155-AS529 | AAACUGUACAGUGUCUU | 155 | UACAAUAAAAGACACUGUA | 529 |
| | UUAUUGTA | | CAGUUUAA | |
| S156-AS530 | AACUGUACAGUGUCUUU | 156 | AUACAUAAAAAGACACUGU | 530 |
| | UUAUGUAT | | ACAGUUUA | |
| S157-AS531 | ACUGUACAGUGUCUUUU | 157 | UAUACUAAAAAAGACACUG | 531 |
| | UUAGUATA | | UACAGUUU | |
| S158-AS532 | CUGUACAGUGUCUUUUU | 158 | CUAUAUAAAAAAAGACACU | 532 |
| | UUAUAUAG | | GUACAGUU | |
| S159-AS533 | UGUACAGUGUCUUUUUU | 159 | ACUAUUCAAAAAAAGACAC | 533 |
| | UGAAUAGT | | UGUACAGU | |
| S160-AS534 | UACAGUGUCUUUUUUUG | 160 | UAACUUUACAAAAAAAGAC | 534 |
| | UAAAGUTA | | ACUGUACA | |
| S161-AS535 | CAGUGUCUUUUUUUGUA | 161 | GUUAAUUAUACAAAAAAAG | 535 |
| | UAAUUAAC | | ACACUGUA | |
| S162-AS536 | GUGGUAUUUUCAAUAGC | 162 | GGUUAUUGGCUAUUGAAAA | 536 |
| | CAAUAACC | | UACCACCA | |
| S163-AS537 | UGGUAUUUUCAAUAGCC | 163 | AGGUUUGUGGCUAUUGAAA | 537 |
| | ACAAACCT | | AUACCACC | |
| S164-AS538 | GGUAUUUUCAAUAGCCA | 164 | AAGGUUAGUGGCUAUUGAA | 538 |
| | CUAACCTT | | AAUACCAC | |
| S165-AS539 | UAUUUUCAAUAGCCACU | 165 | GCAAGUUUAGUGGCUAUUG | 539 |
| | AAACUUGC | | AAAAUACC | |
| S166-AS540 | AUUUUCAAUAGCCACUA | 166 | GGCAAUGUUAGUGGCUAUU | 540 |
| | ACAUUGCC | | GAAAAUAC | |
| S167-AS541 | UUUUCAAUAGCCACUAA | 167 | AGGCAUGGUUAGUGGCUAU | 541 |
| | CCAUGCCT | | UGAAAAUA | |
| S168-AS542 | UUUCAAUAGCCACUAAC | 168 | CAGGCUAGGUUAGUGGCUA | 542 |
| | CUAGCCTG | | UUGAAAAU | |
| S169-AS543 | UUCAAUAGCCACUAACC | 169 | CCAGGUAAGGUUAGUGGCU | 543 |
| | UUACCUGG | | AUUGAAAA | |
| S170-AS544 | UCAAUAGCCACUAACCU | 170 | ACCAGUCAAGGUUAGUGGC | 544 |
| | UGACUGGT | | UAUUGAAA | |
| S171-AS545 | CAAUAGCCACUAACCUU | 171 | UACCAUGCAAGGUUAGUGG | 545 |
| | GCAUGGTA | | CUAUUGAA | |
| S172-AS546 | AAUAGCCACUAACCUUG | 172 | GUACCUGGCAAGGUUAGUG | 546 |
| | CCAGGUAC | | GCUAUUGA | |
| S173-AS547 | AUAGCCACUAACCUUGC | 173 | UGUACUAGGCAAGGUUAGU | 547 |
| | CUAGUACA | | GGCUAUUG | |
| S174-AS548 | UAGCCACUAACCUUGCC | 174 | CUGUAUCAGGCAAGGUUAG | 548 |
| | UGAUACAG | | UGGCUAUU | |
| S175-AS549 | AGCCACUAACCUUGCCU | 175 | ACUGUUCCAGGCAAGGUUA | 549 |
| | GGAACAGT | | GUGGCUAU | |
| S176-AS550 | GCCACUAACCUUGCCUG | 176 | UACUGUACCAGGCAAGGUU | 550 |
| | GUACAGTA | | AGUGGCUA | |
| S177-AS551 | CCACUAACCUUGCCUGG | 177 | AUACUUUACCAGGCAAGGU | 551 |
| | UAAAGUAT | | UAGUGGCU | |
| S178-AS552 | CACUAACCUUGCCUGGU | 178 | CAUACUGUACCAGGCAAGG | 552 |
| | ACAGUATG | | UUAGUGGC | |
| S179-AS553 | ACUAACCUUGCCUGGUA | 179 | CCAUAUUGUACCAGGCAAG | 553 |
| | CAAUAUGG | | GUUAGUGG | |
| S180-AS554 | GGGUUGUAAAUUGGCAU | 180 | AAAUUUCCAUGCCAAUUUA | 554 |
| | GGAAAUTT | | CAACCCCC | |
| S181-AS555 | GGUUGUAAAUUGGCAUG | 181 | UAAAUUUCCAUGCCAAUUU | 555 |
| | GAAAUUTA | | ACAACCCC | |
| S182-AS556 | GUUGUAAAUUGGCAUGG | 182 | UUAAAUUUCCAUGCCAAUU | 556 |
| | AAAUUUAA | | UACAACCC | |
| S183-AS557 | UUGUAAAUUGGCAUGGA | 183 | UUUAAUUUUCCAUGCCAAU | 557 |
| | AAAUUAAA | | UUACAACC | |
| S184-AS558 | UGUAAAUUGGCAUGGAA | 184 | CUUUAUAUUUCCAUGCCAA | 558 |
| | AUAUAAAG | | UUUACAAC | |
| S185-AS559 | GUAAAUUGGCAUGGAAA | 185 | GCUUUUAAUUUCCAUGCCA | 559 |
| | UUAAAAGC | | AUUUACAA | |
| S186-AS560 | UAAAUUGGCAUGGAAAU | 186 | UGCUUUAAAUUUCCAUGCC | 560 |
| | UUAAAGCA | | AAUUUACA | |
| S187-AS561 | AAAUUGGCAUGGAAAUU | 187 | CUGCUUUAAAUUUCCAUGC | 561 |
| | UAAAGCAG | | CAAUUUAC | |
| S188-AS562 | AAUUGGCAUGGAAAUUU | 188 | CCUGCUUUAAAUUUCCAUG | 562 |
| | AAAGCAGG | | CCAAUUUA | |
| S189-AS563 | AUUGGCAUGGAAAUUUA | 189 | ACCUGUUUUAAAUUUCCAU | 563 |
| | AAACAGGT | | GCCAAUUU | |
| S190-AS564 | UUGGCAUGGAAAUUUAA | 190 | AACCUUCUUUAAAUUUCCA | 564 |
| | AGAAGGTT | | UGCCAAUU | |
| S191-AS565 | UGGCAUGGAAAUUUAAA | 191 | GAACCUGCUUUAAAUUUCC | 565 |
| | GCAGGUTC | | AUGCCAAU | |
| S192-AS566 | GGCAUGGAAAUUUAAAG | 192 | AGAACUUGCUUUAAAUUUC | 566 |
| | CAAGUUCT | | CAUGCCAA | |
| S193-AS567 | GCAUGGAAAUUUAAAGC | 193 | AAGAAUCUGCUUUAAAUUU | 567 |
| | AGAUUCTT | | CCAUGCCA | |
| S194-AS568 | CAUGGAAAUUUAAAGCA | 194 | CAAGAUCCUGCUUUAAAUU | 568 |
| | GGAUCUTG | | UCCAUGCC | |
| S195-AS569 | AUGGAAAUUUAAAGCAG | 195 | ACAAGUACCUGCUUUAAAU | 569 |
| | GUACUUGT | | UUCCAUGC | |
| S196-AS570 | UGGAAAUUUAAAGCAGG | 196 | AACAAUAACCUGCUUUAAA | 570 |
| | UUAUUGTT | | UUUCCAUG | |
| S197-AS571 | GGAAAUUUAAAGCAGGU | 197 | CAACAUGAACCUGCUUUAA | 571 |
| | UCAUGUTG | | AUUUCCAU | |
| S198-AS572 | GAAAUUUAAAGCAGGUU | 198 | CCAACUAGAACCUGCUUUA | 572 |
| | CUAGUUGG | | AAUUUCCA | |
| S199-AS573 | AAAUUUAAAGCAGGUUC | 199 | ACCAAUAAGAACCUGCUUU | 573 |
| | UUAUUGGT | | AAAUUUCC | |
| S200-AS574 | AAUUUAAAGCAGGUUCU | 200 | CACCAUCAAGAACCUGCUU | 574 |
| | UGAUGGTG | | UAAAUUUC | |
| S201-AS575 | AUUUAAAGCAGGUUCUU | 201 | GCACCUACAAGAACCUGCU | 575 |
| | GUAGGUGC | | UUAAAUUU | |
| S202-AS576 | UUUAAAGCAGGUUCUUG | 202 | UGCACUAACAAGAACCUGC | 576 |
| | UUAGUGCA | | UUUAAAUU | |
| S203-AS577 | UUAAAGCAGGUUCUUGU | 203 | GUGCAUCAACAAGAACCUG | 577 |
| | UGAUGCAC | | CUUUAAAU | |
| S204-AS578 | UAAAGCAGGUUCUUGUU | 204 | UGUGCUCCAACAAGAACCU | 578 |
| | GGAGCACA | | GCUUUAAA | |
| S205-AS579 | AAAGCAGGUUCUUGUUG | 205 | CUGUGUACCAACAAGAACC | 579 |
| | GUACACAG | | UGCUUUAA | |
| S206-AS580 | AAGCAGGUUCUUGUUGG | 206 | GCUGUUCACCAACAAGAAC | 580 |
| | UGAACAGC | | CUGCUUUA | |
| S207-AS581 | AGCAGGUUCUUGUUGGU | 207 | UGCUGUGCACCAACAAGAA | 581 |
| | GCACAGCA | | CCUGCUUU | |
| S208-AS582 | GCAGGUUCUUGUUGGUG | 208 | GUGCUUUGCACCAACAAGA | 582 |
| | CAAAGCAC | | ACCUGCUU | |
| S209-AS583 | CAGGUUCUUGUUGGUGC | 209 | UGUGCUGUGCACCAACAAG | 583 |
| | ACAGCACA | | AACCUGCU | |
| S210-AS584 | AGGUUCUUGUUGGUGCA | 210 | UUGUGUUGUGCACCAACAA | 584 |
| | CAACACAA | | GAACCUGC | |
| S211-AS585 | GGUUCUUGUUGGUGCAC | 211 | UUUGUUCUGUGCACCAACA | 585 |
| | AGAACAAA | | AGAACCUG | |
| S212-AS586 | GUUCUUGUUGGUGCACA | 212 | AUUUGUGCUGUGCACCAAC | 586 |
| | GCACAAAT | | AAGAACCU | |
| S213-AS587 | UUCUUGUUGGUGCACAG | 213 | AAUUUUUGCUGUGCACCAA | 587 |
| | CAAAAATT | | CAAGAACC | |
| S214-AS588 | UCUUGUUGGUGCACAGC | 214 | UAAUUUGUGCUGUGCACCA | 588 |
| | ACAAAUTA | | ACAAGAAC | |
| S215-AS589 | CUUGUUGGUGCACAGCA | 215 | CUAAUUUGUGCUGUGCACC | 589 |
| | CAAAUUAG | | AACAAGAA | |
| S216-AS590 | UUGUUGGUGCACAGCAC | 216 | ACUAAUUUGUGCUGUGCAC | 590 |
| | AAAUUAGT | | CAACAAGA | |
| S217-AS591 | UGUUGGUGCACAGCACA | 217 | AACUAUUUUGUGCUGUGCA | 591 |
| | AAAUAGTT | | CCAACAAG | |
| S218-AS592 | GUUGGUGCACAGCACAA | 218 | UAACUUAUUUGUGCUGUGC | 592 |
| | AUAAGUTA | | ACCAACAA | |
| S219-AS593 | UUGGUGCACAGCACAAA | 219 | AUAACUAAUUUGUGCUGUG | 593 |
| | UUAGUUAT | | CACCAACA | |
| S220-AS594 | UGGUGCACAGCACAAAU | 220 | UAUAAUUAAUUUGUGCUGU | 594 |
| | UAAUUATA | | GCACCAAC | |
| S221-AS595 | GGUGCACAGCACAAAUU | 221 | AUAUAUCUAAUUUGUGCUG | 595 |
| | AGAUAUAT | | UGCACCAA | |
| S222-AS596 | UUUUUUCAUCUUCAGUU | 222 | UCAGAUACAACUGAAGAUG | 596 |
| | GUAUCUGA | | AAAAAACU | |
| S223-AS597 | UUUUUCAUCUUCAGUUG | 223 | AUCAGUGACAACUGAAGAU | 597 |
| | UCACUGAT | | GAAAAAAC | |
| S224-AS598 | UUUUCAUCUUCAGUUGU | 224 | CAUCAUAGACAACUGAAGA | 598 |
| | CUAUGATG | | UGAAAAAA | |
| S225-AS599 | UUUCAUCUUCAGUUGUC | 225 | GCAUCUGAGACAACUGAAG | 599 |
| | UCAGAUGC | | AUGAAAAA | |
| S226-AS600 | UUCAUCUUCAGUUGUCU | 226 | UGCAUUAGAGACAACUGAA | 600 |
| | CUAAUGCA | | GAUGAAAA | |
| S227-AS601 | UCAUCUUCAGUUGUCUC | 227 | CUGCAUCAGAGACAACUGA | 601 |
| | UGAUGCAG | | AGAUGAAA | |
| S228-AS602 | CAUCUUCAGUUGUCUCU | 228 | GCUGCUUCAGAGACAACUG | 602 |
| | GAAGCAGC | | AAGAUGAA | |
| S229-AS603 | AUCUUCAGUUGUCUCUG | 229 | AGCUGUAUCAGAGACAACU | 603 |
| | AUACAGCT | | GAAGAUGA | |
| S230-AS604 | UCUGAUGCAGCUUAUAC | 230 | AUUAUUUCGUAUAAGCUGC | 604 |
| | GAAAUAAT | | AUCAGAGA | |
| S231-AS605 | CUGAUGCAGCUUAUACG | 231 | AAUUAUUUCGUAUAAGCUG | 605 |
| | AAAUAATT | | CAUCAGAG | |
| S232-AS606 | CAGCUUAUACGAAAUAA | 232 | AACAAUAAUUAUUUCGUAU | 606 |
| | UUAUUGTT | | AAGCUGCA | |
| S233-AS607 | AGCUUAUACGAAAUAAU | 233 | GAACAUCAAUUAUUUCGUA | 607 |
| | UGAUGUTC | | UAAGCUGC | |
| S234-AS608 | GCUUAUACGAAAUAAUU | 234 | AGAACUACAAUUAUUUCGU | 608 |
| | GUAGUUCT | | AUAAGCUG | |
| S235-AS609 | CUUAUACGAAAUAAUUG | 235 | CAGAAUAACAAUUAUUUCG | 609 |
| | UUAUUCTG | | UAUAAGCU | |
| S236-AS610 | UAUACGAAAUAAUUGUU | 236 | AACAGUACAACAAUUAUUU | 610 |
| | GUACUGTT | | CGUAUAAG | |
| S237-AS611 | AUACGAAAUAAUUGUUG | 237 | UAACAUAACAACAAUUAUU | 611 |
| | UUAUGUTA | | UCGUAUAA | |
| S238-AS612 | UACGAAAUAAUUGUUGU | 238 | UUAACUGAACAACAAUUAU | 612 |
| | UCAGUUAA | | UUCGUAUA | |
| S239-AS613 | ACGAAAUAAUUGUUGUU | 239 | GUUAAUAGAACAACAAUUA | 613 |
| | CUAUUAAC | | UUUCGUAU | |
| S240-AS614 | CGAAAUAAUUGUUGUUC | 240 | AGUUAUCAGAACAACAAUU | 614 |
| | UGAUAACT | | AUUUCGUA | |
| S241-AS615 | GAAAUAAUUGUUGUUCU | 241 | CAGUUUACAGAACAACAAU | 615 |
| | GUAAACTG | | UAUUUCGU | |
| S242-AS616 | AAAUAAUUGUUGUUCUG | 242 | UCAGUUAACAGAACAACAA | 616 |
| | UUAACUGA | | UUAUUUCG | |
| S243-AS617 | AAUAAUUGUUGUUCUGU | 243 | UUCAGUUAACAGAACAACA | 617 |
| | UAACUGAA | | AUUAUUUC | |
| S244-AS618 | AAUUGUUGUUCUGUUAA | 244 | GUAUUUAGUUAACAGAACA | 618 |
| | CUAAAUAC | | ACAAUUAU | |
| S245-AS619 | AUUGUUGUUCUGUUAAC | 245 | GGUAUUCAGUUAACAGAAC | 619 |
| | UGAAUACC | | AACAAUUA | |
| S246-AS620 | UGUUGUUCUGUUAACUG | 246 | GUGGUUUUCAGUUAACAGA | 620 |
| | AAAACCAC | | ACAACAAU | |
| S247-AS621 | GUUGUUCUGUUAACUGA | 247 | AGUGGUAUUCAGUUAACAG | 621 |
| | AUACCACT | | AACAACAA | |
| S248-AS622 | UGUUCUGUUAACUGAAU | 248 | AGAGUUGUAUUCAGUUAAC | 622 |
| | ACAACUCT | | AGAACAAC | |
| S249-AS623 | UCUGUUAACUGAAUACC | 249 | UACAGUGUGGUAUUCAGUU | 623 |
| | ACACUGTA | | AACAGAAC | |
| S250-AS624 | CUGUUAACUGAAUACCA | 250 | UUACAUAGUGGUAUUCAGU | 624 |
| | CUAUGUAA | | UAACAGAA | |
| S251-AS625 | GUUAACUGAAUACCACU | 251 | AAUUAUAGAGUGGUAUUCA | 625 |
| | CUAUAATT | | GUUAACAG | |
| S252-AS626 | AACUGAAUACCACUCUG | 252 | UGCAAUUACAGAGUGGUAU | 626 |
| | UAAUUGCA | | UCAGUUAA | |
| S253-AS627 | ACUGAAUACCACUCUGU | 253 | UUGCAUUUACAGAGUGGUA | 627 |
| | AAAUGCAA | | UUCAGUUA | |
| S254-AS628 | CUGAAUACCACUCUGUA | 254 | UUUGCUAUUACAGAGUGGU | 628 |
| | AUAGCAAA | | AUUCAGUU | |
| S255-AS629 | UGAAUACCACUCUGUAA | 255 | UUUUGUAAUUACAGAGUGG | 629 |
| | UUACAAAA | | UAUUCAGU | |
| S256-AS630 | GAAUACCACUCUGUAAU | 256 | UUUUUUCAAUUACAGAGUG | 630 |
| | UGAAAAAA | | GUAUUCAG | |
| S257-AS631 | AAUACCACUCUGUAAUU | 257 | UUUUUUGCAAUUACAGAGU | 631 |
| | GCAAAAAA | | GGUAUUCA | |
| S258-AS632 | AAAAAGUUGCAGCUGUU | 258 | GUCAAUAAAACAGCUGCAA | 632 |
| | UUAUUGAC | | CUUUUUUU | |
| S259-AS633 | AAAGUUGCAGCUGUUUU | 259 | AUGUCUACAAAACAGCUGC | 633 |
| | GUAGACAT | | AACUUUUU | |
| S260-AS634 | AAGUUGCAGCUGUUUUG | 260 | AAUGUUAACAAAACAGCUG | 634 |
| | UUAACATT | | CAACUUUU | |
| S261-AS635 | AGUUGCAGCUGUUUUGU | 261 | GAAUGUCAACAAAACAGCU | 635 |
| | UGACAUTC | | GCAACUUU | |
| S262-AS636 | GUUGCAGCUGUUUUGUU | 262 | AGAAUUUCAACAAAACAGC | 636 |
| | GAAAUUCT | | UGCAACUU | |
| S263-AS637 | UGCAGCUGUUUUGUUGA | 263 | UCAGAUUGUCAACAAAACA | 637 |
| | CAAUCUGA | | GCUGCAAC | |
| S264-AS638 | GCAGCUGUUUUGUUGAC | 264 | UUCAGUAUGUCAACAAAAC | 638 |
| | AUACUGAA | | AGCUGCAA | |
| S265-AS639 | CAGCUGUUUUGUUGACA | 265 | AUUCAUAAUGUCAACAAAA | 639 |
| | UUAUGAAT | | CAGCUGCA | |
| S266-AS640 | AGCUGUUUUGUUGACAU | 266 | CAUUCUGAAUGUCAACAAA | 640 |
| | UCAGAATG | | ACAGCUGC | |
| S267-AS641 | GCUGUUUUGUUGACAUU | 267 | GCAUUUAGAAUGUCAACAA | 641 |
| | CUAAAUGC | | AACAGCUG | |
| S268-AS642 | UGUUUUGUUGACAUUCU | 268 | AAGCAUUCAGAAUGUCAAC | 642 |
| | GAAUGCTT | | AAAACAGC | |
| S269-AS643 | UUUGUUGACAUUCUGAA | 269 | UAGAAUCAUUCAGAAUGUC | 643 |
| | UGAUUCTA | | AACAAAAC | |
| S270-AS644 | UUGUUGACAUUCUGAAU | 270 | UUAGAUGCAUUCAGAAUGU | 644 |
| | GCAUCUAA | | CAACAAAA | |
| S271-AS645 | UGUUGACAUUCUGAAUG | 271 | CUUAGUAGCAUUCAGAAUG | 645 |
| | CUACUAAG | | UCAACAAA | |
| S272-AS646 | GUUGACAUUCUGAAUGC | 272 | ACUUAUAAGCAUUCAGAAU | 646 |
| | UUAUAAGT | | GUCAACAA | |
| S273-AS647 | UUGACAUUCUGAAUGCU | 273 | UACUUUGAAGCAUUCAGAA | 647 |
| | UCAAAGTA | | UGUCAACA | |
| S274-AS648 | UGACAUUCUGAAUGCUU | 274 | UUACUUAGAAGCAUUCAGA | 648 |
| | CUAAGUAA | | AUGUCAAC | |
| S275-AS649 | GACAUUCUGAAUGCUUC | 275 | UUUACUUAGAAGCAUUCAG | 649 |
| | UAAGUAAA | | AAUGUCAA | |
| S276-AS650 | CAUUCUGAAUGCUUCUA | 276 | UAUUUUCUUAGAAGCAUUC | 650 |
| | AGAAAATA | | AGAAUGUC | |
| S277-AS651 | AUUCUGAAUGCUUCUAA | 277 | GUAUUUACUUAGAAGCAUU | 651 |
| | GUAAAUAC | | CAGAAUGU | |
| S278-AS652 | UCUGAAUGCUUCUAAGU | 278 | UUGUAUUUACUUAGAAGCA | 652 |
| | AAAUACAA | | UUCAGAAU | |
| S279-AS653 | UGAAUGCUUCUAAGUAA | 279 | AAUUGUAUUUACUUAGAAG | 653 |
| | AUACAATT | | CAUUCAGA | |
| S280-AS654 | AAUGCUUCUAAGUAAAU | 280 | AAAAUUGUAUUUACUUAGA | 654 |
| | ACAAUUTT | | AGCAUUCA | |
| S281-AS655 | AUGCUUCUAAGUAAAUA | 281 | AAAAAUUGUAUUUACUUAG | 655 |
| | CAAUUUTT | | AAGCAUUC | |
| S282-AS656 | GCUUCUAAGUAAAUACA | 282 | AAAAAUAUUGUAUUUACUU | 656 |
| | AUAUUUTT | | AGAAGCAU | |
| S283-AS657 | UUUUUAUUAGUAUUGUU | 283 | AAAAGUACAACAAUACUAA | 657 |
| | GUACUUTT | | UAAAAAAA | |
| S284-AS658 | UUUUAUUAGUAUUGUUG | 284 | GAAAAUGACAACAAUACUA | 658 |
| | UCAUUUTC | | AUAAAAAA | |
| S285-AS659 | UUUAUUAGUAUUGUUGU | 285 | UGAAAUGGACAACAAUACU | 659 |
| | CCAUUUCA | | AAUAAAAA | |
| S286-AS660 | UUAUUAGUAUUGUUGUC | 286 | AUGAAUAGGACAACAAUAC | 660 |
| | CUAUUCAT | | UAAUAAAA | |
| S287-AS661 | UAUUAGUAUUGUUGUCC | 287 | UAUGAUAAGGACAACAAUA | 661 |
| | UUAUCATA | | CUAAUAAA | |
| S288-AS662 | AUUAGUAUUGUUGUCCU | 288 | CUAUGUAAAGGACAACAAU | 662 |
| | UUACAUAG | | ACUAAUAA | |
| S289-AS663 | UUAGUAUUGUUGUCCUU | 289 | CCUAUUAAAAGGACAACAA | 663 |
| | UUAAUAGG | | UACUAAUA | |
| S290-AS664 | UAGUAUUGUUGUCCUUU | 290 | ACCUAUGAAAAGGACAACA | 664 |
| | UCAUAGGT | | AUACUAAU | |
| S291-AS665 | GUAUUGUUGUCCUUUUC | 291 | AGACCUAUGAAAAGGACAA | 665 |
| | AUAGGUCT | | CAAUACUA | |
| S292-AS666 | UUGUUGUCCUUUUCAUA | 292 | UUCAGUCCUAUGAAAAGGA | 666 |
| | GGACUGAA | | CAACAAUA | |
| S293-AS667 | UGUUGUCCUUUUCAUAG | 293 | UUUCAUACCUAUGAAAAGG | 667 |
| | GUAUGAAA | | ACAACAAU | |
| S294-AS668 | UUGUCCUUUUCAUAGGU | 294 | AAUUUUAGACCUAUGAAAA | 668 |
| | CUAAAATT | | GGACAACA | |
| S295-AS669 | UGUCCUUUUCAUAGGUC | 295 | AAAUUUCAGACCUAUGAAA | 669 |
| | UGAAAUTT | | AGGACAAC | |
| S296-AS670 | GUCCUUUUCAUAGGUCU | 296 | AAAAUUUCAGACCUAUGAA | 670 |
| | GAAAUUTT | | AAGGACAA | |
| S297-AS671 | UCCUUUUCAUAGGUCUG | 297 | AAAAAUUUCAGACCUAUGA | 671 |
| | AAAUUUTT | | AAAGGACA | |
| S298-AS672 | CCUUUUCAUAGGUCUGA | 298 | GAAAAUUUUCAGACCUAUG | 672 |
| | AAAUUUTC | | AAAAGGAC | |
| S299-AS673 | AUAGGUCUGAAAUUUUU | 299 | UCAAGUAGAAAAAUUUCAG | 673 |
| | CUACUUGA | | ACCUAUGA | |
| S300-AS674 | AGGGGAAGCUAGUCUUU | 300 | CAAAAUCAAAAGACUAGCU | 674 |
| | UGAUUUTG | | UCCCCUCA | |
| S301-AS675 | GGGGAAGCUAGUCUUUU | 301 | GCAAAUGCAAAAGACUAGC | 675 |
| | GCAUUUGC | | UUCCCCUC | |
| S302-AS676 | GGGAAGCUAGUCUUUUG | 302 | GGCAAUAGCAAAAGACUAG | 676 |
| | CUAUUGCC | | CUUCCCCU | |
| S303-AS677 | GGAAGCUAGUCUUUUGC | 303 | GGGCAUAAGCAAAAGACUA | 677 |
| | UUAUGCCC | | GCUUCCCC | |
| S304-AS678 | GAAGCUAGUCUUUUGCU | 304 | UGGGCUAAAGCAAAAGACU | 678 |
| | UUAGCCCA | | AGCUUCCC | |
| S305-AS679 | CAGUGUUUAUCCUUUCA | 305 | UAACUUUAUGAAAGGAUAA | 679 |
| | UAAAGUTA | | ACACUGUA | |
| S306-AS680 | GUGUUUAUCCUUUCAUA | 306 | GCUAAUUAUAUGAAAGGAU | 680 |
| | UAAUUAGC | | AAACACUG | |
| S307-AS681 | UUAUCCUUUCAUAUAGU | 307 | AUUAGUUAACUAUAUGAAA | 681 |
| | UAACUAAT | | GGAUAAAC | |
| S308-AS682 | GCUAAUAAAAAGCUUUU | 308 | GUGUAUACAAAAGCUUUUU | 682 |
| | GUAUACAC | | AUUAGCUA | |
| S309-AS683 | GUAAAGUUAAGUUGAGA | 309 | GAAAAUUAUCUCAACUUAA | 683 |
| | UAAUUUTC | | CUUUACCC | |
| S310-AS684 | AAAGUUAAGUUGAGAUA | 310 | AUGAAUACUAUCUCAACUU | 684 |
| | GUAUUCAT | | AACUUUAC | |
| S311-AS685 | GUUAAGUUGAGAUAGUU | 311 | UGGAUUAAAACUAUCUCAA | 685 |
| | UUAAUCCA | | CUUAACUU | |
| S312-AS686 | UAAGUUGAGAUAGUUUU | 312 | UAUGGUUGAAAACUAUCUC | 686 |
| | CAACCATA | | AACUUAAC | |
| S313-AS687 | AAGUUGAGAUAGUUUUC | 313 | UUAUGUAUGAAAACUAUCU | 687 |
| | AUACAUAA | | CAACUUAA | |
| S314-AS688 | AGUUGAGAUAGUUUUCA | 314 | GUUAUUGAUGAAAACUAUC | 688 |
| | UCAAUAAC | | UCAACUUA | |
| S315-AS689 | GUUGAGAUAGUUUUCAU | 315 | AGUUAUGGAUGAAAACUAU | 689 |
| | CCAUAACT | | CUCAACUU | |
| S316-AS690 | UGAGAUAGUUUUCAUCC | 316 | UCAGUUAUGGAUGAAAACU | 690 |
| | AUAACUGA | | AUCUCAAC | |
| S317-AS691 | GAGAUAGUUUUCAUCCA | 317 | UUCAGUUAUGGAUGAAAAC | 691 |
| | UAACUGAA | | UAUCUCAA | |
| S318-AS692 | AGAUAGUUUUCAUCCAU | 318 | GUUCAUUUAUGGAUGAAAA | 692 |
| | AAAUGAAC | | CUAUCUCA | |
| S319-AS693 | GUUUUCAUCCAUAACUG | 319 | UGGAUUUUCAGUUAUGGAU | 693 |
| | AAAAUCCA | | GAAAACUA | |
| S320-AS694 | UUCAUCCAUAACUGAAC | 320 | UUUUGUAUGUUCAGUUAUG | 694 |
| | AUACAAAA | | GAUGAAAA | |
| S321-AS695 | UUGAUCAGUUAAGAAAU | 321 | UAUGUUAAAUUUCUUAACU | 695 |
| | UUAACATA | | GAUCAAGA | |
| S322-AS696 | GAUCAGUUAAGAAAUUU | 322 | GCUAUUUGAAAUUUCUUAA | 696 |
| | CAAAUAGC | | CUGAUCAA | |
| S323-AS697 | CAUUUACAAACUGAAGA | 323 | UUGAUUACUCUUCAGUUUG | 697 |
| | GUAAUCAA | | UAAAUGUA | |
| S324-AS698 | AUUUACAAACUGAAGAG | 324 | AUUGAUUACUCUUCAGUUU | 698 |
| | UAAUCAAT | | GUAAAUGU | |
| S325-AS699 | ACAAACUGAAGAGUAAU | 325 | GUAGAUUGAUUACUCUUCA | 699 |
| | CAAUCUAC | | GUUUGUAA | |
| S326-AS700 | AAACAUUUUGAAAGUCU | 326 | UCAAGUACAGACUUUCAAA | 700 |
| | GUACUUGA | | AUGUUUGA | |
| S327-AS701 | AAGGACUAAUAGAAAAG | 327 | AGAACUUACUUUUCUAUUA | 701 |
| | UAAGUUCT | | GUCCUUCA | |
| S328-AS702 | ACUAAUAGAAAAGUAUG | 328 | GGUUAUAACAUACUUUUCU | 702 |
| | UUAUAACC | | AUUAGUCC | |
| S329-AS703 | AGAAAAGUAUGUUCUAA | 329 | UGUAAUGGUUAGAACAUAC | 703 |
| | CCAUUACA | | UUUUCUAU | |
| S330-AS704 | GAAAAGUAUGUUCUAAC | 330 | AUGUAUAGGUUAGAACAUA | 704 |
| | CUAUACAT | | CUUUUCUA | |
| S331-AS705 | AAGUAUGUUCUAACCUU | 331 | CUCAUUUAAAGGUUAGAAC | 705 |
| | UAAAUGAG | | AUACUUUU | |
| S332-AS706 | GUAAUGGCAGUUAUAUU | 332 | AACUGUAAAAUAUAACUGC | 706 |
| | UUACAGTT | | CAUUACAU | |
| S333-AS707 | UAAUGGCAGUUAUAUUU | 333 | GAACUUCAAAAUAUAACUG | 707 |
| | UGAAGUTC | | CCAUUACA | |
| S334-AS708 | UAAAGAAGACCUGAGAA | 334 | GGGAUUCAUUCUCAGGUCU | 708 |
| | UGAAUCCC | | UCUUUAAU | |
| S335-AS709 | AAAGAAGACCUGAGAAU | 335 | GGGGAUACAUUCUCAGGUC | 709 |
| | GUAUCCCC | | UUCUUUAA | |
| S336-AS710 | GAAGACCUGAGAAUGUA | 336 | UUUGGUGAUACAUUCUCAG | 710 |
| | UCACCAAA | | GUCUUCUU | |
| S337-AS711 | AAGACCUGAGAAUGUAU | 337 | UUUUGUGGAUACAUUCUCA | 711 |
| | CCACAAAA | | GGUCUUCU | |
| S338-AS712 | AGACCUGAGAAUGUAUC | 338 | CUUUUUGGGAUACAUUCUC | 712 |
| | CCAAAAAG | | AGGUCUUC | |
| S339-AS713 | GACCUGAGAAUGUAUCC | 339 | GCUUUUGGGGAUACAUUCU | 713 |
| | CCAAAAGC | | CAGGUCUU | |
| S340-AS715 | ACCUGAGAAUGUAUCCC | 340 | CGCUUUUGGGGAUACAUUC | 714 |
| | CAAAAGCG | | UCAGGUCU | |
| S341-AS715 | CCUGAGAAUGUAUCCCC | 341 | ACGCUUUUGGGGAUACAUU | 715 |
| | AAAAGCGT | | CUCAGGUC | |
| S342-AS716 | CUGAGAAUGUAUCCCCA | 342 | CACGCUUUUGGGGAUACAU | 716 |
| | AAAGCGTG | | UCUCAGGU | |
| S343-AS717 | UGAGAAUGUAUCCCCAA | 343 | UCACGUUUUUGGGGAUACA | 717 |
| | AAACGUGA | | UUCUCAGG | |
| S344-AS718 | GAGAAUGUAUCCCCAAA | 344 | CUCACUCUUUUGGGGAUAC | 718 |
| | AGAGUGAG | | AUUCUCAG | |
| S345-AS719 | GCCAUAUUAAAUUUUUU | 345 | AUGUCUACAAAAAAUUUAA | 719 |
| | GUAGACAT | | UAUGGCAG | |
| S346-AS720 | CCAUAUUAAAUUUUUUG | 346 | AAUGUUAACAAAAAAUUUA | 720 |
| | UUAACATT | | AUAUGGCA | |
| S347-AS721 | CAUAUUAAAUUUUUUGU | 347 | UAAUGUCAACAAAAAAUUU | 721 |
| | UGACAUTA | | AAUAUGGC | |
| S348-AS722 | AUAUUAAAUUUUUUGUU | 348 | CUAAUUUCAACAAAAAAUU | 722 |
| | GAAAUUAG | | UAAUAUGG | |
| S349-AS723 | AUUAAAUUUUUUGUUGA | 349 | GACUAUUGUCAACAAAAAA | 723 |
| | CAAUAGTC | | UUUAAUAU | |
| S350-AS724 | AAAUUUUUUGUUGACAU | 350 | UGAGAUUAAUGUCAACAAA | 724 |
| | UAAUCUCA | | AAAUUUAA | |
| S351-AS725 | AAUUUUUUGUUGACAUU | 351 | CUGAGUCUAAUGUCAACAA | 725 |
| | AGACUCAG | | AAAAUUUA | |
| S352-AS726 | AUUUUUUGUUGACAUUA | 352 | ACUGAUACUAAUGUCAACA | 726 |
| | GUAUCAGT | | AAAAAUUU | |
| S353-AS727 | GAAGACUAUGAAAAUGC | 353 | UAUAGUCAGCAUUUUCAUA | 727 |
| | UGACUATA | | GUCUUCAC | |
| S354-AS728 | AGACUUUCCAUUACAAG | 354 | UAAAAUUACUUGUAAUGGA | 728 |
| | UAAUUUTA | | AAGUCUCG | |
| S355-AS729 | ACUUUGCAUCUCAGUAU | 355 | AAUAAUUCAUACUGAGAUG | 729 |
| | GAAUUATT | | CAAAGUUU | |
| S356-AS730 | CUUUGCAUCUCAGUAUG | 356 | GAAUAUUUCAUACUGAGAU | 730 |
| | AAAUAUTC | | GCAAAGUU | |
| S357-AS731 | UUGCAUCUCAGUAUGAA | 357 | UUGAAUAAUUCAUACUGAG | 731 |
| | UUAUUCAA | | AUGCAAAG | |
| S358-AS732 | GCAUCUCAGUAUGAAUU | 358 | AAUUGUAUAAUUCAUACUG | 732 |
| | AUACAATT | | AGAUGCAA | |
| S359-AS733 | CAUCUCAGUAUGAAUUA | 359 | AAAUUUAAUAAUUCAUACU | 733 |
| | UUAAAUTT | | GAGAUGCA | |
| S360-AS734 | GAAUGAUUUUUCUUUAC | 360 | UUUGUUUUGUAAAGAAAAA | 734 |
| | AAAACAAA | | UCAUUCAA | |
| S361-AS735 | AGUUUAGGGAACAAUUU | 361 | AAAUUUCCAAAUUGUUCCC | 735 |
| | GGAAAUTT | | UAAACUCC | |
| S362-AS736 | GUUUAGGGAACAAUUUG | 362 | AAAAUUGCCAAAUUGUUCC | 736 |
| | GCAAUUTT | | CUAAACUC | |
| S363-AS737 | UUUAGGGAACAAUUUGG | 363 | CAAAAUUGCCAAAUUGUUC | 737 |
| | CAAUUUTG | | CCUAAACU | |
| S364-AS738 | UUAGGGAACAAUUUGGC | 364 | ACAAAUUUGCCAAAUUGUU | 738 |
| | AAAUUUGT | | CCCUAAAC | |
| S365-AS739 | UAGGGAACAAUUUGGCA | 365 | CACAAUAUUGCCAAAUUGU | 739 |
| | AUAUUGTG | | UCCCUAAA | |
| S366-AS740 | AGGGAACAAUUUGGCAA | 366 | CCACAUAAUUGCCAAAUUG | 740 |
| | UUAUGUGG | | UUCCCUAA | |
| S367-AS741 | GGGAACAAUUUGGCAAU | 367 | ACCACUAAAUUGCCAAAUU | 741 |
| | UUAGUGGT | | GUUCCCUA | |
| S368-AS742 | GGAACAAUUUGGCAAUU | 368 | AACCAUAAAAUUGCCAAAU | 742 |
| | UUAUGGTT | | UGUUCCCU | |
| S369-AS743 | GAACAAUUUGGCAAUUU | 369 | AAACCUCAAAAUUGCCAAA | 743 |
| | UGAGGUTT | | UUGUUCCC | |
| S370-AS744 | AACAAUUUGGCAAUUUU | 370 | AAAACUACAAAAUUGCCAA | 744 |
| | GUAGUUTT | | AUUGUUCC | |
| S371-AS745 | ACAAUUUGGCAAUUUUG | 371 | GAAAAUCACAAAAUUGCCA | 745 |
| | UGAUUUTC | | AAUUGUUC | |
| S372-AS746 | CAAUUUGGCAAUUUUGU | 372 | CGAAAUCCACAAAAUUGCC | 746 |
| | GGAUUUCG | | AAAUUGUU | |
| S373-AS747 | AAAUAGCGUUCUUGUAA | 373 | GUGUAUAAUUACAAGAACG | 747 |
| | UUAUACAC | | CUAUUUUA | |
| S374-AS748 | AAUAGCGUUCUUGUAAU | 374 | CGUGUUAAAUUACAAGAAC | 748 |
| | UUAACACG | | GCUAUUUU | |
| S375-AS749 | GCGUUCUUGUAAUUUUA | 375 | AAAGCUUGUAAAAUUACAA | 749 |
| | CAAGCUTT | | GAACGCUA | |
| S376-AS750 | UAAUUUUACACGCUUUU | 376 | UCCAUUACAAAAGCGUGUA | 750 |
| | GUAAUGGA | | AAAUUACA | |
| S377-AS751 | CGCUUUUGUGAUGGAGU | 377 | AAAACUGCACUCCAUCACA | 751 |
| | GCAGUUTT | | AAAGCGUG | |
| S378-AS752 | GCUUUUGUGAUGGAGUG | 378 | CAAAAUAGCACUCCAUCAC | 752 |
| | CUAUUUTG | | AAAAGCGU | |
| S379-AS753 | CUUUUGUGAUGGAGUGC | 379 | ACAAAUCAGCACUCCAUCA | 753 |
| | UGAUUUGT | | CAAAAGCG | |
| S380-AS754 | UUUUGUGAUGGAGUGCU | 380 | AACAAUACAGCACUCCAUC | 754 |
| | GUAUUGTT | | ACAAAAGC | |
| S381-AS755 | UUUGUGAUGGAGUGCUG | 381 | UAACAUAACAGCACUCCAU | 755 |
| | UUAUGUTA | | CACAAAAG | |
| S382-AS756 | UGUGAUGGAGUGCUGUU | 382 | UAUAAUAAAACAGCACUCC | 756 |
| | UUAUUATA | | AUCACAAA | |
| S383-AS757 | UGAUGGAGUGCUGUUUU | 383 | UAUAUUACAAAACAGCACU | 757 |
| | GUAAUATA | | CCAUCACA | |
| S384-AS758 | GAUGGAGUGCUGUUUUG | 384 | UUAUAUAACAAAACAGCAC | 758 |
| | UUAUAUAA | | UCCAUCAC | |
| S385-AS759 | AUGGAGUGCUGUUUUGU | 385 | AUUAUUUAACAAAACAGCA | 759 |
| | UAAAUAAT | | CUCCAUCA | |
| S386-AS760 | UGGAGUGCUGUUUUGUU | 386 | AAUUAUAUAACAAAACAGC | 760 |
| | AUAUAATT | | ACUCCAUC | |
| S387-AS761 | GGAGUGCUGUUUUGUUA | 387 | AAAUUUUAUAACAAAACAG | 761 |
| | UAAAAUTT | | CACUCCAU | |
| S388-AS762 | GAGUGCUGUUUUGUUAU | 388 | UAAAUUAUAUAACAAAACA | 762 |
| | AUAAUUTA | | GCACUCCA | |
| S389-AS763 | AGUGCUGUUUUGUUAUA | 389 | CUAAAUUAUAUAACAAAAC | 763 |
| | UAAUUUAG | | AGCACUCC | |
| S390-AS764 | GUGCUGUUUUGUUAUAU | 390 | UCUAAUUUAUAUAACAAAA | 764 |
| | AAAUUAGA | | CAGCACUC | |
| S391-AS765 | UGCUGUUUUGUUAUAUA | 391 | GUCUAUAUUAUAUAACAAA | 765 |
| | AUAUAGAC | | ACAGCACU | |
| S392-AS766 | GCUGUUUUGUUAUAUAA | 392 | AGUCUUAAUUAUAUAACAA | 766 |
| | UUAAGACT | | AACAGCAC | |
| S393-AS767 | CUGUUUUGUUAUAUAAU | 393 | AAGUCUAAAUUAUAUAACA | 767 |
| | UUAGACTT | | AAACAGCA | |
| S394-AS768 | AUUUGCAUUUGUUUAUG | 394 | UGAAAUUACAUAAACAAAU | 768 |
| | UAAUUUCA | | GCAAAUGG | |
| S395-AS769 | GUUUAUGUAAUUUCAGG | 395 | GUAUUUCUCCUGAAAUUAC | 769 |
| | AGAAAUAC | | AUAAACAA | |
| S396-AS770 | AUGUAAUUUCAGGAGGA | 396 | UUCAGUAUUCCUCCUGAAA | 770 |
| | AUACUGAA | | UUACAUAA | |
| S397-AS771 | GAAUACUGAACAUCUGA | 397 | UCCAGUACUCAGAUGUUCA | 771 |
| | GUACUGGA | | GUAUUCCU | |
| S398-AS772 | CAUCUGAGUCCUGGAUG | 398 | AUUAGUAUCAUCCAGGACU | 772 |
| | AUACUAAT | | CAGAUGUU | |
| S399-AS773 | AUCUGAGUCCUGGAUGA | 399 | UAUUAUUAUCAUCCAGGAC | 773 |
| | UAAUAATA | | UCAGAUGU | |
| S400-AS774 | UGAGUCCUGGAUGAUAC | 400 | GUUUAUUAGUAUCAUCCAG | 774 |
| | UAAUAAAC | | GACUCAGA | |
| S401-AS775 | AGUCCUGGAUGAUACUA | 401 | UAGUUUAUUAGUAUCAUCC | 775 |
| | AUAAACTA | | AGGACUCA | |
| S402-AS776 | GUCCUGGAUGAUACUAA | 402 | UUAGUUUAUUAGUAUCAUC | 776 |
| | UAAACUAA | | CAGGACUC | |
| S403-AS777 | UCCUGGAUGAUACUAAU | 403 | AUUAGUUUAUUAGUAUCAU | 777 |
| | AAACUAAT | | CCAGGACU | |
| S404-AS778 | CCUGGAUGAUACUAAUA | 404 | UAUUAUUUUAUUAGUAUCA | 778 |
| | AAAUAATA | | UCCAGGAC | |
| S405-AS779 | CUGGAUGAUACUAAUAA | 405 | UUAUUUGUUUAUUAGUAUC | 779 |
| | ACAAAUAA | | AUCCAGGA | |
| S406-AS780 | UGGAUGAUACUAAUAAA | 406 | AUUAUUAGUUUAUUAGUAU | 780 |
| | CUAAUAAT | | CAUCCAGG | |
| S407-AS781 | GGAUGAUACUAAUAAAC | 407 | AAUUAUUAGUUUAUUAGUA | 781 |
| | UAAUAATT | | UCAUCCAG | |
| S408-AS782 | GAUGAUACUAAUAAACU | 408 | CAAUUUUUAGUUUAUUAGU | 782 |
| | AAAAAUTG | | AUCAUCCA | |
| S409-AS783 | AUGAUACUAAUAAACUA | 409 | GCAAUUAUUAGUUUAUUAG | 783 |
| | AUAAUUGC | | UAUCAUCC | |
| S410-AS784 | UGAUACUAAUAAACUAA | 410 | UGCAAUUAUUAGUUUAUUA | 784 |
| | UAAUUGCA | | GUAUCAUC | |
| S411-AS785 | GAUACUAAUAAACUAAU | 411 | CUGCAUUUAUUAGUUUAUU | 785 |
| | AAAUGCAG | | AGUAUCAU | |
| S412-AS786 | AUACUAAUAAACUAAUA | 412 | UCUGCUAUUAUUAGUUUAU | 786 |
| | AUAGCAGA | | UAGUAUCA | |
| S413-AS787 | UACUAAUAAACUAAUAA | 413 | CUCUGUAAUUAUUAGUUUA | 787 |
| | UUACAGAG | | UUAGUAUC | |
| S866-AS887 | UGGGCAAAGGAGAUCCU | 866 | GGCUUUUUAGGAUCUCCUU | 887 |
| | AAAAAGCC | | UGCCCAUG | |
| S867-AS876 | AAAGAGAAAUGAAAACC | 867 | GGGAUUUAGGUUUUCAUUU | 876 |
| | UAAAUCCC | | CUCUUUCA | |
| S868-AS877 | AAGAAGAUGAUGAUGAU | 868 | ACUUAUUCAUCAUCAUCAU | 877 |
| | GAAUAAGT | | CUUCUUCU | |
| S869-AS878 | AUGAUGAUGAUGAAUAA | 869 | GAACCUACUUAUUCAUCAU | 878 |
| | GUAGGUTC | | CAUCAUCU | |
| S870-AS879 | GAUGAUGAAUAAGUUGG | 870 | CGCUAUAACCAACUUAUUC | 879 |
| | UUAUAGCG | | AUCAUCAU | |
| S871-AS880 | AGAAAAAAAUUGAAAUG | 871 | CAGCCUUACAUUUCAAUUU | 880 |
| | UAAGGCTG | | UUUUCUUU | |
| S872-AS881 | UUGUUGUUCUGUUAACU | 872 | UGGUAUUCAGUUAACAGAA | 881 |
| | GAAUACCA | | CAACAAUU | |
| S873-AS882 | UUCUGAAUGCUUCUAAG | 873 | UGUAUUUACUUAGAAGCAU | 882 |
| | UAAAUACA | | UCAGAAUG | |
| S874-AS883 | CUGAAUGCUUCUAAGUA | 874 | AUUGUUUUUACUUAGAAGC | 883 |
| | AAAACAAT | | AUUCAGAA | |
| S875-AS884 | GAAUGCUUCUAAGUAAA | 875 | AAAUUUUAUUUACUUAGAA | 884 |
| | UAAAAUTT | | GCAUUCAG | |

TABLE 5
Additinoal GalNAc-conjugated HMGB1 oligonucleotide sequences with
modifications (tested in FIG. 5).

| GalNAc- | | | | | | |
|---|---|---|---|---|---|---|
| conjugated | Sense | | | Antisense | | |
| oligo- | sequence | Sense sequence | SEQ | sequence | Antisense sequence | SEQ |
| nucleotides | (unmodified) | (modified) | ID NO | (unmodified) | (unmodified) | ID NO |
| S840- | CUAAACAUGGG | [mCs][mU][mA][mA][mA] | 840 | UCUCCUUUGCCC | [MePhosphonate-4O- | 853 |
| AS853-M2 | CAAAGGAGAGC | [mC][mA][fU][fG][fG] | | AUGUUUAGGG | [mUs][FCS][fU][mC] | |
| | AGCCGAAGGCU | [mC][mA][mA][mA][mG] | | | [fC][mU][fU][mU] | |
| | GC | [mG][mA][mG][mA][mG] | | | [mG][fC][mC][mC] | |
| | | [mC][mA][mG][mC][mC] | | | [mA][fU][mG][mU] | |
| | | [mG][ademA-GalNAc] | | | [mU][mU][mA][mGs] | |
| | | [ademA-GalNAc][mG] | | | [mGs][mG] | |
| | | [mG][mC][mU][mG][mC] | | | | |
| S840- | CUAAACAUGGG | [mCs][mU][fA][mA][mA] | 840 | UCUCCUUUGCCC | [MePhosphonate-4O- | 853 |
| AS853-M3 | CAAAGGAGAGC | [mC][mA][fU][mG][fG] | | AUGUUUAGGG | [mUs][FCS][fU][fC] | |
| | AGCCGAAAGGC | [mG][fC][fA][mA][mA] | | | [GC][mU][fU][mU] | |
| | UGC | [mG][fG][mA][mG][mA] | | | [mG][fC][mC][fC] | |
| | | [mG][mC][mA][mG][mC] | | | [mA][fU][mG][fU] | |
| | | [mC][mG][ademA- | | | [fU][mU][fA][mGs] | |
| | | GalNAc][ademA-GalNAc] | | | [mGs][mG] | |
| | | [ademAGalNAc][mG][mG] | | | | |
| | | [mC][mU][mG][mC] | | | | |
| S841- | UAAACAUGGGC | [mUs][mA][mA][mA][mC] | 841 | UUCUCCUUUGCC | [MePhosphonate-4O- | 854 |
| AS854-M2 | AAAGGAGAAGC | [mA][mU][fG][fG][fG] | | CAUGUUUAGG | [mUs][fUs][fC][mU] | |
| | AGCCGAAAGGC | [fC][mA][mA][mA][mG] | | | [fC][mC][fU][mU] | |
| | UGC | [mG][mA][mG][mA][mA] | | | [mU][fG][mC][mC] | |
| | | [mG][mC][mA][mG][mC] | | | [mC][fA][mU][mG] | |
| | | [mC][mG][ademA- | | | [mU][mU][mU][mAs] | |
| | | GalNAc][ademA-GalNAc] | | | [mGs][mG] | |
| | | [ademAGalNAc][mG][mG] | | | | |
| | | [mC][mU][mG][mC] | | | | |
| S841- | UAAACAUGGGC | [mUs][mA][fA][mA][mC] | 841 | UUCUCCUUUGCC | [MePhosphonate-4O- | 854 |
| AS854-M3 | AAAGGAGAAGC | [mA][mU][fG][mG][fG] | | CAUGUUUAGG | [mUs][fUs][fC][fU] | |
| | AGCCGAAAGGC | [mC][fA][fA][mA][mG] | | (SEQ ID | [fC][mC][fU][mU] | |
| | UGC | [mG][fA][mG][mA][mA] | | NO: 854) | [mU][fG][mC][fC] | |
| | | [mG][mC][mA][mG][mC] | | | [mC][fA][mU][fG] | |
| | | [mC][mG][ademA- | | | [fU][mU][fU][mAs] | |
| | | GalNAc][ademA-GalNAc] | | | [mGs][mG] | |
| | | [ademAGalNAc][mG][mG] | | | | |
| | | [mC][mU][mG][mC] | | | | |
| S842- | AGCCGAGAGGC | [mAs][mG][mC][mC][mG] | 842 | UGACAUUUUGCC | [MePhosphonate-4O- | 855 |
| AS855-M2 | AAAAUGUCAGC | [mA][mG][fA][fG][fG] | | UCUCGGCUGG | [mUs][FGS][fA][mC] | |
| | AGCCGAAAGGC | [fC][mA][mA][mA][mA] | | | [fA][mU][fU][mU] | |
| | UGC | [mU][mG][mU][mC][mA] | | | [mU][fG][mC][mC] | |
| | | [mG][mC][mA][mG][mC] | | | [mU][fC][mU][mC] | |
| | | [mC][mG][ademA- | | | [mG][mG][mC][mUs] | |
| | | GalNAc][ademA-GalNAc] | | | [mGs][mG] | |
| | | [ademAGalNAc][mG][mG] | | | | |
| | | [mC][mU][mG][mC] | | | | |
| S842- | AGCCGAGAGGC | [mAs][mG][fC][mC][mG] | 842 | UGACAUUUUGCC | [MePhosphonate-4O- | 855 |
| AS855-M3 | AAAAUGUCAGC | [mA][mG][fA][mG][fG] | | UCUCGGCUGG | [mUs][FGS][fA][fC] | |
| | AGCCGAAAGG | [mC][fA][fA][mA][mA] | | | [fA][mU][fU][mU] | |
| | | [mU][fG][mU][mC][mA] | | | [mU][fG][mC][fC] | |
| | | [mG][mC][mA][mG][mC] | | | [mU][fC][mU][fC] | |
| | | [mC][mG][ademA- | | | [fG][mG][fC][mUs] | |
| | | GalNAc][ademA-GalNAc] | | | [mGs][mG] | |
| | | [ademAGalNAc][mG][mG] | | | | |
| | | [mC][mU][mG][mC] | | | | |
| S843- | AAGAAGUGCUC | [mAs][mA][mG][mA][mA] | 843 | UACCUCUCUGAG | [MePhosphonate-4O- | 856 |
| AS856-M2 | AGAGAGGUAGC | [mG][mU][fG][fC][fU] | | CACUUCUUGG | [mUs][FAS][fC][mC] | |
| | AGCCGAAAGGC | [fC][mA][mG][mA][mG] | | | [fU][mC][fU][mC] | |
| | UGC | [mA][mG][mG][mU][mA] | | | [mU][fG][mA][mG] | |
| | | [mG][mC][mA][mG][mC] | | | [mC][fA][mC][mU] | |
| | | [mC][mG][ademA- | | | [mU][mC][mU][mUs] | |
| | | GalNAc][ademA-GalNAc] | | | [mGs][mG] | |
| | | [ademAGalNAc][mG][mG] | | | | |
| | | [mC][mU][mG][mC] | | | | |
| S843- | AAGAAGUGCUC | [mAs][mA][fG][mA][mA] | 843 | UACCUCUCUGAG | [MePhosphonate-4O- | 856 |
| AS856-M3 | AGAGAGGUAGC | [mG][mU][fG][mC][fU] | | CACUUCUUGG | [mUs][FAS][fC][fC] | |
| | AGCCGAAAGGC | [mC][fA][fG][mA][mG] | | | [fU][mC][fU][mC] | |
| | UGC | [mA][fG][mG][mU][mA] | | | [mU][fG][mA][fG] | |
| | | [mG][mC][mA][mG][mC] | | | [mC][fA][mC][fU] | |
| | | [mC][mG][ademA- | | | [fU][mC][fU][mUs] | |
| | | GalNAc][ademA-GalNAc] | | | [mGs][mG] | |
| | | [ademAGalNAc][mG][mG] | | | | |
| | | [mC][mU][mG][mC] | | | | |
| S844- | CUCUGAGAGGU | [mCs][mU][mC][mA][mG] | 844 | UGGUCUUCCACC | [MePhosphonate-4O- | 857 |
| AS857-M2 | GGAAGACCAGC | [mA][mG][fA][fG][fG] | | UCUCUGAGGG | [mUs][FGS][fG][mU] | |
| | AGCCGAAAGGC | [fU][mG][mG][mA][mA] | | | [fC][mU][fU][mC] | |
| | UGC | [mG][mA][mC][mC][mA] | | | [mC][fA][mC][mC] | |
| | | [mG][mC][mA][mG][mC] | | | [mU][fC][mU][mC] | |
| | | [mC][mG][ademA- | | | [mU][mG][mA][mGs] | |
| | | GalNAc][ademA-GalNAc] | | | [mGs][mG] | |
| | | [ademAGalNAc][mG][mG] | | | | |
| | | [mC][mU][mG][mC] | | | | |
| S844- | CUCUGAGAGGU | [mCs][mU][fC][mA][mG] | 844 | | [MePhosphonate-4O- | 857 |
| AS857-M3 | GGAAGACCAGC | [mA][mG][fA][mG][fG] | | | [mUs][FGS][fG][fU] | |
| | AGCCGAAAGGC | [mU][fG][fG][mA][mA] | | | [fC][mU][fU][mC] | |
| | UGC | [mG][fA][mC][mC][mA] | | | [mC][fA][mC][fC] | |
| | | [mG][mC][mA][mG][mC] | | | [mU][fC][mU][fC] | |
| | | [mC][mG][ademA- | | | [fU][mG][fA][mGs] | |
| | | GalNAc][ademA-GalNAc] | | | [mGs][mG] | |
| | | [ademAGalNAc][mG][mG] | | | | |
| | | [mC][mU][mG][mC] | | | | |
| S845- | AGGUGGAAGAC | [mCs][mU][fC][mA][mG] | 845 | UCAGACAUGGUC | [MePhosphonate-4O- | 858 |
| AS858-M2 | CAUGUCUGAGC | [mA][mG][fA][mG][fG] | | UUCCACCUGG | [mUs][FCS][fA][mG] | |
| | AGCCGAAAGGC | [mU][fG][fG][mA][mA] | | | [fA][mC][fA][mU] | |
| | UGC | [mG][fA][mC][mC][mA] | | | [mG][fG][mU][mC] | |
| | | [mG][mC][mA][mG][mC] | | | [mU][fU][mC][mC] | |
| | | [mC][mG][ademA- | | | [mA][mC][mC][mUs] | |
| | | GalNAc][ademA-GalNAc] | | | [mGs][mG] | |
| | | [ademAGalNAc][mG][mG] | | | | |
| | | [mC][mU][mG][mC] | | | | |
| S845- | AGGUGGAAGAC | [mAs][mG][fG][mU][mG] | 845 | UCAGACAUGGUC | [MePhosphonate-4O- | 858 |
| AS858- | CAUGUCUGAGC | [mG][mA][fA][mG][fA] | | UUCCACCUGG | [mUs][FCS][fA][fG] | |
| | AGCCGAAAGGC | [mC][fC][fA][mU][mG] | | | [fA][mC][fA][mU] | |
| | UGC | [mU][fC][mU][mG][mA] | | | [mG][fG][mU][fC] | |
| | | [mG][mC][mA][mG][mC] | | | [mU][fU][mC][fC] | |
| | | [mC][mG]demA- | | | [fA][mC][fC][mUs] | |
| | | GalNAc][ademA-GalNAc] | | | [mGs][mG] | |
| | | [ademAGalNAc][mG][mG] | | | | |
| | | [mC][mU][mG][mC] | | | | |
| S885- | AAGACCAUGUC | [mAs][mA][mG][mA][mC] | 885 | UCUUUAGCAGAC | [MePhosphonate-4O- | 886 |
| AS886-M2 | UGCUAAAGAGC | [mC][mA][fU][fG][fU] | | AUGGUCUUGG | [mUs][FCS][fU][mU] | |
| | AGCCGAAAGGC | [fC][mU][mG][mC][mU] | | | [fU][mA][fG][mC] | |
| | UGC | [mA][mA][mA][mG][mA] | | | [mA][fG][mA][mC] | |
| | | [mG][mC][mA][mG][mC] | | | [mA][fU][mG][mG] | |
| | | [mC][mG][ademA- | | | [mU][mC][mU][mUs] | |
| | | GalNAc][ademA-GalNAc] | | | [mGs][mG] | |
| | | [ademAGalNAc][mG][mG] | | | | |
| | | [mC][mU][mG][mC] | | | | |
| S885- | AAGACCAUGUC | [mAs][mA][fG][mA][mC] | | UCUUUAGCAGAC | [MePhosphonate-4O- | 886 |
| AS886-M3 | UGCUAAAGAGC | [mC][mA][fU][mG][fU] | | AUGGUCUUGG | [mUs][FCS][fU][fU] | |
| | AGCCGAAAGGC | [mC][fU][fG][mC][mU] | | | [fU][mA][fG][mC] | |
| | UGC | [mA][fA][mA][mG][mA] | | | [mA][fG][mA][fC] | |
| | | [mG][mC][mA][mG][mC] | | | [mA][fU][mG][fG] | |
| | | [mC][mG][ademA- | | | [fU][mC][fU][mUs] | |
| | | GalNAc][ademA-GalNAc] | | | [mGs][mG] | |
| | | [ademAGalNAc][mG][mG] | | | | |
| | | [mC][mU][mG][mC] | | | | |
| S846- | AAAUUUGAAGA | [mAs][mA][mA][mU][mU] | 846 | UUUGCCAUAUCU | [MePhosphonate-4O- | 859 |
| AS859-M2 | UAUGGCAAAGC | [mU][mG][fA][fA][fG] | | UCAAAUCCGG | [mUs][fUs][fU][mG] | |
| | AGCCGAAAGGC | [fA][mU][mA][mU][mG] | | | [fC][mC][fA][mU] | |
| | UGC | [mG][mC][mA][mA][mA] | | | [mA][fU][mC][mU] | |
| | | [mG][mC][mA][mG][mC] | | | [mU][fC][mA][mA] | |
| | | [mC][mG][ademA- | | | [mA][mU][mU][mUs] | |
| | | GalNAc][ademA-GalNAc] | | | [mGs][mG] | |
| | | [ademAGalNAc][mG][mG] | | | | |
| | | [mC][mU][mG][mC] | | | | |
| S846- | AAAUUUGAAGA | [mAs][mA][fA][mU][mU] | 846 | UUUGCCAUAUCU | [MePhosphonate-4O- | 859 |
| AS859-M3 | UAUGGCAAAGC | [mU][mG][fA][mA][fG] | | UCAAAUUUGG | [mUs][fUs][fU][fG] | |
| | AGCCGAAAGGC | [mA][fU][fA][mU][mG] | | | [fC][mC][fA][mU] | |
| | UGC | [mG][fC][mA][mA][mA] | | | [mA][fU][mC][fU] | |
| | | [mG][mC][mA][mG][mC] | | | [mU][fC][mA][fA] | |
| | | [mC][mG][ademA- | | | [fA][mU][fU][mUs] | |
| | | GalNAc][ademA-GalNAc] | | | [mGs][mG] | |
| | | [ademAGalNAc][mG][mG] | | | | |
| | | [mC][mU][mG][mC] | | | | |
| S847- | AGCAAGAAAAA | [mAs][mG][mC][mA][mA] | 847 | UCUUCCUUCUUU | [MePhosphonate-4O- | 860 |
| AS860-M2 | GAAGGAAGAGC | [mG][mA][fA][fA][fA] | | UUCUUGCUGG | [mUs][FCS][fU][mU] | |
| | AGCCGAAAGGC | [fA][mG][mA][mA][mG] | | | [fC][mC][fU][mU] | |
| | UGC | [mG][mA][mA][mG][mA] | | | [mC][fU][mU][mU] | |
| | | [mG][mC][mA][mG][mC] | | | [mU][fU][mC][mU] | |
| | | [mC][mG][ademA- | | | [mU][mG][mC][mUs] | |
| | | GalNAc][ademA-GalNAc] | | | [mGs][mG] | |
| | | [ademAGalNAc][mG][mG] | | | | |
| | | [mC][mU][mG][mC] | | | | |
| S847- | AGCAAGAAAAA | [mAs][mG][fC][mA][mA] | 847 | UCUUCCUUCUUU | [MePhosphonate-4O- | 860 |
| AS860-M3 | GAAGGAAGAGC | [mG][mA][fA][mA][fA] | | UUCUUGCUGG | [mUs][FCS][fU][fU] | |
| | AGCCGAAAGGC | [mA][fG][fA][mA][mG] | | | [fC][mC][fU][mU] | |
| | UGC | [mG][fA][mA][mG][mA] | | | [mC][fU][mU][fU] | |
| | | [mG][mC][mA][mG][mC] | | | [mU][fU][mC][fU] | |
| | | [mC][mG][ademA- | | | [fU][mG][fC][mUs] | |
| | | GalNAc][ademA-GalNAc] | | | [MGS[mG] | |
| | | [ademAGalNAc][mG][mG] | | | | |
| | | [mC][mU][mG][mC] | | | | |
| S848- | CAAGAAAAAGA | [mCs][mA][mA][mG][mA] | 848 | UCUCUUCCUUCU | [MePhosphonate-4O- | 861 |
| AS861-M2 | AGGAAGAGAGC | [mA][mA][fA][fA][fG] | | UUUUCUUGGG | [mUs][FCS][fU][mC] | |
| | AGCCGAAAGGC | [fA][mA][mG][mG][mA] | | | [fU][mU][fC][mC] | |
| | UGC | [mA][mG][mA][mG][mA] | | | [mU][fU][mC][mU] | |
| | | [mG][mC][mA][mG][mC] | | | [mU][fU][mU][mU] | |
| | | [mC][mG][ademA- | | | [mC][mU][mU][mGs] | |
| | | GalNAc][ademA-GalNAc] | | | [mGs][mG] | |
| | | [ademAGalNAc][mG][mG] | | | | |
| | | [mC][mU][mG][mC] | | | | |
| S848- | CAAGAAAAAGA | [mCs][mA][fA][mG][mA] | 848 | UCUCUUCCUUCU | [MePhosphonate-4O- | 861 |
| AS861-M3 | AGGAAGAGAGC | [mA][mA][fA][mA][fG] | | UUUUCUUGGG | [mUs][FCS][fU][fC] | |
| | AGCCGAAAGGC | [mA][fA][fG][mG][mA] | | | [fU][mU][fC][mC] | |
| | UGC | [mA][fG][mA][mG][mA] | | | [mU][fU][mC][fU] | |
| | | [mG][mC][mA][mG][mC] | | | [mU][fU][mU][fU] | |
| | | [mC][mG][ademA- | | | [fC][mU][fU][mGs] | |
| | | GalNAc][ademA-GalNAc] | | | [mGs][mG] | |
| | | [ademAGalNAc][mG][mG] | | | | |
| | | [mC][mU][mG][mC] | | | | |
| S849- | GAUGAUGAUGA | [mGs][mA][mU][mG][mA] | 849 | UCAACUUAUUCA | [MePhosphonate-4O- | 862 |
| AS862-M2 | AUAAGUUGAGC | [mU][mG][fA][fU][fG] | | UCAUCAUCGG | [mUs][FCS][fA][mA] | |
| | AGCCGAAAGGC | [fA][mA][mU][mA][mA] | | | [fC][mU][fU][mA] | |
| | UGC | [mG][mU][mU][mG][mA] | | | [mU][fU][mC][mA] | |
| | | [mG][mC][mA][mG][mC] | | | [mU][fC][mA][mU] | |
| | | [mC][mG][ademA- | | | [mC][mA][mU][mCs] | |
| | | GalNAc][ademA-GalNAc] | | | [mGs][mG] | |
| | | [ademAGalNAc][mG][mG] | | | | |
| | | [mC][mU][mG][mC] | | | | |
| S849- | GAUGAUGAUGA | [mGs][mA][fU][mG][mA] | 849 | UCAACUUAUUCA | [MePhosphonate-4O- | 862 |
| AS862-M3 | AUAAGUUGAGC | [mU][mG][fA][mU][fG] | | UCAUCAUCGG | [mUs][FCS][fA][fA] | |
| | AGCCGAAAGGC | [mA][fA][fU][mA][mA] | | | [fC][mU][fU][mA] | |
| | UGC | [mG][fU][mU][mG][mA] | | | [mU][fU][mC][fA] | |
| | | [mG][mC][mA][mG][mC] | | | [mU][fC][mA][fU] | |
| | | [mC][mG][ademA- | | | [fC][mA][fU][mCs] | |
| | | GalNAc][ademA-GalNAc] | | | [mGs][mG] | |
| | | [ademAGalNAc][mG][mG] | | | | |
| | | [mC][mU][mG][mC] | | | | |
| S850- | AUGAUGAUGAA | [mAs][mU][mG][mA][mU] | 850 | UCCAACUUAUUC | [MePhosphonate-4O- | 863 |
| AS863-M2 | UAAGUUGGAGC | [mG][mA][fU][fG][fA] | | AUCAUCAUGG | [mUs][FCS][fC][mA] | |
| | AGCCGAAAGGC | [fA][mU][mA][mA][mG] | | | [fA][mC][fU][mU] | |
| | UGS | [mU][mU][mG][mG][mA] | | | [mA][fU][mU][mC] | |
| | | [mG][mC][mA][mG][mC] | | | [mA][fU][mC][mA] | |
| | | [mC][mG][ademA- | | | [mU][mC][mA][mUs] | |
| | | GalNAc][ademA-GalNAc] | | | [mGs][mG] | |
| | | [ademAGalNAc][mG][mG] | | | | |
| | | [mC][mU][mG][mC] | | | | |
| S850- | AUGAUGAUGAA | [mAs][mU][fG][mA][mU] | 850 | UCCAACUUAUUC | [MePhosphonate-4O- | 863 |
| AS863-M3 | UAAGUUGGAGC | [mG][mA][fU][mG][fA] | | AUCAUCAUGG | [mUs][FCS][fC][fA] | |
| | AGCCGAAAGGC | [mA][fU][fA][mA][mG] | | | [fA][mC][fU][mU] | |
| | UGC | [mU][fU][mG][mG][mA] | | | [mA][fU][mU][fC] | |
| | (SEQ ID | [mG][mC][mA][mG][mC] | | | [mA][fU][mC][fA] | |
| | NO: 850) | [mC][mG][ademA- | | | [fU][mC][fA][mUs] | |
| | | GalNAc][ademA-GalNAc] | | | [mGs][mG] | |
| | | [ademAGalNAc][mG][mG] | | | | |
| | | [mC][mU][mG][mC] | | | | |
| S851- | AAGUUGGUUCU | [mAs][mA][mG][mU][mU] | 851 | UACUGCGCUAGA | [MePhosphonate-4O- | 864 |
| AS864-M2 | AGCGCAGUAGC | [mG][mG][fU][fU][fC] | | ACCAACUUGG | [mUs][FAS][fC][mU] | |
| | AGCCGAAAGGC | [fU][mA][mG][mC][mG] | | | [fG][mC][fG][mC] | |
| | UGC | [mC][mA][mG][mU][mA] | | | [mU][fA][mG][mA] | |
| | | [mG][mC][mA][mG][mC] | | | [mA][fC][mC][mA] | |
| | | [mC][mG][ademA- | | | [mA][mC][mU][mUs] | |
| | | GalNAc][ademA-GalNAc] | | | [mGs][mG] | |
| | | [ademAGalNAc][mG][mG] | | | | |
| | | [mC][mU][mG][mC] | | | | |
| S851- | AAGUUGGUUCU | [mAs][mA][fG][mU][mU] | 851 | UACUGCGCUAGA | [MePhosphonate-4O- | 864 |
| AS864-M3 | AGCGCAGUAGC | [mG][mG][fU][mU][fC] | | ACCAACUUGG | [mUs][FAS][fC][fU] | |
| | AGCCGAAAGGC | [mU][fA][fG][mC][mG] | | | [fG][mC][fG][mC] | |
| | UGC | [mC][fA][mG][mU][mA] | | | [mU][fA][mG][fA] | |
| | | [mG][mC][mA][mG][mC] | | | [mA][fC][mC][fA] | |
| | | [mC][mG][ademA- | | | [fA][mC][fU][mUs] | |
| | | GalNAc][ademA-GalNAc] | | | [mGs][mG] | |
| | | [ademAGalNAc][mG][mG] | | | | |
| | | [mC][mU][mG][mC] | | | | |
| S852- | UUAUUAGUAUU | [mUs][mU][mA][mU][mU] | 852 | UAGGACAACAAU | [MePhosphonate-4O- | 865 |
| AS865-M2 | GUUGUCCUAGC | [mA][mG][fU][fA][fU] | | ACUAAUAAGG | [mUs][FAS][fG][mG] | |
| | AGCCGAAAGGC | [fU][mG][mU][mU][mG] | | | [fA][mC][fA][mA] | |
| | UGC | [mU][mC][mC][mU][mA] | | | [mC][fA][mA][mU] | |
| | | [mG][mC][mA][mG][mC] | | | [mA][fC][mU][mA] | |
| | | [mC][mG][ademA- | | | [mA][mU][mA][mAs] | |
| | | GalNAc][ademA-GalNAc] | | | [mGs][mG] | |
| | | [ademAGalNAc][mG][mG] | | | | |
| | | [mC][mU][mG][mC] | | | | |
| S852- | UUAUUAGUAUU | [mUs][mU][fA][mU][mU] | 852 | UAGGACAACAAU | [MePhosphonate-4O- | 865 |
| AS865-M3 | GUUGUCCUA | [mA][mG][fU][mA][fU] | | ACUAAUAAGG | [mUs][FAS][fG][fG] | |
| | | [mU][fG][fU][mU][mG] | | | [fA][mC][fA][mA] | |
| | | [mU][fC][mC][mU][mA] | | | [mC][fA][mA][fU] | |
| | | [mG][mC][mA][mG][mC] | | | [mA][fC][mU][fA] | |
| | | [mC][mG][ademA- | | | [fA][mU][fA][mAs] | |
| | | GalNAc][ademA-GalNAc] | | | [mGs][mG] | |
| | | [ademAGalNAc][mG][mG] | | | | |
| | | [mC][mU][mG][mC] | | | | |

The disclosure illustratively described herein suitably can be practiced in the absence of any element or elements, limitation or limitations that are not specifically disclosed herein. Thus, for example, in each instance herein any of the terms “comprising”, “consisting essentially of”, and “consisting of” may be replaced with either of the other two terms. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments, optional features, modification and variation of the concepts herein disclosed may be resorted to by those skilled in the art, and that such modifications and variations are considered to be within the scope of this invention as defined by the description and the appended claims.

In addition, where features or aspects of the invention are described in terms of Markush groups or other grouping of alternatives, those skilled in the art will recognize that the invention is also thereby described in terms of any individual member or subgroup of members of the Markush group or other group.

The use of the terms “a” and “an” and “the” and similar referents in the context of describing the invention (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Embodiments of this invention are described herein, including the best mode known to the inventors for carrying out the invention. Variations of those embodiments may become apparent to those of ordinary skill in the art upon reading the foregoing description.

The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context. Those skilled in the art will recognize or be able to ascertain using no more than routine experimentation, many equivalents to the specific embodiments of the invention described herein. Such equivalents are intended to be encompassed by the following claims.

What is claimed is:

1. An oligonucleotide for reducing expression of HMGB1, the oligonucleotide comprising a sense strand of 15 to 50 nucleotides in length and an antisense strand of 15 to 30 nucleotides in length, wherein the sense strand forms a duplex region with the antisense strand, wherein the sense strand comprises a sequence as set forth in any one of SEQ ID NOs.: 1-13 and wherein the antisense strand comprises a complementary sequence selected from SEQ ID NOs: 14-26.

2. The oligonucleotide of claim 1, wherein the sense strand sequence comprises or consists of a sequence as set forth in any one of SEQ ID NOs: 27-39.

3. The oligonucleotide of claim 1 or claim A2, wherein the antisense strand sequence consists of a sequence as set forth in any one of SEQ ID NOs: 14-26.

4. The oligonucleotide of claim 1, wherein the sense strand sequence comprises a sequence as set forth in SEQ ID NO: 27 and the antisense strand sequence comprises a sequence as set forth in SEQ ID NO: 14.

5. The oligonucleotide of claim 1, wherein the sense strand sequence comprises a sequence as set forth in SEQ ID NO: 28 and the antisense strand sequence comprises a sequence as set forth in SEQ ID NO: 15.

6. The oligonucleotide of claim 1, wherein the sense strand sequence comprises a sequence as set forth in SEQ ID NO: 29 and the antisense strand sequence comprises a sequence as set forth in SEQ ID NO: 16.

7. The oligonucleotide of claim 1, wherein the sense strand sequence comprises a sequence as set forth in SEQ ID NO: 30 and the antisense strand sequence comprises a sequence as set forth in SEQ ID NO: 17.

8. The oligonucleotide of claim 1, wherein the sense strand sequence comprises a sequence as set forth in SEQ ID NO: 31 and the antisense strand sequence comprises a sequence as set forth in SEQ ID NO: 18.

9. The oligonucleotide of claim 1, wherein the sense strand sequence comprises a sequence as set forth in SEQ ID NO: 32 and the antisense strand sequence comprises a sequence as set forth in SEQ ID NO: 19.

10. The oligonucleotide of claim 1, wherein the sense strand sequence comprises a sequence as set forth in SEQ ID NO: 33 and the antisense strand sequence comprises a sequence as set forth in SEQ ID NO: 20.

11. The oligonucleotide of claim 1, wherein the sense strand sequence comprises a sequence as set forth in SEQ ID NO: 34 and the antisense strand sequence comprises a sequence as set forth in SEQ ID NO: 21.

12. The oligonucleotide of claim 1, wherein the sense strand sequence comprises a sequence as set forth in SEQ ID NO: 35 and the antisense strand sequence comprises a sequence as set forth in SEQ ID NO: 22.

13. The oligonucleotide of claim 1, wherein the sense strand sequence comprises a sequence as set forth in SEQ ID NO: 36 and the antisense strand sequence comprises a sequence as set forth in SEQ ID NO: 23.

14. The oligonucleotide of claim 1, wherein the sense strand sequence comprises a sequence as set forth in SEQ ID NO: 37 and the antisense strand sequence comprises a sequence as set forth in SEQ ID NO: 24.

15. The oligonucleotide of claim 1, wherein the sense strand sequence comprises a sequence as set forth in SEQ ID NO: 38 and the antisense strand sequence comprises a sequence as set forth in SEQ ID NO: 25.

16. The oligonucleotide of claim 1, wherein the sense strand sequence comprises a sequence as set forth in SEQ ID NO: 39 and the antisense strand sequence comprises a sequence as set forth in SEQ ID NO: 26.

17. The oligonucleotide of any one of claims 1-16, wherein the oligonucleotide comprises at least one modified nucleotide.

18. The oligonucleotide of claim 17, wherein all the nucleotides of the oligonucleotide are modified.

19. The oligonucleotide of claim 17 or claim 18, wherein the modified nucleotide comprises a 2′-modification.

20. The oligonucleotide of claim 19, wherein the 2′-modification is a 2′-fluoro or 2′-O-methyl.

21. The oligonucleotide of any one of claims 17-20, wherein one or more of positions 1, 2, 4, 6, 7, 12, 14, 16, 18-27, and 31-36 of the sense strand, and/or one or more of positions 1, 4, 6, 8, 9, 11, 13, 15, 18, and 20-22 of the antisense strand are modified with a 2′-O-methyl.

22. The oligonucleotide of claim 21, wherein all of positions 1, 2, 4, 6, 7, 12, 14, 16, 18-27, and 31-36 of the sense strand, and all of positions 1, 4, 6, 8, 9, 11, 13, 15, 18, and 20-22 of the antisense strand are modified with a 2′-O-methyl.

23. The oligonucleotide of any one of claims 17-22, wherein one or more of positions 3, 5, 8-11, 13, 15, and 17 of the sense strand, and/or one or more of positions 2, 3, 5, 7, 10, 12, 14, 16, 17, and 19 of the antisense strand are modified with a 2′-fluoro.

24. The oligonucleotide of claim 23, wherein all of positions 3, 5, 8-11, 13, 15, and 17 of the sense strand, and all of positions 2, 3, 5, 7, 10, 12, 14, 16, 17, and 19 of the antisense strand are modified with a 2′-fluoro.

25. The oligonucleotide of any one of claims 17-20, wherein one or more of positions 1-7, 12-27, and 31-36 of the sense strand, and/or one or more of positions 1, 4, 6, 8, 9, 11-13, and 15-22 of the antisense strand are modified with a 2′-O-methyl.

26. The oligonucleotide of claim 25, wherein all of positions 1-7, 12-27, and 31-36 of the sense strand, and all of positions 1, 4, 6, 8, 9, 11-13, and 15-22 of the antisense strand are modified with a 2′-O-methyl.

27. The oligonucleotide of any one of claims 17-20, 25 and 26, wherein one or more of positions 8-11 of the sense strand, and/or one or more of positions 2, 3, 5, 7, 10, and 14 of the antisense strand are modified with a 2′-fluoro.

28. The oligonucleotide of claim 27, wherein all of positions 8-11 of the sense strand, and all of positions 2, 3, 5, 7, 10, and 14 of the antisense strand are modified with a 2′-fluoro.

29. The oligonucleotide of any one of claims 17-20, wherein one or more of positions 1, 2, 4-7, 9, 11, 14-16, 18-27, and 31-36 of the sense strand, and/or one or more of positions 1, 6, 8, 9, 11, 13, 15, 18, and 20-22 of the antisense strand are modified with a 2′-O-methyl.

30. The oligonucleotide of claim 29, wherein all of positions 1, 2, 4-7, 9, 11, 14-16, 18-27, and 31-36 of the sense strand, and all of positions 1, 6, 8, 9, 11, 13, 15, 18, and 20-22 of the antisense strand are modified with a 2′-O-methyl.

31. The oligonucleotide of any one of claims 17-20, 29, and 30, wherein one or more of positions 3, 8, 10, 12, 13, and 17 of the sense strand, and/or one or more of positions 2-5, 7, 10, 12, 14, 16, 17, and 19 of the antisense strand are modified with a 2′-fluoro.

32. The oligonucleotide of claim 31, wherein all of positions 3, 8, 10, 12, 13, and 17 of the sense strand, and all of positions 2-5, 7, 10, 12, 14, 16, 17, and 19 of the antisense strand are modified with a 2′-fluoro.

33. The oligonucleotide of any one of claims 1-32, wherein the oligonucleotide comprises at least one modified internucleotide linkage.

34. The oligonucleotide of claim 33, wherein the at least one modified internucleotide linkage is a phosphorothioate linkage.

35. The oligonucleotide of claim 33 or claim 34, wherein the oligonucleotide has a phosphorothioate linkage between one or more of: positions 1 and 2 of the sense strand, positions 1 and 2 of the antisense strand, positions 2 and 3 of the antisense strand, positions 3 and 4 of the antisense strand, positions 20 and 21 of the antisense strand, and positions 21 and 22 of the antisense strand.

36. The oligonucleotide of claim 35, wherein the oligonucleotide has a phosphorothioate linkage between each of: positions 1 and 2 of the sense strand, positions 1 and 2 of the antisense strand, positions 2 and 3 of the antisense strand, positions 20 and 21 of the antisense strand, and positions 21 and 22 of the antisense strand.

37. The oligonucleotide of any one of claims 1-36, wherein the uridine at the first position of the antisense strand comprises a phosphate analog.

38. The oligonucleotide of claim 37, comprising the following structure at position 1 of the antisense strand:

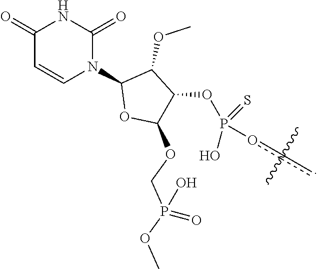

39. The oligonucleotide of any one claims 1-38, wherein one or more of the nucleotides of the -AAA- sequence at positions 28-30 on the sense strand is conjugated to a monovalent GalNAc moiety.

40. The oligonucleotide of claim 39, wherein each of the nucleotides of the -AAA- sequence at positions 28-30 on the sense strand is conjugated to a monovalent GalNAc moiety.

41. The oligonucleotide of claim 40, wherein the -AAA- motif at positions 28-30 on the sense strand comprises the structure: 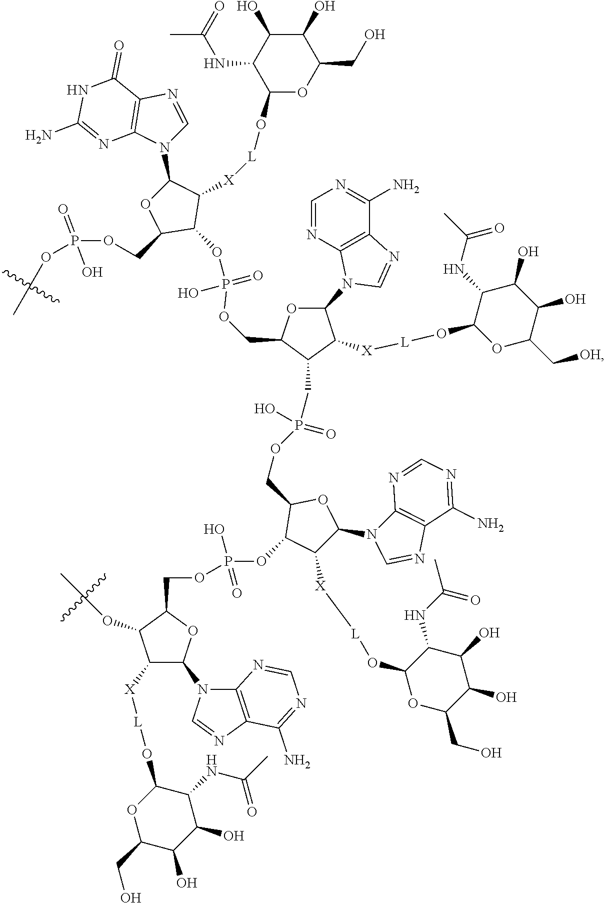
wherein:
L represents a bond, click chemistry handle, or a linker of 1 to 20, inclusive, consecutive, covalently bonded atoms in length, selected from the group consisting of substituted and unsubstituted alkylene, substituted and unsubstituted alkenylene, substituted and unsubstituted alkynylene, substituted and unsubstituted heteroalkylene, substituted and unsubstituted heteroalkenylene, substituted and unsubstituted heteroalkynylene, and combinations thereof; and
X is O, S, or N.

42. The oligonucleotide of claim 41, wherein L is an acetal linker.

43. The oligonucleotide of claim 41 or claim 42, wherein X is O.

44. The oligonucleotide of any one of claims 41-43, wherein the -AAA- sequence at positions 28-30 on the sense strand comprises the structure:

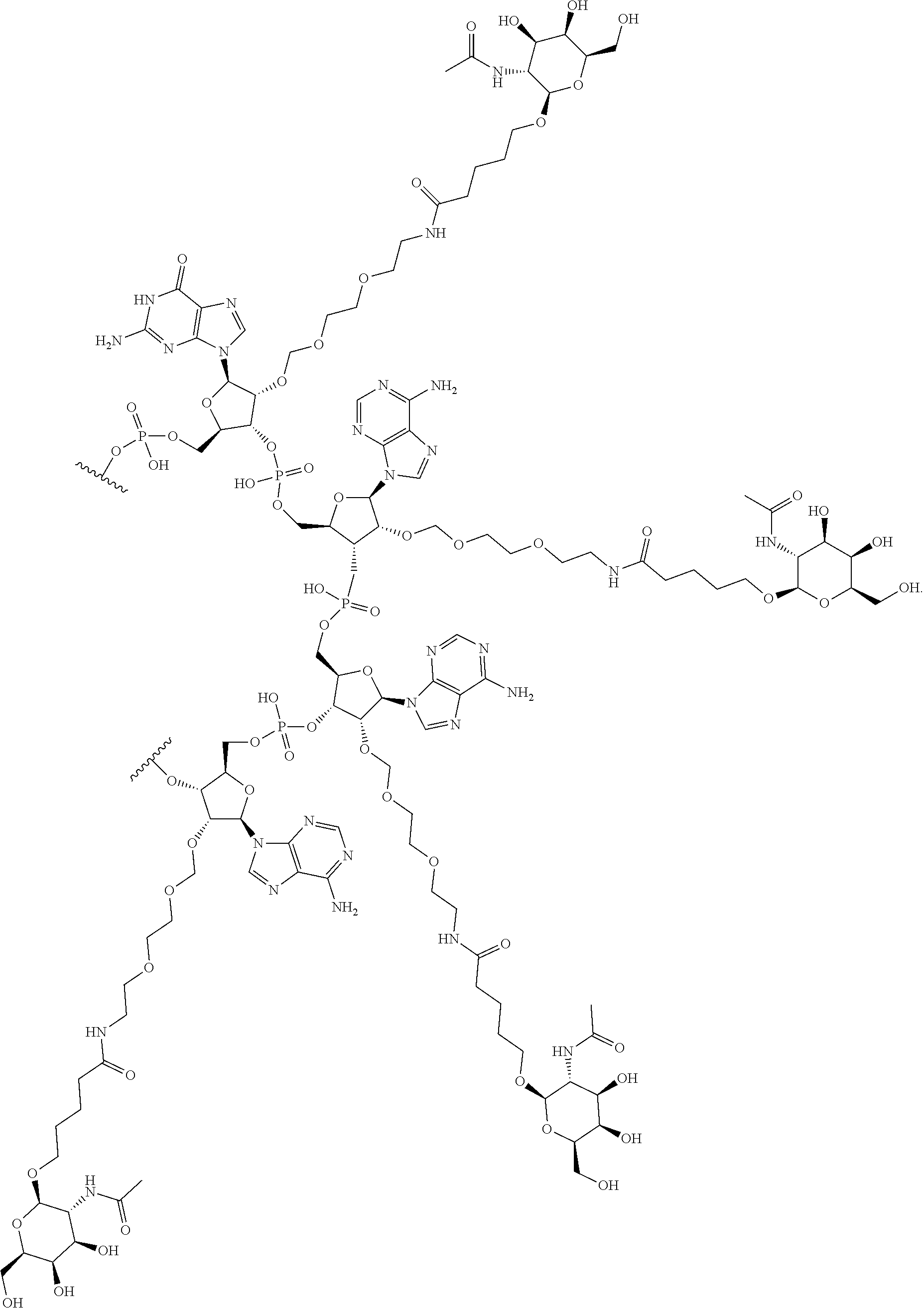 [SMILES: *O[C@H]1[C@@H](OCOCCOCCNC(=O)CCCCO[C@@H]2O[C@H](CO)[C@H](O)[C@H](O)[C@H]2NC(C)=O)[C@H](n2cnc3c(N)ncnc32)O[C@@H]1COP(=O)(O)O[C@H]1[C@@H](OCOCCOCCNC(=O)CCCCO[C@@H]2O[C@H](CO)[C@H](O)[C@H](O)[C@H]2NC(C)=O)[C@H](n2cnc3c(N)ncnc32)O[C@@H]1COP(=O)(O)C[C@H]1[C@@H](OCOCCOCCNC(=O)CCCCO[C@@H]2O[C@H](CO)[C@H](O)[C@H](O)[C@H]2NC(C)=O)[C@H](n2cnc3c(N)ncnc32)O[C@@H]1COP(=O)(O)O[C@H]1[C@@H](OCOCCOCCNC(=O)CCCCO[C@@H]2O[C@H](CO)[C@H](O)[C@H](O)[C@H]2NC(C)=O)[C@H](n2cnc3c(=O)[nH]c(N)nc32)O[C@@H]1COP(=O)(O)O*]

45. An oligonucleotide for reducing expression of HMGB1, the oligonucleotide comprising an antisense strand of 15 to 30 nucleotides in length, wherein the antisense strand has a region of complementarity to HMGB1 that is complementary to at least 15 contiguous nucleotides of a sequence as set forth in any one of SEQ ID NOs: 1-13.

46. The oligonucleotide of claim 45, wherein the antisense strand is 19 to 27 nucleotides in length.

47. The oligonucleotide of claim 45, wherein the antisense strand is 22 nucleotides in length.

48. The oligonucleotide of any one of claims 45 to 47, further comprising a sense strand of 15 to 50 nucleotides in length, wherein the sense strand forms a duplex region with the antisense strand.

49. The oligonucleotide of claim 48, wherein the sense strand is 19 to 50 nucleotides in length.

50. The oligonucleotide of claim 48 or 49, wherein the duplex region is 20 nucleotides in length.

51. An oligonucleotide for reducing expression of HMGB1 comprising a sense strand and an antisense strand, wherein:
(a) the sense strand comprises a sequence as set forth in SEQ ID NO: 788 and the antisense strand comprises a sequence as set forth in SEQ ID NO: 814;
(b) the sense strand comprises a sequence as set forth in SEQ ID NO: 789 and the antisense strand comprises a sequence as set forth in SEQ ID NO: 815;
(c) the sense strand comprises a sequence as set forth in SEQ ID NO: 790 and the antisense strand comprises a sequence as set forth in SEQ ID NO: 816;
(d) the sense strand comprises a sequence as set forth in SEQ ID NO: 791 and the antisense strand comprises a sequence as set forth in SEQ ID NO: 817;
(e) the sense strand comprises a sequence as set forth in SEQ ID NO: 792 and the antisense strand comprises a sequence as set forth in SEQ ID NO: 818;
(f) the sense strand comprises a sequence as set forth in SEQ ID NO: 793 and the antisense strand comprises a sequence as set forth in SEQ ID NO: 819;
(g) the sense strand comprises a sequence as set forth in SEQ ID NO: 794 and the antisense strand comprises a sequence as set forth in SEQ ID NO: 820;
(h) the sense strand comprises a sequence as set forth in SEQ ID NO: 795 and the antisense strand comprises a sequence as set forth in SEQ ID NO: 821;
(i) the sense strand comprises a sequence as set forth in SEQ ID NO: 796 and the antisense strand comprises a sequence as set forth in SEQ ID NO: 822;
(j) the sense strand comprises a sequence as set forth in SEQ ID NO: 797 and the antisense strand comprises a sequence as set forth in SEQ ID NO: 823;
(k) the sense strand comprises a sequence as set forth in SEQ ID NO: 798 and the antisense strand comprises a sequence as set forth in SEQ ID NO: 824;
(l) the sense strand comprises a sequence as set forth in SEQ ID NO: 799 and the antisense strand comprises a sequence as set forth in SEQ ID NO: 825;
(m) the sense strand comprises a sequence as set forth in SEQ ID NO: 800 and the antisense strand comprises a sequence as set forth in SEQ ID NO: 826;
(n) the sense strand comprises a sequence as set forth in SEQ ID NO: 801 and the antisense strand comprises a sequence as set forth in SEQ ID NO: 827;
(o) the sense strand comprises a sequence as set forth in SEQ ID NO: 802 and the antisense strand comprises a sequence as set forth in SEQ ID NO: 828;
(p) the sense strand comprises a sequence as set forth in SEQ ID NO: 803 and the antisense strand comprises a sequence as set forth in SEQ ID NO: 829;
(q) the sense strand comprises a sequence as set forth in SEQ ID NO: 804 and the antisense strand comprises a sequence as set forth in SEQ ID NO: 830;
(r) the sense strand comprises a sequence as set forth in SEQ ID NO: 805 and the antisense strand comprises a sequence as set forth in SEQ ID NO: 831;
(s) the sense strand comprises a sequence as set forth in SEQ ID NO: 806 and the antisense strand comprises a sequence as set forth in SEQ ID NO: 832;
(t) the sense strand comprises a sequence as set forth in SEQ ID NO: 807 and the antisense strand comprises a sequence as set forth in SEQ ID NO: 833;
(u) the sense strand comprises a sequence as set forth in SEQ ID NO: 808 and the antisense strand comprises a sequence as set forth in SEQ ID NO: 834;
(v) the sense strand comprises a sequence as set forth in SEQ ID NO: 809 and the antisense strand comprises a sequence as set forth in SEQ ID NO: 835;
(w) the sense strand comprises a sequence as set forth in SEQ ID NO: 810 and the antisense strand comprises a sequence as set forth in SEQ ID NO: 836;
(x) the sense strand comprises a sequence as set forth in SEQ ID NO: 811 and the antisense strand comprises a sequence as set forth in SEQ ID NO: 837;
(y) the sense strand comprises a sequence as set forth in SEQ ID NO:812 and the antisense strand comprises a sequence as set forth in SEQ ID NO: 838; or
(z) the sense strand comprises a sequence as set forth in SEQ ID NO: 813 and the antisense strand comprises a sequence as set forth in SEQ ID NO: 839.

52. A composition comprising the oligonucleotide of any one of claims 1-51 and an excipient.

53. A method of delivering an oligonucleotide to a subject, the method comprising administering the composition of claim 52 to the subject.

54. The method of claim 53, wherein the subject has or is at risk of having liver fibrosis.

55. The method of claim 54, wherein the subject has cholestatic or autoimmune liver disease.

56. The method of any one of claims 53-55, wherein expression of HMGB1 protein is reduced by administering to the subject the oligonucleotide.

57. A method of treating a subject having or at risk of having liver fibrosis, the method comprising administering to the subject an oligonucleotide of any one of claims 1-51.

58. The method of claim 57, wherein the subject has cholestatic or autoimmune liver disease.

59. The method of claim 58, wherein the subject has nonalcoholic steatohepatitis (NASH).

60. The method of any one of claims 57-59, wherein the oligonucleotide is administered prior to exposure of the subject to a hepatotoxic agent.

61. The method of any one of claims 57-59, wherein the oligonucleotide is administered subsequent to exposure of the subject to a hepatotoxic agent.

62. The method of any one of claims 57-59, wherein the oligonucleotide is administered simultaneously with the subject's exposure to a hepatotoxic agent.

63. The method of any one of claims 57-62, wherein the administration results in a reduction in liver HMGB1 levels.

64. The method of any one of claims 57-63, wherein the administration results in a reduction in serum HMGB1 levels.

65. Use of an oligonucleotide of any one of claims 1-51 for treating a subject having or at risk of having liver fibrosis.

66. The use of claim 65, wherein the subject has cholestatic or autoimmune liver disease.

67. The use of claim 65, wherein the subject has nonalcoholic steatohepatitis (NASH).


SEQUENCE LISTING (887 sequences)

SEQ ID NO: 1 - RNA, 20 nt, Artificial Sequence
  Synthetic Polynucleotide
ugggcaaaggagauccuaaa

SEQ ID NO: 2 - RNA, 20 nt, Artificial Sequence
  Synthetic Polynucleotide
aaagagaaaugaaaaccuaa

SEQ ID NO: 3 - RNA, 20 nt, Artificial Sequence
  Synthetic Polynucleotide
aagaagaugaugaugaugaa

SEQ ID NO: 4 - RNA, 20 nt, Artificial Sequence
  Synthetic Polynucleotide
augaugaugaugaauaagua

SEQ ID NO: 5 - RNA, 20 nt, Artificial Sequence
  Synthetic Polynucleotide
gaugaugaauaaguugguuc

SEQ ID NO: 6 - RNA, 20 nt, Artificial Sequence
  Synthetic Polynucleotide
ugaauaaguugguucuagca

SEQ ID NO: 7 - RNA, 20 nt, Artificial Sequence
  Synthetic Polynucleotide
aauaaguugguucuagcgca

SEQ ID NO: 8 - RNA, 20 nt, Artificial Sequence
  Synthetic Polynucleotide
auaaguugguucuagcgcaa

SEQ ID NO: 9 - RNA, 20 nt, Artificial Sequence
  Synthetic Polynucleotide
agaaaaaaauugaaauguaa

SEQ ID NO: 10 - RNA, 20 nt, Artificial Sequence
  Synthetic Polynucleotide
uuguuguucuguuaacugaa

SEQ ID NO: 11 - RNA, 20 nt, Artificial Sequence
  Synthetic Polynucleotide
uucugaaugcuucuaaguaa

SEQ ID NO: 12 - RNA, 20 nt, Artificial Sequence
  Synthetic Polynucleotide
cugaaugcuucuaaguaaaa

SEQ ID NO: 13 - RNA, 20 nt, Artificial Sequence
  Synthetic Polynucleotide
gaaugcuucuaaguaaauaa

SEQ ID NO: 14 - RNA, 22 nt, Artificial Sequence
  Synthetic Polynucleotide
uuuaggaucuccuuugcccagg

SEQ ID NO: 15 - RNA, 22 nt, Artificial Sequence
  Synthetic Polynucleotide
uuagguuuucauuucucuuugg

SEQ ID NO: 16 - RNA, 22 nt, Artificial Sequence
  Synthetic Polynucleotide
uucaucaucaucaucuucuugg

SEQ ID NO: 17 - RNA, 22 nt, Artificial Sequence
  Synthetic Polynucleotide
uacuuauucaucaucaucaugg

SEQ ID NO: 18 - RNA, 22 nt, Artificial Sequence
  Synthetic Polynucleotide
gaaccaacuuauucaucaucgg

SEQ ID NO: 19 - RNA, 22 nt, Artificial Sequence
  Synthetic Polynucleotide
ugcuagaaccaacuuauucagg

SEQ ID NO: 20 - RNA, 22 nt, Artificial Sequence
  Synthetic Polynucleotide
ugcgcuagaaccaacuuauugg

SEQ ID NO: 21 - RNA, 22 nt, Artificial Sequence
  Synthetic Polynucleotide
uugcgcuagaaccaacuuaugg

SEQ ID NO: 22 - RNA, 22 nt, Artificial Sequence
  Synthetic Polynucleotide
uuacauuucaauuuuuuucugg

SEQ ID NO: 23 - RNA, 22 nt, Artificial Sequence
  Synthetic Polynucleotide
uucaguuaacagaacaacaagg

SEQ ID NO: 24 - RNA, 22 nt, Artificial Sequence
  Synthetic Polynucleotide
uuacuuagaagcauucagaagg

SEQ ID NO: 25 - RNA, 22 nt, Artificial Sequence
  Synthetic Polynucleotide
uuuuacuuagaagcauucaggg

SEQ ID NO: 26 - RNA, 22 nt, Artificial Sequence
  Synthetic Polynucleotide
uuauuuacuuagaagcauucgg

SEQ ID NO: 27 - RNA, 36 nt, Artificial Sequence
  Synthetic Polynucleotide
ugggcaaaggagauccuaaagcagccgaaaggcugc

SEQ ID NO: 28 - RNA, 36 nt, Artificial Sequence
  Synthetic Polynucleotide
aaagagaaaugaaaaccuaagcagccgaaaggcugc

SEQ ID NO: 29 - RNA, 36 nt, Artificial Sequence
  Synthetic Polynucleotide
aagaagaugaugaugaugaagcagccgaaaggcugc

SEQ ID NO: 30 - RNA, 36 nt, Artificial Sequence
  Synthetic Polynucleotide
augaugaugaugaauaaguagcagccgaaaggcugc

SEQ ID NO: 31 - RNA, 36 nt, Artificial Sequence
  Synthetic Polynucleotide
gaugaugaauaaguugguuagcagccgaaaggcugc

SEQ ID NO: 32 - RNA, 36 nt, Artificial Sequence
  Synthetic Polynucleotide
ugaauaaguugguucuagcagcagccgaaaggcugc

SEQ ID NO: 33 - RNA, 36 nt, Artificial Sequence
  Synthetic Polynucleotide
aauaaguugguucuagcgcagcagccgaaaggcugc

SEQ ID NO: 34 - RNA, 36 nt, Artificial Sequence
  Synthetic Polynucleotide
auaaguugguucuagcgcaagcagccgaaaggcugc

SEQ ID NO: 35 - RNA, 36 nt, Artificial Sequence
  Synthetic Polynucleotide
agaaaaaaauugaaauguaagcagccgaaaggcugc

SEQ ID NO: 36 - RNA, 36 nt, Artificial Sequence
  Synthetic Polynucleotide
uuguuguucuguuaacugaagcagccgaaaggcugc

SEQ ID NO: 37 - RNA, 36 nt, Artificial Sequence
  Synthetic Polynucleotide
uucugaaugcuucuaaguaagcagccgaaaggcugc

SEQ ID NO: 38 - RNA, 36 nt, Artificial Sequence
  Synthetic Polynucleotide
cugaaugcuucuaaguaaaagcagccgaaaggcugc

SEQ ID NO: 39 - RNA, 36 nt, Artificial Sequence
  Synthetic Polynucleotide
gaaugcuucuaaguaaauaagcagccgaaaggcugc

SEQ ID NO: 40 - RNA, 25 nt, Artificial Sequence
  Synthetic Polynucleotide
aacuaaacaugggcaaaggagaucc

SEQ ID NO: 41 - DNA, 25 bp, Artificial Sequence
  Synthetic Polynucleotide
acuaaacaugggcaaaggaaaucct

SEQ ID NO: 42 - DNA, 25 bp, Artificial Sequence
  Synthetic Polynucleotide
cuaaacaugggcaaaggagauccta

SEQ ID NO: 43 - RNA, 25 nt, Artificial Sequence
  Synthetic Polynucleotide
uaaacaugggcaaaggagaaccuaa

SEQ ID NO: 44 - RNA, 25 nt, Artificial Sequence
  Synthetic Polynucleotide
aaacaugggcaaaggagauacuaag

SEQ ID NO: 45 - RNA, 25 nt, Artificial Sequence
  Synthetic Polynucleotide
aacaugggcaaaggagaucauaaga

SEQ ID NO: 46 - RNA, 25 nt, Artificial Sequence
  Synthetic Polynucleotide
acaugggcaaaggagauccaaagaa

SEQ ID NO: 47 - RNA, 25 nt, Artificial Sequence
  Synthetic Polynucleotide
caugggcaaaggagauccuaagaag

SEQ ID NO: 48 - RNA, 25 nt, Artificial Sequence
  Synthetic Polynucleotide
augggcaaaggagauccuaagaagc

SEQ ID NO: 49 - DNA, 25 bp, Artificial Sequence
  Synthetic Polynucleotide
aagccgagaggcaaaauguaaucat

SEQ ID NO: 50 - DNA, 25 bp, Artificial Sequence
  Synthetic Polynucleotide
agccgagaggcaaaaugucaucata

SEQ ID NO: 51 - DNA, 25 bp, Artificial Sequence
  Synthetic Polynucleotide
aaaugucaucauaugcauuauuugt

SEQ ID NO: 52 - RNA, 25 nt, Artificial Sequence
  Synthetic Polynucleotide
caucauaugcauuuuuuguacaaac

SEQ ID NO: 53 - DNA, 25 bp, Artificial Sequence
  Synthetic Polynucleotide
aucauaugcauuuuuugugaaaact

SEQ ID NO: 54 - DNA, 25 bp, Artificial Sequence
  Synthetic Polynucleotide
auaugcauuuuuugugcaaacuugt

SEQ ID NO: 55 - RNA, 25 nt, Artificial Sequence
  Synthetic Polynucleotide
gucaacuucucagaguuuuauaaga

SEQ ID NO: 56 - RNA, 25 nt, Artificial Sequence
  Synthetic Polynucleotide
aagaagugcucagagagguagaaga

SEQ ID NO: 57 - RNA, 25 nt, Artificial Sequence
  Synthetic Polynucleotide
agaagugcucagagaggugaaagac

SEQ ID NO: 58 - RNA, 25 nt, Artificial Sequence
  Synthetic Polynucleotide
gaagugcucagagagguggaagacc

SEQ ID NO: 59 - RNA, 25 nt, Artificial Sequence
  Synthetic Polynucleotide
aagugcucagagagguggaagacca

SEQ ID NO: 60 - DNA, 25 bp, Artificial Sequence
  Synthetic Polynucleotide
agugcucagagagguggaaaaccat

SEQ ID NO: 61 - DNA, 25 bp, Artificial Sequence
  Synthetic Polynucleotide
gugcucagagagguggaagaccatg

SEQ ID NO: 62 - DNA, 25 bp, Artificial Sequence
  Synthetic Polynucleotide
ugcucagagagguggaagaacaugt

SEQ ID NO: 63 - DNA, 25 bp, Artificial Sequence
  Synthetic Polynucleotide
gcucagagagguggaagacaaugtc

SEQ ID NO: 64 - DNA, 25 bp, Artificial Sequence
  Synthetic Polynucleotide
cucagagagguggaagaccauguct

SEQ ID NO: 65 - DNA, 25 bp, Artificial Sequence
  Synthetic Polynucleotide
ucagagagguggaagaccaaguctg

SEQ ID NO: 66 - RNA, 25 nt, Artificial Sequence
  Synthetic Polynucleotide
cagagagguggaagaccauaucugc

SEQ ID NO: 67 - DNA, 25 bp, Artificial Sequence
  Synthetic Polynucleotide
agagagguggaagaccaugacugct

SEQ ID NO: 68 - DNA, 25 bp, Artificial Sequence
  Synthetic Polynucleotide
gagagguggaagaccauguaugcta

SEQ ID NO: 69 - RNA, 25 nt, Artificial Sequence
  Synthetic Polynucleotide
agagguggaagaccaugucagcuaa

SEQ ID NO: 70 - RNA, 25 nt, Artificial Sequence
  Synthetic Polynucleotide
gagguggaagaccaugucuacuaaa

SEQ ID NO: 71 - RNA, 25 nt, Artificial Sequence
  Synthetic Polynucleotide
agguggaagaccaugucugauaaag

SEQ ID NO: 72 - RNA, 25 nt, Artificial Sequence
  Synthetic Polynucleotide
gguggaagaccaugucugcaaaaga

SEQ ID NO: 73 - RNA, 25 nt, Artificial Sequence
  Synthetic Polynucleotide
guggaagaccaugucugcuaaagag

SEQ ID NO: 74 - RNA, 25 nt, Artificial Sequence
  Synthetic Polynucleotide
uggaagaccaugucugcuaaagaga

SEQ ID NO: 75 - RNA, 25 nt, Artificial Sequence
  Synthetic Polynucleotide
ggaagaccaugucugcuaaagagaa

SEQ ID NO: 76 - RNA, 25 nt, Artificial Sequence
  Synthetic Polynucleotide
gaagaccaugucugcuaaaaagaaa

SEQ ID NO: 77 - RNA, 25 nt, Artificial Sequence
  Synthetic Polynucleotide
aagaccaugucugcuaaagagaaag

SEQ ID NO: 78 - RNA, 25 nt, Artificial Sequence
  Synthetic Polynucleotide
agaccaugucugcuaaagaaaaagg

SEQ ID NO: 79 - RNA, 25 nt, Artificial Sequence
  Synthetic Polynucleotide
aaauuugaagauauggcaaaagcgg

SEQ ID NO: 80 - RNA, 25 nt, Artificial Sequence
  Synthetic Polynucleotide
aauuugaagauauggcaaaagcgga

SEQ ID NO: 81 - RNA, 25 nt, Artificial Sequence
  Synthetic Polynucleotide
gaaagagaaaugaaaaccuauaucc

SEQ ID NO: 82 - DNA, 25 bp, Artificial Sequence
  Synthetic Polynucleotide
aaaaaagaaguucaaggauaccaat

SEQ ID NO: 83 - DNA, 25 bp, Artificial Sequence
  Synthetic Polynucleotide
cccaaugcacccaagaggcaucctt

SEQ ID NO: 84 - DNA, 25 bp, Artificial Sequence
  Synthetic Polynucleotide
ccaaugcacccaagaggccaccutc

SEQ ID NO: 85 - RNA, 25 nt, Artificial Sequence
  Synthetic Polynucleotide
caaugcacccaagaggccuacuucg

SEQ ID NO: 86 - RNA, 25 nt, Artificial Sequence
  Synthetic Polynucleotide
aaugcacccaagaggccucauucgg

SEQ ID NO: 87 - RNA, 25 nt, Artificial Sequence
  Synthetic Polynucleotide
augcacccaagaggccuccaucggc

SEQ ID NO: 88 - RNA, 25 nt, Artificial Sequence
  Synthetic Polynucleotide
ugcacccaagaggccuccuacggcc

SEQ ID NO: 89 - DNA, 25 bp, Artificial Sequence
  Synthetic Polynucleotide
gcacccaagaggccuccuuaggcct

SEQ ID NO: 90 - DNA, 25 bp, Artificial Sequence
  Synthetic Polynucleotide
cacccaagaggccuccuucagcctt

SEQ ID NO: 91 - DNA, 25 bp, Artificial Sequence
  Synthetic Polynucleotide
acccaagaggccuccuucgaccutc

SEQ ID NO: 92 - DNA, 25 bp, Artificial Sequence
  Synthetic Polynucleotide
cccaagaggccuccuucggacuuct

SEQ ID NO: 93 - DNA, 25 bp, Artificial Sequence
  Synthetic Polynucleotide
ccaagaggccuccuucggcauuctt

SEQ ID NO: 94 - DNA, 25 bp, Artificial Sequence
  Synthetic Polynucleotide
caagaggccuccuucggccaucutc

SEQ ID NO: 95 - RNA, 25 nt, Artificial Sequence
  Synthetic Polynucleotide
aagaggccuccuucggccuacuucc

SEQ ID NO: 96 - DNA, 25 bp, Artificial Sequence
  Synthetic Polynucleotide
agaggccuccuucggccuuauucct

SEQ ID NO: 97 - DNA, 25 bp, Artificial Sequence
  Synthetic Polynucleotide
gaggccuccuucggccuucaucctc

SEQ ID NO: 98 - DNA, 25 bp, Artificial Sequence
  Synthetic Polynucleotide
aggccuccuucggccuucuaccuct

SEQ ID NO: 99 - DNA, 25 bp, Artificial Sequence
  Synthetic Polynucleotide
ggccuccuucggccuucuuacuctt

SEQ ID NO: 100 - DNA, 25 bp, Artificial Sequence
  Synthetic Polynucleotide
gccuccuucggccuucuucaucutc

SEQ ID NO: 101 - RNA, 25 nt, Artificial Sequence
  Synthetic Polynucleotide
ggaauaacacugcugcagaagacaa

SEQ ID NO: 102 - RNA, 25 nt, Artificial Sequence
  Synthetic Polynucleotide
gaugacaagcagccuuaugaaaaga

SEQ ID NO: 103 - RNA, 25 nt, Artificial Sequence
  Synthetic Polynucleotide
ggauauugcugcauaucgaacuaaa

SEQ ID NO: 104 - RNA, 25 nt, Artificial Sequence
  Synthetic Polynucleotide
agcaagaaaaagaaggaagaggagg

SEQ ID NO: 105 - RNA, 25 nt, Artificial Sequence
  Synthetic Polynucleotide
gcaagaaaaagaaggaagaagagga

SEQ ID NO: 106 - RNA, 25 nt, Artificial Sequence
  Synthetic Polynucleotide
caagaaaaagaaggaagagaaggaa

SEQ ID NO: 107 - RNA, 25 nt, Artificial Sequence
  Synthetic Polynucleotide
aagaaaaagaaggaagaggaggaag

SEQ ID NO: 108 - RNA, 25 nt, Artificial Sequence
  Synthetic Polynucleotide
agaaaaagaaggaagaggaagaaga

SEQ ID NO: 109 - RNA, 25 nt, Artificial Sequence
  Synthetic Polynucleotide
gaagaagaugaugaugaugaauaag

SEQ ID NO: 110 - DNA, 25 bp, Artificial Sequence
  Synthetic Polynucleotide
gaugaugaugaugaauaagauggtt

SEQ ID NO: 111 - DNA, 25 bp, Artificial Sequence
  Synthetic Polynucleotide
gaugaugaugaauaaguugauucta

SEQ ID NO: 112 - RNA, 25 nt, Artificial Sequence
  Synthetic Polynucleotide
augaugaugaauaaguuggaucuag

SEQ ID NO: 113 - RNA, 25 nt, Artificial Sequence
  Synthetic Polynucleotide
gaugaauaaguugguucuaacgcag

SEQ ID NO: 114 - DNA, 25 bp, Artificial Sequence
  Synthetic Polynucleotide
ugaauaaguugguucuagcacagtt

SEQ ID NO: 115 - DNA, 25 bp, Artificial Sequence
  Synthetic Polynucleotide
gaauaaguugguucuagcgaagutt

SEQ ID NO: 116 - DNA, 25 bp, Artificial Sequence
  Synthetic Polynucleotide
aauaaguugguucuagcgcaguutt

SEQ ID NO: 117 - DNA, 25 bp, Artificial Sequence
  Synthetic Polynucleotide
auaaguugguucuagcgcaauuutt

SEQ ID NO: 118 - DNA, 25 bp, Artificial Sequence
  Synthetic Polynucleotide
uaaguugguucuagcgcagauuutt

SEQ ID NO: 119 - DNA, 25 bp, Artificial Sequence
  Synthetic Polynucleotide
aaguugguucuagcgcaguauuutt

SEQ ID NO: 120 - RNA, 25 nt, Artificial Sequence
  Synthetic Polynucleotide
uuuuucuugucuauaaagcauuuaa

SEQ ID NO: 121 - RNA, 25 nt, Artificial Sequence
  Synthetic Polynucleotide
uuuucuugucuauaaagcaauuaac

SEQ ID NO: 122 - RNA, 25 nt, Artificial Sequence
  Synthetic Polynucleotide
uuucuugucuauaaagcauauaacc

SEQ ID NO: 123 - RNA, 25 nt, Artificial Sequence
  Synthetic Polynucleotide
uucuugucuauaaagcauuaaaccc

SEQ ID NO: 124 - RNA, 25 nt, Artificial Sequence
  Synthetic Polynucleotide
ucuugucuauaaagcauuuaacccc

SEQ ID NO: 125 - RNA, 25 nt, Artificial Sequence
  Synthetic Polynucleotide
caacucacuccuuuuaaagaaaaaa

SEQ ID NO: 126 - RNA, 25 nt, Artificial Sequence
  Synthetic Polynucleotide
aacucacuccuuuuaaagaaaaaaa

SEQ ID NO: 127 - DNA, 25 bp, Artificial Sequence
  Synthetic Polynucleotide
acucacuccuuuuaaagaaaaaaat

SEQ ID NO: 128 - DNA, 25 bp, Artificial Sequence
  Synthetic Polynucleotide
cucacuccuuuuaaagaaaaaaatt

SEQ ID NO: 129 - DNA, 25 bp, Artificial Sequence
  Synthetic Polynucleotide
ucacuccuuuuaaagaaaaaaautg

SEQ ID NO: 130 - RNA, 25 nt, Artificial Sequence
  Synthetic Polynucleotide
cacuccuuuuaaagaaaaaaauuga

SEQ ID NO: 131 - RNA, 25 nt, Artificial Sequence
  Synthetic Polynucleotide
acuccuuuuaaagaaaaaaauugaa

SEQ ID NO: 132 - RNA, 25 nt, Artificial Sequence
  Synthetic Polynucleotide
cuccuuuuaaagaaaaaaaaugaaa

SEQ ID NO: 133 - DNA, 25 bp, Artificial Sequence
  Synthetic Polynucleotide
uccuuuuaaagaaaaaaauagaaat

SEQ ID NO: 134 - DNA, 25 bp, Artificial Sequence
  Synthetic Polynucleotide
ccuuuuaaagaaaaaaauuaaaatg

SEQ ID NO: 135 - DNA, 25 bp, Artificial Sequence
  Synthetic Polynucleotide
cuuuuaaagaaaaaaauugaaaugt

SEQ ID NO: 136 - DNA, 25 bp, Artificial Sequence
  Synthetic Polynucleotide
uuuuaaagaaaaaaauugaaaugta

SEQ ID NO: 137 - RNA, 25 nt, Artificial Sequence
  Synthetic Polynucleotide
uuuaaagaaaaaaauugaaauguaa

SEQ ID NO: 138 - RNA, 25 nt, Artificial Sequence
  Synthetic Polynucleotide
uuaaagaaaaaaauugaaaaguaag

SEQ ID NO: 139 - RNA, 25 nt, Artificial Sequence
  Synthetic Polynucleotide
uaaagaaaaaaauugaaauauaagg

SEQ ID NO: 140 - RNA, 25 nt, Artificial Sequence
  Synthetic Polynucleotide
aaagaaaaaaauugaaaugaaaggc

SEQ ID NO: 141 - DNA, 25 bp, Artificial Sequence
  Synthetic Polynucleotide
gaaaaaaauugaaauguaaagcugt

SEQ ID NO: 142 - DNA, 25 bp, Artificial Sequence
  Synthetic Polynucleotide
aaaaaaauugaaauguaagacugtg

SEQ ID NO: 143 - RNA, 25 nt, Artificial Sequence
  Synthetic Polynucleotide
guaagauuuguuuuuaaacaguaca

SEQ ID NO: 144 - RNA, 25 nt, Artificial Sequence
  Synthetic Polynucleotide
uaagauuuguuuuuaaacuauacag

SEQ ID NO: 145 - DNA, 25 bp, Artificial Sequence
  Synthetic Polynucleotide
aagauuuguuuuuaaacugaacagt

SEQ ID NO: 146 - DNA, 25 bp, Artificial Sequence
  Synthetic Polynucleotide
auuuguuuuuaaacuguacagugtc

SEQ ID NO: 147 - DNA, 25 bp, Artificial Sequence
  Synthetic Polynucleotide
uuguuuuuaaacuguacagaguctt

SEQ ID NO: 148 - DNA, 25 bp, Artificial Sequence
  Synthetic Polynucleotide
uguuuuuaaacuguacaguaucutt

SEQ ID NO: 149 - DNA, 25 bp, Artificial Sequence
  Synthetic Polynucleotide
guuuuuaaacuguacagugacuutt

SEQ ID NO: 150 - DNA, 25 bp, Artificial Sequence
  Synthetic Polynucleotide
uuuuuaaacuguacaguguauuutt

SEQ ID NO: 151 - DNA, 25 bp, Artificial Sequence
  Synthetic Polynucleotide
uuuuaaacuguacagugucauuutt

SEQ ID NO: 152 - DNA, 25 bp, Artificial Sequence
  Synthetic Polynucleotide
uuuaaacuguacagugucuauuutt

SEQ ID NO: 153 - DNA, 25 bp, Artificial Sequence
  Synthetic Polynucleotide
uuaaacuguacagugucuuauuutg

SEQ ID NO: 154 - DNA, 25 bp, Artificial Sequence
  Synthetic Polynucleotide
uaaacuguacagugucuuuauuugt

SEQ ID NO: 155 - DNA, 25 bp, Artificial Sequence
  Synthetic Polynucleotide
aaacuguacagugucuuuuauugta

SEQ ID NO: 156 - DNA, 25 bp, Artificial Sequence
  Synthetic Polynucleotide
aacuguacagugucuuuuuauguat

SEQ ID NO: 157 - DNA, 25 bp, Artificial Sequence
  Synthetic Polynucleotide
acuguacagugucuuuuuuaguata

SEQ ID NO: 158 - RNA, 25 nt, Artificial Sequence
  Synthetic Polynucleotide
cuguacagugucuuuuuuuauauag

SEQ ID NO: 159 - DNA, 25 bp, Artificial Sequence
  Synthetic Polynucleotide
uguacagugucuuuuuuugaauagt

SEQ ID NO: 160 - DNA, 25 bp, Artificial Sequence
  Synthetic Polynucleotide
uacagugucuuuuuuuguaaaguta

SEQ ID NO: 161 - RNA, 25 nt, Artificial Sequence
  Synthetic Polynucleotide
cagugucuuuuuuuguauaauuaac

SEQ ID NO: 162 - RNA, 25 nt, Artificial Sequence
  Synthetic Polynucleotide
gugguauuuucaauagccaauaacc

SEQ ID NO: 163 - DNA, 25 bp, Artificial Sequence
  Synthetic Polynucleotide
ugguauuuucaauagccacaaacct

SEQ ID NO: 164 - DNA, 25 bp, Artificial Sequence
  Synthetic Polynucleotide
gguauuuucaauagccacuaacctt

SEQ ID NO: 165 - RNA, 25 nt, Artificial Sequence
  Synthetic Polynucleotide
uauuuucaauagccacuaaacuugc

SEQ ID NO: 166 - RNA, 25 nt, Artificial Sequence
  Synthetic Polynucleotide
auuuucaauagccacuaacauugcc

SEQ ID NO: 167 - DNA, 25 bp, Artificial Sequence
  Synthetic Polynucleotide
uuuucaauagccacuaaccaugcct

SEQ ID NO: 168 - DNA, 25 bp, Artificial Sequence
  Synthetic Polynucleotide
uuucaauagccacuaaccuagcctg

SEQ ID NO: 169 - RNA, 25 nt, Artificial Sequence
  Synthetic Polynucleotide
uucaauagccacuaaccuuaccugg

SEQ ID NO: 170 - DNA, 25 bp, Artificial Sequence
  Synthetic Polynucleotide
ucaauagccacuaaccuugacuggt

SEQ ID NO: 171 - DNA, 25 bp, Artificial Sequence
  Synthetic Polynucleotide
caauagccacuaaccuugcauggta

SEQ ID NO: 172 - RNA, 25 nt, Artificial Sequence
  Synthetic Polynucleotide
aauagccacuaaccuugccagguac

SEQ ID NO: 173 - RNA, 25 nt, Artificial Sequence
  Synthetic Polynucleotide
auagccacuaaccuugccuaguaca

SEQ ID NO: 174 - RNA, 25 nt, Artificial Sequence
  Synthetic Polynucleotide
uagccacuaaccuugccugauacag

SEQ ID NO: 175 - DNA, 25 bp, Artificial Sequence
  Synthetic Polynucleotide
agccacuaaccuugccuggaacagt

SEQ ID NO: 176 - DNA, 25 bp, Artificial Sequence
  Synthetic Polynucleotide
gccacuaaccuugccugguacagta

SEQ ID NO: 177 - DNA, 25 bp, Artificial Sequence
  Synthetic Polynucleotide
ccacuaaccuugccugguaaaguat

SEQ ID NO: 178 - DNA, 25 bp, Artificial Sequence
  Synthetic Polynucleotide
cacuaaccuugccugguacaguatg

SEQ ID NO: 179 - RNA, 25 nt, Artificial Sequence
  Synthetic Polynucleotide
acuaaccuugccugguacaauaugg

SEQ ID NO: 180 - DNA, 25 bp, Artificial Sequence
  Synthetic Polynucleotide
ggguuguaaauuggcauggaaautt

SEQ ID NO: 181 - DNA, 25 bp, Artificial Sequence
  Synthetic Polynucleotide
gguuguaaauuggcauggaaauuta

SEQ ID NO: 182 - RNA, 25 nt, Artificial Sequence
  Synthetic Polynucleotide
guuguaaauuggcauggaaauuuaa

SEQ ID NO: 183 - RNA, 25 nt, Artificial Sequence
  Synthetic Polynucleotide
uuguaaauuggcauggaaaauuaaa

SEQ ID NO: 184 - RNA, 25 nt, Artificial Sequence
  Synthetic Polynucleotide
uguaaauuggcauggaaauauaaag

SEQ ID NO: 185 - RNA, 25 nt, Artificial Sequence
  Synthetic Polynucleotide
guaaauuggcauggaaauuaaaagc

SEQ ID NO: 186 - RNA, 25 nt, Artificial Sequence
  Synthetic Polynucleotide
uaaauuggcauggaaauuuaaagca

SEQ ID NO: 187 - RNA, 25 nt, Artificial Sequence
  Synthetic Polynucleotide
aaauuggcauggaaauuuaaagcag

SEQ ID NO: 188 - RNA, 25 nt, Artificial Sequence
  Synthetic Polynucleotide
aauuggcauggaaauuuaaagcagg

SEQ ID NO: 189 - DNA, 25 bp, Artificial Sequence
  Synthetic Polynucleotide
auuggcauggaaauuuaaaacaggt

SEQ ID NO: 190 - DNA, 25 bp, Artificial Sequence
  Synthetic Polynucleotide
uuggcauggaaauuuaaagaaggtt

SEQ ID NO: 191 - DNA, 25 bp, Artificial Sequence
  Synthetic Polynucleotide
uggcauggaaauuuaaagcaggutc

SEQ ID NO: 192 - DNA, 25 bp, Artificial Sequence
  Synthetic Polynucleotide
ggcauggaaauuuaaagcaaguuct

SEQ ID NO: 193 - DNA, 25 bp, Artificial Sequence
  Synthetic Polynucleotide
gcauggaaauuuaaagcagauuctt

SEQ ID NO: 194 - DNA, 25 bp, Artificial Sequence
  Synthetic Polynucleotide
cauggaaauuuaaagcaggaucutg

SEQ ID NO: 195 - DNA, 25 bp, Artificial Sequence
  Synthetic Polynucleotide
auggaaauuuaaagcagguacuugt

SEQ ID NO: 196 - DNA, 25 bp, Artificial Sequence
  Synthetic Polynucleotide
uggaaauuuaaagcagguuauugtt

SEQ ID NO: 197 - DNA, 25 bp, Artificial Sequence
  Synthetic Polynucleotide
ggaaauuuaaagcagguucaugutg

SEQ ID NO: 198 - RNA, 25 nt, Artificial Sequence
  Synthetic Polynucleotide
gaaauuuaaagcagguucuaguugg

SEQ ID NO: 199 - DNA, 25 bp, Artificial Sequence
  Synthetic Polynucleotide
aaauuuaaagcagguucuuauuggt

SEQ ID NO: 200 - DNA, 25 bp, Artificial Sequence
  Synthetic Polynucleotide
aauuuaaagcagguucuugauggtg

SEQ ID NO: 201 - RNA, 25 nt, Artificial Sequence
  Synthetic Polynucleotide
auuuaaagcagguucuuguaggugc

SEQ ID NO: 202 - RNA, 25 nt, Artificial Sequence
  Synthetic Polynucleotide
uuuaaagcagguucuuguuagugca

SEQ ID NO: 203 - RNA, 25 nt, Artificial Sequence
  Synthetic Polynucleotide
uuaaagcagguucuuguugaugcac

SEQ ID NO: 204 - RNA, 25 nt, Artificial Sequence
  Synthetic Polynucleotide
uaaagcagguucuuguuggagcaca

SEQ ID NO: 205 - RNA, 25 nt, Artificial Sequence
  Synthetic Polynucleotide
aaagcagguucuuguugguacacag

SEQ ID NO: 206 - RNA, 25 nt, Artificial Sequence
  Synthetic Polynucleotide
aagcagguucuuguuggugaacagc

SEQ ID NO: 207 - RNA, 25 nt, Artificial Sequence
  Synthetic Polynucleotide
agcagguucuuguuggugcacagca

SEQ ID NO: 208 - RNA, 25 nt, Artificial Sequence
  Synthetic Polynucleotide
gcagguucuuguuggugcaaagcac

SEQ ID NO: 209 - RNA, 25 nt, Artificial Sequence
  Synthetic Polynucleotide
cagguucuuguuggugcacagcaca

SEQ ID NO: 210 - RNA, 25 nt, Artificial Sequence
  Synthetic Polynucleotide
agguucuuguuggugcacaacacaa

SEQ ID NO: 211 - RNA, 25 nt, Artificial Sequence
  Synthetic Polynucleotide
gguucuuguuggugcacagaacaaa

SEQ ID NO: 212 - DNA, 25 bp, Artificial Sequence
  Synthetic Polynucleotide
guucuuguuggugcacagcacaaat

SEQ ID NO: 213 - DNA, 25 bp, Artificial Sequence
  Synthetic Polynucleotide
uucuuguuggugcacagcaaaaatt

SEQ ID NO: 214 - DNA, 25 bp, Artificial Sequence
  Synthetic Polynucleotide
ucuuguuggugcacagcacaaauta

SEQ ID NO: 215 - RNA, 25 nt, Artificial Sequence
  Synthetic Polynucleotide
cuuguuggugcacagcacaaauuag

SEQ ID NO: 216 - DNA, 25 bp, Artificial Sequence
  Synthetic Polynucleotide
uuguuggugcacagcacaaauuagt

SEQ ID NO: 217 - DNA, 25 bp, Artificial Sequence
  Synthetic Polynucleotide
uguuggugcacagcacaaaauagtt

SEQ ID NO: 218 - DNA, 25 bp, Artificial Sequence
  Synthetic Polynucleotide
guuggugcacagcacaaauaaguta

SEQ ID NO: 219 - DNA, 25 bp, Artificial Sequence
  Synthetic Polynucleotide
uuggugcacagcacaaauuaguuat

SEQ ID NO: 220 - DNA, 25 bp, Artificial Sequence
  Synthetic Polynucleotide
uggugcacagcacaaauuaauuata

SEQ ID NO: 221 - DNA, 25 bp, Artificial Sequence
  Synthetic Polynucleotide
ggugcacagcacaaauuagauauat

SEQ ID NO: 222 - RNA, 25 nt, Artificial Sequence
  Synthetic Polynucleotide
uuuuuucaucuucaguuguaucuga

SEQ ID NO: 223 - DNA, 25 bp, Artificial Sequence
  Synthetic Polynucleotide
uuuuucaucuucaguugucacugat

SEQ ID NO: 224 - DNA, 25 bp, Artificial Sequence
  Synthetic Polynucleotide
uuuucaucuucaguugucuaugatg

SEQ ID NO: 225 - RNA, 25 nt, Artificial Sequence
  Synthetic Polynucleotide
uuucaucuucaguugucucagaugc

SEQ ID NO: 226 - RNA, 25 nt, Artificial Sequence
  Synthetic Polynucleotide
uucaucuucaguugucucuaaugca

SEQ ID NO: 227 - RNA, 25 nt, Artificial Sequence
  Synthetic Polynucleotide
ucaucuucaguugucucugaugcag

SEQ ID NO: 228 - RNA, 25 nt, Artificial Sequence
  Synthetic Polynucleotide
caucuucaguugucucugaagcagc

SEQ ID NO: 229 - DNA, 25 bp, Artificial Sequence
  Synthetic Polynucleotide
aucuucaguugucucugauacagct

SEQ ID NO: 230 - DNA, 25 bp, Artificial Sequence
  Synthetic Polynucleotide
ucugaugcagcuuauacgaaauaat

SEQ ID NO: 231 - DNA, 25 bp, Artificial Sequence
  Synthetic Polynucleotide
cugaugcagcuuauacgaaauaatt

SEQ ID NO: 232 - DNA, 25 bp, Artificial Sequence
  Synthetic Polynucleotide
cagcuuauacgaaauaauuauugtt

SEQ ID NO: 233 - DNA, 25 bp, Artificial Sequence
  Synthetic Polynucleotide
agcuuauacgaaauaauugaugutc

SEQ ID NO: 234 - DNA, 25 bp, Artificial Sequence
  Synthetic Polynucleotide
gcuuauacgaaauaauuguaguuct

SEQ ID NO: 235 - DNA, 25 bp, Artificial Sequence
  Synthetic Polynucleotide
cuuauacgaaauaauuguuauuctg

SEQ ID NO: 236 - DNA, 25 bp, Artificial Sequence
  Synthetic Polynucleotide
uauacgaaauaauuguuguacugtt

SEQ ID NO: 237 - DNA, 25 bp, Artificial Sequence
  Synthetic Polynucleotide
auacgaaauaauuguuguuauguta

SEQ ID NO: 238 - RNA, 25 nt, Artificial Sequence
  Synthetic Polynucleotide
uacgaaauaauuguuguucaguuaa

SEQ ID NO: 239 - RNA, 25 nt, Artificial Sequence
  Synthetic Polynucleotide
acgaaauaauuguuguucuauuaac

SEQ ID NO: 240 - DNA, 25 bp, Artificial Sequence
  Synthetic Polynucleotide
cgaaauaauuguuguucugauaact

SEQ ID NO: 241 - DNA, 25 bp, Artificial Sequence
  Synthetic Polynucleotide
gaaauaauuguuguucuguaaactg

SEQ ID NO: 242 - RNA, 25 nt, Artificial Sequence
  Synthetic Polynucleotide
aaauaauuguuguucuguuaacuga

SEQ ID NO: 243 - RNA, 25 nt, Artificial Sequence
  Synthetic Polynucleotide
aauaauuguuguucuguuaacugaa

SEQ ID NO: 244 - RNA, 25 nt, Artificial Sequence
  Synthetic Polynucleotide
aauuguuguucuguuaacuaaauac

SEQ ID NO: 245 - RNA, 25 nt, Artificial Sequence
  Synthetic Polynucleotide
auuguuguucuguuaacugaauacc

SEQ ID NO: 246 - RNA, 25 nt, Artificial Sequence
  Synthetic Polynucleotide
uguuguucuguuaacugaaaaccac

SEQ ID NO: 247 - DNA, 25 bp, Artificial Sequence
  Synthetic Polynucleotide
guuguucuguuaacugaauaccact

SEQ ID NO: 248 - DNA, 25 bp, Artificial Sequence
  Synthetic Polynucleotide
uguucuguuaacugaauacaacuct

SEQ ID NO: 249 - DNA, 25 bp, Artificial Sequence
  Synthetic Polynucleotide
ucuguuaacugaauaccacacugta

SEQ ID NO: 250 - RNA, 25 nt, Artificial Sequence
  Synthetic Polynucleotide
cuguuaacugaauaccacuauguaa

SEQ ID NO: 251 - DNA, 25 bp, Artificial Sequence
  Synthetic Polynucleotide
guuaacugaauaccacucuauaatt

SEQ ID NO: 252 - RNA, 25 nt, Artificial Sequence
  Synthetic Polynucleotide
aacugaauaccacucuguaauugca

SEQ ID NO: 253 - RNA, 25 nt, Artificial Sequence
  Synthetic Polynucleotide
acugaauaccacucuguaaaugcaa

SEQ ID NO: 254 - RNA, 25 nt, Artificial Sequence
  Synthetic Polynucleotide
cugaauaccacucuguaauagcaaa

SEQ ID NO: 255 - RNA, 25 nt, Artificial Sequence
  Synthetic Polynucleotide
ugaauaccacucuguaauuacaaaa

SEQ ID NO: 256 - RNA, 25 nt, Artificial Sequence
  Synthetic Polynucleotide
gaauaccacucuguaauugaaaaaa

SEQ ID NO: 257 - RNA, 25 nt, Artificial Sequence
  Synthetic Polynucleotide
aauaccacucuguaauugcaaaaaa

SEQ ID NO: 258 - RNA, 25 nt, Artificial Sequence
  Synthetic Polynucleotide
aaaaaguugcagcuguuuuauugac

SEQ ID NO: 259 - DNA, 25 bp, Artificial Sequence
  Synthetic Polynucleotide
aaaguugcagcuguuuuguagacat

SEQ ID NO: 260 - DNA, 25 bp, Artificial Sequence
  Synthetic Polynucleotide
aaguugcagcuguuuuguuaacatt

SEQ ID NO: 261 - DNA, 25 bp, Artificial Sequence
  Synthetic Polynucleotide
aguugcagcuguuuuguugacautc

SEQ ID NO: 262 - DNA, 25 bp, Artificial Sequence
  Synthetic Polynucleotide
guugcagcuguuuuguugaaauuct

SEQ ID NO: 263 - RNA, 25 nt, Artificial Sequence
  Synthetic Polynucleotide
ugcagcuguuuuguugacaaucuga

SEQ ID NO: 264 - RNA, 25 nt, Artificial Sequence
  Synthetic Polynucleotide
gcagcuguuuuguugacauacugaa

SEQ ID NO: 265 - DNA, 25 bp, Artificial Sequence
  Synthetic Polynucleotide
cagcuguuuuguugacauuaugaat

SEQ ID NO: 266 - DNA, 25 bp, Artificial Sequence
  Synthetic Polynucleotide
agcuguuuuguugacauucagaatg

SEQ ID NO: 267 - RNA, 25 nt, Artificial Sequence
  Synthetic Polynucleotide
gcuguuuuguugacauucuaaaugc

SEQ ID NO: 268 - DNA, 25 bp, Artificial Sequence
  Synthetic Polynucleotide
uguuuuguugacauucugaaugctt

SEQ ID NO: 269 - DNA, 25 bp, Artificial Sequence
  Synthetic Polynucleotide
uuuguugacauucugaaugauucta

SEQ ID NO: 270 - RNA, 25 nt, Artificial Sequence
  Synthetic Polynucleotide
uuguugacauucugaaugcaucuaa

SEQ ID NO: 271 - RNA, 25 nt, Artificial Sequence
  Synthetic Polynucleotide
uguugacauucugaaugcuacuaag

SEQ ID NO: 272 - DNA, 25 bp, Artificial Sequence
  Synthetic Polynucleotide
guugacauucugaaugcuuauaagt

SEQ ID NO: 273 - DNA, 25 bp, Artificial Sequence
  Synthetic Polynucleotide
uugacauucugaaugcuucaaagta

SEQ ID NO: 274 - RNA, 25 nt, Artificial Sequence
  Synthetic Polynucleotide
ugacauucugaaugcuucuaaguaa

SEQ ID NO: 275 - RNA, 25 nt, Artificial Sequence
  Synthetic Polynucleotide
gacauucugaaugcuucuaaguaaa

SEQ ID NO: 276 - DNA, 25 bp, Artificial Sequence
  Synthetic Polynucleotide
cauucugaaugcuucuaagaaaata

SEQ ID NO: 277 - RNA, 25 nt, Artificial Sequence
  Synthetic Polynucleotide
auucugaaugcuucuaaguaaauac

SEQ ID NO: 278 - RNA, 25 nt, Artificial Sequence
  Synthetic Polynucleotide
ucugaaugcuucuaaguaaauacaa

SEQ ID NO: 279 - DNA, 25 bp, Artificial Sequence
  Synthetic Polynucleotide
ugaaugcuucuaaguaaauacaatt

SEQ ID NO: 280 - DNA, 25 bp, Artificial Sequence
  Synthetic Polynucleotide
aaugcuucuaaguaaauacaauutt

SEQ ID NO: 281 - DNA, 25 bp, Artificial Sequence
  Synthetic Polynucleotide
augcuucuaaguaaauacaauuutt

SEQ ID NO: 282 - DNA, 25 bp, Artificial Sequence
  Synthetic Polynucleotide
gcuucuaaguaaauacaauauuutt

SEQ ID NO: 283 - DNA, 25 bp, Artificial Sequence
  Synthetic Polynucleotide
uuuuuauuaguauuguuguacuutt

SEQ ID NO: 284 - DNA, 25 bp, Artificial Sequence
  Synthetic Polynucleotide
uuuuauuaguauuguugucauuutc

SEQ ID NO: 285 - RNA, 25 nt, Artificial Sequence
  Synthetic Polynucleotide
uuuauuaguauuguuguccauuuca

SEQ ID NO: 286 - DNA, 25 bp, Artificial Sequence
  Synthetic Polynucleotide
uuauuaguauuguuguccuauucat

SEQ ID NO: 287 - DNA, 25 bp, Artificial Sequence
  Synthetic Polynucleotide
uauuaguauuguuguccuuaucata

SEQ ID NO: 288 - RNA, 25 nt, Artificial Sequence
  Synthetic Polynucleotide
auuaguauuguuguccuuuacauag

SEQ ID NO: 289 - RNA, 25 nt, Artificial Sequence
  Synthetic Polynucleotide
uuaguauuguuguccuuuuaauagg

SEQ ID NO: 290 - DNA, 25 bp, Artificial Sequence
  Synthetic Polynucleotide
uaguauuguuguccuuuucauaggt

SEQ ID NO: 291 - DNA, 25 bp, Artificial Sequence
  Synthetic Polynucleotide
guauuguuguccuuuucauagguct

SEQ ID NO: 292 - RNA, 25 nt, Artificial Sequence
  Synthetic Polynucleotide
uuguuguccuuuucauaggacugaa

SEQ ID NO: 293 - RNA, 25 nt, Artificial Sequence
  Synthetic Polynucleotide
uguuguccuuuucauagguaugaaa

SEQ ID NO: 294 - DNA, 25 bp, Artificial Sequence
  Synthetic Polynucleotide
uuguccuuuucauaggucuaaaatt

SEQ ID NO: 295 - DNA, 25 bp, Artificial Sequence
  Synthetic Polynucleotide
uguccuuuucauaggucugaaautt

SEQ ID NO: 296 - DNA, 25 bp, Artificial Sequence
  Synthetic Polynucleotide
guccuuuucauaggucugaaauutt

SEQ ID NO: 297 - DNA, 25 bp, Artificial Sequence
  Synthetic Polynucleotide
uccuuuucauaggucugaaauuutt

SEQ ID NO: 298 - DNA, 25 bp, Artificial Sequence
  Synthetic Polynucleotide
ccuuuucauaggucugaaaauuutc

SEQ ID NO: 299 - RNA, 25 nt, Artificial Sequence
  Synthetic Polynucleotide
auaggucugaaauuuuucuacuuga

SEQ ID NO: 300 - DNA, 25 bp, Artificial Sequence
  Synthetic Polynucleotide
aggggaagcuagucuuuugauuutg

SEQ ID NO: 301 - RNA, 25 nt, Artificial Sequence
  Synthetic Polynucleotide
ggggaagcuagucuuuugcauuugc

SEQ ID NO: 302 - RNA, 25 nt, Artificial Sequence
  Synthetic Polynucleotide
gggaagcuagucuuuugcuauugcc

SEQ ID NO: 303 - RNA, 25 nt, Artificial Sequence
  Synthetic Polynucleotide
ggaagcuagucuuuugcuuaugccc

SEQ ID NO: 304 - RNA, 25 nt, Artificial Sequence
  Synthetic Polynucleotide
gaagcuagucuuuugcuuuagccca

SEQ ID NO: 305 - DNA, 25 bp, Artificial Sequence
  Synthetic Polynucleotide
caguguuuauccuuucauaaaguta

SEQ ID NO: 306 - RNA, 25 nt, Artificial Sequence
  Synthetic Polynucleotide
guguuuauccuuucauauaauuagc

SEQ ID NO: 307 - DNA, 25 bp, Artificial Sequence
  Synthetic Polynucleotide
uuauccuuucauauaguuaacuaat

SEQ ID NO: 308 - RNA, 25 nt, Artificial Sequence
  Synthetic Polynucleotide
gcuaauaaaaagcuuuuguauacac

SEQ ID NO: 309 - DNA, 25 bp, Artificial Sequence
  Synthetic Polynucleotide
guaaaguuaaguugagauaauuutc

SEQ ID NO: 310 - DNA, 25 bp, Artificial Sequence
  Synthetic Polynucleotide
aaaguuaaguugagauaguauucat

SEQ ID NO: 311 - RNA, 25 nt, Artificial Sequence
  Synthetic Polynucleotide
guuaaguugagauaguuuuaaucca

SEQ ID NO: 312 - DNA, 25 bp, Artificial Sequence
  Synthetic Polynucleotide
uaaguugagauaguuuucaaccata

SEQ ID NO: 313 - RNA, 25 nt, Artificial Sequence
  Synthetic Polynucleotide
aaguugagauaguuuucauacauaa

SEQ ID NO: 314 - RNA, 25 nt, Artificial Sequence
  Synthetic Polynucleotide
aguugagauaguuuucaucaauaac

SEQ ID NO: 315 - DNA, 25 bp, Artificial Sequence
  Synthetic Polynucleotide
guugagauaguuuucauccauaact

SEQ ID NO: 316 - RNA, 25 nt, Artificial Sequence
  Synthetic Polynucleotide
ugagauaguuuucauccauaacuga

SEQ ID NO: 317 - RNA, 25 nt, Artificial Sequence
  Synthetic Polynucleotide
gagauaguuuucauccauaacugaa

SEQ ID NO: 318 - RNA, 25 nt, Artificial Sequence
  Synthetic Polynucleotide
agauaguuuucauccauaaaugaac

SEQ ID NO: 319 - RNA, 25 nt, Artificial Sequence
  Synthetic Polynucleotide
guuuucauccauaacugaaaaucca

SEQ ID NO: 320 - RNA, 25 nt, Artificial Sequence
  Synthetic Polynucleotide
uucauccauaacugaacauacaaaa

SEQ ID NO: 321 - DNA, 25 bp, Artificial Sequence
  Synthetic Polynucleotide
uugaucaguuaagaaauuuaacata

SEQ ID NO: 322 - RNA, 25 nt, Artificial Sequence
  Synthetic Polynucleotide
gaucaguuaagaaauuucaaauagc

SEQ ID NO: 323 - RNA, 25 nt, Artificial Sequence
  Synthetic Polynucleotide
cauuuacaaacugaagaguaaucaa

SEQ ID NO: 324 - DNA, 25 bp, Artificial Sequence
  Synthetic Polynucleotide
auuuacaaacugaagaguaaucaat

SEQ ID NO: 325 - RNA, 25 nt, Artificial Sequence
  Synthetic Polynucleotide
acaaacugaagaguaaucaaucuac

SEQ ID NO: 326 - RNA, 25 nt, Artificial Sequence
  Synthetic Polynucleotide
aaacauuuugaaagucuguacuuga

SEQ ID NO: 327 - DNA, 25 bp, Artificial Sequence
  Synthetic Polynucleotide
aaggacuaauagaaaaguaaguuct

SEQ ID NO: 328 - RNA, 25 nt, Artificial Sequence
  Synthetic Polynucleotide
acuaauagaaaaguauguuauaacc

SEQ ID NO: 329 - RNA, 25 nt, Artificial Sequence
  Synthetic Polynucleotide
agaaaaguauguucuaaccauuaca

SEQ ID NO: 330 - DNA, 25 bp, Artificial Sequence
  Synthetic Polynucleotide
gaaaaguauguucuaaccuauacat

SEQ ID NO: 331 - RNA, 25 nt, Artificial Sequence
  Synthetic Polynucleotide
aaguauguucuaaccuuuaaaugag

SEQ ID NO: 332 - DNA, 25 bp, Artificial Sequence
  Synthetic Polynucleotide
guaauggcaguuauauuuuacagtt

SEQ ID NO: 333 - DNA, 25 bp, Artificial Sequence
  Synthetic Polynucleotide
uaauggcaguuauauuuugaagutc

SEQ ID NO: 334 - RNA, 25 nt, Artificial Sequence
  Synthetic Polynucleotide
uaaagaagaccugagaaugaauccc

SEQ ID NO: 335 - RNA, 25 nt, Artificial Sequence
  Synthetic Polynucleotide
aaagaagaccugagaauguaucccc

SEQ ID NO: 336 - RNA, 25 nt, Artificial Sequence
  Synthetic Polynucleotide
gaagaccugagaauguaucaccaaa

SEQ ID NO: 337 - RNA, 25 nt, Artificial Sequence
  Synthetic Polynucleotide
aagaccugagaauguauccacaaaa

SEQ ID NO: 338 - RNA, 25 nt, Artificial Sequence
  Synthetic Polynucleotide
agaccugagaauguaucccaaaaag

SEQ ID NO: 339 - RNA, 25 nt, Artificial Sequence
  Synthetic Polynucleotide
gaccugagaauguauccccaaaagc

SEQ ID NO: 340 - RNA, 25 nt, Artificial Sequence
  Synthetic Polynucleotide
accugagaauguauccccaaaagcg

SEQ ID NO: 341 - DNA, 25 bp, Artificial Sequence
  Synthetic Polynucleotide
ccugagaauguauccccaaaagcgt

SEQ ID NO: 342 - DNA, 25 bp, Artificial Sequence
  Synthetic Polynucleotide
cugagaauguauccccaaaagcgtg

SEQ ID NO: 343 - RNA, 25 nt, Artificial Sequence
  Synthetic Polynucleotide
ugagaauguauccccaaaaacguga

SEQ ID NO: 344 - RNA, 25 nt, Artificial Sequence
  Synthetic Polynucleotide
gagaauguauccccaaaagagugag

SEQ ID NO: 345 - DNA, 25 bp, Artificial Sequence
  Synthetic Polynucleotide
gccauauuaaauuuuuuguagacat

SEQ ID NO: 346 - DNA, 25 bp, Artificial Sequence
  Synthetic Polynucleotide
ccauauuaaauuuuuuguuaacatt

SEQ ID NO: 347 - DNA, 25 bp, Artificial Sequence
  Synthetic Polynucleotide
cauauuaaauuuuuuguugacauta

SEQ ID NO: 348 - RNA, 25 nt, Artificial Sequence
  Synthetic Polynucleotide
auauuaaauuuuuuguugaaauuag

SEQ ID NO: 349 - DNA, 25 bp, Artificial Sequence
  Synthetic Polynucleotide
auuaaauuuuuuguugacaauagtc

SEQ ID NO: 350 - RNA, 25 nt, Artificial Sequence
  Synthetic Polynucleotide
aaauuuuuuguugacauuaaucuca

SEQ ID NO: 351 - RNA, 25 nt, Artificial Sequence
  Synthetic Polynucleotide
aauuuuuuguugacauuagacucag

SEQ ID NO: 352 - DNA, 25 bp, Artificial Sequence
  Synthetic Polynucleotide
auuuuuuguugacauuaguaucagt

SEQ ID NO: 353 - DNA, 25 bp, Artificial Sequence
  Synthetic Polynucleotide
gaagacuaugaaaaugcugacuata

SEQ ID NO: 354 - DNA, 25 bp, Artificial Sequence
  Synthetic Polynucleotide
agacuuuccauuacaaguaauuuta

SEQ ID NO: 355 - DNA, 25 bp, Artificial Sequence
  Synthetic Polynucleotide
acuuugcaucucaguaugaauuatt

SEQ ID NO: 356 - DNA, 25 bp, Artificial Sequence
  Synthetic Polynucleotide
cuuugcaucucaguaugaaauautc

SEQ ID NO: 357 - RNA, 25 nt, Artificial Sequence
  Synthetic Polynucleotide
uugcaucucaguaugaauuauucaa

SEQ ID NO: 358 - DNA, 25 bp, Artificial Sequence
  Synthetic Polynucleotide
gcaucucaguaugaauuauacaatt

SEQ ID NO: 359 - DNA, 25 bp, Artificial Sequence
  Synthetic Polynucleotide
caucucaguaugaauuauuaaautt

SEQ ID NO: 360 - RNA, 25 nt, Artificial Sequence
  Synthetic Polynucleotide
gaaugauuuuucuuuacaaaacaaa

SEQ ID NO: 361 - DNA, 25 bp, Artificial Sequence
  Synthetic Polynucleotide
aguuuagggaacaauuuggaaautt

SEQ ID NO: 362 - DNA, 25 bp, Artificial Sequence
  Synthetic Polynucleotide
guuuagggaacaauuuggcaauutt

SEQ ID NO: 363 - DNA, 25 bp, Artificial Sequence
  Synthetic Polynucleotide
uuuagggaacaauuuggcaauuutg

SEQ ID NO: 364 - DNA, 25 bp, Artificial Sequence
  Synthetic Polynucleotide
uuagggaacaauuuggcaaauuugt

SEQ ID NO: 365 - DNA, 25 bp, Artificial Sequence
  Synthetic Polynucleotide
uagggaacaauuuggcaauauugtg

SEQ ID NO: 366 - RNA, 25 nt, Artificial Sequence
  Synthetic Polynucleotide
agggaacaauuuggcaauuaugugg

SEQ ID NO: 367 - DNA, 25 bp, Artificial Sequence
  Synthetic Polynucleotide
gggaacaauuuggcaauuuaguggt

SEQ ID NO: 368 - DNA, 25 bp, Artificial Sequence
  Synthetic Polynucleotide
ggaacaauuuggcaauuuuauggtt

SEQ ID NO: 369 - DNA, 25 bp, Artificial Sequence
  Synthetic Polynucleotide
gaacaauuuggcaauuuugaggutt

SEQ ID NO: 370 - DNA, 25 bp, Artificial Sequence
  Synthetic Polynucleotide
aacaauuuggcaauuuuguaguutt

SEQ ID NO: 371 - DNA, 25 bp, Artificial Sequence
  Synthetic Polynucleotide
acaauuuggcaauuuugugauuutc

SEQ ID NO: 372 - RNA, 25 nt, Artificial Sequence
  Synthetic Polynucleotide
caauuuggcaauuuuguggauuucg

SEQ ID NO: 373 - RNA, 25 nt, Artificial Sequence
  Synthetic Polynucleotide
aaauagcguucuuguaauuauacac

SEQ ID NO: 374 - RNA, 25 nt, Artificial Sequence
  Synthetic Polynucleotide
aauagcguucuuguaauuuaacacg

SEQ ID NO: 375 - DNA, 25 bp, Artificial Sequence
  Synthetic Polynucleotide
gcguucuuguaauuuuacaagcutt

SEQ ID NO: 376 - RNA, 25 nt, Artificial Sequence
  Synthetic Polynucleotide
uaauuuuacacgcuuuuguaaugga

SEQ ID NO: 377 - DNA, 25 bp, Artificial Sequence
  Synthetic Polynucleotide
cgcuuuugugauggagugcaguutt

SEQ ID NO: 378 - DNA, 25 bp, Artificial Sequence
  Synthetic Polynucleotide
gcuuuugugauggagugcuauuutg

SEQ ID NO: 379 - DNA, 25 bp, Artificial Sequence
  Synthetic Polynucleotide
cuuuugugauggagugcugauuugt

SEQ ID NO: 380 - DNA, 25 bp, Artificial Sequence
  Synthetic Polynucleotide
uuuugugauggagugcuguauugtt

SEQ ID NO: 381 - DNA, 25 bp, Artificial Sequence
  Synthetic Polynucleotide
uuugugauggagugcuguuauguta

SEQ ID NO: 382 - DNA, 25 bp, Artificial Sequence
  Synthetic Polynucleotide
ugugauggagugcuguuuuauuata

SEQ ID NO: 383 - DNA, 25 bp, Artificial Sequence
  Synthetic Polynucleotide
ugauggagugcuguuuuguaauata

SEQ ID NO: 384 - RNA, 25 nt, Artificial Sequence
  Synthetic Polynucleotide
gauggagugcuguuuuguuauauaa

SEQ ID NO: 385 - DNA, 25 bp, Artificial Sequence
  Synthetic Polynucleotide
auggagugcuguuuuguuaaauaat

SEQ ID NO: 386 - DNA, 25 bp, Artificial Sequence
  Synthetic Polynucleotide
uggagugcuguuuuguuauauaatt

SEQ ID NO: 387 - DNA, 25 bp, Artificial Sequence
  Synthetic Polynucleotide
ggagugcuguuuuguuauaaaautt

SEQ ID NO: 388 - DNA, 25 bp, Artificial Sequence
  Synthetic Polynucleotide
gagugcuguuuuguuauauaauuta

SEQ ID NO: 389 - RNA, 25 nt, Artificial Sequence
  Synthetic Polynucleotide
agugcuguuuuguuauauaauuuag

SEQ ID NO: 390 - RNA, 25 nt, Artificial Sequence
  Synthetic Polynucleotide
gugcuguuuuguuauauaaauuaga

SEQ ID NO: 391 - RNA, 25 nt, Artificial Sequence
  Synthetic Polynucleotide
ugcuguuuuguuauauaauauagac

SEQ ID NO: 392 - DNA, 25 bp, Artificial Sequence
  Synthetic Polynucleotide
gcuguuuuguuauauaauuaagact

SEQ ID NO: 393 - DNA, 25 bp, Artificial Sequence
  Synthetic Polynucleotide
cuguuuuguuauauaauuuagactt

SEQ ID NO: 394 - RNA, 25 nt, Artificial Sequence
  Synthetic Polynucleotide
auuugcauuuguuuauguaauuuca

SEQ ID NO: 395 - RNA, 25 nt, Artificial Sequence
  Synthetic Polynucleotide
guuuauguaauuucaggagaaauac

SEQ ID NO: 396 - RNA, 25 nt, Artificial Sequence
  Synthetic Polynucleotide
auguaauuucaggaggaauacugaa

SEQ ID NO: 397 - RNA, 25 nt, Artificial Sequence
  Synthetic Polynucleotide
gaauacugaacaucugaguacugga

SEQ ID NO: 398 - DNA, 25 bp, Artificial Sequence
  Synthetic Polynucleotide
caucugaguccuggaugauacuaat

SEQ ID NO: 399 - DNA, 25 bp, Artificial Sequence
  Synthetic Polynucleotide
aucugaguccuggaugauaauaata

SEQ ID NO: 400 - RNA, 25 nt, Artificial Sequence
  Synthetic Polynucleotide
ugaguccuggaugauacuaauaaac

SEQ ID NO: 401 - DNA, 25 bp, Artificial Sequence
  Synthetic Polynucleotide
aguccuggaugauacuaauaaacta

SEQ ID NO: 402 - RNA, 25 nt, Artificial Sequence
  Synthetic Polynucleotide
guccuggaugauacuaauaaacuaa

SEQ ID NO: 403 - DNA, 25 bp, Artificial Sequence
  Synthetic Polynucleotide
uccuggaugauacuaauaaacuaat

SEQ ID NO: 404 - DNA, 25 bp, Artificial Sequence
  Synthetic Polynucleotide
ccuggaugauacuaauaaaauaata

SEQ ID NO: 405 - RNA, 25 nt, Artificial Sequence
  Synthetic Polynucleotide
cuggaugauacuaauaaacaaauaa

SEQ ID NO: 406 - DNA, 25 bp, Artificial Sequence
  Synthetic Polynucleotide
uggaugauacuaauaaacuaauaat

SEQ ID NO: 407 - DNA, 25 bp, Artificial Sequence
  Synthetic Polynucleotide
ggaugauacuaauaaacuaauaatt

SEQ ID NO: 408 - DNA, 25 bp, Artificial Sequence
  Synthetic Polynucleotide
gaugauacuaauaaacuaaaaautg

SEQ ID NO: 409 - RNA, 25 nt, Artificial Sequence
  Synthetic Polynucleotide
augauacuaauaaacuaauaauugc

SEQ ID NO: 410 - RNA, 25 nt, Artificial Sequence
  Synthetic Polynucleotide
ugauacuaauaaacuaauaauugca

SEQ ID NO: 411 - RNA, 25 nt, Artificial Sequence
  Synthetic Polynucleotide
gauacuaauaaacuaauaaaugcag

SEQ ID NO: 412 - RNA, 25 nt, Artificial Sequence
  Synthetic Polynucleotide
auacuaauaaacuaauaauagcaga

SEQ ID NO: 413 - RNA, 25 nt, Artificial Sequence
  Synthetic Polynucleotide
uacuaauaaacuaauaauuacagag

SEQ ID NO: 414 - RNA, 27 nt, Artificial Sequence
  Synthetic Polynucleotide
ggaucuccuuugcccauguuuaguuau

SEQ ID NO: 415 - RNA, 27 nt, Artificial Sequence
  Synthetic Polynucleotide
aggauuuccuuugcccauguuuaguua

SEQ ID NO: 416 - RNA, 27 nt, Artificial Sequence
  Synthetic Polynucleotide
uaggaucuccuuugcccauguuuaguu

SEQ ID NO: 417 - RNA, 27 nt, Artificial Sequence
  Synthetic Polynucleotide
uuagguucuccuuugcccauguuuagu

SEQ ID NO: 418 - RNA, 27 nt, Artificial Sequence
  Synthetic Polynucleotide
cuuaguaucuccuuugcccauguuuag

SEQ ID NO: 419 - RNA, 27 nt, Artificial Sequence
  Synthetic Polynucleotide
ucuuaugaucuccuuugcccauguuua

SEQ ID NO: 420 - RNA, 27 nt, Artificial Sequence
  Synthetic Polynucleotide
uucuuuggaucuccuuugcccauguuu

SEQ ID NO: 421 - RNA, 27 nt, Artificial Sequence
  Synthetic Polynucleotide
cuucuuaggaucuccuuugcccauguu

SEQ ID NO: 422 - RNA, 27 nt, Artificial Sequence
  Synthetic Polynucleotide
gcuucuuaggaucuccuuugcccaugu

SEQ ID NO: 423 - RNA, 27 nt, Artificial Sequence
  Synthetic Polynucleotide
augauuacauuuugccucucggcuucu

SEQ ID NO: 424 - RNA, 27 nt, Artificial Sequence
  Synthetic Polynucleotide
uaugaugacauuuugccucucggcuuc

SEQ ID NO: 425 - RNA, 27 nt, Artificial Sequence
  Synthetic Polynucleotide
acaaauaaugcauaugaugacauuuug

SEQ ID NO: 426 - RNA, 27 nt, Artificial Sequence
  Synthetic Polynucleotide
guuuguacaaaaaaugcauaugaugac

SEQ ID NO: 427 - RNA, 27 nt, Artificial Sequence
  Synthetic Polynucleotide
aguuuucacaaaaaaugcauaugauga

SEQ ID NO: 428 - RNA, 27 nt, Artificial Sequence
  Synthetic Polynucleotide
acaaguuugcacaaaaaaugcauauga

SEQ ID NO: 429 - RNA, 27 nt, Artificial Sequence
  Synthetic Polynucleotide
ucuuauaaaacucugagaaguugacug

SEQ ID NO: 430 - RNA, 27 nt, Artificial Sequence
  Synthetic Polynucleotide
ucuucuaccucucugagcacuucuuag

SEQ ID NO: 431 - RNA, 27 nt, Artificial Sequence
  Synthetic Polynucleotide
gucuuucaccucucugagcacuucuua

SEQ ID NO: 432 - RNA, 27 nt, Artificial Sequence
  Synthetic Polynucleotide
ggucuuccaccucucugagcacuucuu

SEQ ID NO: 433 - RNA, 27 nt, Artificial Sequence
  Synthetic Polynucleotide
uggucuuccaccucucugagcacuucu

SEQ ID NO: 434 - RNA, 27 nt, Artificial Sequence
  Synthetic Polynucleotide
augguuuuccaccucucugagcacuuc

SEQ ID NO: 435 - RNA, 27 nt, Artificial Sequence
  Synthetic Polynucleotide
cauggucuuccaccucucugagcacuu

SEQ ID NO: 436 - RNA, 27 nt, Artificial Sequence
  Synthetic Polynucleotide
acauguucuuccaccucucugagcacu

SEQ ID NO: 437 - RNA, 27 nt, Artificial Sequence
  Synthetic Polynucleotide
gacauugucuuccaccucucugagcac

SEQ ID NO: 438 - RNA, 27 nt, Artificial Sequence
  Synthetic Polynucleotide
agacauggucuuccaccucucugagca

SEQ ID NO: 439 - RNA, 27 nt, Artificial Sequence
  Synthetic Polynucleotide
cagacuuggucuuccaccucucugagc

SEQ ID NO: 440 - RNA, 27 nt, Artificial Sequence
  Synthetic Polynucleotide
gcagauauggucuuccaccucucugag

SEQ ID NO: 441 - RNA, 27 nt, Artificial Sequence
  Synthetic Polynucleotide
agcagucauggucuuccaccucucuga

SEQ ID NO: 442 - RNA, 27 nt, Artificial Sequence
  Synthetic Polynucleotide
uagcauacauggucuuccaccucucug

SEQ ID NO: 443 - RNA, 27 nt, Artificial Sequence
  Synthetic Polynucleotide
uuagcugacauggucuuccaccucucu

SEQ ID NO: 444 - RNA, 27 nt, Artificial Sequence
  Synthetic Polynucleotide
uuuaguagacauggucuuccaccucuc

SEQ ID NO: 445 - RNA, 27 nt, Artificial Sequence
  Synthetic Polynucleotide
cuuuaucagacauggucuuccaccucu

SEQ ID NO: 446 - RNA, 27 nt, Artificial Sequence
  Synthetic Polynucleotide
ucuuuugcagacauggucuuccaccuc

SEQ ID NO: 447 - RNA, 27 nt, Artificial Sequence
  Synthetic Polynucleotide
cucuuuagcagacauggucuuccaccu

SEQ ID NO: 448 - RNA, 27 nt, Artificial Sequence
  Synthetic Polynucleotide
ucucuuuagcagacauggucuuccacc

SEQ ID NO: 449 - RNA, 27 nt, Artificial Sequence
  Synthetic Polynucleotide
uucucuuuagcagacauggucuuccac

SEQ ID NO: 450 - RNA, 27 nt, Artificial Sequence
  Synthetic Polynucleotide
uuucuuuuuagcagacauggucuucca

SEQ ID NO: 451 - RNA, 27 nt, Artificial Sequence
  Synthetic Polynucleotide
cuuucucuuuagcagacauggucuucc

SEQ ID NO: 452 - RNA, 27 nt, Artificial Sequence
  Synthetic Polynucleotide
ccuuuuucuuuagcagacauggucuuc

SEQ ID NO: 453 - RNA, 27 nt, Artificial Sequence
  Synthetic Polynucleotide
ccgcuuuugccauaucuucaaauuuuc

SEQ ID NO: 454 - RNA, 27 nt, Artificial Sequence
  Synthetic Polynucleotide
uccgcuuuugccauaucuucaaauuuu

SEQ ID NO: 455 - RNA, 27 nt, Artificial Sequence
  Synthetic Polynucleotide
ggauauagguuuucauuucucuuucau

SEQ ID NO: 456 - RNA, 27 nt, Artificial Sequence
  Synthetic Polynucleotide
auugguauccuugaacuucuuuuuugu

SEQ ID NO: 457 - RNA, 27 nt, Artificial Sequence
  Synthetic Polynucleotide
aaggaugccucuugggugcauugggau

SEQ ID NO: 458 - RNA, 27 nt, Artificial Sequence
  Synthetic Polynucleotide
gaagguggccucuugggugcauuggga

SEQ ID NO: 459 - RNA, 27 nt, Artificial Sequence
  Synthetic Polynucleotide
cgaaguaggccucuugggugcauuggg

SEQ ID NO: 460 - RNA, 27 nt, Artificial Sequence
  Synthetic Polynucleotide
ccgaaugaggccucuugggugcauugg

SEQ ID NO: 461 - RNA, 27 nt, Artificial Sequence
  Synthetic Polynucleotide
gccgauggaggccucuugggugcauug

SEQ ID NO: 462 - RNA, 27 nt, Artificial Sequence
  Synthetic Polynucleotide
ggccguaggaggccucuugggugcauu

SEQ ID NO: 463 - RNA, 27 nt, Artificial Sequence
  Synthetic Polynucleotide
aggccuaaggaggccucuugggugcau

SEQ ID NO: 464 - RNA, 27 nt, Artificial Sequence
  Synthetic Polynucleotide
aaggcugaaggaggccucuugggugca

SEQ ID NO: 465 - RNA, 27 nt, Artificial Sequence
  Synthetic Polynucleotide
gaaggucgaaggaggccucuugggugc

SEQ ID NO: 466 - RNA, 27 nt, Artificial Sequence
  Synthetic Polynucleotide
agaaguccgaaggaggccucuugggug

SEQ ID NO: 467 - RNA, 27 nt, Artificial Sequence
  Synthetic Polynucleotide
aagaaugccgaaggaggccucuugggu

SEQ ID NO: 468 - RNA, 27 nt, Artificial Sequence
  Synthetic Polynucleotide
gaagauggccgaaggaggccucuuggg

SEQ ID NO: 469 - RNA, 27 nt, Artificial Sequence
  Synthetic Polynucleotide
ggaaguaggccgaaggaggccucuugg

SEQ ID NO: 470 - RNA, 27 nt, Artificial Sequence
  Synthetic Polynucleotide
aggaauaaggccgaaggaggccucuug

SEQ ID NO: 471 - RNA, 27 nt, Artificial Sequence
  Synthetic Polynucleotide
gaggaugaaggccgaaggaggccucuu

SEQ ID NO: 472 - RNA, 27 nt, Artificial Sequence
  Synthetic Polynucleotide
agagguagaaggccgaaggaggccucu

SEQ ID NO: 473 - RNA, 27 nt, Artificial Sequence
  Synthetic Polynucleotide
aagaguaagaaggccgaaggaggccuc

SEQ ID NO: 474 - RNA, 27 nt, Artificial Sequence
  Synthetic Polynucleotide
gaagaugaagaaggccgaaggaggccu

SEQ ID NO: 475 - RNA, 27 nt, Artificial Sequence
  Synthetic Polynucleotide
uugucuucugcagcaguguuauuccac

SEQ ID NO: 476 - RNA, 27 nt, Artificial Sequence
  Synthetic Polynucleotide
ucuuuucauaaggcugcuugucaucug

SEQ ID NO: 477 - RNA, 27 nt, Artificial Sequence
  Synthetic Polynucleotide
uuuaguucgauaugcagcaauauccuu

SEQ ID NO: 478 - RNA, 27 nt, Artificial Sequence
  Synthetic Polynucleotide
ccuccucuuccuucuuuuucuugcuuu

SEQ ID NO: 479 - RNA, 27 nt, Artificial Sequence
  Synthetic Polynucleotide
uccucuucuuccuucuuuuucuugcuu

SEQ ID NO: 480 - RNA, 27 nt, Artificial Sequence
  Synthetic Polynucleotide
uuccuucucuuccuucuuuuucuugcu

SEQ ID NO: 481 - RNA, 27 nt, Artificial Sequence
  Synthetic Polynucleotide
cuuccuccucuuccuucuuuuucuugc

SEQ ID NO: 482 - RNA, 27 nt, Artificial Sequence
  Synthetic Polynucleotide
ucuucuuccucuuccuucuuuuucuug

SEQ ID NO: 483 - RNA, 27 nt, Artificial Sequence
  Synthetic Polynucleotide
cuuauucaucaucaucaucuucuucuu

SEQ ID NO: 484 - RNA, 27 nt, Artificial Sequence
  Synthetic Polynucleotide
aaccaucuuauucaucaucaucaucuu

SEQ ID NO: 485 - RNA, 27 nt, Artificial Sequence
  Synthetic Polynucleotide
uagaaucaacuuauucaucaucaucau

SEQ ID NO: 486 - RNA, 27 nt, Artificial Sequence
  Synthetic Polynucleotide
cuagauccaacuuauucaucaucauca

SEQ ID NO: 487 - RNA, 27 nt, Artificial Sequence
  Synthetic Polynucleotide
cugcguuagaaccaacuuauucaucau

SEQ ID NO: 488 - RNA, 27 nt, Artificial Sequence
  Synthetic Polynucleotide
aacugugcuagaaccaacuuauucauc

SEQ ID NO: 489 - RNA, 27 nt, Artificial Sequence
  Synthetic Polynucleotide
aaacuucgcuagaaccaacuuauucau

SEQ ID NO: 490 - RNA, 27 nt, Artificial Sequence
  Synthetic Polynucleotide
aaaacugcgcuagaaccaacuuauuca

SEQ ID NO: 491 - RNA, 27 nt, Artificial Sequence
  Synthetic Polynucleotide
aaaaauugcgcuagaaccaacuuauuc

SEQ ID NO: 492 - RNA, 27 nt, Artificial Sequence
  Synthetic Polynucleotide
aaaaaucugcgcuagaaccaacuuauu

SEQ ID NO: 493 - RNA, 27 nt, Artificial Sequence
  Synthetic Polynucleotide
aaaaauacugcgcuagaaccaacuuau

SEQ ID NO: 494 - RNA, 27 nt, Artificial Sequence
  Synthetic Polynucleotide
uuaaaugcuuuauagacaagaaaaaaa

SEQ ID NO: 495 - RNA, 27 nt, Artificial Sequence
  Synthetic Polynucleotide
guuaauugcuuuauagacaagaaaaaa

SEQ ID NO: 496 - RNA, 27 nt, Artificial Sequence
  Synthetic Polynucleotide
gguuauaugcuuuauagacaagaaaaa

SEQ ID NO: 497 - RNA, 27 nt, Artificial Sequence
  Synthetic Polynucleotide
ggguuuaaugcuuuauagacaagaaaa

SEQ ID NO: 498 - RNA, 27 nt, Artificial Sequence
  Synthetic Polynucleotide
gggguuaaaugcuuuauagacaagaaa

SEQ ID NO: 499 - RNA, 27 nt, Artificial Sequence
  Synthetic Polynucleotide
uuuuuucuuuaaaaggagugaguugug

SEQ ID NO: 500 - RNA, 27 nt, Artificial Sequence
  Synthetic Polynucleotide
uuuuuuucuuuaaaaggagugaguugu

SEQ ID NO: 501 - RNA, 27 nt, Artificial Sequence
  Synthetic Polynucleotide
auuuuuuucuuuaaaaggagugaguug

SEQ ID NO: 502 - RNA, 27 nt, Artificial Sequence
  Synthetic Polynucleotide
aauuuuuuucuuuaaaaggagugaguu

SEQ ID NO: 503 - RNA, 27 nt, Artificial Sequence
  Synthetic Polynucleotide
caauuuuuuucuuuaaaaggagugagu

SEQ ID NO: 504 - RNA, 27 nt, Artificial Sequence
  Synthetic Polynucleotide
ucaauuuuuuucuuuaaaaggagugag

SEQ ID NO: 505 - RNA, 27 nt, Artificial Sequence
  Synthetic Polynucleotide
uucaauuuuuuucuuuaaaaggaguga

SEQ ID NO: 506 - RNA, 27 nt, Artificial Sequence
  Synthetic Polynucleotide
uuucauuuuuuuucuuuaaaaggagug

SEQ ID NO: 507 - RNA, 27 nt, Artificial Sequence
  Synthetic Polynucleotide
auuucuauuuuuuucuuuaaaaggagu

SEQ ID NO: 508 - RNA, 27 nt, Artificial Sequence
  Synthetic Polynucleotide
cauuuuaauuuuuuucuuuaaaaggag

SEQ ID NO: 509 - RNA, 27 nt, Artificial Sequence
  Synthetic Polynucleotide
acauuucaauuuuuuucuuuaaaagga

SEQ ID NO: 510 - RNA, 27 nt, Artificial Sequence
  Synthetic Polynucleotide
uacauuucaauuuuuuucuuuaaaagg

SEQ ID NO: 511 - RNA, 27 nt, Artificial Sequence
  Synthetic Polynucleotide
uuacauuucaauuuuuuucuuuaaaag

SEQ ID NO: 512 - RNA, 27 nt, Artificial Sequence
  Synthetic Polynucleotide
cuuacuuuucaauuuuuuucuuuaaaa

SEQ ID NO: 513 - RNA, 27 nt, Artificial Sequence
  Synthetic Polynucleotide
ccuuauauuucaauuuuuuucuuuaaa

SEQ ID NO: 514 - RNA, 27 nt, Artificial Sequence
  Synthetic Polynucleotide
gccuuucauuucaauuuuuuucuuuaa

SEQ ID NO: 515 - RNA, 27 nt, Artificial Sequence
  Synthetic Polynucleotide
acagcuuuacauuucaauuuuuuucuu

SEQ ID NO: 516 - RNA, 27 nt, Artificial Sequence
  Synthetic Polynucleotide
cacagucuuacauuucaauuuuuuucu

SEQ ID NO: 517 - RNA, 27 nt, Artificial Sequence
  Synthetic Polynucleotide
uguacuguuuaaaaacaaaucuuacac

SEQ ID NO: 518 - RNA, 27 nt, Artificial Sequence
  Synthetic Polynucleotide
cuguauaguuuaaaaacaaaucuuaca

SEQ ID NO: 519 - RNA, 27 nt, Artificial Sequence
  Synthetic Polynucleotide
acuguucaguuuaaaaacaaaucuuac

SEQ ID NO: 520 - RNA, 27 nt, Artificial Sequence
  Synthetic Polynucleotide
gacacuguacaguuuaaaaacaaaucu

SEQ ID NO: 521 - RNA, 27 nt, Artificial Sequence
  Synthetic Polynucleotide
aagacucuguacaguuuaaaaacaaau

SEQ ID NO: 522 - RNA, 27 nt, Artificial Sequence
  Synthetic Polynucleotide
aaagauacuguacaguuuaaaaacaaa

SEQ ID NO: 523 - RNA, 27 nt, Artificial Sequence
  Synthetic Polynucleotide
aaaagucacuguacaguuuaaaaacaa

SEQ ID NO: 524 - RNA, 27 nt, Artificial Sequence
  Synthetic Polynucleotide
aaaaauacacuguacaguuuaaaaaca

SEQ ID NO: 525 - RNA, 27 nt, Artificial Sequence
  Synthetic Polynucleotide
aaaaaugacacuguacaguuuaaaaac

SEQ ID NO: 526 - RNA, 27 nt, Artificial Sequence
  Synthetic Polynucleotide
aaaaauagacacuguacaguuuaaaaa

SEQ ID NO: 527 - RNA, 27 nt, Artificial Sequence
  Synthetic Polynucleotide
caaaauaagacacuguacaguuuaaaa

SEQ ID NO: 528 - RNA, 27 nt, Artificial Sequence
  Synthetic Polynucleotide
acaaauaaagacacuguacaguuuaaa

SEQ ID NO: 529 - RNA, 27 nt, Artificial Sequence
  Synthetic Polynucleotide
uacaauaaaagacacuguacaguuuaa

SEQ ID NO: 530 - RNA, 27 nt, Artificial Sequence
  Synthetic Polynucleotide
auacauaaaaagacacuguacaguuua

SEQ ID NO: 531 - RNA, 27 nt, Artificial Sequence
  Synthetic Polynucleotide
uauacuaaaaaagacacuguacaguuu

SEQ ID NO: 532 - RNA, 27 nt, Artificial Sequence
  Synthetic Polynucleotide
cuauauaaaaaaagacacuguacaguu

SEQ ID NO: 533 - RNA, 27 nt, Artificial Sequence
  Synthetic Polynucleotide
acuauucaaaaaaagacacuguacagu

SEQ ID NO: 534 - RNA, 27 nt, Artificial Sequence
  Synthetic Polynucleotide
uaacuuuacaaaaaaagacacuguaca

SEQ ID NO: 535 - RNA, 27 nt, Artificial Sequence
  Synthetic Polynucleotide
guuaauuauacaaaaaaagacacugua

SEQ ID NO: 536 - RNA, 27 nt, Artificial Sequence
  Synthetic Polynucleotide
gguuauuggcuauugaaaauaccacca

SEQ ID NO: 537 - RNA, 27 nt, Artificial Sequence
  Synthetic Polynucleotide
agguuuguggcuauugaaaauaccacc

SEQ ID NO: 538 - RNA, 27 nt, Artificial Sequence
  Synthetic Polynucleotide
aagguuaguggcuauugaaaauaccac

SEQ ID NO: 539 - RNA, 27 nt, Artificial Sequence
  Synthetic Polynucleotide
gcaaguuuaguggcuauugaaaauacc

SEQ ID NO: 540 - RNA, 27 nt, Artificial Sequence
  Synthetic Polynucleotide
ggcaauguuaguggcuauugaaaauac

SEQ ID NO: 541 - RNA, 27 nt, Artificial Sequence
  Synthetic Polynucleotide
aggcaugguuaguggcuauugaaaaua

SEQ ID NO: 542 - RNA, 27 nt, Artificial Sequence
  Synthetic Polynucleotide
caggcuagguuaguggcuauugaaaau

SEQ ID NO: 543 - RNA, 27 nt, Artificial Sequence
  Synthetic Polynucleotide
ccagguaagguuaguggcuauugaaaa

SEQ ID NO: 544 - RNA, 27 nt, Artificial Sequence
  Synthetic Polynucleotide
accagucaagguuaguggcuauugaaa

SEQ ID NO: 545 - RNA, 27 nt, Artificial Sequence
  Synthetic Polynucleotide
uaccaugcaagguuaguggcuauugaa

SEQ ID NO: 546 - RNA, 27 nt, Artificial Sequence
  Synthetic Polynucleotide
guaccuggcaagguuaguggcuauuga

SEQ ID NO: 547 - RNA, 27 nt, Artificial Sequence
  Synthetic Polynucleotide
uguacuaggcaagguuaguggcuauug

SEQ ID NO: 548 - RNA, 27 nt, Artificial Sequence
  Synthetic Polynucleotide
cuguaucaggcaagguuaguggcuauu

SEQ ID NO: 549 - RNA, 27 nt, Artificial Sequence
  Synthetic Polynucleotide
acuguuccaggcaagguuaguggcuau

SEQ ID NO: 550 - RNA, 27 nt, Artificial Sequence
  Synthetic Polynucleotide
uacuguaccaggcaagguuaguggcua

SEQ ID NO: 551 - RNA, 27 nt, Artificial Sequence
  Synthetic Polynucleotide
auacuuuaccaggcaagguuaguggcu

SEQ ID NO: 552 - RNA, 27 nt, Artificial Sequence
  Synthetic Polynucleotide
cauacuguaccaggcaagguuaguggc

SEQ ID NO: 553 - RNA, 27 nt, Artificial Sequence
  Synthetic Polynucleotide
ccauauuguaccaggcaagguuagugg

SEQ ID NO: 554 - RNA, 27 nt, Artificial Sequence
  Synthetic Polynucleotide
aaauuuccaugccaauuuacaaccccc

SEQ ID NO: 555 - RNA, 27 nt, Artificial Sequence
  Synthetic Polynucleotide
uaaauuuccaugccaauuuacaacccc

SEQ ID NO: 556 - RNA, 27 nt, Artificial Sequence
  Synthetic Polynucleotide
uuaaauuuccaugccaauuuacaaccc

SEQ ID NO: 557 - RNA, 27 nt, Artificial Sequence
  Synthetic Polynucleotide
uuuaauuuuccaugccaauuuacaacc

SEQ ID NO: 558 - RNA, 27 nt, Artificial Sequence
  Synthetic Polynucleotide
cuuuauauuuccaugccaauuuacaac

SEQ ID NO: 559 - RNA, 27 nt, Artificial Sequence
  Synthetic Polynucleotide
gcuuuuaauuuccaugccaauuuacaa

SEQ ID NO: 560 - RNA, 27 nt, Artificial Sequence
  Synthetic Polynucleotide
ugcuuuaaauuuccaugccaauuuaca

SEQ ID NO: 561 - RNA, 27 nt, Artificial Sequence
  Synthetic Polynucleotide
cugcuuuaaauuuccaugccaauuuac

SEQ ID NO: 562 - RNA, 27 nt, Artificial Sequence
  Synthetic Polynucleotide
ccugcuuuaaauuuccaugccaauuua

SEQ ID NO: 563 - RNA, 27 nt, Artificial Sequence
  Synthetic Polynucleotide
accuguuuuaaauuuccaugccaauuu

SEQ ID NO: 564 - RNA, 27 nt, Artificial Sequence
  Synthetic Polynucleotide
aaccuucuuuaaauuuccaugccaauu

SEQ ID NO: 565 - RNA, 27 nt, Artificial Sequence
  Synthetic Polynucleotide
gaaccugcuuuaaauuuccaugccaau

SEQ ID NO: 566 - RNA, 27 nt, Artificial Sequence
  Synthetic Polynucleotide
agaacuugcuuuaaauuuccaugccaa

SEQ ID NO: 567 - RNA, 27 nt, Artificial Sequence
  Synthetic Polynucleotide
aagaaucugcuuuaaauuuccaugcca

SEQ ID NO: 568 - RNA, 27 nt, Artificial Sequence
  Synthetic Polynucleotide
caagauccugcuuuaaauuuccaugcc

SEQ ID NO: 569 - RNA, 27 nt, Artificial Sequence
  Synthetic Polynucleotide
acaaguaccugcuuuaaauuuccaugc

SEQ ID NO: 570 - RNA, 27 nt, Artificial Sequence
  Synthetic Polynucleotide
aacaauaaccugcuuuaaauuuccaug

SEQ ID NO: 571 - RNA, 27 nt, Artificial Sequence
  Synthetic Polynucleotide
caacaugaaccugcuuuaaauuuccau

SEQ ID NO: 572 - RNA, 27 nt, Artificial Sequence
  Synthetic Polynucleotide
ccaacuagaaccugcuuuaaauuucca

SEQ ID NO: 573 - RNA, 27 nt, Artificial Sequence
  Synthetic Polynucleotide
accaauaagaaccugcuuuaaauuucc

SEQ ID NO: 574 - RNA, 27 nt, Artificial Sequence
  Synthetic Polynucleotide
caccaucaagaaccugcuuuaaauuuc

SEQ ID NO: 575 - RNA, 27 nt, Artificial Sequence
  Synthetic Polynucleotide
gcaccuacaagaaccugcuuuaaauuu

SEQ ID NO: 576 - RNA, 27 nt, Artificial Sequence
  Synthetic Polynucleotide
ugcacuaacaagaaccugcuuuaaauu

SEQ ID NO: 577 - RNA, 27 nt, Artificial Sequence
  Synthetic Polynucleotide
gugcaucaacaagaaccugcuuuaaau

SEQ ID NO: 578 - RNA, 27 nt, Artificial Sequence
  Synthetic Polynucleotide
ugugcuccaacaagaaccugcuuuaaa

SEQ ID NO: 579 - RNA, 27 nt, Artificial Sequence
  Synthetic Polynucleotide
cuguguaccaacaagaaccugcuuuaa

SEQ ID NO: 580 - RNA, 27 nt, Artificial Sequence
  Synthetic Polynucleotide
gcuguucaccaacaagaaccugcuuua

SEQ ID NO: 581 - RNA, 27 nt, Artificial Sequence
  Synthetic Polynucleotide
ugcugugcaccaacaagaaccugcuuu

SEQ ID NO: 582 - RNA, 27 nt, Artificial Sequence
  Synthetic Polynucleotide
gugcuuugcaccaacaagaaccugcuu

SEQ ID NO: 583 - RNA, 27 nt, Artificial Sequence
  Synthetic Polynucleotide
ugugcugugcaccaacaagaaccugcu

SEQ ID NO: 584 - RNA, 27 nt, Artificial Sequence
  Synthetic Polynucleotide
uuguguugugcaccaacaagaaccugc

SEQ ID NO: 585 - RNA, 27 nt, Artificial Sequence
  Synthetic Polynucleotide
uuuguucugugcaccaacaagaaccug

SEQ ID NO: 586 - RNA, 27 nt, Artificial Sequence
  Synthetic Polynucleotide
auuugugcugugcaccaacaagaaccu

SEQ ID NO: 587 - RNA, 27 nt, Artificial Sequence
  Synthetic Polynucleotide
aauuuuugcugugcaccaacaagaacc

SEQ ID NO: 588 - RNA, 27 nt, Artificial Sequence
  Synthetic Polynucleotide
uaauuugugcugugcaccaacaagaac

SEQ ID NO: 589 - RNA, 27 nt, Artificial Sequence
  Synthetic Polynucleotide
cuaauuugugcugugcaccaacaagaa

SEQ ID NO: 590 - RNA, 27 nt, Artificial Sequence
  Synthetic Polynucleotide
acuaauuugugcugugcaccaacaaga

SEQ ID NO: 591 - RNA, 27 nt, Artificial Sequence
  Synthetic Polynucleotide
aacuauuuugugcugugcaccaacaag

SEQ ID NO: 592 - RNA, 27 nt, Artificial Sequence
  Synthetic Polynucleotide
uaacuuauuugugcugugcaccaacaa

SEQ ID NO: 593 - RNA, 27 nt, Artificial Sequence
  Synthetic Polynucleotide
auaacuaauuugugcugugcaccaaca

SEQ ID NO: 594 - RNA, 27 nt, Artificial Sequence
  Synthetic Polynucleotide
uauaauuaauuugugcugugcaccaac

SEQ ID NO: 595 - RNA, 27 nt, Artificial Sequence
  Synthetic Polynucleotide
auauaucuaauuugugcugugcaccaa

SEQ ID NO: 596 - RNA, 27 nt, Artificial Sequence
  Synthetic Polynucleotide
ucagauacaacugaagaugaaaaaacu

SEQ ID NO: 597 - RNA, 27 nt, Artificial Sequence
  Synthetic Polynucleotide
aucagugacaacugaagaugaaaaaac

SEQ ID NO: 598 - RNA, 27 nt, Artificial Sequence
  Synthetic Polynucleotide
caucauagacaacugaagaugaaaaaa

SEQ ID NO: 599 - RNA, 27 nt, Artificial Sequence
  Synthetic Polynucleotide
gcaucugagacaacugaagaugaaaaa

SEQ ID NO: 600 - RNA, 27 nt, Artificial Sequence
  Synthetic Polynucleotide
ugcauuagagacaacugaagaugaaaa

SEQ ID NO: 601 - RNA, 27 nt, Artificial Sequence
  Synthetic Polynucleotide
cugcaucagagacaacugaagaugaaa

SEQ ID NO: 602 - RNA, 27 nt, Artificial Sequence
  Synthetic Polynucleotide
gcugcuucagagacaacugaagaugaa

SEQ ID NO: 603 - RNA, 27 nt, Artificial Sequence
  Synthetic Polynucleotide
agcuguaucagagacaacugaagauga

SEQ ID NO: 604 - RNA, 27 nt, Artificial Sequence
  Synthetic Polynucleotide
auuauuucguauaagcugcaucagaga

SEQ ID NO: 605 - RNA, 27 nt, Artificial Sequence
  Synthetic Polynucleotide
aauuauuucguauaagcugcaucagag

SEQ ID NO: 606 - RNA, 27 nt, Artificial Sequence
  Synthetic Polynucleotide
aacaauaauuauuucguauaagcugca

SEQ ID NO: 607 - RNA, 27 nt, Artificial Sequence
  Synthetic Polynucleotide
gaacaucaauuauuucguauaagcugc

SEQ ID NO: 608 - RNA, 27 nt, Artificial Sequence
  Synthetic Polynucleotide
agaacuacaauuauuucguauaagcug

SEQ ID NO: 609 - RNA, 27 nt, Artificial Sequence
  Synthetic Polynucleotide
cagaauaacaauuauuucguauaagcu

SEQ ID NO: 610 - RNA, 27 nt, Artificial Sequence
  Synthetic Polynucleotide
aacaguacaacaauuauuucguauaag

SEQ ID NO: 611 - RNA, 27 nt, Artificial Sequence
  Synthetic Polynucleotide
uaacauaacaacaauuauuucguauaa

SEQ ID NO: 612 - RNA, 27 nt, Artificial Sequence
  Synthetic Polynucleotide
uuaacugaacaacaauuauuucguaua

SEQ ID NO: 613 - RNA, 27 nt, Artificial Sequence
  Synthetic Polynucleotide
guuaauagaacaacaauuauuucguau

SEQ ID NO: 614 - RNA, 27 nt, Artificial Sequence
  Synthetic Polynucleotide
aguuaucagaacaacaauuauuucgua

SEQ ID NO: 615 - RNA, 27 nt, Artificial Sequence
  Synthetic Polynucleotide
caguuuacagaacaacaauuauuucgu

SEQ ID NO: 616 - RNA, 27 nt, Artificial Sequence
  Synthetic Polynucleotide
ucaguuaacagaacaacaauuauuucg

SEQ ID NO: 617 - RNA, 27 nt, Artificial Sequence
  Synthetic Polynucleotide
uucaguuaacagaacaacaauuauuuc

SEQ ID NO: 618 - RNA, 27 nt, Artificial Sequence
  Synthetic Polynucleotide
guauuuaguuaacagaacaacaauuau

SEQ ID NO: 619 - RNA, 27 nt, Artificial Sequence
  Synthetic Polynucleotide
gguauucaguuaacagaacaacaauua

SEQ ID NO: 620 - RNA, 27 nt, Artificial Sequence
  Synthetic Polynucleotide
gugguuuucaguuaacagaacaacaau

SEQ ID NO: 621 - RNA, 27 nt, Artificial Sequence
  Synthetic Polynucleotide
agugguauucaguuaacagaacaacaa

SEQ ID NO: 622 - RNA, 27 nt, Artificial Sequence
  Synthetic Polynucleotide
agaguuguauucaguuaacagaacaac

SEQ ID NO: 623 - RNA, 27 nt, Artificial Sequence
  Synthetic Polynucleotide
uacagugugguauucaguuaacagaac

SEQ ID NO: 624 - RNA, 27 nt, Artificial Sequence
  Synthetic Polynucleotide
uuacauagugguauucaguuaacagaa

SEQ ID NO: 625 - RNA, 27 nt, Artificial Sequence
  Synthetic Polynucleotide
aauuauagagugguauucaguuaacag

SEQ ID NO: 626 - RNA, 27 nt, Artificial Sequence
  Synthetic Polynucleotide
ugcaauuacagagugguauucaguuaa

SEQ ID NO: 627 - RNA, 27 nt, Artificial Sequence
  Synthetic Polynucleotide
uugcauuuacagagugguauucaguua

SEQ ID NO: 628 - RNA, 27 nt, Artificial Sequence
  Synthetic Polynucleotide
uuugcuauuacagagugguauucaguu

SEQ ID NO: 629 - RNA, 27 nt, Artificial Sequence
  Synthetic Polynucleotide
uuuuguaauuacagagugguauucagu

SEQ ID NO: 630 - RNA, 27 nt, Artificial Sequence
  Synthetic Polynucleotide
uuuuuucaauuacagagugguauucag

SEQ ID NO: 631 - RNA, 27 nt, Artificial Sequence
  Synthetic Polynucleotide
uuuuuugcaauuacagagugguauuca

SEQ ID NO: 632 - RNA, 27 nt, Artificial Sequence
  Synthetic Polynucleotide
gucaauaaaacagcugcaacuuuuuuu

SEQ ID NO: 633 - RNA, 27 nt, Artificial Sequence
  Synthetic Polynucleotide
augucuacaaaacagcugcaacuuuuu

SEQ ID NO: 634 - RNA, 27 nt, Artificial Sequence
  Synthetic Polynucleotide
aauguuaacaaaacagcugcaacuuuu

SEQ ID NO: 635 - RNA, 27 nt, Artificial Sequence
  Synthetic Polynucleotide
gaaugucaacaaaacagcugcaacuuu

SEQ ID NO: 636 - RNA, 27 nt, Artificial Sequence
  Synthetic Polynucleotide
agaauuucaacaaaacagcugcaacuu

SEQ ID NO: 637 - RNA, 27 nt, Artificial Sequence
  Synthetic Polynucleotide
ucagauugucaacaaaacagcugcaac

SEQ ID NO: 638 - RNA, 27 nt, Artificial Sequence
  Synthetic Polynucleotide
uucaguaugucaacaaaacagcugcaa

SEQ ID NO: 639 - RNA, 27 nt, Artificial Sequence
  Synthetic Polynucleotide
auucauaaugucaacaaaacagcugca

SEQ ID NO: 640 - RNA, 27 nt, Artificial Sequence
  Synthetic Polynucleotide
cauucugaaugucaacaaaacagcugc

SEQ ID NO: 641 - RNA, 27 nt, Artificial Sequence
  Synthetic Polynucleotide
gcauuuagaaugucaacaaaacagcug

SEQ ID NO: 642 - RNA, 27 nt, Artificial Sequence
  Synthetic Polynucleotide
aagcauucagaaugucaacaaaacagc

SEQ ID NO: 643 - RNA, 27 nt, Artificial Sequence
  Synthetic Polynucleotide
uagaaucauucagaaugucaacaaaac

SEQ ID NO: 644 - RNA, 27 nt, Artificial Sequence
  Synthetic Polynucleotide
uuagaugcauucagaaugucaacaaaa

SEQ ID NO: 645 - RNA, 27 nt, Artificial Sequence
  Synthetic Polynucleotide
cuuaguagcauucagaaugucaacaaa

SEQ ID NO: 646 - RNA, 27 nt, Artificial Sequence
  Synthetic Polynucleotide
acuuauaagcauucagaaugucaacaa

SEQ ID NO: 647 - RNA, 27 nt, Artificial Sequence
  Synthetic Polynucleotide
uacuuugaagcauucagaaugucaaca

SEQ ID NO: 648 - RNA, 27 nt, Artificial Sequence
  Synthetic Polynucleotide
uuacuuagaagcauucagaaugucaac

SEQ ID NO: 649 - RNA, 27 nt, Artificial Sequence
  Synthetic Polynucleotide
uuuacuuagaagcauucagaaugucaa

SEQ ID NO: 650 - RNA, 27 nt, Artificial Sequence
  Synthetic Polynucleotide
uauuuucuuagaagcauucagaauguc

SEQ ID NO: 651 - RNA, 27 nt, Artificial Sequence
  Synthetic Polynucleotide
guauuuacuuagaagcauucagaaugu

SEQ ID NO: 652 - RNA, 27 nt, Artificial Sequence
  Synthetic Polynucleotide
uuguauuuacuuagaagcauucagaau

SEQ ID NO: 653 - RNA, 27 nt, Artificial Sequence
  Synthetic Polynucleotide
aauuguauuuacuuagaagcauucaga

SEQ ID NO: 654 - RNA, 27 nt, Artificial Sequence
  Synthetic Polynucleotide
aaaauuguauuuacuuagaagcauuca

SEQ ID NO: 655 - RNA, 27 nt, Artificial Sequence
  Synthetic Polynucleotide
aaaaauuguauuuacuuagaagcauuc

SEQ ID NO: 656 - RNA, 27 nt, Artificial Sequence
  Synthetic Polynucleotide
aaaaauauuguauuuacuuagaagcau

SEQ ID NO: 657 - RNA, 27 nt, Artificial Sequence
  Synthetic Polynucleotide
aaaaguacaacaauacuaauaaaaaaa

SEQ ID NO: 658 - RNA, 27 nt, Artificial Sequence
  Synthetic Polynucleotide
gaaaaugacaacaauacuaauaaaaaa

SEQ ID NO: 659 - RNA, 27 nt, Artificial Sequence
  Synthetic Polynucleotide
ugaaauggacaacaauacuaauaaaaa

SEQ ID NO: 660 - RNA, 27 nt, Artificial Sequence
  Synthetic Polynucleotide
augaauaggacaacaauacuaauaaaa

SEQ ID NO: 661 - RNA, 27 nt, Artificial Sequence
  Synthetic Polynucleotide
uaugauaaggacaacaauacuaauaaa

SEQ ID NO: 662 - RNA, 27 nt, Artificial Sequence
  Synthetic Polynucleotide
cuauguaaaggacaacaauacuaauaa

SEQ ID NO: 663 - RNA, 27 nt, Artificial Sequence
  Synthetic Polynucleotide
ccuauuaaaaggacaacaauacuaaua

SEQ ID NO: 664 - RNA, 27 nt, Artificial Sequence
  Synthetic Polynucleotide
accuaugaaaaggacaacaauacuaau

SEQ ID NO: 665 - RNA, 27 nt, Artificial Sequence
  Synthetic Polynucleotide
agaccuaugaaaaggacaacaauacua

SEQ ID NO: 666 - RNA, 27 nt, Artificial Sequence
  Synthetic Polynucleotide
uucaguccuaugaaaaggacaacaaua

SEQ ID NO: 667 - RNA, 27 nt, Artificial Sequence
  Synthetic Polynucleotide
uuucauaccuaugaaaaggacaacaau

SEQ ID NO: 668 - RNA, 27 nt, Artificial Sequence
  Synthetic Polynucleotide
aauuuuagaccuaugaaaaggacaaca

SEQ ID NO: 669 - RNA, 27 nt, Artificial Sequence
  Synthetic Polynucleotide
aaauuucagaccuaugaaaaggacaac

SEQ ID NO: 670 - RNA, 27 nt, Artificial Sequence
  Synthetic Polynucleotide
aaaauuucagaccuaugaaaaggacaa

SEQ ID NO: 671 - RNA, 27 nt, Artificial Sequence
  Synthetic Polynucleotide
aaaaauuucagaccuaugaaaaggaca

SEQ ID NO: 672 - RNA, 27 nt, Artificial Sequence
  Synthetic Polynucleotide
gaaaauuuucagaccuaugaaaaggac

SEQ ID NO: 673 - RNA, 27 nt, Artificial Sequence
  Synthetic Polynucleotide
ucaaguagaaaaauuucagaccuauga

SEQ ID NO: 674 - RNA, 27 nt, Artificial Sequence
  Synthetic Polynucleotide
caaaaucaaaagacuagcuuccccuca

SEQ ID NO: 675 - RNA, 27 nt, Artificial Sequence
  Synthetic Polynucleotide
gcaaaugcaaaagacuagcuuccccuc

SEQ ID NO: 676 - RNA, 27 nt, Artificial Sequence
  Synthetic Polynucleotide
ggcaauagcaaaagacuagcuuccccu

SEQ ID NO: 677 - RNA, 27 nt, Artificial Sequence
  Synthetic Polynucleotide
gggcauaagcaaaagacuagcuucccc

SEQ ID NO: 678 - RNA, 27 nt, Artificial Sequence
  Synthetic Polynucleotide
ugggcuaaagcaaaagacuagcuuccc

SEQ ID NO: 679 - RNA, 27 nt, Artificial Sequence
  Synthetic Polynucleotide
uaacuuuaugaaaggauaaacacugua

SEQ ID NO: 680 - RNA, 27 nt, Artificial Sequence
  Synthetic Polynucleotide
gcuaauuauaugaaaggauaaacacug

SEQ ID NO: 681 - RNA, 27 nt, Artificial Sequence
  Synthetic Polynucleotide
auuaguuaacuauaugaaaggauaaac

SEQ ID NO: 682 - RNA, 27 nt, Artificial Sequence
  Synthetic Polynucleotide
guguauacaaaagcuuuuuauuagcua

SEQ ID NO: 683 - RNA, 27 nt, Artificial Sequence
  Synthetic Polynucleotide
gaaaauuaucucaacuuaacuuuaccc

SEQ ID NO: 684 - RNA, 27 nt, Artificial Sequence
  Synthetic Polynucleotide
augaauacuaucucaacuuaacuuuac

SEQ ID NO: 685 - RNA, 27 nt, Artificial Sequence
  Synthetic Polynucleotide
uggauuaaaacuaucucaacuuaacuu

SEQ ID NO: 686 - RNA, 27 nt, Artificial Sequence
  Synthetic Polynucleotide
uaugguugaaaacuaucucaacuuaac

SEQ ID NO: 687 - RNA, 27 nt, Artificial Sequence
  Synthetic Polynucleotide
uuauguaugaaaacuaucucaacuuaa

SEQ ID NO: 688 - RNA, 27 nt, Artificial Sequence
  Synthetic Polynucleotide
guuauugaugaaaacuaucucaacuua

SEQ ID NO: 689 - RNA, 27 nt, Artificial Sequence
  Synthetic Polynucleotide
aguuauggaugaaaacuaucucaacuu

SEQ ID NO: 690 - RNA, 27 nt, Artificial Sequence
  Synthetic Polynucleotide
ucaguuauggaugaaaacuaucucaac

SEQ ID NO: 691 - RNA, 27 nt, Artificial Sequence
  Synthetic Polynucleotide
uucaguuauggaugaaaacuaucucaa

SEQ ID NO: 692 - RNA, 27 nt, Artificial Sequence
  Synthetic Polynucleotide
guucauuuauggaugaaaacuaucuca

SEQ ID NO: 693 - RNA, 27 nt, Artificial Sequence
  Synthetic Polynucleotide
uggauuuucaguuauggaugaaaacua

SEQ ID NO: 694 - RNA, 27 nt, Artificial Sequence
  Synthetic Polynucleotide
uuuuguauguucaguuauggaugaaaa

SEQ ID NO: 695 - RNA, 27 nt, Artificial Sequence
  Synthetic Polynucleotide
uauguuaaauuucuuaacugaucaaga

SEQ ID NO: 696 - RNA, 27 nt, Artificial Sequence
  Synthetic Polynucleotide
gcuauuugaaauuucuuaacugaucaa

SEQ ID NO: 697 - RNA, 27 nt, Artificial Sequence
  Synthetic Polynucleotide
uugauuacucuucaguuuguaaaugua

SEQ ID NO: 698 - RNA, 27 nt, Artificial Sequence
  Synthetic Polynucleotide
auugauuacucuucaguuuguaaaugu

SEQ ID NO: 699 - RNA, 27 nt, Artificial Sequence
  Synthetic Polynucleotide
guagauugauuacucuucaguuuguaa

SEQ ID NO: 700 - RNA, 27 nt, Artificial Sequence
  Synthetic Polynucleotide
ucaaguacagacuuucaaaauguuuga

SEQ ID NO: 701 - RNA, 27 nt, Artificial Sequence
  Synthetic Polynucleotide
agaacuuacuuuucuauuaguccuuca

SEQ ID NO: 702 - RNA, 27 nt, Artificial Sequence
  Synthetic Polynucleotide
gguuauaacauacuuuucuauuagucc

SEQ ID NO: 703 - RNA, 27 nt, Artificial Sequence
  Synthetic Polynucleotide
uguaaugguuagaacauacuuuucuau

SEQ ID NO: 704 - RNA, 27 nt, Artificial Sequence
  Synthetic Polynucleotide
auguauagguuagaacauacuuuucua

SEQ ID NO: 705 - RNA, 27 nt, Artificial Sequence
  Synthetic Polynucleotide
cucauuuaaagguuagaacauacuuuu

SEQ ID NO: 706 - RNA, 27 nt, Artificial Sequence
  Synthetic Polynucleotide
aacuguaaaauauaacugccauuacau

SEQ ID NO: 707 - RNA, 27 nt, Artificial Sequence
  Synthetic Polynucleotide
gaacuucaaaauauaacugccauuaca

SEQ ID NO: 708 - RNA, 27 nt, Artificial Sequence
  Synthetic Polynucleotide
gggauucauucucaggucuucuuuaau

SEQ ID NO: 709 - RNA, 27 nt, Artificial Sequence
  Synthetic Polynucleotide
ggggauacauucucaggucuucuuuaa

SEQ ID NO: 710 - RNA, 27 nt, Artificial Sequence
  Synthetic Polynucleotide
uuuggugauacauucucaggucuucuu

SEQ ID NO: 711 - RNA, 27 nt, Artificial Sequence
  Synthetic Polynucleotide
uuuuguggauacauucucaggucuucu

SEQ ID NO: 712 - RNA, 27 nt, Artificial Sequence
  Synthetic Polynucleotide
cuuuuugggauacauucucaggucuuc

SEQ ID NO: 713 - RNA, 27 nt, Artificial Sequence
  Synthetic Polynucleotide
gcuuuuggggauacauucucaggucuu

SEQ ID NO: 714 - RNA, 27 nt, Artificial Sequence
  Synthetic Polynucleotide
cgcuuuuggggauacauucucaggucu

SEQ ID NO: 715 - RNA, 27 nt, Artificial Sequence
  Synthetic Polynucleotide
acgcuuuuggggauacauucucagguc

SEQ ID NO: 716 - RNA, 27 nt, Artificial Sequence
  Synthetic Polynucleotide
cacgcuuuuggggauacauucucaggu

SEQ ID NO: 717 - RNA, 27 nt, Artificial Sequence
  Synthetic Polynucleotide
ucacguuuuuggggauacauucucagg

SEQ ID NO: 718 - RNA, 27 nt, Artificial Sequence
  Synthetic Polynucleotide
cucacucuuuuggggauacauucucag

SEQ ID NO: 719 - RNA, 27 nt, Artificial Sequence
  Synthetic Polynucleotide
augucuacaaaaaauuuaauauggcag

SEQ ID NO: 720 - RNA, 27 nt, Artificial Sequence
  Synthetic Polynucleotide
aauguuaacaaaaaauuuaauauggca

SEQ ID NO: 721 - RNA, 27 nt, Artificial Sequence
  Synthetic Polynucleotide
uaaugucaacaaaaaauuuaauauggc

SEQ ID NO: 722 - RNA, 27 nt, Artificial Sequence
  Synthetic Polynucleotide
cuaauuucaacaaaaaauuuaauaugg

SEQ ID NO: 723 - RNA, 27 nt, Artificial Sequence
  Synthetic Polynucleotide
gacuauugucaacaaaaaauuuaauau

SEQ ID NO: 724 - RNA, 27 nt, Artificial Sequence
  Synthetic Polynucleotide
ugagauuaaugucaacaaaaaauuuaa

SEQ ID NO: 725 - RNA, 27 nt, Artificial Sequence
  Synthetic Polynucleotide
cugagucuaaugucaacaaaaaauuua

SEQ ID NO: 726 - RNA, 27 nt, Artificial Sequence
  Synthetic Polynucleotide
acugauacuaaugucaacaaaaaauuu

SEQ ID NO: 727 - RNA, 27 nt, Artificial Sequence
  Synthetic Polynucleotide
uauagucagcauuuucauagucuucac

SEQ ID NO: 728 - RNA, 27 nt, Artificial Sequence
  Synthetic Polynucleotide
uaaaauuacuuguaauggaaagucucg

SEQ ID NO: 729 - RNA, 27 nt, Artificial Sequence
  Synthetic Polynucleotide
aauaauucauacugagaugcaaaguuu

SEQ ID NO: 730 - RNA, 27 nt, Artificial Sequence
  Synthetic Polynucleotide
gaauauuucauacugagaugcaaaguu

SEQ ID NO: 731 - RNA, 27 nt, Artificial Sequence
  Synthetic Polynucleotide
uugaauaauucauacugagaugcaaag

SEQ ID NO: 732 - RNA, 27 nt, Artificial Sequence
  Synthetic Polynucleotide
aauuguauaauucauacugagaugcaa

SEQ ID NO: 733 - RNA, 27 nt, Artificial Sequence
  Synthetic Polynucleotide
aaauuuaauaauucauacugagaugca

SEQ ID NO: 734 - RNA, 27 nt, Artificial Sequence
  Synthetic Polynucleotide
uuuguuuuguaaagaaaaaucauucaa

SEQ ID NO: 735 - RNA, 27 nt, Artificial Sequence
  Synthetic Polynucleotide
aaauuuccaaauuguucccuaaacucc

SEQ ID NO: 736 - RNA, 27 nt, Artificial Sequence
  Synthetic Polynucleotide
aaaauugccaaauuguucccuaaacuc

SEQ ID NO: 737 - RNA, 27 nt, Artificial Sequence
  Synthetic Polynucleotide
caaaauugccaaauuguucccuaaacu

SEQ ID NO: 738 - RNA, 27 nt, Artificial Sequence
  Synthetic Polynucleotide
acaaauuugccaaauuguucccuaaac

SEQ ID NO: 739 - RNA, 27 nt, Artificial Sequence
  Synthetic Polynucleotide
cacaauauugccaaauuguucccuaaa

SEQ ID NO: 740 - RNA, 27 nt, Artificial Sequence
  Synthetic Polynucleotide
ccacauaauugccaaauuguucccuaa

SEQ ID NO: 741 - RNA, 27 nt, Artificial Sequence
  Synthetic Polynucleotide
accacuaaauugccaaauuguucccua

SEQ ID NO: 742 - RNA, 27 nt, Artificial Sequence
  Synthetic Polynucleotide
aaccauaaaauugccaaauuguucccu

SEQ ID NO: 743 - RNA, 27 nt, Artificial Sequence
  Synthetic Polynucleotide
aaaccucaaaauugccaaauuguuccc

SEQ ID NO: 744 - RNA, 27 nt, Artificial Sequence
  Synthetic Polynucleotide
aaaacuacaaaauugccaaauuguucc

SEQ ID NO: 745 - RNA, 27 nt, Artificial Sequence
  Synthetic Polynucleotide
gaaaaucacaaaauugccaaauuguuc

SEQ ID NO: 746 - RNA, 27 nt, Artificial Sequence
  Synthetic Polynucleotide
cgaaauccacaaaauugccaaauuguu

SEQ ID NO: 747 - RNA, 27 nt, Artificial Sequence
  Synthetic Polynucleotide
guguauaauuacaagaacgcuauuuua

SEQ ID NO: 748 - RNA, 27 nt, Artificial Sequence
  Synthetic Polynucleotide
cguguuaaauuacaagaacgcuauuuu

SEQ ID NO: 749 - RNA, 27 nt, Artificial Sequence
  Synthetic Polynucleotide
aaagcuuguaaaauuacaagaacgcua

SEQ ID NO: 750 - RNA, 27 nt, Artificial Sequence
  Synthetic Polynucleotide
uccauuacaaaagcguguaaaauuaca

SEQ ID NO: 751 - RNA, 27 nt, Artificial Sequence
  Synthetic Polynucleotide
aaaacugcacuccaucacaaaagcgug

SEQ ID NO: 752 - RNA, 27 nt, Artificial Sequence
  Synthetic Polynucleotide
caaaauagcacuccaucacaaaagcgu

SEQ ID NO: 753 - RNA, 27 nt, Artificial Sequence
  Synthetic Polynucleotide
acaaaucagcacuccaucacaaaagcg

SEQ ID NO: 754 - RNA, 27 nt, Artificial Sequence
  Synthetic Polynucleotide
aacaauacagcacuccaucacaaaagc

SEQ ID NO: 755 - RNA, 27 nt, Artificial Sequence
  Synthetic Polynucleotide
uaacauaacagcacuccaucacaaaag

SEQ ID NO: 756 - RNA, 27 nt, Artificial Sequence
  Synthetic Polynucleotide
uauaauaaaacagcacuccaucacaaa

SEQ ID NO: 757 - RNA, 27 nt, Artificial Sequence
  Synthetic Polynucleotide
uauauuacaaaacagcacuccaucaca

SEQ ID NO: 758 - RNA, 27 nt, Artificial Sequence
  Synthetic Polynucleotide
uuauauaacaaaacagcacuccaucac

SEQ ID NO: 759 - RNA, 27 nt, Artificial Sequence
  Synthetic Polynucleotide
auuauuuaacaaaacagcacuccauca

SEQ ID NO: 760 - RNA, 27 nt, Artificial Sequence
  Synthetic Polynucleotide
aauuauauaacaaaacagcacuccauc

SEQ ID NO: 761 - RNA, 27 nt, Artificial Sequence
  Synthetic Polynucleotide
aaauuuuauaacaaaacagcacuccau

SEQ ID NO: 762 - RNA, 27 nt, Artificial Sequence
  Synthetic Polynucleotide
uaaauuauauaacaaaacagcacucca

SEQ ID NO: 763 - RNA, 27 nt, Artificial Sequence
  Synthetic Polynucleotide
cuaaauuauauaacaaaacagcacucc

SEQ ID NO: 764 - RNA, 27 nt, Artificial Sequence
  Synthetic Polynucleotide
ucuaauuuauauaacaaaacagcacuc

SEQ ID NO: 765 - RNA, 27 nt, Artificial Sequence
  Synthetic Polynucleotide
gucuauauuauauaacaaaacagcacu

SEQ ID NO: 766 - RNA, 27 nt, Artificial Sequence
  Synthetic Polynucleotide
agucuuaauuauauaacaaaacagcac

SEQ ID NO: 767 - RNA, 27 nt, Artificial Sequence
  Synthetic Polynucleotide
aagucuaaauuauauaacaaaacagca

SEQ ID NO: 768 - RNA, 27 nt, Artificial Sequence
  Synthetic Polynucleotide
ugaaauuacauaaacaaaugcaaaugg

SEQ ID NO: 769 - RNA, 27 nt, Artificial Sequence
  Synthetic Polynucleotide
guauuucuccugaaauuacauaaacaa

SEQ ID NO: 770 - RNA, 27 nt, Artificial Sequence
  Synthetic Polynucleotide
uucaguauuccuccugaaauuacauaa

SEQ ID NO: 771 - RNA, 27 nt, Artificial Sequence
  Synthetic Polynucleotide
uccaguacucagauguucaguauuccu

SEQ ID NO: 772 - RNA, 27 nt, Artificial Sequence
  Synthetic Polynucleotide
auuaguaucauccaggacucagauguu

SEQ ID NO: 773 - RNA, 27 nt, Artificial Sequence
  Synthetic Polynucleotide
uauuauuaucauccaggacucagaugu

SEQ ID NO: 774 - RNA, 27 nt, Artificial Sequence
  Synthetic Polynucleotide
guuuauuaguaucauccaggacucaga

SEQ ID NO: 775 - RNA, 27 nt, Artificial Sequence
  Synthetic Polynucleotide
uaguuuauuaguaucauccaggacuca

SEQ ID NO: 776 - RNA, 27 nt, Artificial Sequence
  Synthetic Polynucleotide
uuaguuuauuaguaucauccaggacuc

SEQ ID NO: 777 - RNA, 27 nt, Artificial Sequence
  Synthetic Polynucleotide
auuaguuuauuaguaucauccaggacu

SEQ ID NO: 778 - RNA, 27 nt, Artificial Sequence
  Synthetic Polynucleotide
uauuauuuuauuaguaucauccaggac

SEQ ID NO: 779 - RNA, 27 nt, Artificial Sequence
  Synthetic Polynucleotide
uuauuuguuuauuaguaucauccagga

SEQ ID NO: 780 - RNA, 27 nt, Artificial Sequence
  Synthetic Polynucleotide
auuauuaguuuauuaguaucauccagg

SEQ ID NO: 781 - RNA, 27 nt, Artificial Sequence
  Synthetic Polynucleotide
aauuauuaguuuauuaguaucauccag

SEQ ID NO: 782 - RNA, 27 nt, Artificial Sequence
  Synthetic Polynucleotide
caauuuuuaguuuauuaguaucaucca

SEQ ID NO: 783 - RNA, 27 nt, Artificial Sequence
  Synthetic Polynucleotide
gcaauuauuaguuuauuaguaucaucc

SEQ ID NO: 784 - RNA, 27 nt, Artificial Sequence
  Synthetic Polynucleotide
ugcaauuauuaguuuauuaguaucauc

SEQ ID NO: 785 - RNA, 27 nt, Artificial Sequence
  Synthetic Polynucleotide
cugcauuuauuaguuuauuaguaucau

SEQ ID NO: 786 - RNA, 27 nt, Artificial Sequence
  Synthetic Polynucleotide
ucugcuauuauuaguuuauuaguauca

SEQ ID NO: 787 - RNA, 27 nt, Artificial Sequence
  Synthetic Polynucleotide
cucuguaauuauuaguuuauuaguauc

SEQ ID NO: 788 - RNA, 36 nt, Artificial Sequence; identical to SEQ ID NO: 27
  Synthetic Polynucleotide
  modified_base (1)..(1): 2′-O-methyl uridine
  misc_feature (1)..(2): phosphorothioate linkage between the two indicated positions
  modified_base (2)..(2): 2′-O-methyl guanosine
  modified_base (3)..(3): 2′-O-methyl guanosine
  modified_base (4)..(4): 2′-O-methyl guanosine
  modified_base (5)..(5): 2′-O-methyl cytosine
  modified_base (6)..(6): 2′-O-methyl adenosine
  modified_base (7)..(7): 2′-O-methyl adenosine
  modified_base (8)..(8): 2′-fluoro-deoxyadenosine
  modified_base (9)..(9): 2′-fluoro-deoxyguanosine
  modified_base (10)..(10): 2′-fluoro-deoxyguanosine
  modified_base (11)..(11): 2′-fluoro-deoxyadenosine
  modified_base (12)..(12): 2′-O-methyl guanosine
  modified_base (13)..(13): 2′-O-methyl adenosine
  modified_base (14)..(14): 2′-O-methyl uridine
  modified_base (15)..(15): 2′-O-methyl cytosine
  modified_base (16)..(16): 2′-O-methyl cytosine
  modified_base (17)..(17): 2′-O-methyl uridine
  modified_base (18)..(18): 2′-O-methyl adenosine
  modified_base (19)..(19): 2′-O-methyl adenosine
  modified_base (20)..(20): 2′-O-methyl adenosine
  modified_base (21)..(21): 2′-O-methyl guanosine
  modified_base (22)..(22): 2′-O-methyl cytosine
  modified_base (23)..(23): 2′-O-methyl adenosine
  modified_base (24)..(24): 2′-O-methyl guanosine
  modified_base (25)..(25): 2′-O-methyl cytosine
  modified_base (26)..(26): 2′-O-methyl cytosine
  modified_base (27)..(27): 2′-O-methyl guanosine
  modified_base (28)..(28): 2′-aminodiethoxymethanol-GalNAc adenosine
  modified_base (29)..(29): 2′-aminodiethoxymethanol-GalNAc adenosine
  modified_base (30)..(30): 2′-aminodiethoxymethanol-GalNAc adenosine
  modified_base (31)..(31): 2′-O-methyl guanosine
  modified_base (32)..(32): 2′-O-methyl guanosine
  modified_base (33)..(33): 2′-O-methyl cytosine
  modified_base (34)..(34): 2′-O-methyl uridine
  modified_base (35)..(35): 2′-O-methyl guanosine
  modified_base (36)..(36): 2′-O-methyl cytosine

SEQ ID NO: 789 - RNA, 36 nt, Artificial Sequence; identical to SEQ ID NO: 27
  Synthetic Polynucleotide
  modified_base (1)..(1): 2′-O-methyl uridine
  misc_feature (1)..(2): phosphorothioate linkage between the two indicated positions
  modified_base (2)..(2): 2′-O-methyl guanosine
  modified_base (3)..(3): 2′-fluoro-deoxyguanosine
  modified_base (4)..(4): 2′-O-methyl guanosine
  modified_base (5)..(5): 2′-O-methyl cytosine
  modified_base (6)..(6): 2′-O-methyl adenosine
  modified_base (7)..(7): 2′-O-methyl adenosine
  modified_base (8)..(8): 2′-fluoro-deoxyadenosine
  modified_base (9)..(9): 2′-O-methyl guanosine
  modified_base (10)..(10): 2′-fluoro-deoxyguanosine
  modified_base (11)..(11): 2′-O-methyl adenosine
  modified_base (12)..(12): 2′-fluoro-deoxyguanosine
  modified_base (13)..(13): 2′-fluoro-deoxyadenosine
  modified_base (14)..(14): 2′-O-methyl uridine
  modified_base (15)..(15): 2′-O-methyl cytosine
  modified_base (16)..(16): 2′-O-methyl cytosine
  modified_base (17)..(17): 2′-fluoro-uridine
  modified_base (18)..(18): 2′-O-methyl adenosine
  modified_base (19)..(19): 2′-O-methyl adenosine
  modified_base (20)..(20): 2′-O-methyl adenosine
  modified_base (21)..(21): 2′-O-methyl guanosine
  modified_base (22)..(22): 2′-O-methyl cytosine
  modified_base (23)..(23): 2′-O-methyl adenosine
  modified_base (24)..(24): 2′-O-methyl guanosine
  modified_base (25)..(25): 2′-O-methyl cytosine
  modified_base (26)..(26): 2′-O-methyl cytosine
  modified_base (27)..(27): 2′-O-methyl guanosine
  modified_base (28)..(28): 2′-aminodiethoxymethanol-GalNAc adenosine
  modified_base (29)..(29): 2′-aminodiethoxymethanol-GalNAc adenosine
  modified_base (30)..(30): 2′-aminodiethoxymethanol-GalNAc adenosine
  modified_base (31)..(31): 2′-O-methyl guanosine
  modified_base (32)..(32): 2′-O-methyl guanosine
  modified_base (33)..(33): 2′-O-methyl cytosine
  modified_base (34)..(34): 2′-O-methyl uridine
  modified_base (35)..(35): 2′-O-methyl guanosine
  modified_base (36)..(36): 2′-O-methyl cytosine

SEQ ID NO: 790 - RNA, 36 nt, Artificial Sequence; identical to SEQ ID NO: 28
  Synthetic Polynucleotide
  modified_base (1)..(1): 2′-O-methyl adenosine
  misc_feature (1)..(2): phosphorothioate linkage between the two indicated positions
  modified_base (2)..(2): 2′-O-methyl adenosine
  modified_base (3)..(3): 2′-O-methyl adenosine
  modified_base (4)..(4): 2′-O-methyl guanosine
  modified_base (5)..(5): 2′-O-methyl adenosine
  modified_base (6)..(6): 2′-O-methyl guanosine
  modified_base (7)..(7): 2′-O-methyl adenosine
  modified_base (8)..(8): 2′-fluoro-deoxyadenosine
  modified_base (9)..(9): 2′-fluoro-deoxyadenosine
  modified_base (10)..(10): 2′-fluoro-uridine
  modified_base (11)..(11): 2′-fluoro-deoxyguanosine
  modified_base (12)..(12): 2′-O-methyl adenosine
  modified_base (13)..(13): 2′-O-methyl adenosine
  modified_base (14)..(14): 2′-O-methyl adenosine
  modified_base (15)..(15): 2′-O-methyl adenosine
  modified_base (16)..(16): 2′-O-methyl cytosine
  modified_base (17)..(17): 2′-O-methyl cytosine
  modified_base (18)..(18): 2′-O-methyl uridine
  modified_base (19)..(19): 2′-O-methyl adenosine
  modified_base (20)..(20): 2′-O-methyl adenosine
  modified_base (21)..(21): 2′-O-methyl guanosine
  modified_base (22)..(22): 2′-O-methyl cytosine
  modified_base (23)..(23): 2′-O-methyl adenosine
  modified_base (24)..(24): 2′-O-methyl guanosine
  modified_base (25)..(25): 2′-O-methyl cytosine
  modified_base (26)..(26): 2′-O-methyl cytosine
  modified_base (27)..(27): 2′-O-methyl guanosine
  modified_base (28)..(28): 2′-aminodiethoxymethanol-GalNAc adenosine
  modified_base (29)..(29): 2′-aminodiethoxymethanol-GalNAc adenosine
  modified_base (30)..(30): 2′-aminodiethoxymethanol-GalNAc adenosine
  modified_base (31)..(31): 2′-O-methyl guanosine
  modified_base (32)..(32): 2′-O-methyl guanosine
  modified_base (33)..(33): 2′-O-methyl cytosine
  modified_base (34)..(34): 2′-O-methyl uridine
  modified_base (35)..(35): 2′-O-methyl guanosine
  modified_base (36)..(36): 2′-O-methyl cytosine

SEQ ID NO: 791 - RNA, 36 nt, Artificial Sequence; identical to SEQ ID NO: 28
  Synthetic Polynucleotide
  modified_base (1)..(1): 2′-O-methyl adenosine
  misc_feature (1)..(2): phosphorothioate linkage between the two indicated positions
  modified_base (2)..(2): 2′-O-methyl adenosine
  modified_base (3)..(3): 2′-fluoro-deoxyadenosine
  modified_base (4)..(4): 2′-O-methyl guanosine
  modified_base (5)..(5): 2′-O-methyl adenosine
  modified_base (6)..(6): 2′-O-methyl guanosine
  modified_base (7)..(7): 2′-O-methyl adenosine
  modified_base (8)..(8): 2′-fluoro-deoxyadenosine
  modified_base (9)..(9): 2′-O-methyl adenosine
  modified_base (10)..(10): 2′-fluoro-uridine
  modified_base (11)..(11): 2′-O-methyl guanosine
  modified_base (12)..(12): 2′-fluoro-deoxyadenosine
  modified_base (13)..(13): 2′-fluoro-deoxyadenosine
  modified_base (14)..(14): 2′-O-methyl adenosine
  modified_base (15)..(15): 2′-O-methyl adenosine
  modified_base (16)..(16): 2′-O-methyl cytosine
  modified_base (17)..(17): 2′-fluoro-deoxycytosine
  modified_base (18)..(18): 2′-O-methyl uridine
  modified_base (19)..(19): 2′-O-methyl adenosine
  modified_base (20)..(20): 2′-O-methyl adenosine
  modified_base (21)..(21): 2′-O-methyl guanosine
  modified_base (22)..(22): 2′-O-methyl cytosine
  modified_base (23)..(23): 2′-O-methyl adenosine
  modified_base (24)..(24): 2′-O-methyl guanosine
  modified_base (25)..(25): 2′-O-methyl cytosine
  modified_base (26)..(26): 2′-O-methyl cytosine
  modified_base (27)..(27): 2′-O-methyl guanosine
  modified_base (28)..(28): 2′-aminodiethoxymethanol-GalNAc adenosine
  modified_base (29)..(29): 2′-aminodiethoxymethanol-GalNAc adenosine
  modified_base (30)..(30): 2′-aminodiethoxymethanol-GalNAc adenosine
  modified_base (31)..(31): 2′-O-methyl guanosine
  modified_base (32)..(32): 2′-O-methyl guanosine
  modified_base (33)..(33): 2′-O-methyl cytosine
  modified_base (34)..(34): 2′-O-methyl uridine
  modified_base (35)..(35): 2′-O-methyl guanosine
  modified_base (36)..(36): 2′-O-methyl cytosine

SEQ ID NO: 792 - RNA, 36 nt, Artificial Sequence; identical to SEQ ID NO: 29
  Synthetic Polynucleotide
  modified_base (1)..(1): 2′-O-methyl adenosine
  misc_feature (1)..(2): phosphorothioate linkage between the two indicated positions
  modified_base (2)..(2): 2′-O-methyl adenosine
  modified_base (3)..(3): 2′-O-methyl guanosine
  modified_base (4)..(4): 2′-O-methyl adenosine
  modified_base (5)..(5): 2′-O-methyl adenosine
  modified_base (6)..(6): 2′-O-methyl guanosine
  modified_base (7)..(7): 2′-O-methyl adenosine
  modified_base (8)..(8): 2′-fluoro-uridine
  modified_base (9)..(9): 2′-fluoro-deoxyguanosine
  modified_base (10)..(10): 2′-fluoro-deoxyadenosine
  modified_base (11)..(11): 2′-fluoro-uridine
  modified_base (12)..(12): 2′-O-methyl guanosine
  modified_base (13)..(13): 2′-O-methyl adenosine
  modified_base (14)..(14): 2′-O-methyl uridine
  modified_base (15)..(15): 2′-O-methyl guanosine
  modified_base (16)..(16): 2′-O-methyl adenosine
  modified_base (17)..(17): 2′-O-methyl uridine
  modified_base (18)..(18): 2′-O-methyl guanosine
  modified_base (19)..(19): 2′-O-methyl adenosine
  modified_base (20)..(20): 2′-O-methyl adenosine
  modified_base (21)..(21): 2′-O-methyl guanosine
  modified_base (22)..(22): 2′-O-methyl cytosine
  modified_base (23)..(23): 2′-O-methyl adenosine
  modified_base (24)..(24): 2′-O-methyl guanosine
  modified_base (25)..(25): 2′-O-methyl cytosine
  modified_base (26)..(26): 2′-O-methyl cytosine
  modified_base (27)..(27): 2′-O-methyl guanosine
  modified_base (28)..(28): 2′-aminodiethoxymethanol-GalNAc adenosine
  modified_base (29)..(29): 2′-aminodiethoxymethanol-GalNAc adenosine
  modified_base (30)..(30): 2′-aminodiethoxymethanol-GalNAc adenosine
  modified_base (31)..(31): 2′-O-methyl guanosine
  modified_base (32)..(32): 2′-O-methyl guanosine
  modified_base (33)..(33): 2′-O-methyl cytosine
  modified_base (34)..(34): 2′-O-methyl uridine
  modified_base (35)..(35): 2′-O-methyl guanosine
  modified_base (36)..(36): 2′-O-methyl cytosine

SEQ ID NO: 793 - RNA, 36 nt, Artificial Sequence; identical to SEQ ID NO: 29
  Synthetic Polynucleotide
  modified_base (1)..(1): 2′-O-methyl adenosine
  misc_feature (1)..(2): phosphorothioate linkage between the two indicated positions
  modified_base (2)..(2): 2′-O-methyl adenosine
  modified_base (3)..(3): 2′-fluoro-deoxyguanosine
  modified_base (4)..(4): 2′-O-methyl adenosine
  modified_base (5)..(5): 2′-O-methyl adenosine
  modified_base (6)..(6): 2′-O-methyl guanosine
  modified_base (7)..(7): 2′-O-methyl adenosine
  modified_base (8)..(8): 2′-fluoro-uridine
  modified_base (9)..(9): 2′-O-methyl guanosine
  modified_base (10)..(10): 2′-fluoro-deoxyadenosine
  modified_base (11)..(11): 2′-O-methyl uridine
  modified_base (12)..(12): 2′-fluoro-deoxyguanosine
  modified_base (13)..(13): 2′-fluoro-deoxyadenosine
  modified_base (14)..(14): 2′-O-methyl uridine
  modified_base (15)..(15): 2′-O-methyl guanosine
  modified_base (16)..(16): 2′-O-methyl adenosine
  modified_base (17)..(17): 2′-fluoro-uridine
  modified_base (18)..(18): 2′-O-methyl guanosine
  modified_base (19)..(19): 2′-O-methyl adenosine
  modified_base (20)..(20): 2′-O-methyl adenosine
  modified_base (21)..(21): 2′-O-methyl guanosine
  modified_base (22)..(22): 2′-O-methyl cytosine
  modified_base (23)..(23): 2′-O-methyl adenosine
  modified_base (24)..(24): 2′-O-methyl guanosine
  modified_base (25)..(25): 2′-O-methyl cytosine
  modified_base (26)..(26): 2′-O-methyl cytosine
  modified_base (27)..(27): 2′-O-methyl guanosine
  modified_base (28)..(28): 2′-aminodiethoxymethanol-GalNAc adenosine
  modified_base (29)..(29): 2′-aminodiethoxymethanol-GalNAc adenosine
  modified_base (30)..(30): 2′-aminodiethoxymethanol-GalNAc adenosine
  modified_base (31)..(31): 2′-O-methyl guanosine
  modified_base (32)..(32): 2′-O-methyl guanosine
  modified_base (33)..(33): 2′-O-methyl cytosine
  modified_base (34)..(34): 2′-O-methyl uridine
  modified_base (35)..(35): 2′-O-methyl guanosine
  modified_base (36)..(36): 2′-O-methyl cytosine

SEQ ID NO: 794 - RNA, 36 nt, Artificial Sequence; identical to SEQ ID NO: 30
  Synthetic Polynucleotide
  modified_base (1)..(1): 2′-O-methyl adenosine
  misc_feature (1)..(2): phosphorothioate linkage between the two indicated positions
  modified_base (2)..(2): 2′-O-methyl uridine
  modified_base (3)..(3): 2′-O-methyl guanosine
  modified_base (4)..(4): 2′-O-methyl adenosine
  modified_base (5)..(5): 2′-O-methyl uridine
  modified_base (6)..(6): 2′-O-methyl guanosine
  modified_base (7)..(7): 2′-O-methyl adenosine
  modified_base (8)..(8): 2′-fluoro-uridine
  modified_base (9)..(9): 2′-fluoro-deoxyguanosine
  modified_base (10)..(10): 2′-fluoro-deoxyadenosine
  modified_base (11)..(11): 2′-fluoro-uridine
  modified_base (12)..(12): 2′-O-methyl guanosine
  modified_base (13)..(13): 2′-O-methyl adenosine
  modified_base (14)..(14): 2′-O-methyl adenosine
  modified_base (15)..(15): 2′-O-methyl uridine
  modified_base (16)..(16): 2′-O-methyl adenosine
  modified_base (17)..(17): 2′-O-methyl adenosine
  modified_base (18)..(18): 2′-O-methyl guanosine
  modified_base (19)..(19): 2′-O-methyl uridine
  modified_base (20)..(20): 2′-O-methyl adenosine
  modified_base (21)..(21): 2′-O-methyl guanosine
  modified_base (22)..(22): 2′-O-methyl cytosine
  modified_base (23)..(23): 2′-O-methyl adenosine
  modified_base (24)..(24): 2′-O-methyl guanosine
  modified_base (25)..(25): 2′-O-methyl cytosine
  modified_base (26)..(26): 2′-O-methyl cytosine
  modified_base (27)..(27): 2′-O-methyl guanosine
  modified_base (28)..(28): 2′-aminodiethoxymethanol-GalNAc adenosine
  modified_base (29)..(29): 2′-aminodiethoxymethanol-GalNAc adenosine
  modified_base (30)..(30): 2′-aminodiethoxymethanol-GalNAc adenosine
  modified_base (31)..(31): 2′-O-methyl guanosine
  modified_base (32)..(32): 2′-O-methyl guanosine
  modified_base (33)..(33): 2′-O-methyl cytosine
  modified_base (34)..(34): 2′-O-methyl uridine
  modified_base (35)..(35): 2′-O-methyl guanosine
  modified_base (36)..(36): 2′-O-methyl cytosine

SEQ ID NO: 795 - RNA, 36 nt, Artificial Sequence; identical to SEQ ID NO: 30
  Synthetic Polynucleotide
  modified_base (1)..(1): 2′-O-methyl adenosine
  misc_feature (1)..(2): phosphorothioate linkage between the two indicated positions
  modified_base (2)..(2): 2′-O-methyl uridine
  modified_base (3)..(3): 2′-fluoro-deoxyguanosine
  modified_base (4)..(4): 2′-O-methyl adenosine
  modified_base (5)..(5): 2′-O-methyl uridine
  modified_base (6)..(6): 2′-O-methyl guanosine
  modified_base (7)..(7): 2′-O-methyl adenosine
  modified_base (8)..(8): 2′-fluoro-uridine
  modified_base (9)..(9): 2′-O-methyl guanosine
  modified_base (10)..(10): 2′-fluoro-deoxyadenosine
  modified_base (11)..(11): 2′-O-methyl uridine
  modified_base (12)..(12): 2′-fluoro-deoxyguanosine
  modified_base (13)..(13): 2′-fluoro-deoxyadenosine
  modified_base (14)..(14): 2′-O-methyl adenosine
  modified_base (15)..(15): 2′-O-methyl uridine
  modified_base (16)..(16): 2′-O-methyl adenosine
  modified_base (17)..(17): 2′-fluoro-deoxyadenosine
  modified_base (18)..(18): 2′-O-methyl guanosine
  modified_base (19)..(19): 2′-O-methyl uridine
  modified_base (20)..(20): 2′-O-methyl adenosine
  modified_base (21)..(21): 2′-O-methyl guanosine
  modified_base (22)..(22): 2′-O-methyl cytosine
  modified_base (23)..(23): 2′-O-methyl adenosine
  modified_base (24)..(24): 2′-O-methyl guanosine
  modified_base (25)..(25): 2′-O-methyl cytosine
  modified_base (26)..(26): 2′-O-methyl cytosine
  modified_base (27)..(27): 2′-O-methyl guanosine
  modified_base (28)..(28): 2′-aminodiethoxymethanol-GalNAc adenosine
  modified_base (29)..(29): 2′-aminodiethoxymethanol-GalNAc adenosine
  modified_base (30)..(30): 2′-aminodiethoxymethanol-GalNAc adenosine
  modified_base (31)..(31): 2′-O-methyl guanosine
  modified_base (32)..(32): 2′-O-methyl guanosine
  modified_base (33)..(33): 2′-O-methyl cytosine
  modified_base (34)..(34): 2′-O-methyl uridine
  modified_base (35)..(35): 2′-O-methyl guanosine
  modified_base (36)..(36): 2′-O-methyl cytosine

SEQ ID NO: 796 - RNA, 36 nt, Artificial Sequence; identical to SEQ ID NO: 31
  Synthetic Polynucleotide
  modified_base (1)..(1): 2′-O-methyl guanosine
  misc_feature (1)..(2): phosphorothioate linkage between the two indicated positions
  modified_base (2)..(2): 2′-O-methyl adenosine
  modified_base (3)..(3): 2′-fluoro-uridine
  modified_base (4)..(4): 2′-O-methyl guanosine
  modified_base (5)..(5): 2′-fluoro-deoxyadenosine
  modified_base (6)..(6): 2′-O-methyl uridine
  modified_base (7)..(7): 2′-O-methyl guanosine
  modified_base (8)..(8): 2′-fluoro-deoxyadenosine
  modified_base (9)..(9): 2′-fluoro-deoxyadenosine
  modified_base (10)..(10): 2′-fluoro-uridine
  modified_base (11)..(11): 2′-fluoro-deoxyadenosine
  modified_base (12)..(12): 2′-O-methyl adenosine
  modified_base (13)..(13): 2′-fluoro-deoxyguanosine
  modified_base (14)..(14): 2′-O-methyl uridine
  modified_base (15)..(15): 2′-fluoro-uridine
  modified_base (16)..(16): 2′-O-methyl guanosine
  modified_base (17)..(17): 2′-fluoro-deoxyguanosine
  modified_base (18)..(18): 2′-O-methyl uridine
  modified_base (19)..(19): 2′-O-methyl uridine
  modified_base (20)..(20): 2′-O-methyl adenosine
  modified_base (21)..(21): 2′-O-methyl guanosine
  modified_base (22)..(22): 2′-O-methyl cytosine
  modified_base (23)..(23): 2′-O-methyl adenosine
  modified_base (24)..(24): 2′-O-methyl guanosine
  modified_base (25)..(25): 2′-O-methyl cytosine
  modified_base (26)..(26): 2′-O-methyl cytosine
  modified_base (27)..(27): 2′-O-methyl guanosine
  modified_base (28)..(28): 2′-aminodiethoxymethanol-GalNAc adenosine
  modified_base (29)..(29): 2′-aminodiethoxymethanol-GalNAc adenosine
  modified_base (30)..(30): 2′-aminodiethoxymethanol-GalNAc adenosine
  modified_base (31)..(31): 2′-O-methyl guanosine
  modified_base (32)..(32): 2′-O-methyl guanosine
  modified_base (33)..(33): 2′-O-methyl cytosine
  modified_base (34)..(34): 2′-O-methyl uridine
  modified_base (35)..(35): 2′-O-methyl guanosine
  modified_base (36)..(36): 2′-O-methyl cytosine

SEQ ID NO: 797 - RNA, 36 nt, Artificial Sequence; identical to SEQ ID NO: 31
  Synthetic Polynucleotide
  modified_base (1)..(1): 2′-O-methyl guanosine
  misc_feature (1)..(2): phosphorothioate linkage between the two indicated positions
  modified_base (2)..(2): 2′-O-methyl adenosine
  modified_base (3)..(3): 2′-O-methyl uridine
  modified_base (4)..(4): 2′-O-methyl guanosine
  modified_base (5)..(5): 2′-O-methyl adenosine
  modified_base (6)..(6): 2′-O-methyl uridine
  modified_base (7)..(7): 2′-O-methyl guanosine
  modified_base (8)..(8): 2′-fluoro-deoxyadenosine
  modified_base (9)..(9): 2′-fluoro-deoxyadenosine
  modified_base (10)..(10): 2′-fluoro-uridine
  modified_base (11)..(11): 2′-fluoro-deoxyadenosine
  modified_base (12)..(12): 2′-O-methyl adenosine
  modified_base (13)..(13): 2′-O-methyl guanosine
  modified_base (14)..(14): 2′-O-methyl uridine
  modified_base (15)..(15): 2′-O-methyl uridine
  modified_base (16)..(16): 2′-O-methyl guanosine
  modified_base (17)..(17): 2′-O-methyl guanosine
  modified_base (18)..(18): 2′-O-methyl uridine
  modified_base (19)..(19): 2′-O-methyl uridine
  modified_base (20)..(20): 2′-O-methyl adenosine
  modified_base (21)..(21): 2′-O-methyl guanosine
  modified_base (22)..(22): 2′-O-methyl cytosine
  modified_base (23)..(23): 2′-O-methyl adenosine
  modified_base (24)..(24): 2′-O-methyl guanosine
  modified_base (25)..(25): 2′-O-methyl cytosine
  modified_base (26)..(26): 2′-O-methyl cytosine
  modified_base (27)..(27): 2′-O-methyl guanosine
  modified_base (28)..(28): 2′-aminodiethoxymethanol-GalNAc adenosine
  modified_base (29)..(29): 2′-aminodiethoxymethanol-GalNAc adenosine
  modified_base (30)..(30): 2′-aminodiethoxymethanol-GalNAc adenosine
  modified_base (31)..(31): 2′-O-methyl guanosine
  modified_base (32)..(32): 2′-O-methyl guanosine
  modified_base (33)..(33): 2′-O-methyl cytosine
  modified_base (34)..(34): 2′-O-methyl uridine
  modified_base (35)..(35): 2′-O-methyl guanosine
  modified_base (36)..(36): 2′-O-methyl cytosine

SEQ ID NO: 798 - RNA, 36 nt, Artificial Sequence; identical to SEQ ID NO: 31
  Synthetic Polynucleotide
  modified_base (1)..(1): 2′-O-methyl guanosine
  misc_feature (1)..(2): phosphorothioate linkage between the two indicated positions
  modified_base (2)..(2): 2′-O-methyl adenosine
  modified_base (3)..(3): 2′-fluoro-uridine
  modified_base (4)..(4): 2′-O-methyl guanosine
  modified_base (5)..(5): 2′-O-methyl adenosine
  modified_base (6)..(6): 2′-O-methyl uridine
  modified_base (7)..(7): 2′-O-methyl guanosine
  modified_base (8)..(8): 2′-fluoro-deoxyadenosine
  modified_base (9)..(9): 2′-O-methyl adenosine
  modified_base (10)..(10): 2′-fluoro-uridine
  modified_base (11)..(11): 2′-O-methyl adenosine
  modified_base (12)..(12): 2′-fluoro-deoxyadenosine
  modified_base (13)..(13): 2′-fluoro-deoxyguanosine
  modified_base (14)..(14): 2′-O-methyl uridine
  modified_base (15)..(15): 2′-O-methyl uridine
  modified_base (16)..(16): 2′-O-methyl guanosine
  modified_base (17)..(17): 2′-fluoro-deoxyguanosine
  modified_base (18)..(18): 2′-O-methyl uridine
  modified_base (19)..(19): 2′-O-methyl uridine
  modified_base (20)..(20): 2′-O-methyl adenosine
  modified_base (21)..(21): 2′-O-methyl guanosine
  modified_base (22)..(22): 2′-O-methyl cytosine
  modified_base (23)..(23): 2′-O-methyl adenosine
  modified_base (24)..(24): 2′-O-methyl guanosine
  modified_base (25)..(25): 2′-O-methyl cytosine
  modified_base (26)..(26): 2′-O-methyl cytosine
  modified_base (27)..(27): 2′-O-methyl guanosine
  modified_base (28)..(28): 2′-aminodiethoxymethanol-GalNAc adenosine
  modified_base (29)..(29): 2′-aminodiethoxymethanol-GalNAc adenosine
  modified_base (30)..(30): 2′-aminodiethoxymethanol-GalNAc adenosine
  modified_base (31)..(31): 2′-O-methyl guanosine
  modified_base (32)..(32): 2′-O-methyl guanosine
  modified_base (33)..(33): 2′-O-methyl cytosine
  modified_base (34)..(34): 2′-O-methyl uridine
  modified_base (35)..(35): 2′-O-methyl guanosine
  modified_base (36)..(36): 2′-O-methyl cytosine

SEQ ID NO: 799 - RNA, 36 nt, Artificial Sequence; identical to SEQ ID NO: 32
  Synthetic Polynucleotide
  modified_base (1)..(1): 2′-O-methyl uridine
  misc_feature (1)..(2): phosphorothioate linkage between the two indicated positions
  modified_base (2)..(2): 2′-O-methyl guanosine
  modified_base (3)..(3): 2′-O-methyl adenosine
  modified_base (4)..(4): 2′-O-methyl adenosine
  modified_base (5)..(5): 2′-O-methyl uridine
  modified_base (6)..(6): 2′-O-methyl adenosine
  modified_base (7)..(7): 2′-O-methyl adenosine
  modified_base (8)..(8): 2′-fluoro-deoxyguanosine
  modified_base (9)..(9): 2′-fluoro-uridine
  modified_base (10)..(10): 2′-fluoro-uridine
  modified_base (11)..(11): 2′-fluoro-deoxyguanosine
  modified_base (12)..(12): 2′-O-methyl guanosine
  modified_base (13)..(13): 2′-O-methyl uridine
  modified_base (14)..(14): 2′-O-methyl uridine
  modified_base (15)..(15): 2′-O-methyl cytosine
  modified_base (16)..(16): 2′-O-methyl uridine
  modified_base (17)..(17): 2′-O-methyl adenosine
  modified_base (18)..(18): 2′-O-methyl guanosine
  modified_base (19)..(19): 2′-O-methyl cytosine
  modified_base (20)..(20): 2′-O-methyl adenosine
  modified_base (21)..(21): 2′-O-methyl guanosine
  modified_base (22)..(22): 2′-O-methyl cytosine
  modified_base (23)..(23): 2′-O-methyl adenosine
  modified_base (24)..(24): 2′-O-methyl guanosine
  modified_base (25)..(25): 2′-O-methyl cytosine
  modified_base (26)..(26): 2′-O-methyl cytosine
  modified_base (27)..(27): 2′-O-methyl guanosine
  modified_base (28)..(28): 2′-aminodiethoxymethanol-GalNAc adenosine
  modified_base (29)..(29): 2′-aminodiethoxymethanol-GalNAc adenosine
  modified_base (30)..(30): 2′-aminodiethoxymethanol-GalNAc adenosine
  modified_base (31)..(31): 2′-O-methyl guanosine
  modified_base (32)..(32): 2′-O-methyl guanosine
  modified_base (33)..(33): 2′-O-methyl cytosine
  modified_base (34)..(34): 2′-O-methyl uridine
  modified_base (35)..(35): 2′-O-methyl guanosine
  modified_base (36)..(36): 2′-O-methyl cytosine

SEQ ID NO: 800 - RNA, 36 nt, Artificial Sequence; identical to SEQ ID NO: 32
  Synthetic Polynucleotide
  modified_base (1)..(1): 2′-O-methyl uridine
  misc_feature (1)..(2): phosphorothioate linkage between the two indicated positions
  modified_base (2)..(2): 2′-O-methyl guanosine
  modified_base (3)..(3): 2′-fluoro-deoxyadenosine
  modified_base (4)..(4): 2′-O-methyl adenosine
  modified_base (5)..(5): 2′-O-methyl uridine
  modified_base (6)..(6): 2′-O-methyl adenosine
  modified_base (7)..(7): 2′-O-methyl adenosine
  modified_base (8)..(8): 2′-fluoro-deoxyguanosine
  modified_base (9)..(9): 2′-O-methyl uridine
  modified_base (10)..(10): 2′-fluoro-uridine
  modified_base (11)..(11): 2′-O-methyl guanosine
  modified_base (12)..(12): 2′-fluoro-deoxyguanosine
  modified_base (13)..(13): 2′-fluoro-uridine
  modified_base (14)..(14): 2′-O-methyl uridine
  modified_base (15)..(15): 2′-O-methyl cytosine
  modified_base (16)..(16): 2′-O-methyl uridine
  modified_base (17)..(17): 2′-fluoro-deoxyadenosine
  modified_base (18)..(18): 2′-O-methyl guanosine
  modified_base (19)..(19): 2′-O-methyl cytosine
  modified_base (20)..(20): 2′-O-methyl adenosine
  modified_base (21)..(21): 2′-O-methyl guanosine
  modified_base (22)..(22): 2′-O-methyl cytosine
  modified_base (23)..(23): 2′-O-methyl adenosine
  modified_base (24)..(24): 2′-O-methyl guanosine
  modified_base (25)..(25): 2′-O-methyl cytosine
  modified_base (26)..(26): 2′-O-methyl cytosine
  modified_base (27)..(27): 2′-O-methyl guanosine
  modified_base (28)..(28): 2′-aminodiethoxymethanol-GalNAc adenosine
  modified_base (29)..(29): 2′-aminodiethoxymethanol-GalNAc adenosine
  modified_base (30)..(30): 2′-aminodiethoxymethanol-GalNAc adenosine
  modified_base (31)..(31): 2′-O-methyl guanosine
  modified_base (32)..(32): 2′-O-methyl guanosine
  modified_base (33)..(33): 2′-O-methyl cytosine
  modified_base (34)..(34): 2′-O-methyl uridine
  modified_base (35)..(35): 2′-O-methyl guanosine
  modified_base (36)..(36): 2′-O-methyl cytosine

SEQ ID NO: 801 - RNA, 36 nt, Artificial Sequence; identical to SEQ ID NO: 33
  Synthetic Polynucleotide
  modified_base (1)..(1): 2′-O-methyl adenosine
  misc_feature (1)..(2): phosphorothioate linkage between the two indicated positions
  modified_base (2)..(2): 2′-O-methyl adenosine
  modified_base (3)..(3): 2′-O-methyl uridine
  modified_base (4)..(4): 2′-O-methyl adenosine
  modified_base (5)..(5): 2′-O-methyl adenosine
  modified_base (6)..(6): 2′-O-methyl guanosine
  modified_base (7)..(7): 2′-O-methyl uridine
  modified_base (8)..(8): 2′-fluoro-uridine
  modified_base (9)..(9): 2′-fluoro-deoxyguanosine
  modified_base (10)..(10): 2′-fluoro-deoxyguanosine
  modified_base (11)..(11): 2′-fluoro-uridine
  modified_base (12)..(12): 2′-O-methyl uridine
  modified_base (13)..(13): 2′-O-methyl cytosine
  modified_base (14)..(14): 2′-O-methyl uridine
  modified_base (15)..(15): 2′-O-methyl adenosine
  modified_base (16)..(16): 2′-O-methyl guanosine
  modified_base (17)..(17): 2′-O-methyl cytosine
  modified_base (18)..(18): 2′-O-methyl guanosine
  modified_base (19)..(19): 2′-O-methyl cytosine
  modified_base (20)..(20): 2′-O-methyl adenosine
  modified_base (21)..(21): 2′-O-methyl guanosine
  modified_base (22)..(22): 2′-O-methyl cytosine
  modified_base (23)..(23): 2′-O-methyl adenosine
  modified_base (24)..(24): 2′-O-methyl guanosine
  modified_base (25)..(25): 2′-O-methyl cytosine
  modified_base (26)..(26): 2′-O-methyl cytosine
  modified_base (27)..(27): 2′-O-methyl guanosine
  modified_base (28)..(28): 2′-aminodiethoxymethanol-GalNAc adenosine
  modified_base (29)..(29): 2′-aminodiethoxymethanol-GalNAc adenosine
  modified_base (30)..(30): 2′-aminodiethoxymethanol-GalNAc adenosine
  modified_base (31)..(31): 2′-O-methyl guanosine
  modified_base (32)..(32): 2′-O-methyl guanosine
  modified_base (33)..(33): 2′-O-methyl cytosine
  modified_base (34)..(34): 2′-O-methyl uridine
  modified_base (35)..(35): 2′-O-methyl guanosine
  modified_base (36)..(36): 2′-O-methyl cytosine

SEQ ID NO: 802 - RNA, 36 nt, Artificial Sequence; identical to SEQ ID NO: 33
  Synthetic Polynucleotide
  modified_base (1)..(1): 2′-O-methyl adenosine
  misc_feature (1)..(2): phosphorothioate linkage between the two indicated positions
  modified_base (2)..(2): 2′-O-methyl adenosine
  modified_base (3)..(3): 2′-fluoro-uridine
  modified_base (4)..(4): 2′-O-methyl adenosine
  modified_base (5)..(5): 2′-O-methyl adenosine
  modified_base (6)..(6): 2′-O-methyl guanosine
  modified_base (7)..(7): 2′-O-methyl uridine
  modified_base (8)..(8): 2′-fluoro-uridine
  modified_base (9)..(9): 2′-O-methyl guanosine
  modified_base (10)..(10): 2′-fluoro-deoxyguanosine
  modified_base (11)..(11): 2′-O-methyl uridine
  modified_base (12)..(12): 2′-fluoro-uridine
  modified_base (13)..(13): 2′-fluoro-deoxycytosine
  modified_base (14)..(14): 2′-O-methyl uridine
  modified_base (15)..(15): 2′-O-methyl adenosine
  modified_base (16)..(16): 2′-O-methyl guanosine
  modified_base (17)..(17): 2′-fluoro-deoxycytosine
  modified_base (18)..(18): 2′-O-methyl guanosine
  modified_base (19)..(19): 2′-O-methyl cytosine
  modified_base (20)..(20): 2′-O-methyl adenosine
  modified_base (21)..(21): 2′-O-methyl guanosine
  modified_base (22)..(22): 2′-O-methyl cytosine
  modified_base (23)..(23): 2′-O-methyl adenosine
  modified_base (24)..(24): 2′-O-methyl guanosine
  modified_base (25)..(25): 2′-O-methyl cytosine
  modified_base (26)..(26): 2′-O-methyl cytosine
  modified_base (27)..(27): 2′-O-methyl guanosine
  modified_base (28)..(28): 2′-aminodiethoxymethanol-GalNAc adenosine
  modified_base (29)..(29): 2′-aminodiethoxymethanol-GalNAc adenosine
  modified_base (30)..(30): 2′-aminodiethoxymethanol-GalNAc adenosine
  modified_base (31)..(31): 2′-O-methyl guanosine
  modified_base (32)..(32): 2′-O-methyl guanosine
  modified_base (33)..(33): 2′-O-methyl cytosine
  modified_base (34)..(34): 2′-O-methyl uridine
  modified_base (35)..(35): 2′-O-methyl guanosine
  modified_base (36)..(36): 2′-O-methyl cytosine

SEQ ID NO: 803 - RNA, 36 nt, Artificial Sequence; identical to SEQ ID NO: 34
  Synthetic Polynucleotide
  modified_base (1)..(1): 2′-O-methyl adenosine
  misc_feature (1)..(2): phosphorothioate linkage between the two indicated positions
  modified_base (2)..(2): 2′-O-methyl uridine
  modified_base (3)..(3): 2′-O-methyl adenosine
  modified_base (4)..(4): 2′-O-methyl adenosine
  modified_base (5)..(5): 2′-O-methyl guanosine
  modified_base (6)..(6): 2′-O-methyl uridine
  modified_base (7)..(7): 2′-O-methyl uridine
  modified_base (8)..(8): 2′-fluoro-deoxyguanosine
  modified_base (9)..(9): 2′-fluoro-deoxyguanosine
  modified_base (10)..(10): 2′-fluoro-uridine
  modified_base (11)..(11): 2′-fluoro-uridine
  modified_base (12)..(12): 2′-O-methyl cytosine
  modified_base (13)..(13): 2′-O-methyl uridine
  modified_base (14)..(14): 2′-O-methyl adenosine
  modified_base (15)..(15): 2′-O-methyl guanosine
  modified_base (16)..(16): 2′-O-methyl cytosine
  modified_base (17)..(17): 2′-O-methyl guanosine
  modified_base (18)..(18): 2′-O-methyl cytosine
  modified_base (19)..(19): 2′-O-methyl adenosine
  modified_base (20)..(20): 2′-O-methyl adenosine
  modified_base (21)..(21): 2′-O-methyl guanosine
  modified_base (22)..(22): 2′-O-methyl cytosine
  modified_base (23)..(23): 2′-O-methyl adenosine
  modified_base (24)..(24): 2′-O-methyl guanosine
  modified_base (25)..(25): 2′-O-methyl cytosine
  modified_base (26)..(26): 2′-O-methyl cytosine
  modified_base (27)..(27): 2′-O-methyl guanosine
  modified_base (28)..(28): 2′-aminodiethoxymethanol-GalNAc adenosine
  modified_base (29)..(29): 2′-aminodiethoxymethanol-GalNAc adenosine
  modified_base (30)..(30): 2′-aminodiethoxymethanol-GalNAc adenosine
  modified_base (31)..(31): 2′-O-methyl guanosine
  modified_base (32)..(32): 2′-O-methyl guanosine
  modified_base (33)..(33): 2′-O-methyl cytosine
  modified_base (34)..(34): 2′-O-methyl uridine
  modified_base (35)..(35): 2′-O-methyl guanosine
  modified_base (36)..(36): 2′-O-methyl cytosine

SEQ ID NO: 804 - RNA, 36 nt, Artificial Sequence; identical to SEQ ID NO: 34
  Synthetic Polynucleotide
  modified_base (1)..(1): 2′-O-methyl adenosine
  misc_feature (1)..(2): phosphorothioate linkage between the two indicated positions
  modified_base (2)..(2): 2′-O-methyl uridine
  modified_base (3)..(3): 2′-fluoro-deoxyadenosine
  modified_base (4)..(4): 2′-O-methyl adenosine
  modified_base (5)..(5): 2′-O-methyl guanosine
  modified_base (6)..(6): 2′-O-methyl uridine
  modified_base (7)..(7): 2′-O-methyl uridine
  modified_base (8)..(8): 2′-fluoro-deoxyguanosine
  modified_base (9)..(9): 2′-O-methyl guanosine
  modified_base (10)..(10): 2′-fluoro-uridine
  modified_base (11)..(11): 2′-O-methyl uridine
  modified_base (12)..(12): 2′-fluoro-deoxycytosine
  modified_base (13)..(13): 2′-fluoro-uridine
  modified_base (14)..(14): 2′-O-methyl adenosine
  modified_base (15)..(15): 2′-O-methyl guanosine
  modified_base (16)..(16): 2′-O-methyl cytosine
  modified_base (17)..(17): 2′-fluoro-deoxyguanosine
  modified_base (18)..(18): 2′-O-methyl cytosine
  modified_base (19)..(19): 2′-O-methyl adenosine
  modified_base (20)..(20): 2′-O-methyl adenosine
  modified_base (21)..(21): 2′-O-methyl guanosine
  modified_base (22)..(22): 2′-O-methyl cytosine
  modified_base (23)..(23): 2′-O-methyl adenosine
  modified_base (24)..(24): 2′-O-methyl guanosine
  modified_base (25)..(25): 2′-O-methyl cytosine
  modified_base (26)..(26): 2′-O-methyl cytosine
  modified_base (27)..(27): 2′-O-methyl guanosine
  modified_base (28)..(28): 2′-aminodiethoxymethanol-GalNAc adenosine
  modified_base (29)..(29): 2′-aminodiethoxymethanol-GalNAc adenosine
  modified_base (30)..(30): 2′-aminodiethoxymethanol-GalNAc adenosine
  modified_base (31)..(31): 2′-O-methyl guanosine
  modified_base (32)..(32): 2′-O-methyl guanosine
  modified_base (33)..(33): 2′-O-methyl cytosine
  modified_base (34)..(34): 2′-O-methyl uridine
  modified_base (35)..(35): 2′-O-methyl guanosine
  modified_base (36)..(36): 2′-O-methyl cytosine

SEQ ID NO: 805 - RNA, 36 nt, Artificial Sequence; identical to SEQ ID NO: 35
  Synthetic Polynucleotide
  modified_base (1)..(1): 2′-O-methyl adenosine
  misc_feature (1)..(2): phosphorothioate linkage between the two indicated positions
  modified_base (2)..(2): 2′-O-methyl guanosine
  modified_base (3)..(3): 2′-fluoro-deoxyadenosine
  modified_base (4)..(4): 2′-O-methyl adenosine
  modified_base (5)..(5): 2′-fluoro-deoxyadenosine
  modified_base (6)..(6): 2′-O-methyl adenosine
  modified_base (7)..(7): 2′-O-methyl adenosine
  modified_base (8)..(8): 2′-fluoro-deoxyadenosine
  modified_base (9)..(9): 2′-fluoro-deoxyadenosine
  modified_base (10)..(10): 2′-fluoro-uridine
  modified_base (11)..(11): 2′-fluoro-uridine
  modified_base (12)..(12): 2′-O-methyl guanosine
  modified_base (13)..(13): 2′-fluoro-deoxyadenosine
  modified_base (14)..(14): 2′-O-methyl adenosine
  modified_base (15)..(15): 2′-fluoro-deoxyadenosine
  modified_base (16)..(16): 2′-O-methyl uridine
  modified_base (17)..(17): 2′-fluoro-deoxyguanosine
  modified_base (18)..(18): 2′-O-methyl uridine
  modified_base (19)..(19): 2′-O-methyl adenosine
  modified_base (20)..(20): 2′-O-methyl adenosine
  modified_base (21)..(21): 2′-O-methyl guanosine
  modified_base (22)..(22): 2′-O-methyl cytosine
  modified_base (23)..(23): 2′-O-methyl adenosine
  modified_base (24)..(24): 2′-O-methyl guanosine
  modified_base (25)..(25): 2′-O-methyl cytosine
  modified_base (26)..(26): 2′-O-methyl cytosine
  modified_base (27)..(27): 2′-O-methyl guanosine
  modified_base (28)..(28): 2′-aminodiethoxymethanol-GalNAc adenosine
  modified_base (29)..(29): 2′-aminodiethoxymethanol-GalNAc adenosine
  modified_base (30)..(30): 2′-aminodiethoxymethanol-GalNAc adenosine
  modified_base (31)..(31): 2′-O-methyl guanosine
  modified_base (32)..(32): 2′-O-methyl guanosine
  modified_base (33)..(33): 2′-O-methyl cytosine
  modified_base (34)..(34): 2′-O-methyl uridine
  modified_base (35)..(35): 2′-O-methyl guanosine
  modified_base (36)..(36): 2′-O-methyl cytosine

SEQ ID NO: 806 - RNA, 36 nt, Artificial Sequence; identical to SEQ ID NO: 35
  Synthetic Polynucleotide
  modified_base (1)..(1): 2′-O-methyl adenosine
  misc_feature (1)..(2): phosphorothioate linkage between the two indicated positions
  modified_base (2)..(2): 2′-O-methyl guanosine
  modified_base (3)..(3): 2′-O-methyl adenosine
  modified_base (4)..(4): 2′-O-methyl adenosine
  modified_base (5)..(5): 2′-O-methyl adenosine
  modified_base (6)..(6): 2′-O-methyl adenosine
  modified_base (7)..(7): 2′-O-methyl adenosine
  modified_base (8)..(8): 2′-fluoro-deoxyadenosine
  modified_base (9)..(9): 2′-fluoro-deoxyadenosine
  modified_base (10)..(10): 2′-fluoro-uridine
  modified_base (11)..(11): 2′-fluoro-uridine
  modified_base (12)..(12): 2′-O-methyl guanosine
  modified_base (13)..(13): 2′-O-methyl adenosine
  modified_base (14)..(14): 2′-O-methyl adenosine
  modified_base (15)..(15): 2′-O-methyl adenosine
  modified_base (16)..(16): 2′-O-methyl uridine
  modified_base (17)..(17): 2′-O-methyl guanosine
  modified_base (18)..(18): 2′-O-methyl uridine
  modified_base (19)..(19): 2′-O-methyl adenosine
  modified_base (20)..(20): 2′-O-methyl adenosine
  modified_base (21)..(21): 2′-O-methyl guanosine
  modified_base (22)..(22): 2′-O-methyl cytosine
  modified_base (23)..(23): 2′-O-methyl adenosine
  modified_base (24)..(24): 2′-O-methyl guanosine
  modified_base (25)..(25): 2′-O-methyl cytosine
  modified_base (26)..(26): 2′-O-methyl cytosine
  modified_base (27)..(27): 2′-O-methyl guanosine
  modified_base (28)..(28): 2′-aminodiethoxymethanol-GalNAc adenosine
  modified_base (29)..(29): 2′-aminodiethoxymethanol-GalNAc adenosine
  modified_base (30)..(30): 2′-aminodiethoxymethanol-GalNAc adenosine
  modified_base (31)..(31): 2′-O-methyl guanosine
  modified_base (32)..(32): 2′-O-methyl guanosine
  modified_base (33)..(33): 2′-O-methyl cytosine
  modified_base (34)..(34): 2′-O-methyl uridine
  modified_base (35)..(35): 2′-O-methyl guanosine
  modified_base (36)..(36): 2′-O-methyl cytosine

SEQ ID NO: 807 - RNA, 36 nt, Artificial Sequence; identical to SEQ ID NO: 35
  Synthetic Polynucleotide
  modified_base (1)..(1): 2′-O-methyl adenosine
  misc_feature (1)..(2): phosphorothioate linkage between the two indicated positions
  modified_base (2)..(2): 2′-O-methyl guanosine
  modified_base (3)..(3): 2′-fluoro-deoxyadenosine
  modified_base (4)..(4): 2′-O-methyl adenosine
  modified_base (5)..(5): 2′-O-methyl adenosine
  modified_base (6)..(6): 2′-O-methyl adenosine
  modified_base (7)..(7): 2′-O-methyl adenosine
  modified_base (8)..(8): 2′-fluoro-deoxyadenosine
  modified_base (9)..(9): 2′-O-methyl adenosine
  modified_base (10)..(10): 2′-fluoro-uridine
  modified_base (11)..(11): 2′-O-methyl uridine
  modified_base (12)..(12): 2′-fluoro-deoxyguanosine
  modified_base (13)..(13): 2′-fluoro-deoxyadenosine
  modified_base (14)..(14): 2′-O-methyl adenosine
  modified_base (15)..(15): 2′-O-methyl adenosine
  modified_base (16)..(16): 2′-O-methyl uridine
  modified_base (17)..(17): 2′-fluoro-deoxyguanosine
  modified_base (18)..(18): 2′-O-methyl uridine
  modified_base (19)..(19): 2′-O-methyl adenosine
  modified_base (20)..(20): 2′-O-methyl adenosine
  modified_base (21)..(21): 2′-O-methyl guanosine
  modified_base (22)..(22): 2′-O-methyl cytosine
  modified_base (23)..(23): 2′-O-methyl adenosine
  modified_base (24)..(24): 2′-O-methyl guanosine
  modified_base (25)..(25): 2′-O-methyl cytosine
  modified_base (26)..(26): 2′-O-methyl cytosine
  modified_base (27)..(27): 2′-O-methyl guanosine
  modified_base (28)..(28): 2′-aminodiethoxymethanol-GalNAc adenosine
  modified_base (29)..(29): 2′-aminodiethoxymethanol-GalNAc adenosine
  modified_base (30)..(30): 2′-aminodiethoxymethanol-GalNAc adenosine
  modified_base (31)..(31): 2′-O-methyl guanosine
  modified_base (32)..(32): 2′-O-methyl guanosine
  modified_base (33)..(33): 2′-O-methyl cytosine
  modified_base (34)..(34): 2′-O-methyl uridine
  modified_base (35)..(35): 2′-O-methyl guanosine
  modified_base (36)..(36): 2′-O-methyl cytosine

SEQ ID NO: 808 - RNA, 36 nt, Artificial Sequence; identical to SEQ ID NO: 36
  Synthetic Polynucleotide
  modified_base (1)..(1): 2′-O-methyl uridine
  misc_feature (1)..(2): phosphorothioate linkage between the two indicated positions
  modified_base (2)..(2): 2′-O-methyl uridine
  modified_base (3)..(3): 2′-fluoro-deoxyguanosine
  modified_base (4)..(4): 2′-O-methyl uridine
  modified_base (5)..(5): 2′-fluoro-uridine
  modified_base (6)..(6): 2′-O-methyl guanosine
  modified_base (7)..(7): 2′-O-methyl uridine
  modified_base (8)..(8): 2′-fluoro-uridine
  modified_base (9)..(9): 2′-fluoro-deoxycytosine
  modified_base (10)..(10): 2′-fluoro-uridine
  modified_base (11)..(11): 2′-fluoro-deoxyguanosine
  modified_base (12)..(12): 2′-O-methyl uridine
  modified_base (13)..(13): 2′-fluoro-uridine
  modified_base (14)..(14): 2′-O-methyl adenosine
  modified_base (15)..(15): 2′-fluoro-deoxyadenosine
  modified_base (16)..(16): 2′-O-methyl cytosine
  modified_base (17)..(17): 2′-fluoro-uridine
  modified_base (18)..(18): 2′-O-methyl guanosine
  modified_base (19)..(19): 2′-O-methyl adenosine
  modified_base (20)..(20): 2′-O-methyl adenosine
  modified_base (21)..(21): 2′-O-methyl guanosine
  modified_base (22)..(22): 2′-O-methyl cytosine
  modified_base (23)..(23): 2′-O-methyl adenosine
  modified_base (24)..(24): 2′-O-methyl guanosine
  modified_base (25)..(25): 2′-O-methyl cytosine
  modified_base (26)..(26): 2′-O-methyl cytosine
  modified_base (27)..(27): 2′-O-methyl guanosine
  modified_base (28)..(28): 2′-aminodiethoxymethanol-GalNAc adenosine
  modified_base (29)..(29): 2′-aminodiethoxymethanol-GalNAc adenosine
  modified_base (30)..(30): 2′-aminodiethoxymethanol-GalNAc adenosine
  modified_base (31)..(31): 2′-O-methyl guanosine
  modified_base (32)..(32): 2′-O-methyl guanosine
  modified_base (33)..(33): 2′-O-methyl cytosine
  modified_base (34)..(34): 2′-O-methyl uridine
  modified_base (35)..(35): 2′-O-methyl guanosine
  modified_base (36)..(36): 2′-O-methyl cytosine

SEQ ID NO: 809 - RNA, 36 nt, Artificial Sequence; identical to SEQ ID NO: 36
  Synthetic Polynucleotide
  modified_base (1)..(1): 2′-O-methyl uridine
  misc_feature (1)..(2): phosphorothioate linkage between the two indicated positions
  modified_base (2)..(2): 2′-O-methyl uridine
  modified_base (3)..(3): 2′-O-methyl guanosine
  modified_base (4)..(4): 2′-O-methyl uridine
  modified_base (5)..(5): 2′-O-methyl uridine
  modified_base (6)..(6): 2′-O-methyl guanosine
  modified_base (7)..(7): 2′-O-methyl uridine
  modified_base (8)..(8): 2′-fluoro-uridine
  modified_base (9)..(9): 2′-fluoro-deoxycytosine
  modified_base (10)..(10): 2′-fluoro-uridine
  modified_base (11)..(11): 2′-fluoro-deoxyguanosine
  modified_base (12)..(12): 2′-O-methyl uridine
  modified_base (13)..(13): 2′-O-methyl uridine
  modified_base (14)..(14): 2′-O-methyl adenosine
  modified_base (15)..(15): 2′-O-methyl adenosine
  modified_base (16)..(16): 2′-O-methyl cytosine
  modified_base (17)..(17): 2′-O-methyl uridine
  modified_base (18)..(18): 2′-O-methyl guanosine
  modified_base (19)..(19): 2′-O-methyl adenosine
  modified_base (20)..(20): 2′-O-methyl adenosine
  modified_base (21)..(21): 2′-O-methyl guanosine
  modified_base (22)..(22): 2′-O-methyl cytosine
  modified_base (23)..(23): 2′-O-methyl adenosine
  modified_base (24)..(24): 2′-O-methyl guanosine
  modified_base (25)..(25): 2′-O-methyl cytosine
  modified_base (26)..(26): 2′-O-methyl cytosine
  modified_base (27)..(27): 2′-O-methyl guanosine
  modified_base (28)..(28): 2′-aminodiethoxymethanol-GalNAc adenosine
  modified_base (29)..(29): 2′-aminodiethoxymethanol-GalNAc adenosine
  modified_base (30)..(30): 2′-aminodiethoxymethanol-GalNAc adenosine
  modified_base (31)..(31): 2′-O-methyl guanosine
  modified_base (32)..(32): 2′-O-methyl guanosine
  modified_base (33)..(33): 2′-O-methyl cytosine
  modified_base (34)..(34): 2′-O-methyl uridine
  modified_base (35)..(35): 2′-O-methyl guanosine
  modified_base (36)..(36): 2′-O-methyl cytosine

SEQ ID NO: 810 - RNA, 36 nt, Artificial Sequence; identical to SEQ ID NO: 36
  Synthetic Polynucleotide
  modified_base (1)..(1): 2′-O-methyl uridine
  misc_feature (1)..(2): phosphorothioate linkage between the two indicated positions
  modified_base (2)..(2): 2′-O-methyl uridine
  modified_base (3)..(3): 2′-fluoro-deoxyguanosine
  modified_base (4)..(4): 2′-O-methyl uridine
  modified_base (5)..(5): 2′-O-methyl uridine
  modified_base (6)..(6): 2′-O-methyl guanosine
  modified_base (7)..(7): 2′-O-methyl uridine
  modified_base (8)..(8): 2′-fluoro-uridine
  modified_base (9)..(9): 2′-O-methyl cytosine
  modified_base (10)..(10): 2′-fluoro-uridine
  modified_base (11)..(11): 2′-O-methyl guanosine
  modified_base (12)..(12): 2′-fluoro-uridine
  modified_base (13)..(13): 2′-fluoro-uridine
  modified_base (14)..(14): 2′-O-methyl adenosine
  modified_base (15)..(15): 2′-O-methyl adenosine
  modified_base (16)..(16): 2′-O-methyl cytosine
  modified_base (17)..(17): 2′-fluoro-uridine
  modified_base (18)..(18): 2′-O-methyl guanosine
  modified_base (19)..(19): 2′-O-methyl adenosine
  modified_base (20)..(20): 2′-O-methyl adenosine
  modified_base (21)..(21): 2′-O-methyl guanosine
  modified_base (22)..(22): 2′-O-methyl cytosine
  modified_base (23)..(23): 2′-O-methyl adenosine
  modified_base (24)..(24): 2′-O-methyl guanosine
  modified_base (25)..(25): 2′-O-methyl cytosine
  modified_base (26)..(26): 2′-O-methyl cytosine
  modified_base (27)..(27): 2′-O-methyl guanosine
  modified_base (28)..(28): 2′-aminodiethoxymethanol-GalNAc adenosine
  modified_base (29)..(29): 2′-aminodiethoxymethanol-GalNAc adenosine
  modified_base (30)..(30): 2′-aminodiethoxymethanol-GalNAc adenosine
  modified_base (31)..(31): 2′-O-methyl guanosine
  modified_base (32)..(32): 2′-O-methyl guanosine
  modified_base (33)..(33): 2′-O-methyl cytosine
  modified_base (34)..(34): 2′-O-methyl uridine
  modified_base (35)..(35): 2′-O-methyl guanosine
  modified_base (36)..(36): 2′-O-methyl cytosine

SEQ ID NO: 811 - RNA, 36 nt, Artificial Sequence; identical to SEQ ID NO: 37
  Synthetic Polynucleotide
  modified_base (1)..(1): 2′-O-methyl uridine
  misc_feature (1)..(2): phosphorothioate linkage between the two indicated positions
  modified_base (2)..(2): 2′-O-methyl uridine
  modified_base (3)..(3): 2′-O-methyl cytosine
  modified_base (4)..(4): 2′-O-methyl uridine
  modified_base (5)..(5): 2′-O-methyl guanosine
  modified_base (6)..(6): 2′-O-methyl adenosine
  modified_base (7)..(7): 2′-O-methyl adenosine
  modified_base (8)..(8): 2′-fluoro-uridine
  modified_base (9)..(9): 2′-fluoro-deoxyguanosine
  modified_base (10)..(10): 2′-fluoro-deoxycytosine
  modified_base (11)..(11): 2′-fluoro-uridine
  modified_base (12)..(12): 2′-O-methyl uridine
  modified_base (13)..(13): 2′-O-methyl cytosine
  modified_base (14)..(14): 2′-O-methyl uridine
  modified_base (15)..(15): 2′-O-methyl adenosine
  modified_base (16)..(16): 2′-O-methyl adenosine
  modified_base (17)..(17): 2′-O-methyl guanosine
  modified_base (18)..(18): 2′-O-methyl uridine
  modified_base (19)..(19): 2′-O-methyl adenosine
  modified_base (20)..(20): 2′-O-methyl adenosine
  modified_base (21)..(21): 2′-O-methyl guanosine
  modified_base (22)..(22): 2′-O-methyl cytosine
  modified_base (23)..(23): 2′-O-methyl adenosine
  modified_base (24)..(24): 2′-O-methyl guanosine
  modified_base (25)..(25): 2′-O-methyl cytosine
  modified_base (26)..(26): 2′-O-methyl cytosine
  modified_base (27)..(27): 2′-O-methyl guanosine
  modified_base (28)..(28): 2′-aminodiethoxymethanol-GalNAc adenosine
  modified_base (29)..(29): 2′-aminodiethoxymethanol-GalNAc adenosine
  modified_base (30)..(30): 2′-aminodiethoxymethanol-GalNAc adenosine
  modified_base (31)..(31): 2′-O-methyl guanosine
  modified_base (32)..(32): 2′-O-methyl guanosine
  modified_base (33)..(33): 2′-O-methyl cytosine
  modified_base (34)..(34): 2′-O-methyl uridine
  modified_base (35)..(35): 2′-O-methyl guanosine
  modified_base (36)..(36): 2′-O-methyl cytosine

SEQ ID NO: 812 - RNA, 36 nt, Artificial Sequence; identical to SEQ ID NO: 38
  Synthetic Polynucleotide
  modified_base (1)..(1): 2′-O-methyl cytosine
  misc_feature (1)..(2): phosphorothioate linkage between the two indicated positions
  modified_base (2)..(2): 2′-O-methyl uridine
  modified_base (3)..(3): 2′-O-methyl guanosine
  modified_base (4)..(4): 2′-O-methyl adenosine
  modified_base (5)..(5): 2′-O-methyl adenosine
  modified_base (6)..(6): 2′-O-methyl uridine
  modified_base (7)..(7): 2′-O-methyl guanosine
  modified_base (8)..(8): 2′-fluoro-deoxycytosine
  modified_base (9)..(9): 2′-fluoro-uridine
  modified_base (10)..(10): 2′-fluoro-uridine
  modified_base (11)..(11): 2′-fluoro-deoxycytosine
  modified_base (12)..(12): 2′-O-methyl uridine
  modified_base (13)..(13): 2′-O-methyl adenosine
  modified_base (14)..(14): 2′-O-methyl adenosine
  modified_base (15)..(15): 2′-O-methyl guanosine
  modified_base (16)..(16): 2′-O-methyl uridine
  modified_base (17)..(17): 2′-O-methyl adenosine
  modified_base (18)..(18): 2′-O-methyl adenosine
  modified_base (19)..(19): 2′-O-methyl adenosine
  modified_base (20)..(20): 2′-O-methyl adenosine
  modified_base (21)..(21): 2′-O-methyl guanosine
  modified_base (22)..(22): 2′-O-methyl cytosine
  modified_base (23)..(23): 2′-O-methyl adenosine
  modified_base (24)..(24): 2′-O-methyl guanosine
  modified_base (25)..(25): 2′-O-methyl cytosine
  modified_base (26)..(26): 2′-O-methyl cytosine
  modified_base (27)..(27): 2′-O-methyl guanosine
  modified_base (28)..(28): 2′-aminodiethoxymethanol-GalNAc adenosine
  modified_base (29)..(29): 2′-aminodiethoxymethanol-GalNAc adenosine
  modified_base (30)..(30): 2′-aminodiethoxymethanol-GalNAc adenosine
  modified_base (31)..(31): 2′-O-methyl guanosine
  modified_base (32)..(32): 2′-O-methyl guanosine
  modified_base (33)..(33): 2′-O-methyl cytosine
  modified_base (34)..(34): 2′-O-methyl uridine
  modified_base (35)..(35): 2′-O-methyl guanosine
  modified_base (36)..(36): 2′-O-methyl cytosine

SEQ ID NO: 813 - RNA, 36 nt, Artificial Sequence; identical to SEQ ID NO: 39
  Synthetic Polynucleotide
  modified_base (1)..(1): 2′-O-methyl guanosine
  misc_feature (1)..(2): phosphorothioate linkage between the two indicated positions
  modified_base (2)..(2): 2′-O-methyl adenosine
  modified_base (3)..(3): 2′-O-methyl adenosine
  modified_base (4)..(4): 2′-O-methyl uridine
  modified_base (5)..(5): 2′-O-methyl guanosine
  modified_base (6)..(6): 2′-O-methyl cytosine
  modified_base (7)..(7): 2′-O-methyl uridine
  modified_base (8)..(8): 2′-fluoro-uridine
  modified_base (9)..(9): 2′-fluoro-deoxycytosine
  modified_base (10)..(10): 2′-fluoro-uridine
  modified_base (11)..(11): 2′-fluoro-deoxyadenosine
  modified_base (12)..(12): 2′-O-methyl adenosine
  modified_base (13)..(13): 2′-O-methyl guanosine
  modified_base (14)..(14): 2′-O-methyl uridine
  modified_base (15)..(15): 2′-O-methyl adenosine
  modified_base (16)..(16): 2′-O-methyl adenosine
  modified_base (17)..(17): 2′-O-methyl adenosine
  modified_base (18)..(18): 2′-O-methyl uridine
  modified_base (19)..(19): 2′-O-methyl adenosine
  modified_base (20)..(20): 2′-O-methyl adenosine
  modified_base (21)..(21): 2′-O-methyl guanosine
  modified_base (22)..(22): 2′-O-methyl cytosine
  modified_base (23)..(23): 2′-O-methyl adenosine
  modified_base (24)..(24): 2′-O-methyl guanosine
  modified_base (25)..(25): 2′-O-methyl cytosine
  modified_base (26)..(26): 2′-O-methyl cytosine
  modified_base (27)..(27): 2′-O-methyl guanosine
  modified_base (28)..(28): 2′-aminodiethoxymethanol-GalNAc adenosine
  modified_base (29)..(29): 2′-aminodiethoxymethanol-GalNAc adenosine
  modified_base (30)..(30): 2′-aminodiethoxymethanol-GalNAc adenosine
  modified_base (31)..(31): 2′-O-methyl guanosine
  modified_base (32)..(32): 2′-O-methyl guanosine
  modified_base (33)..(33): 2′-O-methyl cytosine
  modified_base (34)..(34): 2′-O-methyl uridine
  modified_base (35)..(35): 2′-O-methyl guanosine
  modified_base (36)..(36): 2′-O-methyl cytosine

SEQ ID NO: 814 - RNA, 22 nt, Artificial Sequence
  Synthetic Polynucleotide
  modified_base (1)..(1): Methyl-4′-O-methylphosphonate-2′-O-methyl uridine
  misc_feature (1)..(2): Phosphorothioate linkage between the two indicated positions
  modified_base (2)..(2): 2′-fluoro-uridine
  misc_feature (2)..(3): Phosphorothioate linkage between the two indicated positions
  modified_base (3)..(3): 2′-fluoro-uridine
  modified_base (4)..(4): 2′-O-methyl adenosine
  modified_base (5)..(5): 2′-fluoro-deoxyguanosine
  modified_base (6)..(6): 2′-O-methyl guanosine
  modified_base (7)..(7): 2′-fluoro-deoxyadenosine
  modified_base (8)..(8): 2′-O-methyl uridine
  modified_base (9)..(9): 2′-O-methyl cytosine
  modified_base (10)..(10): 2′-fluoro-uridine
  modified_base (11)..(11): 2′-O-methyl cytosine
  modified_base (12)..(12): 2′-O-methyl cytosine
  modified_base (13)..(13): 2′-O-methyl uridine
  modified_base (14)..(14): 2′-fluoro-uridine
  modified_base (15)..(15): 2′-O-methyl uridine
  modified_base (16)..(16): 2′-O-methyl guanosine
  modified_base (17)..(17): 2′-O-methyl cytosine
  modified_base (18)..(18): 2′-O-methyl cytosine
  modified_base (19)..(19): 2′-O-methyl cytosine
  modified_base (20)..(20): 2′-O-methyl adenosine
  misc_feature (20)..(21): Phosphorothioate linkage between the two indicated positions
  modified_base (21)..(21): 2′-O-methyl guanosine
  misc_feature (21)..(22): Phosphorothioate linkage between the two indicated positions
  modified_base (22)..(22): 2′-O-methyl guanosine
uuuaggaucuccuuugcccagg

SEQ ID NO: 815 - RNA, 22 nt, Artificial Sequence
  Synthetic Polynucleotide
  modified_base (1)..(1): Methyl-4′-O-methylphosphonate-2′-O-methyl uridine
  misc_feature (1)..(2): Phosphorothioate linkage between the two indicated positions
  modified_base (2)..(2): 2′-fluoro-uridine
  misc_feature (2)..(3): Phosphorothioate linkage between the two indicated positions
  modified_base (3)..(3): 2′-fluoro-uridine
  modified_base (4)..(4): 2′-fluoro-deoxyadenosine
  modified_base (5)..(5): 2′-fluoro-deoxyguanosine
  modified_base (6)..(6): 2′-O-methyl guanosine
  modified_base (7)..(7): 2′-fluoro-deoxyadenosine
  modified_base (8)..(8): 2′-O-methyl uridine
  modified_base (9)..(9): 2′-O-methyl cytosine
  modified_base (10)..(10): 2′-fluoro-uridine
  modified_base (11)..(11): 2′-O-methyl cytosine
  modified_base (12)..(12): 2′-fluoro-deoxycytosine
  modified_base (13)..(13): 2′-O-methyl uridine
  modified_base (14)..(14): 2′-fluoro-uridine
  modified_base (15)..(15): 2′-O-methyl uridine
  modified_base (16)..(16): 2′-fluoro-deoxyguanosine
  modified_base (17)..(17): 2′-fluoro-deoxycytosine
  modified_base (18)..(18): 2′-O-methyl cytosine
  modified_base (19)..(19): 2′-fluoro-deoxycytosine
  modified_base (20)..(20): 2′-O-methyl adenosine
  misc_feature (20)..(21): Phosphorothioate linkage between the two indicated positions
  modified_base (21)..(21): 2′-O-methyl guanosine
  misc_feature (21)..(22): Phosphorothioate linkage between the two indicated positions
  modified_base (22)..(22): 2′-O-methyl guanosine
uuuaggaucuccuuugcccagg

SEQ ID NO: 816 - RNA, 22 nt, Artificial Sequence
  Synthetic Polynucleotide
  modified_base (1)..(1): Methyl-4′-O-methylphosphonate-2′-O-methyl uridine
  misc_feature (1)..(2): Phosphorothioate linkage between the two indicated positions
  modified_base (2)..(2): 2′-fluoro-uridine
  misc_feature (2)..(3): Phosphorothioate linkage between the two indicated positions
  modified_base (3)..(3): 2′-fluoro-deoxyadenosine
  modified_base (4)..(4): 2′-O-methyl guanosine
  modified_base (5)..(5): 2′-fluoro-deoxyguanosine
  modified_base (6)..(6): 2′-O-methyl uridine
  modified_base (7)..(7): 2′-fluoro-uridine
  modified_base (8)..(8): 2′-O-methyl uridine
  modified_base (9)..(9): 2′-O-methyl uridine
  modified_base (10)..(10): 2′-fluoro-deoxycytosine
  modified_base (11)..(11): 2′-O-methyl adenosine
  modified_base (12)..(12): 2′-O-methyl uridine
  modified_base (13)..(13): 2′-O-methyl uridine
  modified_base (14)..(14): 2′-fluoro-uridine
  modified_base (15)..(15): 2′-O-methyl cytosine
  modified_base (16)..(16): 2′-O-methyl uridine
  modified_base (17)..(17): 2′-O-methyl cytosine
  modified_base (18)..(18): 2′-O-methyl uridine
  modified_base (19)..(19): 2′-O-methyl uridine
  modified_base (20)..(20): 2′-O-methyl uridine
  misc_feature (20)..(21): Phosphorothioate linkage between the two indicated positions
  modified_base (21)..(21): 2′-O-methyl guanosine
  misc_feature (21)..(22): Phosphorothioate linkage between the two indicated positions
  modified_base (22)..(22): 2′-O-methyl guanosine
uuagguuuucauuucucuuugg

SEQ ID NO: 817 - RNA, 22 nt, Artificial Sequence
  Synthetic Polynucleotide
  modified_base (1)..(1): Methyl-4′-O-methylphosphonate-2′-O-methyl uridine
  misc_feature (1)..(2): Phosphorothioate linkage between the two indicated positions
  modified_base (2)..(2): 2′-fluoro-uridine
  misc_feature (2)..(3): Phosphorothioate linkage between the two indicated positions
  modified_base (3)..(3): 2′-fluoro-deoxyadenosine
  modified_base (4)..(4): 2′-fluoro-deoxyguanosine
  modified_base (5)..(5): 2′-fluoro-deoxyguanosine
  modified_base (6)..(6): 2′-O-methyl uridine
  modified_base (7)..(7): 2′-fluoro-uridine
  modified_base (8)..(8): 2′-O-methyl uridine
  modified_base (9)..(9): 2′-O-methyl uridine
  modified_base (10)..(10): 2′-fluoro-deoxycytosine
  modified_base (11)..(11): 2′-O-methyl adenosine
  modified_base (12)..(12): 2′-fluoro-uridine
  modified_base (13)..(13): 2′-O-methyl uridine
  modified_base (14)..(14): 2′-fluoro-uridine
  modified_base (15)..(15): 2′-O-methyl cytosine
  modified_base (16)..(16): 2′-fluoro-uridine
  modified_base (17)..(17): 2′-fluoro-deoxycytosine
  modified_base (18)..(18): 2′-O-methyl uridine
  modified_base (19)..(19): 2′-fluoro-uridine
  modified_base (20)..(20): 2′-O-methyl uridine
  misc_feature (20)..(21): Phosphorothioate linkage between the two indicated positions
  modified_base (21)..(21): 2′-O-methyl guanosine
  misc_feature (21)..(22): Phosphorothioate linkage between the two indicated positions
  modified_base (22)..(22): 2′-O-methyl guanosine
uuagguuuucauuucucuuugg

SEQ ID NO: 818 - RNA, 22 nt, Artificial Sequence
  Synthetic Polynucleotide
  modified_base (1)..(1): Methyl-4′-O-methylphosphonate-2′-O-methyl uridine
  misc_feature (1)..(2): Phosphorothioate linkage between the two indicated positions
  modified_base (2)..(2): 2′-fluoro-uridine
  misc_feature (2)..(3): Phosphorothioate linkage between the two indicated positions
  modified_base (3)..(3): 2′-fluoro-deoxycytosine
  modified_base (4)..(4): 2′-O-methyl adenosine
  modified_base (5)..(5): 2′-fluoro-uridine
  modified_base (6)..(6): 2′-O-methyl cytosine
  modified_base (7)..(7): 2′-fluoro-deoxyadenosine
  modified_base (8)..(8): 2′-O-methyl uridine
  modified_base (9)..(9): 2′-O-methyl cytosine
  modified_base (10)..(10): 2′-fluoro-deoxyadenosine
  modified_base (11)..(11): 2′-O-methyl uridine
  modified_base (12)..(12): 2′-O-methyl cytosine
  modified_base (13)..(13): 2′-O-methyl adenosine
  modified_base (14)..(14): 2′-fluoro-uridine
  modified_base (15)..(15): 2′-O-methyl cytosine
  modified_base (16)..(16): 2′-O-methyl uridine
  modified_base (17)..(17): 2′-O-methyl uridine
  modified_base (18)..(18): 2′-O-methyl cytosine
  modified_base (19)..(19): 2′-O-methyl uridine
  modified_base (20)..(20): 2′-O-methyl uridine
  misc_feature (20)..(21): Phosphorothioate linkage between the two indicated positions
  modified_base (21)..(21): 2′-O-methyl guanosine
  misc_feature (21)..(22): Phosphorothioate linkage between the two indicated positions
  modified_base (22)..(22): 2′-O-methyl guanosine
uucaucaucaucaucuucuugg

SEQ ID NO: 819 - RNA, 22 nt, Artificial Sequence
  Synthetic Polynucleotide
  modified_base (1)..(1): Methyl-4′-O-methylphosphonate-2′-O-methyl uridine
  misc_feature (1)..(2): Phosphorothioate linkage between the two indicated positions
  modified_base (2)..(2): 2′-fluoro-uridine
  misc_feature (2)..(3): Phosphorothioate linkage between the two indicated positions
  modified_base (3)..(3): 2′-fluoro-deoxycytosine
  modified_base (4)..(4): 2′-fluoro-deoxyadenosine
  modified_base (5)..(5): 2′-fluoro-uridine
  modified_base (6)..(6): 2′-O-methyl cytosine
  modified_base (7)..(7): 2′-fluoro-deoxyadenosine
  modified_base (8)..(8): 2′-O-methyl uridine
  modified_base (9)..(9): 2′-O-methyl cytosine
  modified_base (10)..(10): 2′-fluoro-deoxyadenosine
  modified_base (11)..(11): 2′-O-methyl uridine
  modified_base (12)..(12): 2′-fluoro-deoxycytosine
  modified_base (13)..(13): 2′-O-methyl adenosine
  modified_base (14)..(14): 2′-fluoro-uridine
  modified_base (15)..(15): 2′-O-methyl cytosine
  modified_base (16)..(16): 2′-fluoro-uridine
  modified_base (17)..(17): 2′-fluoro-uridine
  modified_base (18)..(18): 2′-O-methyl cytosine
  modified_base (19)..(19): 2′-fluoro-uridine
  modified_base (20)..(20): 2′-O-methyl uridine
  misc_feature (20)..(21): Phosphorothioate linkage between the two indicated positions
  modified_base (21)..(21): 2′-O-methyl guanosine
  misc_feature (21)..(22): Phosphorothioate linkage between the two indicated positions
  modified_base (22)..(22): 2′-O-methyl guanosine
uucaucaucaucaucuucuugg

SEQ ID NO: 820 - RNA, 22 nt, Artificial Sequence
  Synthetic Polynucleotide
  modified_base (1)..(1): Methyl-4′-O-methylphosphonate-2′-O-methyl uridine
  misc_feature (1)..(2): Phosphorothioate linkage between the two indicated positions
  modified_base (2)..(2): 2′-fluoro-deoxyadenosine
  misc_feature (2)..(3): Phosphorothioate linkage between the two indicated positions
  modified_base (3)..(3): 2′-fluoro-deoxycytosine
  modified_base (4)..(4): 2′-O-methyl uridine
  modified_base (5)..(5): 2′-fluoro-uridine
  modified_base (6)..(6): 2′-O-methyl adenosine
  modified_base (7)..(7): 2′-fluoro-uridine
  modified_base (8)..(8): 2′-O-methyl uridine
  modified_base (9)..(9): 2′-O-methyl cytosine
  modified_base (10)..(10): 2′-fluoro-deoxyadenosine
  modified_base (11)..(11): 2′-O-methyl uridine
  modified_base (12)..(12): 2′-O-methyl cytosine
  modified_base (13)..(13): 2′-O-methyl adenosine
  modified_base (14)..(14): 2′-fluoro-uridine
  modified_base (15)..(15): 2′-O-methyl cytosine
  modified_base (16)..(16): 2′-O-methyl adenosine
  modified_base (17)..(17): 2′-O-methyl uridine
  modified_base (18)..(18): 2′-O-methyl cytosine
  modified_base (19)..(19): 2′-O-methyl adenosine
  modified_base (20)..(20): 2′-O-methyl uridine
  misc_feature (20)..(21): Phosphorothioate linkage between the two indicated positions
  modified_base (21)..(21): 2′-O-methyl guanosine
  misc_feature (21)..(22): Phosphorothioate linkage between the two indicated positions
  modified_base (22)..(22): 2′-O-methyl guanosine
uacuuauucaucaucaucaugg

SEQ ID NO: 821 - RNA, 22 nt, Artificial Sequence
  Synthetic Polynucleotide
  modified_base (1)..(1): Methyl-4′-O-methylphosphonate-2′-O-methyl uridine
  misc_feature (1)..(2): Phosphorothioate linkage between the two indicated positions
  modified_base (2)..(2): 2′-fluoro-deoxyadenosine
  misc_feature (2)..(3): Phosphorothioate linkage between the two indicated positions
  modified_base (3)..(3): 2′-fluoro-deoxycytosine
  modified_base (4)..(4): 2′-fluoro-uridine
  modified_base (5)..(5): 2′-fluoro-uridine
  modified_base (6)..(6): 2′-O-methyl adenosine
  modified_base (7)..(7): 2′-fluoro-uridine
  modified_base (8)..(8): 2′-O-methyl uridine
  modified_base (9)..(9): 2′-O-methyl cytosine
  modified_base (10)..(10): 2′-fluoro-deoxyadenosine
  modified_base (11)..(11): 2′-O-methyl uridine
  modified_base (12)..(12): 2′-fluoro-deoxycytosine
  modified_base (13)..(13): 2′-O-methyl adenosine
  modified_base (14)..(14): 2′-fluoro-uridine
  modified_base (15)..(15): 2′-O-methyl cytosine
  modified_base (16)..(16): 2′-fluoro-deoxyadenosine
  modified_base (17)..(17): 2′-fluoro-uridine
  modified_base (18)..(18): 2′-O-methyl cytosine
  modified_base (19)..(19): 2′-fluoro-deoxyadenosine
  modified_base (20)..(20): 2′-O-methyl uridine
  misc_feature (20)..(21): Phosphorothioate linkage between the two indicated positions
  modified_base (21)..(21): 2′-O-methyl guanosine
  misc_feature (21)..(22): Phosphorothioate linkage between the two indicated positions
  modified_base (22)..(22): 2′-O-methyl guanosine
uacuuauucaucaucaucaugg

SEQ ID NO: 822 - RNA, 22 nt, Artificial Sequence
  Synthetic Polynucleotide
  modified_base (1)..(1): Methyl-4′-O-methylphosphonate-2′-O-methyl uridine
  misc_feature (1)..(2): Phosphorothioate linkage between the two indicated positions
  modified_base (2)..(2): 2′-fluoro-deoxyadenosine
  misc_feature (2)..(3): Phosphorothioate linkage between the two indicated positions
  modified_base (3)..(3): 2′-fluoro-deoxyadenosine
  modified_base (4)..(4): 2′-O-methyl cytosine
  modified_base (5)..(5): 2′-fluoro-deoxycytosine
  modified_base (6)..(6): 2′-O-methyl adenosine
  modified_base (7)..(7): 2′-fluoro-deoxyadenosine
  modified_base (8)..(8): 2′-O-methyl cytosine
  modified_base (9)..(9): 2′-O-methyl uridine
  modified_base (10)..(10): 2′-fluoro-uridine
  modified_base (11)..(11): 2′-O-methyl adenosine
  modified_base (12)..(12): 2′-fluoro-uridine
  modified_base (13)..(13): 2′-O-methyl uridine
  modified_base (14)..(14): 2′-fluoro-deoxycytosine
  modified_base (15)..(15): 2′-O-methyl adenosine
  modified_base (16)..(16): 2′-fluoro-uridine
  modified_base (17)..(17): 2′-fluoro-deoxycytosine
  modified_base (18)..(18): 2′-O-methyl adenosine
  modified_base (19)..(19): 2′-fluoro-uridine
  modified_base (20)..(20): 2′-O-methyl cytosine
  misc_feature (20)..(21): Phosphorothioate linkage between the two indicated positions
  modified_base (21)..(21): 2′-O-methyl guanosine
  misc_feature (21)..(22): Phosphorothioate linkage between the two indicated positions
  modified_base (22)..(22): 2′-O-methyl guanosine
gaaccaacuuauucaucaucgg

SEQ ID NO: 823 - RNA, 22 nt, Artificial Sequence
  Synthetic Polynucleotide
  modified_base (1)..(1): Methyl-4′-O-methylphosphonate-2′-O-methyl uridine
  misc_feature (1)..(2): Phosphorothioate linkage between the two indicated positions
  modified_base (2)..(2): 2′-fluoro-deoxyadenosine
  misc_feature (2)..(3): Phosphorothioate linkage between the two indicated positions
  modified_base (3)..(3): 2′-fluoro-deoxyadenosine
  modified_base (4)..(4): 2′-O-methyl cytosine
  modified_base (5)..(5): 2′-fluoro-deoxycytosine
  modified_base (6)..(6): 2′-O-methyl adenosine
  modified_base (7)..(7): 2′-fluoro-deoxyadenosine
  modified_base (8)..(8): 2′-O-methyl cytosine
  modified_base (9)..(9): 2′-O-methyl uridine
  modified_base (10)..(10): 2′-fluoro-uridine
  modified_base (11)..(11): 2′-O-methyl adenosine
  modified_base (12)..(12): 2′-O-methyl uridine
  modified_base (13)..(13): 2′-O-methyl uridine
  modified_base (14)..(14): 2′-fluoro-deoxycytosine
  modified_base (15)..(15): 2′-O-methyl adenosine
  modified_base (16)..(16): 2′-O-methyl uridine
  modified_base (17)..(17): 2′-O-methyl cytosine
  modified_base (18)..(18): 2′-O-methyl adenosine
  modified_base (19)..(19): 2′-O-methyl uridine
  modified_base (20)..(20): 2′-O-methyl cytosine
  misc_feature (20)..(21): Phosphorothioate linkage between the two indicated positions
  modified_base (21)..(21): 2′-O-methyl guanosine
  misc_feature (21)..(22): Phosphorothioate linkage between the two indicated positions
  modified_base (22)..(22): 2′-O-methyl guanosine
gaaccaacuuauucaucaucgg

SEQ ID NO: 824 - RNA, 22 nt, Artificial Sequence
  Synthetic Polynucleotide
  modified_base (1)..(1): Methyl-4′-O-methylphosphonate-2′-O-methyl uridine
  misc_feature (1)..(2): Phosphorothioate linkage between the two indicated positions
  modified_base (2)..(2): 2′-fluoro-deoxyadenosine
  misc_feature (2)..(3): Phosphorothioate linkage between the two indicated positions
  modified_base (3)..(3): 2′-fluoro-deoxyadenosine
  modified_base (4)..(4): 2′-fluoro-deoxycytosine
  modified_base (5)..(5): 2′-fluoro-deoxycytosine
  modified_base (6)..(6): 2′-O-methyl adenosine
  modified_base (7)..(7): 2′-fluoro-deoxyadenosine
  modified_base (8)..(8): 2′-O-methyl cytosine
  modified_base (9)..(9): 2′-O-methyl uridine
  modified_base (10)..(10): 2′-fluoro-uridine
  modified_base (11)..(11): 2′-O-methyl adenosine
  modified_base (12)..(12): 2′-fluoro-uridine
  modified_base (13)..(13): 2′-O-methyl uridine
  modified_base (14)..(14): 2′-fluoro-deoxycytosine
  modified_base (15)..(15): 2′-O-methyl adenosine
  modified_base (16)..(16): 2′-fluoro-uridine
  modified_base (17)..(17): 2′-fluoro-deoxycytosine
  modified_base (18)..(18): 2′-O-methyl adenosine
  modified_base (19)..(19): 2′-fluoro-uridine
  modified_base (20)..(20): 2′-O-methyl cytosine
  misc_feature (20)..(21): Phosphorothioate linkage between the two indicated positions
  modified_base (21)..(21): 2′-O-methyl guanosine
  misc_feature (21)..(22): Phosphorothioate linkage between the two indicated positions
  modified_base (22)..(22): 2′-O-methyl guanosine
gaaccaacuuauucaucaucgg

SEQ ID NO: 825 - RNA, 22 nt, Artificial Sequence
  Synthetic Polynucleotide
  modified_base (1)..(1): Methyl-4′-O-methylphosphonate-2′-O-methyl uridine
  misc_feature (1)..(2): Phosphorothioate linkage between the two indicated positions
  modified_base (2)..(2): 2′-fluoro-deoxyguanosine
  misc_feature (2)..(3): Phosphorothioate linkage between the two indicated positions
  modified_base (3)..(3): 2′-fluoro-deoxycytosine
  modified_base (4)..(4): 2′-O-methyl uridine
  modified_base (5)..(5): 2′-fluoro-deoxyadenosine
  modified_base (6)..(6): 2′-O-methyl guanosine
  modified_base (7)..(7): 2′-fluoro-deoxyadenosine
  modified_base (8)..(8): 2′-O-methyl adenosine
  modified_base (9)..(9): 2′-O-methyl cytosine
  modified_base (10)..(10): 2′-fluoro-deoxycytosine
  modified_base (11)..(11): 2′-O-methyl adenosine
  modified_base (12)..(12): 2′-O-methyl adenosine
  modified_base (13)..(13): 2′-O-methyl cytosine
  modified_base (14)..(14): 2′-fluoro-uridine
  modified_base (15)..(15): 2′-O-methyl uridine
  modified_base (16)..(16): 2′-O-methyl adenosine
  modified_base (17)..(17): 2′-O-methyl uridine
  modified_base (18)..(18): 2′-O-methyl uridine
  modified_base (19)..(19): 2′-O-methyl cytosine
  modified_base (20)..(20): 2′-O-methyl adenosine
  misc_feature (20)..(21): Phosphorothioate linkage between the two indicated positions
  modified_base (21)..(21): 2′-O-methyl guanosine
  misc_feature (21)..(22): Phosphorothioate linkage between the two indicated positions
  modified_base (22)..(22): 2′-O-methyl guanosine
ugcuagaaccaacuuauucagg

SEQ ID NO: 826 - RNA, 22 nt, Artificial Sequence
  Synthetic Polynucleotide
  modified_base (1)..(1): Methyl-4′-O-methylphosphonate-2′-O-methyl uridine
  misc_feature (1)..(2): Phosphorothioate linkage between the two indicated positions
  modified_base (2)..(2): 2′-fluoro-deoxyguanosine
  misc_feature (2)..(3): Phosphorothioate linkage between the two indicated positions
  modified_base (3)..(3): 2′-fluoro-deoxycytosine
  modified_base (4)..(4): 2′-fluoro-uridine
  modified_base (5)..(5): 2′-fluoro-deoxyadenosine
  modified_base (6)..(6): 2′-O-methyl guanosine
  modified_base (7)..(7): 2′-fluoro-deoxyadenosine
  modified_base (8)..(8): 2′-O-methyl adenosine
  modified_base (9)..(9): 2′-O-methyl cytosine
  modified_base (10)..(10): 2′-fluoro-deoxycytosine
  modified_base (11)..(11): 2′-O-methyl adenosine
  modified_base (12)..(12): 2′-fluoro-deoxyadenosine
  modified_base (13)..(13): 2′-O-methyl cytosine
  modified_base (14)..(14): 2′-fluoro-uridine
  modified_base (15)..(15): 2′-O-methyl uridine
  modified_base (16)..(16): 2′-fluoro-deoxyadenosine
  modified_base (17)..(17): 2′-fluoro-uridine
  modified_base (18)..(18): 2′-O-methyl uridine
  modified_base (19)..(19): 2′-fluoro-deoxycytosine
  modified_base (20)..(20): 2′-O-methyl adenosine
  misc_feature (20)..(21): Phosphorothioate linkage between the two indicated positions
  modified_base (21)..(21): 2′-O-methyl guanosine
  misc_feature (21)..(22): Phosphorothioate linkage between the two indicated positions
  modified_base (22)..(22): 2′-O-methyl guanosine
ugcuagaaccaacuuauucagg

SEQ ID NO: 827 - RNA, 22 nt, Artificial Sequence
  Synthetic Polynucleotide
  modified_base (1)..(1): Methyl-4′-O-methylphosphonate-2′-O-methyl uridine
  misc_feature (1)..(2): Phosphorothioate linkage between the two indicated positions
  modified_base (2)..(2): 2′-fluoro-deoxyguanosine
  misc_feature (2)..(3): Phosphorothioate linkage between the two indicated positions
  modified_base (3)..(3): 2′-fluoro-deoxycytosine
  modified_base (4)..(4): 2′-O-methyl guanosine
  modified_base (5)..(5): 2′-fluoro-deoxycytosine
  modified_base (6)..(6): 2′-O-methyl uridine
  modified_base (7)..(7): 2′-fluoro-deoxyadenosine
  modified_base (8)..(8): 2′-O-methyl guanosine
  modified_base (9)..(9): 2′-O-methyl adenosine
  modified_base (10)..(10): 2′-fluoro-deoxyadenosine
  modified_base (11)..(11): 2′-O-methyl cytosine
  modified_base (12)..(12): 2′-O-methyl cytosine
  modified_base (13)..(13): 2′-O-methyl adenosine
  modified_base (14)..(14): 2′-fluoro-deoxyadenosine
  modified_base (15)..(15): 2′-O-methyl cytosine
  modified_base (16)..(16): 2′-O-methyl uridine
  modified_base (17)..(17): 2′-O-methyl uridine
  modified_base (18)..(18): 2′-O-methyl adenosine
  modified_base (19)..(19): 2′-O-methyl uridine
  modified_base (20)..(20): 2′-O-methyl uridine
  misc_feature (20)..(21): Phosphorothioate linkage between the two indicated positions
  modified_base (21)..(21): 2′-O-methyl guanosine
  misc_feature (21)..(22): Phosphorothioate linkage between the two indicated positions
  modified_base (22)..(22): 2′-O-methyl guanosine
ugcgcuagaaccaacuuauugg

SEQ ID NO: 828 - RNA, 22 nt, Artificial Sequence
  Synthetic Polynucleotide
  modified_base (1)..(1): Methyl-4′-O-methylphosphonate-2′-O-methyl uridine
  misc_feature (1)..(2): Phosphorothioate linkage between the two indicated positions
  modified_base (2)..(2): 2′-fluoro-deoxyguanosine
  misc_feature (2)..(3): Phosphorothioate linkage between the two indicated positions
  modified_base (3)..(3): 2′-fluoro-deoxycytosine
  modified_base (4)..(4): 2′-fluoro-deoxyguanosine
  modified_base (5)..(5): 2′-fluoro-deoxycytosine
  modified_base (6)..(6): 2′-O-methyl uridine
  modified_base (7)..(7): 2′-fluoro-deoxyadenosine
  modified_base (8)..(8): 2′-O-methyl guanosine
  modified_base (9)..(9): 2′-O-methyl adenosine
  modified_base (10)..(10): 2′-fluoro-deoxyadenosine
  modified_base (11)..(11): 2′-O-methyl cytosine
  modified_base (12)..(12): 2′-fluoro-deoxycytosine
  modified_base (13)..(13): 2′-O-methyl adenosine
  modified_base (14)..(14): 2′-fluoro-deoxyadenosine
  modified_base (15)..(15): 2′-O-methyl cytosine
  modified_base (16)..(16): 2′-fluoro-uridine
  modified_base (17)..(17): 2′-fluoro-uridine
  modified_base (18)..(18): 2′-O-methyl adenosine
  modified_base (19)..(19): 2′-fluoro-uridine
  modified_base (20)..(20): 2′-O-methyl uridine
  misc_feature (20)..(21): Phosphorothioate linkage between the two indicated positions
  modified_base (21)..(21): 2′-O-methyl guanosine
  misc_feature (21)..(22): Phosphorothioate linkage between the two indicated positions
  modified_base (22)..(22): 2′-O-methyl guanosine
ugcgcuagaaccaacuuauugg

SEQ ID NO: 829 - RNA, 22 nt, Artificial Sequence
  Synthetic Polynucleotide
  modified_base (1)..(1): Methyl-4′-O-methylphosphonate-2′-O-methyl uridine
  misc_feature (1)..(2): Phosphorothioate linkage between the two indicated positions
  modified_base (2)..(2): 2′-fluoro-uridine
  misc_feature (2)..(3): Phosphorothioate linkage between the two indicated positions
  modified_base (3)..(3): 2′-fluoro-deoxyguanosine
  modified_base (4)..(4): 2′-O-methyl cytosine
  modified_base (5)..(5): 2′-fluoro-deoxyguanosine
  modified_base (6)..(6): 2′-O-methyl cytosine
  modified_base (7)..(7): 2′-fluoro-uridine
  modified_base (8)..(8): 2′-O-methyl adenosine
  modified_base (9)..(9): 2′-O-methyl guanosine
  modified_base (10)..(10): 2′-fluoro-deoxyadenosine
  modified_base (11)..(11): 2′-O-methyl adenosine
  modified_base (12)..(12): 2′-O-methyl cytosine
  modified_base (13)..(13): 2′-O-methyl cytosine
  modified_base (14)..(14): 2′-fluoro-deoxyadenosine
  modified_base (15)..(15): 2′-O-methyl adenosine
  modified_base (16)..(16): 2′-O-methyl cytosine
  modified_base (17)..(17): 2′-O-methyl uridine
  modified_base (18)..(18): 2′-O-methyl uridine
  modified_base (19)..(19): 2′-O-methyl adenosine
  modified_base (20)..(20): 2′-O-methyl uridine
  misc_feature (20)..(21): Phosphorothioate linkage between the two indicated positions
  modified_base (21)..(21): 2′-O-methyl guanosine
  misc_feature (21)..(22): Phosphorothioate linkage between the two indicated positions
  modified_base (22)..(22): 2′-O-methyl guanosine
uugcgcuagaaccaacuuaugg

SEQ ID NO: 830 - RNA, 22 nt, Artificial Sequence
  Synthetic Polynucleotide
  modified_base (1)..(1): Methyl-4′-O-methylphosphonate-2′-O-methyl uridine
  misc_feature (1)..(2): Phosphorothioate linkage between the two indicated positions
  modified_base (2)..(2): 2′-fluoro-uridine
  misc_feature (2)..(3): Phosphorothioate linkage between the two indicated positions
  modified_base (3)..(3): 2′-fluoro-deoxyguanosine
  modified_base (4)..(4): 2′-fluoro-deoxycytosine
  modified_base (5)..(5): 2′-fluoro-deoxyguanosine
  modified_base (6)..(6): 2′-O-methyl cytosine
  modified_base (7)..(7): 2′-fluoro-uridine
  modified_base (8)..(8): 2′-O-methyl adenosine
  modified_base (9)..(9): 2′-O-methyl guanosine
  modified_base (10)..(10): 2′-fluoro-deoxyadenosine
  modified_base (11)..(11): 2′-O-methyl adenosine
  modified_base (12)..(12): 2′-fluoro-deoxycytosine
  modified_base (13)..(13): 2′-O-methyl cytosine
  modified_base (14)..(14): 2′-fluoro-deoxyadenosine
  modified_base (15)..(15): 2′-O-methyl adenosine
  modified_base (16)..(16): 2′-fluoro-deoxycytosine
  modified_base (17)..(17): 2′-fluoro-uridine
  modified_base (18)..(18): 2′-O-methyl uridine
  modified_base (19)..(19): 2′-fluoro-deoxyadenosine
  modified_base (20)..(20): 2′-O-methyl uridine
  misc_feature (20)..(21): Phosphorothioate linkage between the two indicated positions
  modified_base (21)..(21): 2′-O-methyl guanosine
  misc_feature (21)..(22): Phosphorothioate linkage between the two indicated positions
  modified_base (22)..(22): 2′-O-methyl guanosine
uugcgcuagaaccaacuuaugg

SEQ ID NO: 831 - RNA, 22 nt, Artificial Sequence
  Synthetic Polynucleotide
  modified_base (1)..(1): Methyl-4′-O-methylphosphonate-2′-O-methyl uridine
  misc_feature (1)..(2): Phosphorothioate linkage between the two indicated positions
  modified_base (2)..(2): 2′-fluoro-uridine
  misc_feature (2)..(3): Phosphorothioate linkage between the two indicated positions
  modified_base (3)..(3): 2′-fluoro-deoxyadenosine
  modified_base (4)..(4): 2′-O-methyl cytosine
  modified_base (5)..(5): 2′-fluoro-deoxyadenosine
  modified_base (6)..(6): 2′-O-methyl uridine
  modified_base (7)..(7): 2′-fluoro-uridine
  modified_base (8)..(8): 2′-O-methyl uridine
  modified_base (9)..(9): 2′-O-methyl cytosine
  modified_base (10)..(10): 2′-fluoro-deoxyadenosine
  modified_base (11)..(11): 2′-O-methyl adenosine
  modified_base (12)..(12): 2′-fluoro-uridine
  modified_base (13)..(13): 2′-O-methyl uridine
  modified_base (14)..(14): 2′-fluoro-uridine
  modified_base (15)..(15): 2′-O-methyl uridine
  modified_base (16)..(16): 2′-fluoro-uridine
  modified_base (17)..(17): 2′-fluoro-uridine
  modified_base (18)..(18): 2′-O-methyl uridine
  modified_base (19)..(19): 2′-fluoro-deoxycytosine
  modified_base (20)..(20): 2′-O-methyl uridine
  misc_feature (20)..(21): Phosphorothioate linkage between the two indicated positions
  modified_base (21)..(21): 2′-O-methyl guanosine
  misc_feature (21)..(22): Phosphorothioate linkage between the two indicated positions
  modified_base (22)..(22): 2′-O-methyl guanosine
uuacauuucaauuuuuuucugg

SEQ ID NO: 832 - RNA, 22 nt, Artificial Sequence
  Synthetic Polynucleotide
  modified_base (1)..(1): Methyl-4′-O-methylphosphonate-2′-O-methyl uridine
  misc_feature (1)..(2): Phosphorothioate linkage between the two indicated positions
  modified_base (2)..(2): 2′-fluoro-uridine
  misc_feature (2)..(3): Phosphorothioate linkage between the two indicated positions
  modified_base (3)..(3): 2′-fluoro-deoxyadenosine
  modified_base (4)..(4): 2′-O-methyl cytosine
  modified_base (5)..(5): 2′-fluoro-deoxyadenosine
  modified_base (6)..(6): 2′-O-methyl uridine
  modified_base (7)..(7): 2′-fluoro-uridine
  modified_base (8)..(8): 2′-O-methyl uridine
  modified_base (9)..(9): 2′-O-methyl cytosine
  modified_base (10)..(10): 2′-fluoro-deoxyadenosine
  modified_base (11)..(11): 2′-O-methyl adenosine
  modified_base (12)..(12): 2′-O-methyl uridine
  modified_base (13)..(13): 2′-O-methyl uridine
  modified_base (14)..(14): 2′-fluoro-uridine
  modified_base (15)..(15): 2′-O-methyl uridine
  modified_base (16)..(16): 2′-O-methyl uridine
  modified_base (17)..(17): 2′-O-methyl uridine
  modified_base (18)..(18): 2′-O-methyl uridine
  modified_base (19)..(19): 2′-O-methyl cytosine
  modified_base (20)..(20): 2′-O-methyl uridine
  misc_feature (20)..(21): Phosphorothioate linkage between the two indicated positions
  modified_base (21)..(21): 2′-O-methyl guanosine
  misc_feature (21)..(22): Phosphorothioate linkage between the two indicated positions
  modified_base (22)..(22): 2′-O-methyl guanosine
uuacauuucaauuuuuuucugg

SEQ ID NO: 833 - RNA, 22 nt, Artificial Sequence
  Synthetic Polynucleotide
  modified_base (1)..(1): Methyl-4′-O-methylphosphonate-2′-O-methyl uridine
  misc_feature (1)..(2): Phosphorothioate linkage between the two indicated positions
  modified_base (2)..(2): 2′-fluoro-uridine
  misc_feature (2)..(3): Phosphorothioate linkage between the two indicated positions
  modified_base (3)..(3): 2′-fluoro-deoxyadenosine
  modified_base (4)..(4): 2′-fluoro-deoxycytosine
  modified_base (5)..(5): 2′-fluoro-deoxyadenosine
  modified_base (6)..(6): 2′-O-methyl uridine
  modified_base (7)..(7): 2′-fluoro-uridine
  modified_base (8)..(8): 2′-O-methyl uridine
  modified_base (9)..(9): 2′-O-methyl cytosine
  modified_base (10)..(10): 2′-fluoro-deoxyadenosine
  modified_base (11)..(11): 2′-O-methyl adenosine
  modified_base (12)..(12): 2′-fluoro-uridine
  modified_base (13)..(13): 2′-O-methyl uridine
  modified_base (14)..(14): 2′-fluoro-uridine
  modified_base (15)..(15): 2′-O-methyl uridine
  modified_base (16)..(16): 2′-fluoro-uridine
  modified_base (17)..(17): 2′-fluoro-uridine
  modified_base (18)..(18): 2′-O-methyl uridine
  modified_base (19)..(19): 2′-fluoro-deoxycytosine
  modified_base (20)..(20): 2′-O-methyl uridine
  misc_feature (20)..(21): Phosphorothioate linkage between the two indicated positions
  modified_base (21)..(21): 2′-O-methyl guanosine
  misc_feature (21)..(22): Phosphorothioate linkage between the two indicated positions
  modified_base (22)..(22): 2′-O-methyl guanosine
uuacauuucaauuuuuuucugg

SEQ ID NO: 834 - RNA, 22 nt, Artificial Sequence
  Synthetic Polynucleotide
  modified_base (1)..(1): Methyl-4′-O-methylphosphonate-2′-O-methyl uridine
  misc_feature (1)..(2): Phosphorothioate linkage between the two indicated positions
  modified_base (2)..(2): 2′-fluoro-uridine
  misc_feature (2)..(3): Phosphorothioate linkage between the two indicated positions
  modified_base (3)..(3): 2′-fluoro-deoxycytosine
  modified_base (4)..(4): 2′-O-methyl adenosine
  modified_base (5)..(5): 2′-fluoro-deoxyguanosine
  modified_base (6)..(6): 2′-O-methyl uridine
  modified_base (7)..(7): 2′-fluoro-uridine
  modified_base (8)..(8): 2′-O-methyl adenosine
  modified_base (9)..(9): 2′-O-methyl adenosine
  modified_base (10)..(10): 2′-fluoro-deoxycytosine
  modified_base (11)..(11): 2′-O-methyl adenosine
  modified_base (12)..(12): 2′-fluoro-deoxyguanosine
  modified_base (13)..(13): 2′-O-methyl adenosine
  modified_base (14)..(14): 2′-fluoro-deoxyadenosine
  modified_base (15)..(15): 2′-O-methyl cytosine
  modified_base (16)..(16): 2′-fluoro-deoxyadenosine
  modified_base (17)..(17): 2′-fluoro-deoxyadenosine
  modified_base (18)..(18): 2′-O-methyl cytosine
  modified_base (19)..(19): 2′-fluoro-deoxyadenosine
  modified_base (20)..(20): 2′-O-methyl adenosine
  misc_feature (20)..(21): Phosphorothioate linkage between the two indicated positions
  modified_base (21)..(21): 2′-O-methyl guanosine
  misc_feature (21)..(22): Phosphorothioate linkage between the two indicated positions
  modified_base (22)..(22): 2′-O-methyl guanosine
uucaguuaacagaacaacaagg

SEQ ID NO: 835 - RNA, 22 nt, Artificial Sequence
  Synthetic Polynucleotide
  modified_base (1)..(1): Methyl-4′-O-methylphosphonate-2′-O-methyl uridine
  misc_feature (1)..(2): Phosphorothioate linkage between the two indicated positions
  modified_base (2)..(2): 2′-fluoro-uridine
  misc_feature (2)..(3): Phosphorothioate linkage between the two indicated positions
  modified_base (3)..(3): 2′-fluoro-deoxycytosine
  modified_base (4)..(4): 2′-O-methyl adenosine
  modified_base (5)..(5): 2′-fluoro-deoxyguanosine
  modified_base (6)..(6): 2′-O-methyl uridine
  modified_base (7)..(7): 2′-fluoro-uridine
  modified_base (8)..(8): 2′-O-methyl adenosine
  modified_base (9)..(9): 2′-O-methyl adenosine
  modified_base (10)..(10): 2′-fluoro-deoxycytosine
  modified_base (11)..(11): 2′-O-methyl adenosine
  modified_base (12)..(12): 2′-O-methyl guanosine
  modified_base (13)..(13): 2′-O-methyl adenosine
  modified_base (14)..(14): 2′-fluoro-deoxyadenosine
  modified_base (15)..(15): 2′-O-methyl cytosine
  modified_base (16)..(16): 2′-O-methyl adenosine
  modified_base (17)..(17): 2′-O-methyl adenosine
  modified_base (18)..(18): 2′-O-methyl cytosine
  modified_base (19)..(19): 2′-O-methyl adenosine
  modified_base (20)..(20): 2′-O-methyl adenosine
  misc_feature (20)..(21): Phosphorothioate linkage between the two indicated positions
  modified_base (21)..(21): 2′-O-methyl guanosine
  misc_feature (21)..(22): Phosphorothioate linkage between the two indicated positions
  modified_base (22)..(22): 2′-O-methyl guanosine
uucaguuaacagaacaacaagg

SEQ ID NO: 836 - RNA, 22 nt, Artificial Sequence
  Synthetic Polynucleotide
  modified_base (1)..(1): Methyl-4′-O-methylphosphonate-2′-O-methyl uridine
  misc_feature (1)..(2): Phosphorothioate linkage between the two indicated positions
  modified_base (2)..(2): 2′-fluoro-uridine
  misc_feature (2)..(3): Phosphorothioate linkage between the two indicated positions
  modified_base (3)..(3): 2′-fluoro-deoxycytosine
  modified_base (4)..(4): 2′-fluoro-deoxyadenosine
  modified_base (5)..(5): 2′-fluoro-deoxyguanosine
  modified_base (6)..(6): 2′-O-methyl uridine
  modified_base (7)..(7): 2′-fluoro-uridine
  modified_base (8)..(8): 2′-O-methyl adenosine
  modified_base (9)..(9): 2′-O-methyl adenosine
  modified_base (10)..(10): 2′-fluoro-deoxycytosine
  modified_base (11)..(11): 2′-O-methyl adenosine
  modified_base (12)..(12): 2′-fluoro-deoxyguanosine
  modified_base (13)..(13): 2′-O-methyl adenosine
  modified_base (14)..(14): 2′-fluoro-deoxyadenosine
  modified_base (15)..(15): 2′-O-methyl cytosine
  modified_base (16)..(16): 2′-fluoro-deoxyadenosine
  modified_base (17)..(17): 2′-fluoro-deoxyadenosine
  modified_base (18)..(18): 2′-O-methyl cytosine
  modified_base (19)..(19): 2′-fluoro-deoxyadenosine
  modified_base (20)..(20): 2′-O-methyl adenosine
  misc_feature (20)..(21): Phosphorothioate linkage between the two indicated positions
  modified_base (21)..(21): 2′-O-methyl guanosine
  misc_feature (21)..(22): Phosphorothioate linkage between the two indicated positions
  modified_base (22)..(22): 2′-O-methyl guanosine
uucaguuaacagaacaacaagg

SEQ ID NO: 837 - RNA, 22 nt, Artificial Sequence
  Synthetic Polynucleotide
  modified_base (1)..(1): Methyl-4′-O-methylphosphonate-2′-O-methyl uridine
  misc_feature (1)..(2): Phosphorothioate linkage between the two indicated positions
  modified_base (2)..(2): 2′-fluoro-uridine
  misc_feature (2)..(3): Phosphorothioate linkage between the two indicated positions
  modified_base (3)..(3): 2′-fluoro-deoxyadenosine
  modified_base (4)..(4): 2′-O-methyl cytosine
  modified_base (5)..(5): 2′-fluoro-uridine
  modified_base (6)..(6): 2′-O-methyl uridine
  modified_base (7)..(7): 2′-fluoro-deoxyadenosine
  modified_base (8)..(8): 2′-O-methyl guanosine
  modified_base (9)..(9): 2′-O-methyl adenosine
  modified_base (10)..(10): 2′-fluoro-deoxyadenosine
  modified_base (11)..(11): 2′-O-methyl guanosine
  modified_base (12)..(12): 2′-O-methyl cytosine
  modified_base (13)..(13): 2′-O-methyl adenosine
  modified_base (14)..(14): 2′-fluoro-uridine
  modified_base (15)..(15): 2′-O-methyl uridine
  modified_base (16)..(16): 2′-O-methyl cytosine
  modified_base (17)..(17): 2′-O-methyl adenosine
  modified_base (18)..(18): 2′-O-methyl guanosine
  modified_base (19)..(19): 2′-O-methyl adenosine
  modified_base (20)..(20): 2′-O-methyl adenosine
  misc_feature (20)..(21): Phosphorothioate linkage between the two indicated positions
  modified_base (21)..(21): 2′-O-methyl guanosine
  misc_feature (21)..(22): Phosphorothioate linkage between the two indicated positions
  modified_base (22)..(22): 2′-O-methyl guanosine
uuacuuagaagcauucagaagg

SEQ ID NO: 838 - RNA, 22 nt, Artificial Sequence
  Synthetic Polynucleotide
  modified_base (1)..(1): Methyl-4′-O-methylphosphonate-2′-O-methyl uridine
  misc_feature (1)..(2): Phosphorothioate linkage between the two indicated positions
  modified_base (2)..(2): 2′-fluoro-uridine
  misc_feature (2)..(3): Phosphorothioate linkage between the two indicated positions
  modified_base (3)..(3): 2′-fluoro-uridine
  modified_base (4)..(4): 2′-O-methyl uridine
  modified_base (5)..(5): 2′-fluoro-deoxyadenosine
  modified_base (6)..(6): 2′-O-methyl cytosine
  modified_base (7)..(7): 2′-fluoro-uridine
  modified_base (8)..(8): 2′-O-methyl uridine
  modified_base (9)..(9): 2′-O-methyl adenosine
  modified_base (10)..(10): 2′-fluoro-deoxyguanosine
  modified_base (11)..(11): 2′-O-methyl adenosine
  modified_base (12)..(12): 2′-O-methyl adenosine
  modified_base (13)..(13): 2′-O-methyl guanosine
  modified_base (14)..(14): 2′-fluoro-deoxycytosine
  modified_base (15)..(15): 2′-O-methyl adenosine
  modified_base (16)..(16): 2′-O-methyl uridine
  modified_base (17)..(17): 2′-O-methyl uridine
  modified_base (18)..(18): 2′-O-methyl cytosine
  modified_base (19)..(19): 2′-O-methyl adenosine
  modified_base (20)..(20): 2′-O-methyl guanosine
  misc_feature (20)..(21): Phosphorothioate linkage between the two indicated positions
  modified_base (21)..(21): 2′-O-methyl guanosine
  misc_feature (21)..(22): Phosphorothioate linkage between the two indicated positions
  modified_base (22)..(22): 2′-O-methyl guanosine
uuuuacuuagaagcauucaggg

SEQ ID NO: 839 - RNA, 22 nt, Artificial Sequence
  Synthetic Polynucleotide
  modified_base (1)..(1): Methyl-4′-O-methylphosphonate-2′-O-methyl uridine
  misc_feature (1)..(2): Phosphorothioate linkage between the two indicated positions
  modified_base (2)..(2): 2′-fluoro-uridine
  misc_feature (2)..(3): Phosphorothioate linkage between the two indicated positions
  modified_base (3)..(3): 2′-fluoro-deoxyadenosine
  modified_base (4)..(4): 2′-O-methyl uridine
  modified_base (5)..(5): 2′-fluoro-uridine
  modified_base (6)..(6): 2′-O-methyl uridine
  modified_base (7)..(7): 2′-fluoro-deoxyadenosine
  modified_base (8)..(8): 2′-O-methyl cytosine
  modified_base (9)..(9): 2′-O-methyl uridine
  modified_base (10)..(10): 2′-fluoro-uridine
  modified_base (11)..(11): 2′-O-methyl adenosine
  modified_base (12)..(12): 2′-O-methyl guanosine
  modified_base (13)..(13): 2′-O-methyl adenosine
  modified_base (14)..(14): 2′-fluoro-deoxyadenosine
  modified_base (15)..(15): 2′-O-methyl guanosine
  modified_base (16)..(16): 2′-O-methyl cytosine
  modified_base (17)..(17): 2′-O-methyl adenosine
  modified_base (18)..(18): 2′-O-methyl uridine
  modified_base (19)..(19): 2′-O-methyl uridine
  modified_base (20)..(20): 2′-O-methyl cytosine
  misc_feature (20)..(21): Phosphorothioate linkage between the two indicated positions
  modified_base (21)..(21): 2′-O-methyl guanosine
  misc_feature (21)..(22): Phosphorothioate linkage between the two indicated positions
  modified_base (22)..(22): 2′-O-methyl guanosine
uuauuuacuuagaagcauucgg

SEQ ID NO: 840 - RNA, 36 nt, Artificial Sequence
  Synthetic Polynucleotide
cuaaacaugggcaaaggagagcagccgaaaggcugc

SEQ ID NO: 841 - RNA, 36 nt, Artificial Sequence
  Synthetic Polynucleotide
uaaacaugggcaaaggagaagcagccgaaaggcugc

SEQ ID NO: 842 - RNA, 36 nt, Artificial Sequence
  Synthetic Polynucleotide
agccgagaggcaaaaugucagcagccgaaaggcugc

SEQ ID NO: 843 - RNA, 36 nt, Artificial Sequence
  Synthetic Polynucleotide
aagaagugcucagagagguagcagccgaaaggcugc

SEQ ID NO: 844 - RNA, 36 nt, Artificial Sequence
  Synthetic Polynucleotide
cucagagagguggaagaccagcagccgaaaggcugc

SEQ ID NO: 845 - RNA, 36 nt, Artificial Sequence
  Synthetic Polynucleotide
agguggaagaccaugucugagcagccgaaaggcugc

SEQ ID NO: 846 - RNA, 36 nt, Artificial Sequence
  Synthetic Polynucleotide
aaauuugaagauauggcaaagcagccgaaaggcugc

SEQ ID NO: 847 - RNA, 36 nt, Artificial Sequence
  Synthetic Polynucleotide
agcaagaaaaagaaggaagagcagccgaaaggcugc

SEQ ID NO: 848 - RNA, 36 nt, Artificial Sequence
  Synthetic Polynucleotide
caagaaaaagaaggaagagagcagccgaaaggcugc

SEQ ID NO: 849 - RNA, 36 nt, Artificial Sequence
  Synthetic Polynucleotide
gaugaugaugaauaaguugagcagccgaaaggcugc

SEQ ID NO: 850 - RNA, 36 nt, Artificial Sequence
  Synthetic Polynucleotide
augaugaugaauaaguuggagcagccgaaaggcugc

SEQ ID NO: 851 - RNA, 36 nt, Artificial Sequence
  Synthetic Polynucleotide
aaguugguucuagcgcaguagcagccgaaaggcugc

SEQ ID NO: 852 - RNA, 36 nt, Artificial Sequence
  Synthetic Polynucleotide
uuauuaguauuguuguccuagcagccgaaaggcugc

SEQ ID NO: 853 - RNA, 22 nt, Artificial Sequence
  Synthetic Polynucleotide
ucuccuuugcccauguuuaggg

SEQ ID NO: 854 - RNA, 22 nt, Artificial Sequence
  Synthetic Polynucleotide
uucuccuuugcccauguuuagg

SEQ ID NO: 855 - RNA, 22 nt, Artificial Sequence
  Synthetic Polynucleotide
ugacauuuugccucucggcugg

SEQ ID NO: 856 - RNA, 22 nt, Artificial Sequence
  Synthetic Polynucleotide
uaccucucugagcacuucuugg

SEQ ID NO: 857 - RNA, 22 nt, Artificial Sequence
  Synthetic Polynucleotide
uggucuuccaccucucugaggg

SEQ ID NO: 858 - RNA, 22 nt, Artificial Sequence
  Synthetic Polynucleotide
ucagacauggucuuccaccugg

SEQ ID NO: 859 - RNA, 22 nt, Artificial Sequence
  Synthetic Polynucleotide
uuugccauaucuucaaauuugg

SEQ ID NO: 860 - RNA, 22 nt, Artificial Sequence
  Synthetic Polynucleotide
ucuuccuucuuuuucuugcugg

SEQ ID NO: 861 - RNA, 22 nt, Artificial Sequence
  Synthetic Polynucleotide
ucucuuccuucuuuuucuuggg

SEQ ID NO: 862 - RNA, 22 nt, Artificial Sequence
  Synthetic Polynucleotide
ucaacuuauucaucaucaucgg

SEQ ID NO: 863 - RNA, 22 nt, Artificial Sequence
  Synthetic Polynucleotide
uccaacuuauucaucaucaugg

SEQ ID NO: 864 - RNA, 22 nt, Artificial Sequence
  Synthetic Polynucleotide
uacugcgcuagaaccaacuugg

SEQ ID NO: 865 - RNA, 22 nt, Artificial Sequence
  Synthetic Polynucleotide
uaggacaacaauacuaauaagg

SEQ ID NO: 866 - RNA, 25 nt, Artificial Sequence
  Synthetic Polynucleotide
ugggcaaaggagauccuaaaaagcc

SEQ ID NO: 867 - RNA, 25 nt, Artificial Sequence
  Synthetic Polynucleotide
aaagagaaaugaaaaccuaaauccc

SEQ ID NO: 868 - DNA, 25 bp, Artificial Sequence
  Synthetic Polynucleotide
aagaagaugaugaugaugaauaagt

SEQ ID NO: 869 - DNA, 25 bp, Artificial Sequence
  Synthetic Polynucleotide
augaugaugaugaauaaguaggutc

SEQ ID NO: 870 - RNA, 25 nt, Artificial Sequence
  Synthetic Polynucleotide
gaugaugaauaaguugguuauagcg

SEQ ID NO: 871 - DNA, 25 bp, Artificial Sequence
  Synthetic Polynucleotide
agaaaaaaauugaaauguaaggctg

SEQ ID NO: 872 - RNA, 25 nt, Artificial Sequence
  Synthetic Polynucleotide
uuguuguucuguuaacugaauacca

SEQ ID NO: 873 - RNA, 25 nt, Artificial Sequence
  Synthetic Polynucleotide
uucugaaugcuucuaaguaaauaca

SEQ ID NO: 874 - DNA, 25 bp, Artificial Sequence
  Synthetic Polynucleotide
cugaaugcuucuaaguaaaaacaat

SEQ ID NO: 875 - DNA, 25 bp, Artificial Sequence
  Synthetic Polynucleotide
gaaugcuucuaaguaaauaaaautt

SEQ ID NO: 876 - RNA, 27 nt, Artificial Sequence
  Synthetic Polynucleotide
gggauuuagguuuucauuucucuuuca

SEQ ID NO: 877 - RNA, 27 nt, Artificial Sequence
  Synthetic Polynucleotide
acuuauucaucaucaucaucuucuucu

SEQ ID NO: 878 - RNA, 27 nt, Artificial Sequence
  Synthetic Polynucleotide
gaaccuacuuauucaucaucaucaucu

SEQ ID NO: 879 - RNA, 27 nt, Artificial Sequence
  Synthetic Polynucleotide
cgcuauaaccaacuuauucaucaucau

SEQ ID NO: 880 - RNA, 27 nt, Artificial Sequence
  Synthetic Polynucleotide
cagccuuacauuucaauuuuuuucuuu

SEQ ID NO: 881 - RNA, 27 nt, Artificial Sequence
  Synthetic Polynucleotide
ugguauucaguuaacagaacaacaauu

SEQ ID NO: 882 - RNA, 27 nt, Artificial Sequence
  Synthetic Polynucleotide
uguauuuacuuagaagcauucagaaug

SEQ ID NO: 883 - RNA, 27 nt, Artificial Sequence
  Synthetic Polynucleotide
auuguuuuuacuuagaagcauucagaa

SEQ ID NO: 884 - RNA, 27 nt, Artificial Sequence
  Synthetic Polynucleotide
aaauuuuauuuacuuagaagcauucag

SEQ ID NO: 885 - RNA, 36 nt, Artificial Sequence
  Synthetic Polynucleotide
aagaccaugucugcuaaagagcagccgaaaggcugc

SEQ ID NO: 886 - RNA, 22 nt, Artificial Sequence
  Synthetic Polynucleotide
ucuuuagcagacauggucuugg

SEQ ID NO: 887 - RNA, 27 nt, Artificial Sequence
  Synthetic Polynucleotide
ggcuuuuuaggaucuccuuugcccaug